SEQUENCE LISTING (22 sequences)

SEQ ID NO: 1 - protein, 9 aa, Artificial Sequence
  Synthetic polypeptide
VVGAVGVGK

SEQ ID NO: 2 - protein, 10 aa, Artificial Sequence
  Synthetic polypeptide
VVVGAVGVGK

SEQ ID NO: 3 - protein, 9 aa, Artificial Sequence
  Synthetic polypeptide
VVGADGVGK

SEQ ID NO: 4 - protein, 10 aa, Artificial Sequence
  Synthetic polypeptide
VVVGADGVGK

SEQ ID NO: 5 - protein, 9 aa, Artificial Sequence
  Synthetic polypeptide
VVGAVGVGW

SEQ ID NO: 6 - protein, 9 aa, Artificial Sequence
  Synthetic polypeptide
VVGAGGVGK

SEQ ID NO: 7 - protein, 10 aa, Artificial Sequence
  Synthetic polypeptide
VVVGAGGVGK

SEQ ID NO: 8 - protein, 10 aa, Artificial Sequence
  Synthetic polypeptide
VVVGAVGVGG

SEQ ID NO: 9 - protein, 10 aa, Artificial Sequence
  Synthetic polypeptide
VVVGAVGVGA

SEQ ID NO: 10 - protein, 10 aa, Artificial Sequence
  Synthetic polypeptide
VVVGAVGVGV

SEQ ID NO: 11 - protein, 10 aa, Artificial Sequence
  Synthetic polypeptide
VVVGAVGVGI

SEQ ID NO: 12 - protein, 10 aa, Artificial Sequence
  Synthetic polypeptide
VVVGAVGVGK

SEQ ID NO: 13 - protein, 10 aa, Artificial Sequence
  Synthetic polypeptide
VVVGAVGVGW

SEQ ID NO: 14 - protein, 9 aa, Artificial Sequence
  Synthetic polypeptide
HSITYLLPV

SEQ ID NO: 15 - protein, 9 aa, Artificial Sequence
  Synthetic polypeptide
LSSPVTKSF

SEQ ID NO: 16 - protein, 9 aa, Artificial Sequence
  Synthetic polypeptide
HMTEVVRHC

SEQ ID NO: 17 - protein, 9 aa, Artificial Sequence
  Synthetic polypeptide
HMTEVVRRC

SEQ ID NO: 18 - protein, 9 aa, Artificial Sequence
  Synthetic polypeptide
HMTEVVRRW

SEQ ID NO: 19 - protein, 9 aa, Artificial Sequence
  Synthetic polypeptide
HMTEVVRHW

SEQ ID NO: 20 - protein, 10 aa, Artificial Sequence
  Synthetic polypeptide
VVVGACGVGK

SEQ ID NO: 21 - protein, 12 aa, Artificial Sequence
  Synthetic polypeptide
QAFWIDLFETIG

SEQ ID NO: 22 - protein, 9 aa, Artificial Sequence
  Synthetic polypeptide
ARAAAAAAA

(19) United States
(12) Patent Application Publication
(10) Pub. No.: US 2023/0111132 A1
(43) Pub. Date: Apr. 13, 2023

(54) STABILIZATION OF MHC COMPLEXES

(71) Applicant: The Regents of the University of California, Oakland, CA (US)

(72) Inventors: Kevan M. Shokat, San Francisco, CA (US); Ziyang Zhang, San Mateo, CA (US)

(21) Appl. No.: 17/787,152

(22) PCT Filed: Dec. 21, 2020
(86) PCT No.: PCT/US2020/066468

§ 371 Date: Jun. 17, 2022

Related U.S. Application Data
(60) Provisional application No. 62/952,800, filed on Dec. 23, 2019.

(51) Int. Cl. A61K 39/00; C07D 403/12; C07D 239/48; C07D 473/18; C07D 473/16; C07D 473/32; C07D 473/24; C07F 9/6561; G01N 33/53

(52) U.S. Cl. CPC A61K 39/0011; C07D 403/12; C07D 239/48; C07D 473/18; C07D 473/16; C07D 473/32; C07D 473/24; C07F 9/65616; G01N 33/5306; A61K 2039/60; A61K 2039/627; G01N 2333/70539; G01N 2500/02

(57) ABSTRACT

Provided, inter alia, are methods and compositions for treating cancer.

### CROSS-REFERENCES TO RELATED APPLICATIONS

This application claims the benefit of U.S. Provisional Application No. 62/952,800, filed Dec. 23, 2019, which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-671001WO_Sequence_Listing_ST25, created Dec. 18, 2020, 4,746 bytes, machine format IBM-PC, MS Windows operating system, is hereby incorporated by reference.

### BACKGROUND

Checkpoint blockade therapies have transformed the landscape of cancer therapy by unleashing tumor specific T cells to attack tumors (1). An essential aspect of the cancer immunity cycle is the availability of tumor specific somatic mutations which are suitable MHC ligands for T cell recognition. The current checkpoint therapies are most effective in tumors with high mutational burden which increases the chance that a good MHC peptide neoantigen will result in a suitable T cell epitope. An ideal cancer specific T cell epitope would be a recurrent mutation such as those found in common oncogenes (KRAS (G12D/V/C), BRAF (V600E), PI3K (ie. PIK3) (E545K/H1047R), etc.). Yet, such peptides are generally poor peptide antigens, greatly limiting the chance for the immune system to be mobilized to target up to 50% of patient tumors containing these driver oncogenes. Thus there is a need in the art for better presentation of antigens of the most common specific hotspot mutations. Provided herein are solutions to these problems and other problems in the art.

### BRIEF SUMMARY

Provided herein, inter alia, are methods and compositions for treating cancer. In one aspect, provided herein is a method of identifying a candidate compound that stabilizes binding of an MHC protein to a peptide antigen. The method includes contacting an MHC protein with a peptide antigen and a candidate compound thereby forming an MHC-peptide-compound complex, and then detecting the increased stability of the MHC-peptide-compound complex relative to the stability of an MHC-peptide complex which does not include the candidate compound. Thus the candidate compound is identified as a compound that stabilizes the binding of the MHC protein to the peptide antigen.

In another aspect, provided herein is a method of treating cancer in a subject in need thereof which includes a. detecting an MHC allele of an MHC protein of the subject, b. detecting a driver oncogene mutation in the subject, and c. administering an effective amount of a MHC-peptide antigen stabilizing compound.

In yet another aspect, provided herein is a method of identifying a modified peptide-MHC protein allele binding pair, which includes contacting a plurality of different modified peptides with a plurality of different MHC protein alleles, and then detecting or computationally predicting binding of a first modified peptide to a first MHC protein allele. Thus a modified peptide-MHC protein allele binding pair is identified.

In another aspect, provided herein is a method of vaccinating a subject for cancer, which includes administering a peptide cancer antigen, and a compound that stabilizes binding of an MHC protein to the peptide cancer antigen.

In another aspect, provided herein is a method of vaccinating a subject for cancer, which includes administering a peptide-compound conjugate that includes a peptide cancer antigen that is linked to a compound via a chemical bond.

In another aspect, provided herein is a composition including an MHC protein, a peptide antigen, and a compound; the MHC protein, the peptide antigen, and the compound are bound to form an MHC-peptide-compound complex and the compound stabilizes the binding of the MHC protein to the peptide antigen relative to the absence of the compound.

In another aspect, provided herein is a compound of formula:

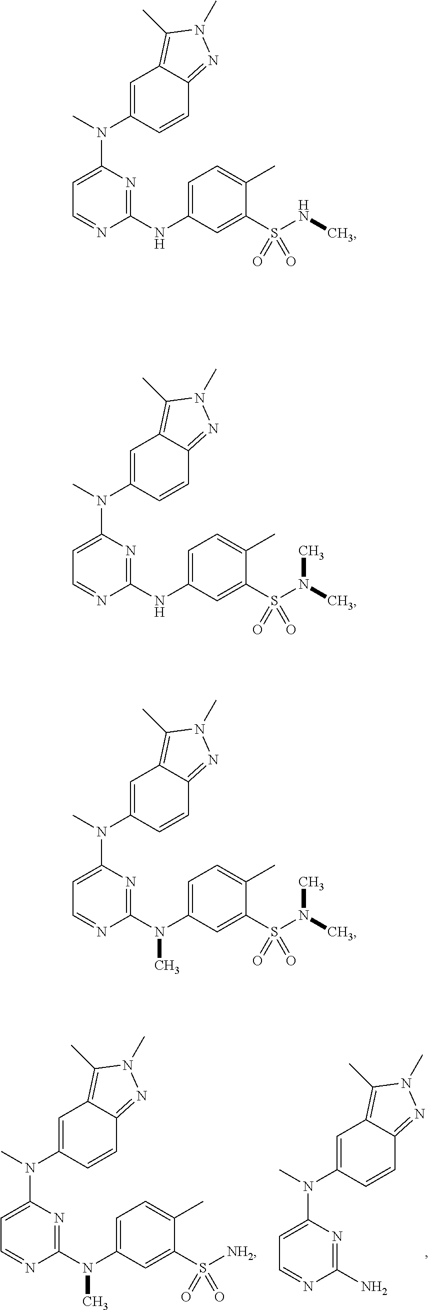

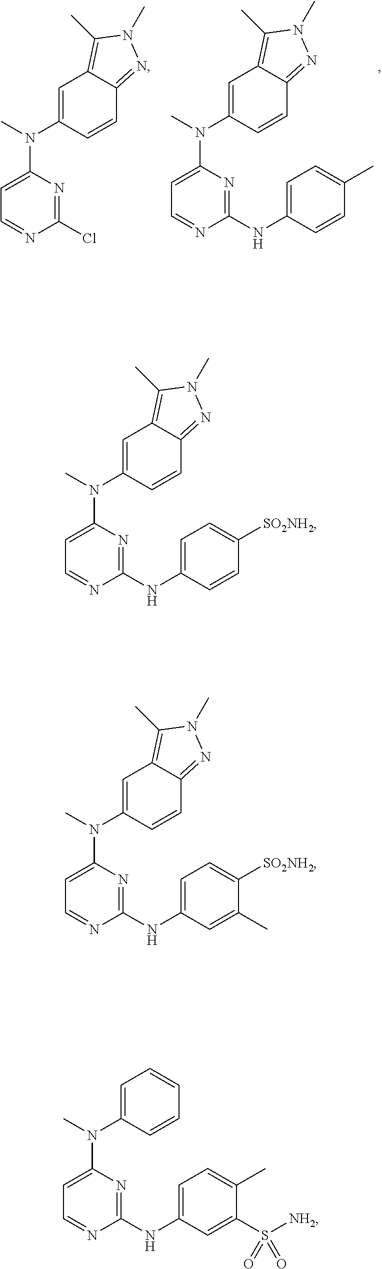 [SMILES: Cc1c2cc(N(C)c3ccnc(Cl)n3)ccc2nn1C ; Cc1ccc(Nc2nccc(N(C)c3ccc4nn(C)c(C)c4c3)n2)cc1 ; Cc1c2cc(N(C)c3ccnc(Nc4ccc(S(N)(=O)=O)cc4)n3)ccc2nn1C ; Cc1cc(Nc2nccc(N(C)c3ccc4nn(C)c(C)c4c3)n2)ccc1S(N)(=O)=O ; Cc1ccc(Nc2nccc(N(C)c3ccccc3)n2)cc1S(N)(=O)=O]

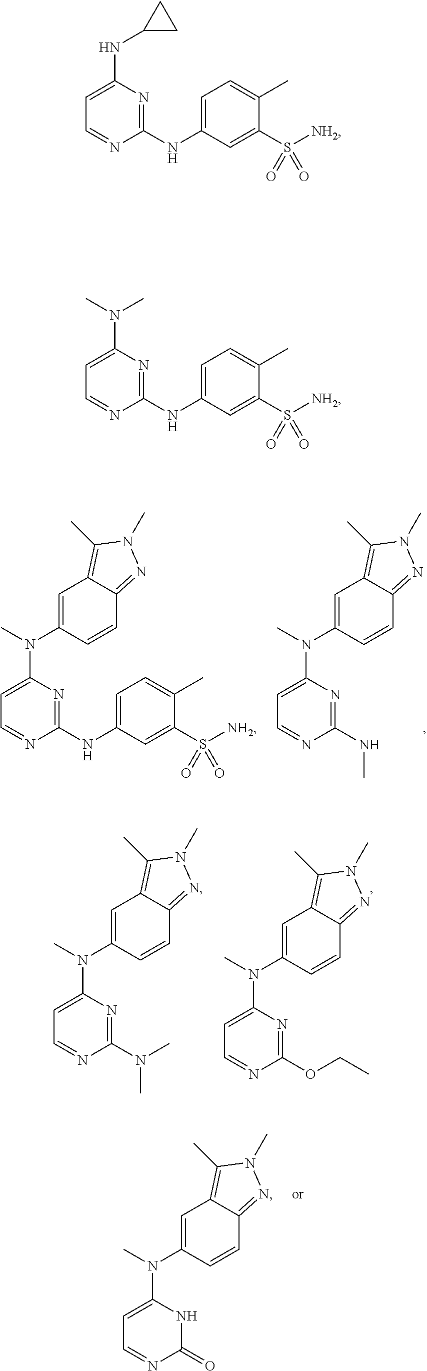

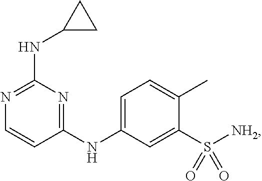 [SMILES: Cc1ccc(Nc2ccnc(NC3CC3)n2)cc1S(N)(=O)=O]

or a salt thereof.

In another aspect, provided herein is a compound of formula:

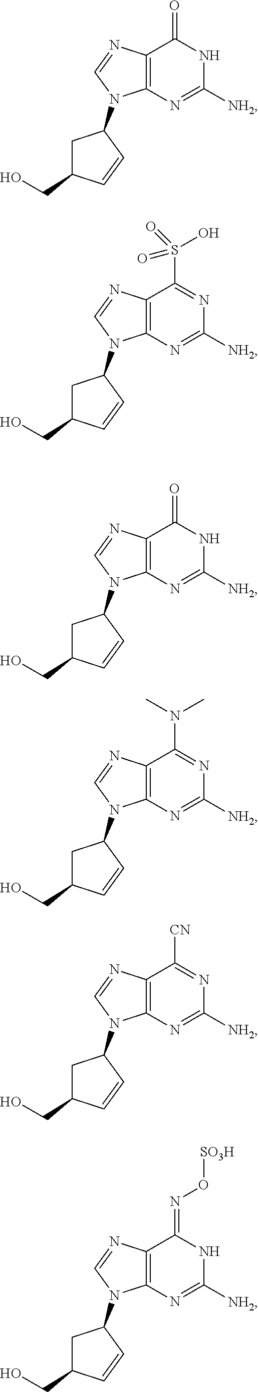 [SMILES: Nc1nc2c(ncn2[C@H]2C=C[C@@H](CO)C2)c(=O)[nH]1 ; Nc1nc(S(=O)(=O)O)c2ncn([C@H]3C=C[C@@H](CO)C3)c2n1 ; Nc1nc2c(ncn2[C@H]2C=C[C@@H](CO)C2)c(=O)[nH]1 ; CN(C)c1nc(N)nc2c1ncn2[C@H]1C=C[C@@H](CO)C1 ; N#Cc1nc(N)nc2c1ncn2[C@H]1C=C[C@@H](CO)C1 ; Nc1nc2c(ncn2[C@H]2C=C[C@@H](CO)C2)/c(=N/OS(=O)(=O)O)[nH]1]

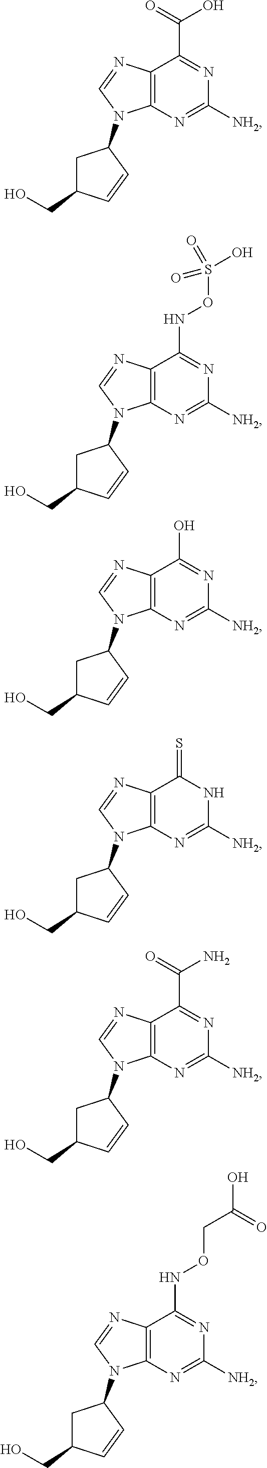 [SMILES: Nc1nc(C(=O)O)c2ncn([C@H]3C=C[C@@H](CO)C3)c2n1 ; Nc1nc(NOS(=O)(=O)O)c2ncn([C@H]3C=C[C@@H](CO)C3)c2n1 ; Nc1nc(O)c2ncn([C@H]3C=C[C@@H](CO)C3)c2n1 ; Nc1nc2c(ncn2[C@H]2C=C[C@@H](CO)C2)c(=S)[nH]1 ; NC(=O)c1nc(N)nc2c1ncn2[C@H]1C=C[C@@H](CO)C1 ; Nc1nc(NOCC(=O)O)c2ncn([C@H]3C=C[C@@H](CO)C3)c2n1]

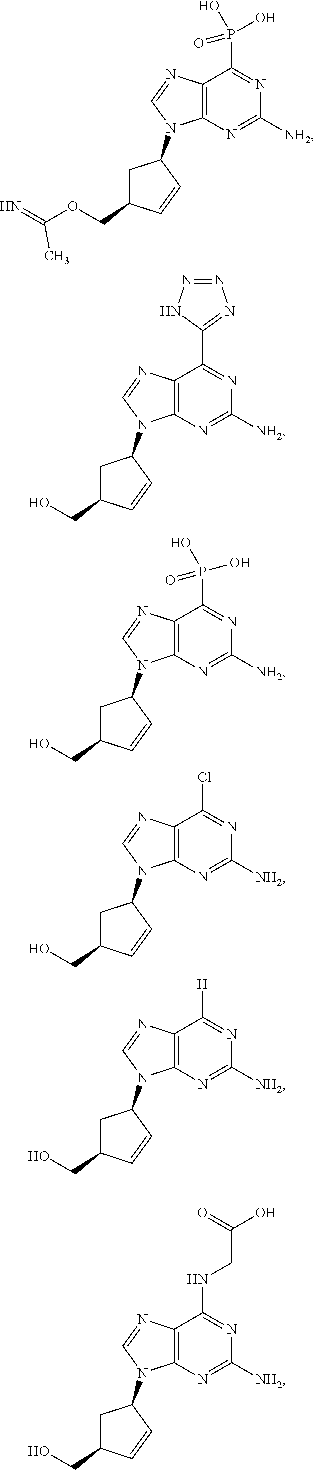 [SMILES: CC(=N)OC[C@@H]1C=C[C@H](n2cnc3c(P(=O)(O)O)nc(N)nc32)C1 ; Nc1nc(-c2nnn[nH]2)c2ncn([C@H]3C=C[C@@H](CO)C3)c2n1 ; Nc1nc(P(=O)(O)O)c2ncn([C@H]3C=C[C@@H](CO)C3)c2n1 ; Nc1nc(Cl)c2ncn([C@H]3C=C[C@@H](CO)C3)c2n1 ; Nc1ncc2ncn([C@H]3C=C[C@@H](CO)C3)c2n1 ; Nc1nc(NCC(=O)O)c2ncn([C@H]3C=C[C@@H](CO)C3)c2n1]

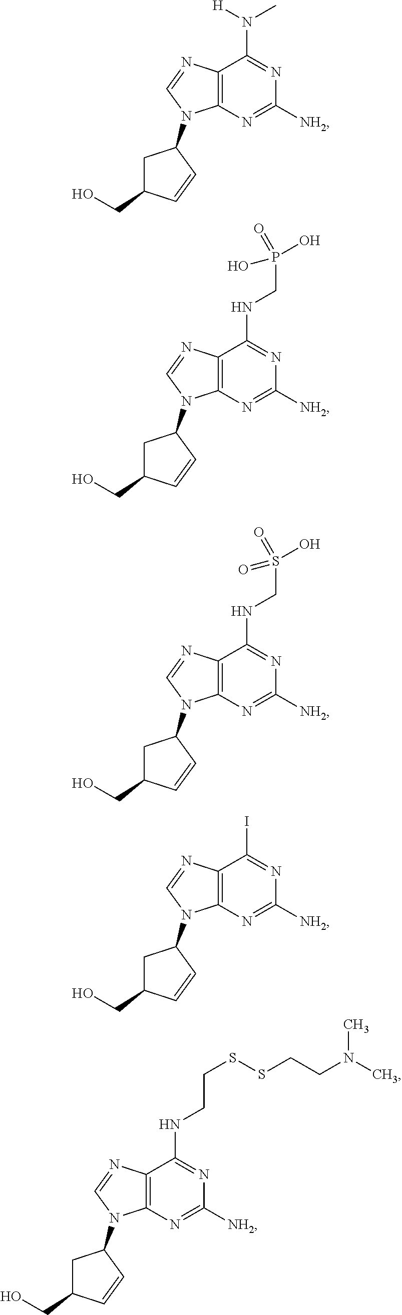 [SMILES: CNc1nc(N)nc2c1ncn2[C@H]1C=C[C@@H](CO)C1 ; Nc1nc(NCP(=O)(O)O)c2ncn([C@H]3C=C[C@@H](CO)C3)c2n1 ; Nc1nc(NCS(=O)(=O)O)c2ncn([C@H]3C=C[C@@H](CO)C3)c2n1 ; Nc1nc(I)c2ncn([C@H]3C=C[C@@H](CO)C3)c2n1 ; CN(C)CCSSCCNc1nc(N)nc2c1ncn2[C@H]1C=C[C@@H](CO)C1]

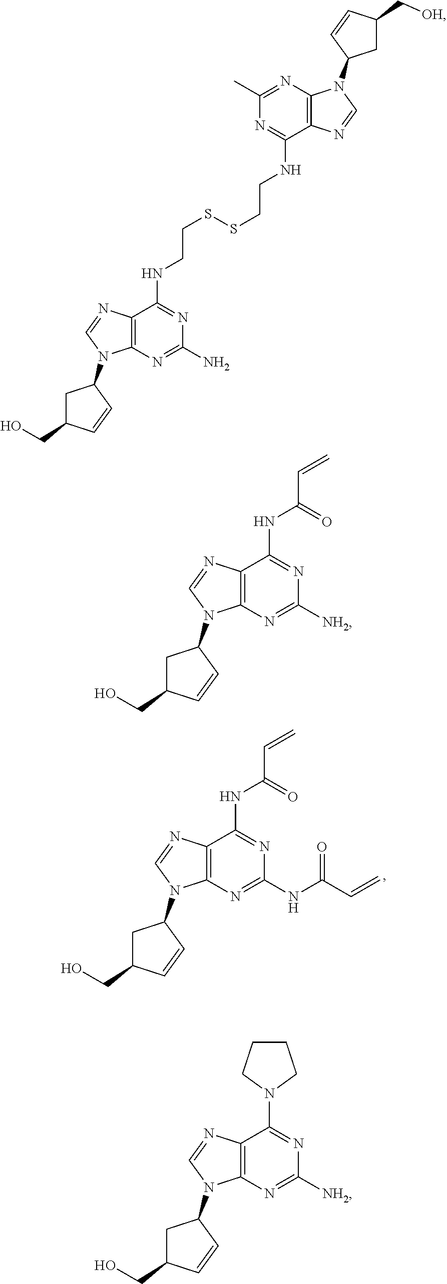 [SMILES: Cc1nc(NCCSSCCNc2nc(N)nc3c2ncn3[C@H]2C=C[C@@H](CO)C2)c2ncn([C@H]3C=C[C@@H](CO)C3)c2n1 ; C=CC(=O)Nc1nc(N)nc2c1ncn2[C@H]1C=C[C@@H](CO)C1 ; C=CC(=O)Nc1nc(NC(=O)C=C)c2ncn([C@H]3C=C[C@@H](CO)C3)c2n1 ; Nc1nc(N2CCCC2)c2ncn([C@H]3C=C[C@@H](CO)C3)c2n1]

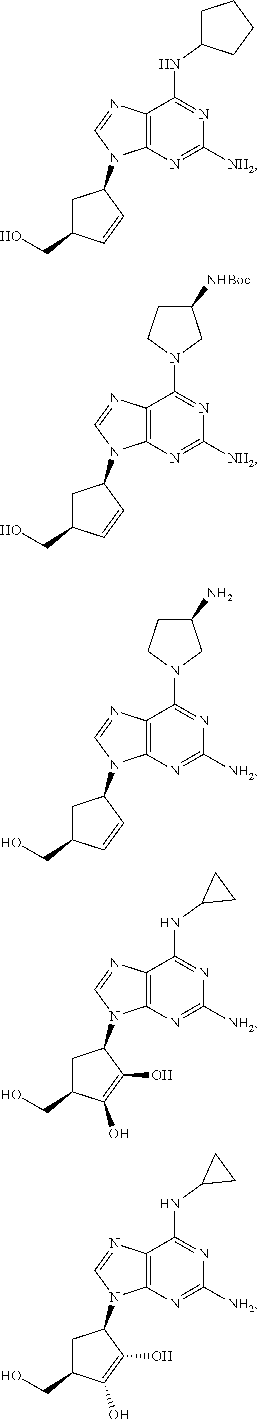

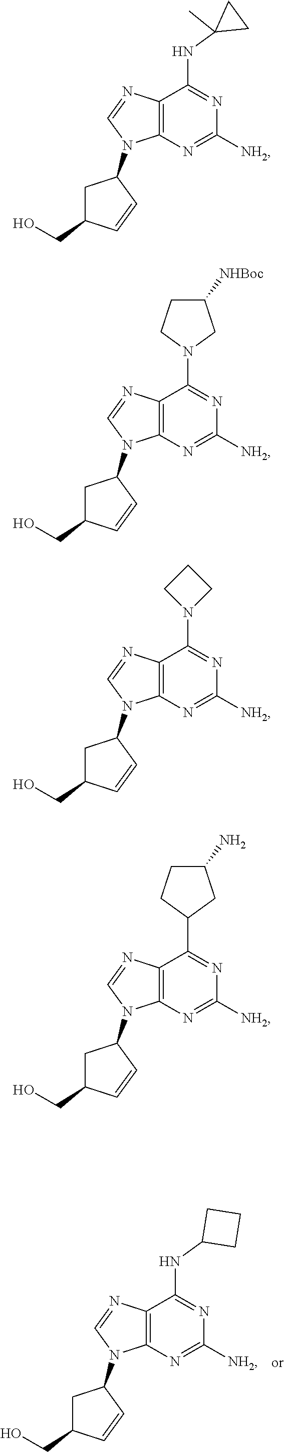 [SMILES: CC1(Nc2nc(N)nc3c2ncn3[C@H]2C=C[C@@H](CO)C2)CC1 ; CC(C)(C)OC(=O)N[C@H]1CCN(c2nc(N)nc3c2ncn3[C@H]2C=C[C@@H](CO)C2)C1 ; Nc1nc(N2CCC2)c2ncn([C@H]3C=C[C@@H](CO)C3)c2n1 ; Nc1nc(C2CC[C@H](N)C2)c2ncn([C@H]3C=C[C@@H](CO)C3)c2n1 ; Nc1nc(NC2CCC2)c2ncn([C@H]3C=C[C@@H](CO)C3)c2n1]

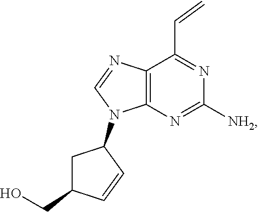 [SMILES: C=Cc1nc(N)nc2c1ncn2[C@H]1C=C[C@@H](CO)C1]

or a salt thereof.

### BRIEF DESCRIPTION OF THE DRAWINGS

FIG. 1. Depicts drug screening of candidate compound for stabilizing binding of MHC protein to a peptide antigen.

FIG. 2. Depicts screening of a kinase inhibitor library for candidate compounds that induce K-Ras peptide presentation.

FIGS. 3A-3B. Shows pazopanib induced stabilization of the presentation of mutant K-Ras peptides by HLA-B*57:01 (FIG. 3A) and by HLA-B*58:01 (FIG. 3B), and sequence of peptides G12V 8-16 (VVGAVGVGK (SEQ ID NO: 1)), G12V 7-16 (VVVGAVGVGK (SEQ ID NO: 2)), G12D 8-16 (VVGADGVGK (SEQ ID NO: 3)), G12D 7-16 (VVVGADGVGK (SEQ ID NO: 4)), and G12V 8-16 W16 (VVGAVGVGW (SEQ ID NO: 5)) (FIG. 3C).

FIG. 4. Shows pazopanib induced stabilization of the presentation of wild-type and mutant K-Ras peptides by HLA-B*57:01 and the sequence of peptides WT 8-16 (VVGAGGVGK (SEQ ID NO: 6)), WT 7-16 (VVVGAGGVGK (SEQ ID NO: 7)), G12V 8-16 (VVGAVGVGK (SEQ ID NO: 1)), G12V 7-16 (VVVGAVGVGK (SEQ ID NO: 2)), G12D 8-16 (VVGADGVGK (SEQ ID NO: 3)), and G12D 7-16 (VVVGADGVGK (SEQ ID NO: 4)).

FIGS. 5A-5B. Shows pazopanib and pazopanib analogs induced stabilization of the presentation of mutant K-Ras peptide (G12V 7-16) by HLA-B*57:01 (FIG. 5A), and after 72 hours (FIG. 5B).

FIG. 6. Shows W-scan of four K-Ras peptides (G12V 8-16 (VVGAVGVGK (SEQ ID NO: 1)), G12V 7-16 (VVVGAVGVGK (SEQ ID NO: 2)), G12D 8-16 (VVGADGVGK (SEQ ID NO: 3)), G12D 7-16 (VVVGADGVGK (SEQ ID NO: 4)) against 69 common MHC class I alleles. The 69 MHC class I alleles, from left to right, are: A0201, A0206, A0301, A1101, A2301, A2402, A2501, A2601, A2902, A3001, A3002, A3101, A3201, A3303, A6801, A6802, A7401, B0702, B0801, B1301, B1302, B1402, B1501, B1502, B1525, B1801, B2702, B2705, B3501, B3503, B3701, B3801, B3901, B4001, B4002, B4402, B4403, B4601, B4801, B4901, B5001, B5101, B5201, B5301, B5501, B5601, B5701, B5801, B5802, C0102, C0202, C0209, C0302, C0303, C0304, C0401, C0501, C0602, C0701, C0702, C0704, C0801, C0802, C1202, C1203, C1402, C1502, C1601, C1701.

FIGS. 7A-7B. Shows W-scan of four K-Ras peptides (G12V 8-16 (VVGAVGVGK (SEQ ID NO: 1)), G12V 7-16 (VVVGAVGVGK (SEQ ID NO: 2)), G12D 8-16 (VVGADGVGK (SEQ ID NO: 3)), G12D 7-16 (VVVGADGVGK (SEQ ID NO: 4)) against four MHC class I alleles. FIG. 7A shows computational prediction of binding of a first modified peptide to a first MHC protein allele and FIG. 7B shows the experimental outcome of the binding.

FIG. 8. Shows pazopanib and pazopanib analogs induced stabilization of the presentation of mutant K-Ras peptides (VVVGAVGVGG (SEQ ID NO: 8), VVVGAVGVGA (SEQ ID NO: 9), VVVGAVGVGV (SEQ ID NO: 10), VVVGAVGVGI (SEQ ID NO: 11), VVVGAVGVGK (SEQ ID NO: 12), VVVGAVGVGW (SEQ ID NO: 13), and HSITYLLPV (SEQ ID NO: 14)) by HLA-B*57:01.

FIG. 9. Shows abacavir and abacavir analogs induced stabilization of the presentation of mutant K-Ras peptides (VVVGAVGVGG (SEQ ID NO: 8), VVVGAVGVGA (SEQ ID NO: 9), VVVGAVGVGV (SEQ ID NO: 10), VVVGAVGVGI (SEQ ID NO: 11), VVVGAVGVGK (SEQ ID NO: 12), VVVGAVGVGW (SEQ ID NO: 13), and HSITYLLPV (SEQ ID NO: 14)) by HLA-B*57:01.

FIG. 10. Shows a peptide-abacavir conjugate (G12V 8-16 (VVGAVGVGK (SEQ ID NO: 1)) refolded with HLA-B*57:01 heavy chain.

FIGS. 11A-11B. Vaccination strategies. FIG. 11A shows using a mixture of small molecule and mutant peptide or mutant protein, and FIG. 11B shows using a covalent drug-peptide conjugate.

FIGS. 12A-12C. Small molecule modulation of antigen presentation. FIG. 12A shows non-cognate oncogene peptide forming a drug-stabilized MHC-peptide complex. FIG. 12B shows a published crystal structure of a Class I MHC-peptide complex stabilized by abacavir. The complex contains PepV: (HSITYLLPV, SEQ ID NO: 14). FIG. 12C shows a published crystal structure of a Class II MHC-peptide complex stabilized by a sodium cation and a beryllium cation. The complex contains QAFWIDLFETIG peptide (SEQ ID NO: 21).

FIG. 13. Primary assay: refolding ELISA. It shows capture ELISA (10-fold diluted reaction mixture).

FIGS. 14A-14B. W-Scan: a computational method to reveal opportunities for drug-induced peptide presentation. FIG. 14A shows an array of Trp-substituted peptides (W-scan peptides) and FIG. 14B shows predicting binding affinity of W-scan peptides to common MHC Class I alleles using NetMHCpan 4.0 algorithm. Peptides whose Trp substitution gives higher binding affinity are potential candidates for drug-induced presentation.

FIG. 15. Engineering a disulfide bridge to increase complex stability.

FIGS. 16A-16B. Suitable disulfide attachment positions that are identified using disulfide by design (G12V 7-16 (VVVGAVGVGK (SEQ ID NO: 2) and G12C 7-16 (QAFWIDLFETIG SEQ ID NO: 21)). FIG. 16A shows no suitable residues are found with B5701-9mer complexes and FIG. 16B shows disulfide formation confirmed by mass spectrometry of B*57:01/K-Ras(G12C) 7-16.

FIG. 17. B5701•LF9 is a representative stable MHC complex without small molecule drug, B5701•PepV045B is a representative unstable MHC complex with a small molecule drug, and B5701-A67C•C7-16•045B is a representative disulfide-bridged MHC complex with a small molecule drug. B5701•C7-16.045B complex, which would be an ideal control, is too unstable to be prepared and tested. LF9: LSSPVTKSF (SEQ ID NO: 15); PepV: HSITYLLPV (SEQ ID NO: 14); G12C 7-16: VVVGACGVGK (SEQ ID NO: 20).

FIGS. 18A-18B. An unsuccessful disulfide engineering. FIG. 18A shows HLA-B*27:05 containing a natural cysteine in the peptide binding groove (ARAAAAAAA (SEQ ID NO: 22). FIG. 18B shows cysteine- and homocysteine-containing peptides refold with HLA-B*27:05, but independent of Cys67. No disulfide formation observed by mass spectrometry for GXF9.

FIG. 19. Covalent anchoring of peptides that have a C-terminal cysteine.

FIGS. 20A-20C. FIGS. 20A-20C. Design of compounds that stabilize peptides with a C-terminal cysteine by covalent bond formation. FIG. 20A shows the structure of B*57:01/ABA/HSITYLLPV (SEQ ID NO: 14). FIG. 20B shows a modeled structure of B*57:01/ABA/HMTEVVRHC (SEQ ID NO: 16). FIG. 20C. Heat map of ELISA assay signals (OD450) in an MHC refolding assay with HLA-B*57:01, which measures the amounts of correctly folded MHC complex. LF9 is a positive control. PepV is a peptide known to be stabilized by abacavir in HLA-B*57:01. Peptides depicted: LSSPVTKSF (SEQ ID NO: 15), HSITYLLPV (SEQ ID NO: 14), HMTEVVRHC (SEQ ID NO: 16)

FIG. 21. Covalent abacavir-peptide conjugates refold with HLA-B*57:01.

FIG. 22. Structural modifications of abacavir confer altered peptide specificity of B*57:01/ABA/HSITYLLPV (HSITYLLPV (SEQ ID NO: 14)).

FIG. 23A-23C. Stablization of Class I MHC-peptides complexes with abacavir analogs. FIG. 23A: VVVGAVGVGG (SEQ ID NO: 8), VVVGAVGVGA (SEQ ID NO: 9), VVVGAVGVGV (SEQ ID NO: 10), VVVGAVGVGI (SEQ ID NO: 11), VVVGAVGVGK (SEQ ID NO: 12), VVVGAVGVGW (SEQ ID NO: 13), and HSITYLLPV (SEQ ID NO: 14). FIG. 23B: VVVGAVGVGG (SEQ ID NO: 8), VVVGAVGVGA (SEQ ID NO: 9), VVVGAVGVGV (SEQ ID NO: 10), VVVGAVGVGI (SEQ ID NO: 11), VVVGAVGVGK (SEQ ID NO: 12), VVVGAVGVGW (SEQ ID NO: 13), and HSITYLLPV (SEQ ID NO: 14). FIG. 23C: LSSPVTKSF (SEQ ID NO: 15), HSITYLLPV (SEQ ID NO: 14), VVVGAVGVGK (SEQ ID NO: 12), HMTEVVRRC (SEQ ID NO: 17), HMTEVVRRW (SEQ ID NO: 18), HMTEVVRHC (SEQ ID NO: 16), and HMTEVVRHW (SEQ ID NO: 19).

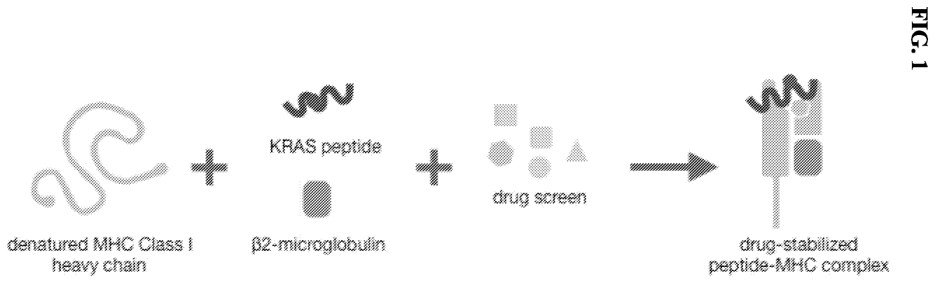

### DETAILED DESCRIPTION

### I. Definitions

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Specific”, “specifically”, “specificity”, or the like of a compound refers to the compound's ability to cause a particular action, such as inhibition, to a particular molecular target with minimal or no action to other proteins in the cell.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

A “regulatory T cell” or “suppressor T cell” is a lymphocyte which modulates the immune system, maintains tolerance to self-antigens, and prevents autoimmune disease.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. In embodiments, an alkenyl includes one or more double bonds. An alkynyl may include more than one triple bond and/or one or more double bonds in addition to the one or more triple bonds. In embodiments, an alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quatemized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. In embodiments, the term “heteroalkenylene,” by itself or as part of another substituent, means, unless otherwise stated, a divalent radical derived from a heteroalkene. In embodiments, the term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. In embodiments, a heteroalkenylene includes one or more double bonds. In embodiments, a heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl. In embodiments, a bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, a bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of 0, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl. In embodiments, the term “heterocycloalkyl” means a monocyclic, bicyclic, or a multicyclic heterocycloalkyl ring system. In embodiments, heterocycloalkyl groups are fully saturated. In embodiments, a bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. In embodiments, a fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). In embodiments, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocycloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

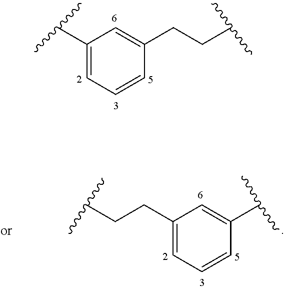 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃ —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or-maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R^{13.D}, etc., wherein each of R^{13.A}, R^{13.B}, R^{13.C}, R^{13.D}, etc. is defined within the scope of the definition of R¹³ and optionally differently.

A “detectable agent” or “detectable moiety” is a composition detectable by appropriate means such as spectroscopic, photochemical, biochemical, immunochemical, chemical, magnetic resonance imaging, or other physical means. In embodiments, a “detectable agent” or “detectable moiety” is a composition, substance, element, or compound; or moiety thereof; detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc ⁹⁹Mo, ¹⁰⁵Pd, ¹⁰⁵Rh ¹¹¹Ag, ¹¹¹In, ¹²³I, ¹²⁴I, ¹²⁵I, ¹³¹I, ¹⁴²Pr, ¹⁴³Pr, ¹⁴⁹Pm ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga, ⁷⁷As, ⁸⁶Y, ⁹⁰Y ⁸⁹Sr, ⁸⁹Zr, ⁹⁴Tc, ⁹⁴Tc, ^{99m}Tc ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety”, and a complementary reactive moiety (i.e. a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g. triflate), tosylates, mesylates, water, alcohols, nitrate, phosphate, thioether, amines, ammonia, fluoride, carboxylate, phenoxides, boronic acid, boronate esters, and alkoxides. In embodiments, two molecules with leaving groups are allowed to contact, and upon a reaction and/or bond formation (e.g., acyloin condensation, aldol condensation, Claisen condensation, Stille reaction) the leaving groups separates from the respective molecule. In embodiments, a leaving group is a bioconjugate reactive moiety. In embodiments, at least two leaving groups (e.g., R¹ and R¹³) are allowed to contact such that the leaving groups are sufficiently proximal to react, interact or physically touch. In embodiments, the leaving groups is designed to facilitate the reaction.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “exogenous” refers to a molecule or substance (e.g., a compound, nucleic acid or protein) that originates from outside a given cell or organism. For example, an “exogenous promoter” as referred to herein is a promoter that does not originate from the plant it is expressed by. Conversely, the term “endogenous” or “endogenous promoter” refers to a molecule or substance that is native to, or originates within, a given cell or organism.

The term “lipid moiety” is used in accordance with its ordinary meaning in chemistry and refers to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 525 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end.

A charged moiety refers to a functional group possessing an abundance of electron density (i.e. electronegative) or is deficient in electron density (i.e. electropositive). Non-limiting examples of a charged moiety includes carboxylic acid, alcohol, phosphate, aldehyde, and sulfonamide. In embodiments, a charged moiety is capable of forming hydrogen bonds.

The term “coupling reagent” is used in accordance with its plain ordinary meaning in the arts and refers to a substance (e.g., a compound or solution) which participates in chemical reaction and results in the formation of a covalent bond (e.g., between bioconjugate reactive moieties, between a bioconjugate reactive moiety and the coupling reagent). In embodiments, the level of reagent is depleted in the course of a chemical reaction. This is in contrast to a solvent, which typically does not get consumed over the course of the chemical reaction. Non-limiting examples of coupling reagents include benzotriazol-1-yl-oxytripyrrolidinophosphonium hexafluorophosphate (PyBOP), 7-Azabenzotriazol-1-yloxy)tripyrrolidinophosphonium hexafluorophosphate (PyAOP), 6-Chloro-benzotriazole-1-yloxy-tris-pyrrolidinophosphonium hexafluorophosphate (PyClock), 1-[Bis(dimethylamino)methylene]-1H-1,2,3-triazolo[4,5-b]pyridinium 3-oxid hexafluorophosphate (HATU), or 2-(1H-benzotriazol-1-yl)-1,1,3,3-tetramethyluronium hexafluorophosphate (HBTU).

The term “solution” is used in accor and refers to a liquid mixture in which the minor component (e.g., a solute or compound) is uniformly distributed within the major component (e.g., a solvent).

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bound atoms or molecules may be bound, e.g., by covalent bond linker (e.g. a first linker or second linker), or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

The term “capable of binding” as used herein refers to a moiety (e.g. a compound as described herein) that is able to measurably bind to a target (e.g., a NF-κB, a Toll-like receptor protein). In embodiments, where a moiety is capable of binding a target, the moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM.

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

The term “non-nucleophilic base” as used herein refers to any sterically hindered base that is a poor nucleophile.

The term “nucleophile” as used herein refers to a chemical species that donates an electron pair to an electrophile to form a chemical bond in relation to a reaction. All molecules or ions with a free pair of electrons or at least one pi bond can act as nucleophiles.

An amino acid residue in a protein “corresponds” to a given residue when it occupies the same essential structural position within the protein as the given residue.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may In embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence. Thus, in embodiments, and as detailed in the next paragraph, the position may correspond to a different numbered position in a corresponding protein that is, for example, homologous and/or contains one or more deletions, insertions, truncations, or fusions.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “amino acid side chain” refers to the functional substituent contained on amino acids. For example, an amino acid side chain may be the side chain of a naturally occurring amino acid. Naturally occurring amino acids are those encoded by the genetic code (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine), as well as those amino acids that are later modified, e.g., hydroxyproline, γ-carboxyglutamate, and O-phosphoserine. In embodiments, the amino acid side chain may be a non-natural amino acid side chain. In embodiments, the amino acid side chain is H,

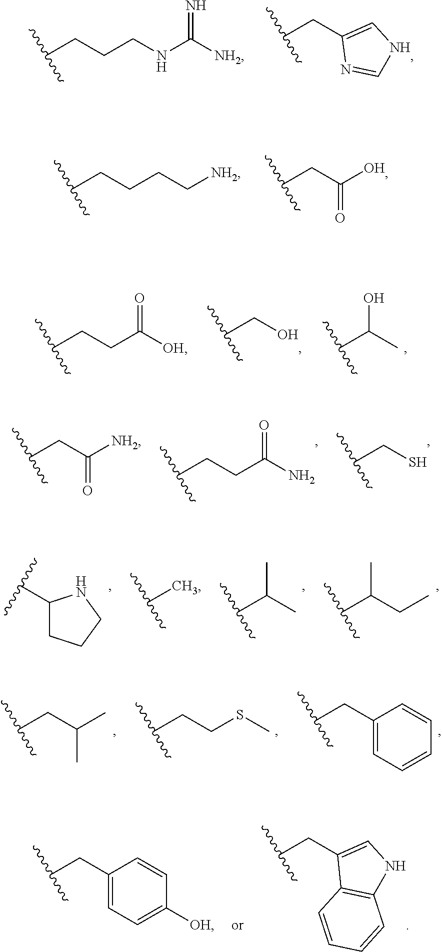

The term “non-natural amino acid side chain” refers to the functional substituent of compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium, allylalanine, 2-aminoisobutryric acid. Non-natural amino acids are non-proteinogenic amino acids that either occur naturally or are chemically synthesized. Such analogs have modified R groups (e.g., norleucine) or modified peptide backbones, but retain the same basic chemical structure as a naturally occurring amino acid. Non-limiting examples include exo-cis-3-Aminobicyclo[2.2.1]hept-5-ene-2-carboxylic acid hydrochloride, cis-2-Aminocycloheptanecarboxylic acid hydrochloride, cis-6-Amino-3-cyclohexene-1-carboxylic acid hydrochloride, cis-2-Amino-2-methylcyclohexanecarboxylic acid hydrochloride, cis-2-Amino-2-methylcyclopentanecarboxylic acid hydrochloride, 2-(Boc-aminomethyl)benzoic acid, 2-(Boc-amino)octanedioic acid, Boc-4,5-dehydro-Leu-OH (dicyclohexylammonium), Boc-4-(Fmoc-amino)-L-phenylalanine, Boc-β-Homopyr-OH, Boc-(2-indanyl)-Gly-OH, 4-Boc-3-morpholineacetic acid, 4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH2)-OH, Boc-Phe(3-NO2)-OH, Boc-Phe(3,5-F2)-OH, 2-(4-Boc-piperazino)-2-(3,4-dimethoxyphenyl)acetic acid purum, 2-(4-Boc-piperazino)-2-(2-fluorophenyl)acetic acid purum, 2-(4-Boc-piperazino)-2-(3-fluorophenyl)acetic acid purum, 2-(4-Boc-piperazino)-2-(4-fluorophenyl)acetic acid purum, 2-(4-Boc-piperazino)-2-(4-methoxyphenyl)acetic acid purum, 2-(4-Boc-piperazino)-2-phenylacetic acid purum, 2-(4-Boc-piperazino)-2-(3-pyridyl)acetic acid purum, 2-(4-Boc-piperazino)-2-[4-(trifluoromethyl)phenyl]acetic acid purum, Boc-β-(2-quinolyl)-Ala-OH, N-Boc-1,2,3,6-tetrahydro-2-pyridinecarboxylic acid, Boc-β-(4-thiazolyl)-Ala-OH, Boc-β-(2-thienyl)-D-Ala-OH, Fmoc-N-(4-Boc-aminobutyl)-Gly-OH, Fmoc-N-(2-Boc-aminoethyl)-Gly-OH, Fmoc-N-(2,4-dimethoxybenzyl)-Gly-OH, Fmoc-(2-indanyl)-Gly-OH, Fmoc-pentafluoro-L-phenylalanine, Fmoc-Pen(Trt)-OH, Fmoc-Phe(2-Br)—OH, Fmoc-Phe(4-Br)—OH, Fmoc-Phe(3,5-F2)-OH, Fmoc-β-(4-thiazolyl)-Ala-OH, Fmoc-β-(2-thienyl)-Ala-OH, 4-(Hydroxymethyl)-D-phenylalanine.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “B-Raf protein” or “B-Raf” as used herein includes any of the recombinant or naturally-occurring forms of the human protein that is encoded by the BRAF gene, or variants or homologs thereof that maintain B-Raf activity (e.g. within at least 50%, 80%, 90%, 95%, 96%, 97%, 98%, 99% or 100% activity compared to B-Raf). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring B-Raf protein. The term “B-Raf” XYZ refers to a nucleotide sequence or protein of a mutant B-Raf wherein the Y numbered amino acid of B-Raf that has an X amino acid in the wildtype instead has a Z amino acid in the mutant (e.g. B-Raf V600E has a V in wildtype protein but an E in the B-Raf V600E mutant protein). In embodiments, the B-Raf protein is substantially identical to the protein identified by the UniProt reference number P15056 or a variant or homolog having substantial identity thereto. In embodiments, the B-Raf protein encoded by the BRAF gene has the amino acid sequence set forth in or corresponding to Entrez 673, UniProt P15056, RefSeq (protein) NP_004324, RefSeq (protein) NP_001341538, RefSeq (protein) NP_001361173, RefSeq (protein) NP_001361187, or RefSeq (protein) NP_001365396. In embodiments, the BRAF gene has the nucleic acid sequence set forth in RefSeq (mRNA) NM_004324, RefSeq (mRNA) NM_001341538, RefSeq (mRNA) NM_001361173, RefSeq (mRNA) NM_001361187, or RefSeq (mRNA) NM_001365396. In embodiments, the amino acid sequence or nucleic acid sequence is the sequence known at the time of filing of the present application. In embodiments, where reference is made to a B-Raf amino acid position, the position corresponds to the numbering system set forth in: https://www.cbioportal.org/. In embodiments, where reference is made to a B-Raf amino acid position, the position corresponds to the numbering system set forth in: https://cancer.sanger.ac.uk/cosmic.

The terms “BRAF gene” or “BRAF” as used herein refer to the any of the recombinant or naturally-occurring forms of the BRAF gene or variants or homologs thereof that code for a B-Raf polypeptide capable of maintaining the activity of the B-Raf polypeptide (e.g., within at least 50%, 80%, 90%, 95%, 96%, 97%, 98%, 99% or 100% activity compared to B-Raf polypeptide). In embodiments, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring BRAF gene.

The term “EGFR protein” as used herein includes any of the recombinant or naturally-occurring forms of epidermal growth factor receptor (EGFR) also known as ErbB-1 or HER1 in humans, or variants or homologs thereof that maintain EGFR protein activity (e.g. within at least 50%, 80%, 90%, 95%, 96%, 97%, 98%, 99% or 100% activity compared to EGFR protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. The term “EGFR” XYZ refers to a nucleotide sequence or protein of a mutant EGFR wherein the Y numbered amino acid of EGFR that has an X amino acid in the wildtype instead has a Z amino acid in the mutant (e.g. EGFR L858R has an L in wildtype protein but an R in the EGFR L858R mutant protein). In embodiments, the EGFR protein is substantially identical to the protein identified by the UniProt reference number P00533 or a variant or homolog having substantial identity thereto. In embodiments, the EGFR protein has the amino acid sequence set forth in or corresponding to Entrez 1956, UniProt P00533, RefSeq (protein) NP_001333826, RefSeq (protein) NP_001333827, RefSeq (protein) NP_001333828, RefSeq (protein) NP_001333829, or RefSeq (protein) NP_001333870. In embodiments, the EGFR gene has the nucleic acid sequence set forth in RefSeq (mRNA) NM_001346897, RefSeq (mRNA) NM_001346898, RefSeq (mRNA) NM_001346899, RefSeq (mRNA) NM_001346900, or RefSeq (mRNA) NM_001346941. In embodiments, the amino acid sequence or nucleic acid sequence is the sequence known at the time of filing of the present application. In embodiments, where reference is made to an EGFR amino acid position, the position corresponds to the numbering system set forth in: https://www.cbioportal.org/. In embodiments, where reference is made to an EGFR amino acid position, the position corresponds to the numbering system set forth in: https://cancer.sanger.ac.uk/cosmic.

The terms “EGFR gene” as used herein refer to the any of the recombinant or naturally-occurring forms of the EGFR gene or variants or homologs thereof that code for a EGFR polypeptide capable of maintaining the activity of the EGFR polypeptide (e.g., within at least 50%, 80%, 90%, 95%, 96%, 97%, 98%, 99% or 100% activity compared to B-Raf polypeptide). In embodiments, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring EGFR gene.

The term “Her2 protein” or “Her2” as used herein includes any of the recombinant or naturally-occurring forms of Receptor tyrosine-protein kinase erbB-2, also known as CD340 (cluster of differentiation 340), proto-oncogene Neu, Erbb2 (rodent), or ERBB2 (human), or variants or homologs thereof that maintain Her2 activity (e.g. within at least 50%, 80%, 90%, 95%, 96%, 97%, 98%, 99% or 100% activity compared to Her2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Her2 protein. In embodiments, the Her2 protein is substantially identical to the protein identified by the UniProt reference number P04626 or a variant or homolog having substantial identity thereto. In embodiments, where reference is made to a Her2 amino acid position, the position corresponds to the numbering system set forth in: https://www.cbioportal.org/. In embodiments, where reference is made to a Her2 amino acid position, the position corresponds to the numbering system set forth in: https://cancer.sanger.ac.uk/cosmic.

The term “Ras” refers to one or more of the family of human Ras GTPase proteins (e.g. K-Ras, H-Ras, N-Ras).

The term “K-Ras” refers to the nucleotide sequences or proteins of human K-Ras (e.g. human K-Ras4A (NP_203524.1), human K-Ras4B (NP_004976.2), or both K-Ras4A and K-Ras4B). The term “K-Ras” includes both the wild-type form of the nucleotide sequences or proteins as well as any mutants thereof. In some embodiments, “K-Ras” is wild-type K-Ras. In some embodiments, “K-Ras” is one or more mutant forms. The term “K-Ras” XYZ refers to a nucleotide sequence or protein of a mutant K-Ras wherein the Y numbered amino acid of K-Ras that has an X amino acid in the wildtype instead has a Z amino acid in the mutant (e.g. K-Ras G12C has a G in wildtype protein but a C in the K-Ras G12C mutant protein). In embodiments, the K-Ras protein is substantially identical to the protein identified by the UniProt reference number P01116 or a variant or homolog having substantial identity thereto. In embodiments, the K-Ras protein encoded by the KRAS gene has the amino acid sequence set forth in or corresponding to Entrez 3845, UniProt P01116, RefSeq (protein) NP_004976, RefSeq (protein) NP_203524, RefSeq (protein) NP_001356715, RefSeq (protein) NP_001356716, or RefSeq (protein) NP_004976.2. In embodiments, the KRAS gene has the nucleic acid sequence set forth in RefSeq (mRNA) NM_004985, RefSeq (mRNA) NM_033360, RefSeq (mRNA) NM_001369786, or RefSeq (mRNA) NM_001369787. In embodiments, the amino acid sequence or nucleic acid sequence is the sequence known at the time of filing of the present application. In embodiments, where reference is made to a K-Ras amino acid position, the position corresponds to the numbering system set forth in: https://www.cbioportal.org/. In embodiments, where reference is made to a K-Ras amino acid position, the position corresponds to the numbering system set forth in: https://cancer.sanger.ac.uk/cosmic.

The terms “KRAS gene” or “KRAS” as used herein refer to the any of the recombinant or naturally-occurring forms of the KRAS gene or variants or homologs thereof that code for a K-Ras polypeptide capable of maintaining the activity of the K-Ras polypeptide (e.g., within at least 50%, 80%, 90%, 95%, 96%, 97%, 98%, 99% or 100% activity compared to K-Ras polypeptide). In embodiments, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring KRAS gene

The term “H-Ras” includes both the wild-type form of the nucleotide sequences or proteins as well as any mutants thereof. In some embodiments, “H-Ras” is wild-type H-Ras. In some embodiments, “H-Ras” is one or more mutant forms. The term “H-Ras” XYZ refers to a nucleotide sequence or protein of a mutant H-Ras wherein the Y numbered amino acid of H-Ras that has an X amino acid in the wildtype instead has a Z amino acid in the mutant (e.g. H-Ras G12C has a G in wildtype protein but a C in the H-Ras G12C mutant protein). In embodiments, the H-Ras protein is substantially identical to the protein identified by the UniProt reference number P01112 or a variant or homolog having substantial identity thereto. In embodiments, the H-Ras protein encoded by the HRAS gene has the amino acid sequence set forth in or corresponding to Entrez 3265, UniProt P01112, RefSeq (protein) NP_001123914, RefSeq (protein) NP_001123915, RefSeq (protein) NP_001123916, RefSeq (protein) NP_001304983, RefSeq (protein) NP_005334, RefSeq (protein) NP_032310, or RefSeq (protein) NP_789765. In embodiments, the HRAS gene has the nucleic acid sequence set forth in RefSeq (mRNA) NM_001130442, NM_001130443, NM_001130444, RefSeq (mRNA) NM_005343, RefSeq (mRNA) NM_176795, RefSeq (mRNA) NM_0013618054, or RefSeq (mRNA) NM_008284. In embodiments, the amino acid sequence or nucleic acid sequence is the sequence known at the time of filing of the present application. In some embodiments, H-Ras refers to the protein NP_005334.1. In embodiments, where reference is made to an H-Ras amino acid position, the position corresponds to the numbering system set forth in: https://www.cbioportal.org/. In embodiments, where reference is made to an H-Ras amino acid position, the position corresponds to the numbering system set forth in: https://cancer.sanger.ac.uk/cosmic.

The term “N-Ras” includes both the wild-type form of the nucleotide sequences or proteins as well as any mutants thereof. In some embodiments, “N-Ras” is wild-type N-Ras. In some embodiments, “N-Ras” is one or more mutant forms. The term “N-Ras” XYZ refers to a nucleotide sequence or protein of a mutant N-Ras wherein the Y numbered amino acid of N-Ras that has an X amino acid in the wildtype instead has a Z amino acid in the mutant (e.g. N-Ras G12C has a G in wildtype protein but a C in the N-Ras G12C mutant protein). In embodiments, the N-Ras protein is substantially identical to the protein identified by the UniProt reference number P01111 or a variant or homolog having substantial identity thereto. In embodiments, the N-Ras protein encoded by the NRAS gene has the amino acid sequence set forth in or corresponding to Entrez 4893, UniProt P01111, or RefSeq (protein) NP_002515. In embodiments, the NRAS gene has the nucleic acid sequence set forth in RefSeq (mRNA) NM_002524, NM_010937, NM_001368638. In embodiments, the amino acid sequence or nucleic acid sequence is the sequence known at the time of filing of the present application. In embodiments, where reference is made to an N-Ras amino acid position, the position corresponds to the numbering system set forth in: https://www.cbioportal.org/. In embodiments, where reference is made to an N-Ras amino acid position, the position corresponds to the numbering system set forth in: https://cancer.sanger.ac.uk/cosmic.

The term “PI3K protein” or “PI3K” as used herein includes any of the recombinant or naturally-occurring forms of the human protein that is encoded by the PIK3 gene, or variants or homologs thereof that maintain PI3K activity (e.g. within at least 50%, 80%, 90%, 95%, 96%, 97%, 98%, 99% or 100% activity compared to PI3K). A “PIK3 gene” or “PIK3” used in the context of a nucleic acid encoding for a PI3K polypeptide, as set forth above and herein, may alternatively be referred to herein as a PI3K gene or PI3K. In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PI3K protein. The term “PI3K” XYZ refers to a nucleotide sequence or protein of a mutant PI3K wherein the Y numbered amino acid of PI3K that has an X amino acid in the wildtype instead has a Z amino acid in the mutant (e.g. PI3K E545K has an E in wildtype protein but a K in the PI3K E545K mutant protein). In embodiments, the PI3K protein is PK3CA. In embodiments, the PI3K protein is substantially identical to the protein identified by the UniProt reference number P42336 or a variant or homolog having substantial identity thereto. In embodiments, where reference is made to a PI3K amino acid position, the position corresponds to the numbering system set forth in: https://www.cbioportal.org/. In embodiments, where reference is made to a PI3K amino acid position, the position corresponds to the numbering system set forth in: https://cancer.sanger.ac.uk/cosmic.

The terms “PIK3 gene” or “PIK3” as used herein refer to the any of the recombinant or naturally-occurring forms of the PIK3 gene or variants or homologs thereof that code for a PI3K polypeptide capable of maintaining the activity of the PI3K polypeptide (e.g., within at least 50%, 80%, 90%, 95%, 96%, 97%, 98%, 99% or 100% activity compared to PI3K polypeptide). In embodiments, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring PIK3 (ie. PI3K) gene.

The term “p53” or “tumor protein p53” as used herein includes any of the recombinant or naturally-occurring forms of the human protein that is encoded by the TP53 gene, or variants or homologs thereof that maintain p53 activity (e.g. within at least 50%, 80%, 90%, 95%, 96%, 97%, 98%, 99% or 100% activity compared to p53). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring p53 protein. The term “p53” XYZ refers to a nucleotide sequence or protein of a mutant p53 wherein the Y numbered amino acid of p53 that has an X amino acid in the wildtype instead has a Z amino acid in the mutant (e.g. p53 R175H has an R in wildtype protein but an H in the p53 E545K mutant protein). In embodiments, the p53 protein is substantially identical to the protein identified by the UniProt reference number P04637 or a variant or homolog having substantial identity thereto. In embodiments, the p53 protein encoded by the TP53 gene has the amino acid sequence set forth in or corresponding to Entrez 7157, UniProt P04637, RefSeq (protein) NP_000537, RefSeq (protein) NP_001119584, RefSeq (protein) NP_001119585, RefSeq (protein) NP_001119586, or RefSeq (protein) NP_001119587. In embodiments, the amino acid sequence or nucleic acid sequence is the sequence known at the time of filing of the present application. In embodiments, where reference is made to a p53 amino acid position, the position corresponds to the numbering system set forth in: https://www.cbioportal.org/. In embodiments, where reference is made to a p53 amino acid position, the position corresponds to the numbering system set forth in: https://cancer.sanger.ac.uk/cosmic.

The terms “TP53 gene” or “TP53” as used herein refer to the any of the recombinant or naturally-occurring forms of the TP53 gene or variants or homologs thereof that code for a p53 polypeptide capable of maintaining the activity of the p53 polypeptide (e.g., within at least 50%, 80%, 90%, 95%, 96%, 97%, 98%, 99% or 100% activity compared to p53 polypeptide). In embodiments, the variants or homologs have at least 90%, 95%, 96%, 97%, 98%, 99% or 100% nucleic acid sequence identity across the whole sequence or a portion of the sequence (e.g., a 50, 100, 150 or 200 continuous nucleic acid portion) compared to a naturally occurring TP53 gene.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The terms “antigen” and “epitope” interchangeably refer to the portion of a molecule (e.g., a polypeptide) which is specifically recognized by a component of the immune system, e.g., an antibody, a T cell receptor, or other immune receptor such as a receptor on natural killer (NK) cells. As used herein, the term “antigen” encompasses antigenic epitopes and antigenic fragments thereof.

The term “vaccine” refers to a composition that can provide active acquired immunity to and/or therapeutic effect (e.g. treatment) of a particular disease or a pathogen. A vaccine typically contains one or more agents that can induce an immune response in a subject against a pathogen or disease, i.e. a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g. preventing or ameliorating the effects of a future infection by any natural or pathogen, or of an anticipated occurrence of cancer in a predisposed subject) or therapeutic (e.g., treating cancer in a subject who has been diagnosed with the cancer). The administration of vaccines is referred to vaccination. In some examples, a vaccine composition can provide nucleic acid, e.g. mRNA that encodes antigenic molecules (e.g. peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g. one or more peptides that are known to be expressed in the pathogen (e.g. pathogenic bacterium or virus). In the context of cancer vaccine, the vaccine composition can provide mRNA encoding certain peptides that are associated with cancer, e.g. peptides that are substantially exclusively or highly expressed in cancer cells as compared to normal cells. The subject, after vaccination with the cancer vaccine composition, can have immunity against the peptides that are associated with cancer and kill the cancer cells with specificity.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

The term “MHC protein” refers to a protein that is encoded by one of the genes that makes up the major histocompatibility complex (MHC). In embodiments, the MHC protein is an MHC class I protein or an MHC class II protein. In embodiments, the MHC protein is an MHC class I protein. In embodiments, the MHC protein is an MHC class II protein.

The term “driver oncogene mutation” refers to a mutation in a normal gene that predisposes a cell to cancer. In embodiments, the driver oncogene mutation is a mutation in a gene that changes the activity of the protein gene product in a way that increases the likelihood that a cell having the driver oncogene mutation becomes cancerous. In embodiments, the driver oncogene mutation is a tumor suppressor mutation resulting in production of a mutated tumor suppressing protein with reduced tumor suppressing activity, thereby increasing the likelihood that a cell having the tumor suppressor mutation becomes cancerous. In embodiments, the driver oncongene mutation is not limited to a point mutation. In embodiments, the driver oncogene mutation is in an EGFR gene. In embodiments, the driver oncogene mutation is in a PDGFR gene. In embodiments, the driver oncogene mutation is in a VEGFR gene. In embodiments, the driver oncogene mutation is in a HER2/neu gene. In embodiments, the driver oncogene mutation is in a BRAF gene. In embodiments, the driver oncogene mutation is in a K-Ras gene. In embodiments, the driver oncogene mutation is in a PI3K (ie. PIK3) gene.

The term “driver oncogene protein” is a protein that is expressed as a result of a driver oncogene mutation (i.e. the protein gene product of a driver oncogene mutation). In embodiments, the driver oncogene protein is an EGFR protein. In embodiments, the driver oncogene protein is a PDGFR protein. In embodiments, the driver oncogene protein is a VEGFR protein. In embodiments, the driver oncogene protein is a HER2/neu protein. In embodiments, the driver oncogene protein is a B-Raf protein. In embodiments, the driver oncogene protein is a K-Ras protein. In embodiments, the driver oncogene protein is KRAS p.G12V. In embodiments, the driver oncogene protein is a PI3K protein.

The term “tumor suppressor gene” is a gene that regulates a cell during cell division and replication. Thus, in embodiments, the tumor suppressor gene encodes a protein having tumor suppressing activity, also referred to herein as a tumor suppressor protein or tumor suppressor (e.g. p53). In embodiments, the tumor suppressor gene contains one or more mutations that results in a loss or reduction in its function as a tumor suppressor. Thus, in embodiments, the tumor suppressor gene contains one or more mutations that encodes for a mutated tumor suppressor protein with reduced or eliminated tumor suppressing function. In embodiments, the tumor suppressor gene mutation is in tumor protein p53 (p53). In embodiments, the mutant p53 protein is R175H, R175G, R175L, R175C, Y220C, G245S, G245D, G245V, G245R, R248Q, R248W, R248L, R273H, R273C, R273L, R282W, or R282G.

The term “EC50” or “half maximal effective concentration” as used herein refers to the concentration of a molecule (e.g., antibody, chimeric antigen receptor or bispecific antibody) capable of inducing a response which is halfway between the baseline response and the maximum response after a specified exposure time. In embodiments, the EC50 is the concentration of a molecule (e.g., antibody, chimeric antigen receptor or bispecific antibody) that produces 50% of the maximal possible effect of that molecule.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

An “inhibitor” refers to a compound (e.g. compounds described herein) that reduces activity when compared to a control, such as absence of the compound or a compound with known inactivity.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

“Stabilizes” as used herein means decreases the Kd of the MHC protein binding to the peptide antigen. Therefore, a compound stabilizes the binding of an MHC protein to the peptide antigen by decreasing the Kd of the MHC protein binding to the peptide antigen relative to the absence of the compound.

The term “irreversible covalent bond” is used in accordance with its plain ordinary meaning in the art and refers to the resulting association between atoms or molecules of (e.g., electrophilic chemical moiety and nucleophilic moiety) wherein the probability of dissociation is low. In embodiments, the irreversible covalent bond does not easily dissociate under normal biological conditions. In embodiments, the irreversible covalent bond is formed through a chemical reaction between two species (e.g., electrophilic chemical moiety and nucleophilic moiety).

The term “electrophilic moiety” is used in accordance with its plain ordinary chemical meaning and refers to a chemical group (e.g., monovalent chemical group) that is electrophilic. In embodiments, the electrophilic chemical moiety is referred to herein as “E.” In embodiments, E is:

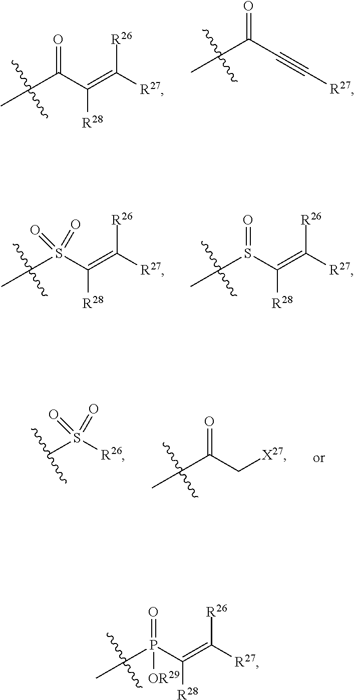

wherein R²⁶, R²⁷, R²⁸, R²⁹, and X²⁷ are as described herein, including in embodiments. In embodiments, an electrophilic moiety is a covalent cysteine modifier moiety.

The term “covalent cysteine modifier moiety” as used herein refers to a monovalent electrophilic moiety that is able to measurably bind to a cysteine amino acid. In embodiments, the covalent cysteine modifier moiety binds via an irreversible covalent bond. In embodiments, the covalent cysteine modifier moiety is capable of binding with a Kd of less than about 10 μM, 5 μM, 1 μM, 500 nM, 250 nM, 100 nM, 75 nM, 50 nM, 25 nM, 15 nM, 10 nM, 5 nM, 1 nM, or about 0.1 nM. In embodiments, the covalent cysteine modifier moiety binds via a covalent bond.

The term “nucleophilic moiety” is used in accordance with its plain ordinary chemical meaning and refers to a chemical group (e.g., monovalent chemical group) that is nucleophilic.

### II. Methods of Use

In one aspect provided herein is a method of identifying a candidate compound that stabilizes binding of an MHC protein to a peptide antigen. The method includes contacting an MHC protein with a peptide antigen and a candidate compound thereby forming an MHC-peptide-compound complex, and then detecting the increased stability of the MHC-peptide-compound complex relative to the stability of an MHC-peptide complex which does not include the candidate compound. Thus the candidate compound is identified as a compound that stabilizes the binding of the MHC protein to the peptide antigen. The method may be performed in vitro.

In embodiments, the MHC protein binds to the peptide antigen with a Kd of greater than 1 micromolar (in the presence of the candidate compound). In embodiments, the MHC protein binds to the peptide antigen with a Kd of greater than 10 nM, 100 nM, 500 nM, 1 μM, 10 μM, 50 μM, 100 μM, 500 μM, or 1 mM (in the presence of the candidate compound). The Kd may be a specific value in a range from 10 nM to 100 nM, 100 nM to 500 nM, 500 nM to 1 μM, 1 μM to 10 μM, 10 μM to 50 μM, 50 μM to 100 μM, 100 M to 500 μM, or 500 M to 1 mM (in the presence of the candidate compound). The specific value may be selected from any 1 nM increment in the selected range (in the presence of the candidate compound). The Kd may be selected from a sub-range within any of the aforementioned Kd ranges (in the presence of the candidate compound). The low endpoint of a sub-range may be the low end of the range or any value selected from 1 nM increments above the low end of the range up to 1 nM less that the high end of the range (in the presence of the candidate compound). The high endpoint of a sub-range may be the high end of the range or any value selected from 1 nM below the high end of the range to 1 nM greater than the low end of the range (in the presence of the candidate compound).

In embodiments, the MHC protein binds to the peptide antigen with a Kd of greater than 10 nM (in the presence of the candidate compound). In embodiments, the MHC protein binds to the peptide antigen with a Kd of greater than 100 nM (in the presence of the candidate compound). In embodiments, the MHC protein binds to the peptide antigen with a Kd of greater than 500 nM (in the presence of the candidate compound). In embodiments, the MHC protein binds to the peptide antigen with a Kd of greater than 1 μM (in the presence of the candidate compound). In embodiments, the MHC protein binds to the peptide antigen with a Kd of greater than 10 μM (in the presence of the candidate compound). In embodiments, the MHC protein binds to the peptide antigen with a Kd of greater than 50 μM (in the presence of the candidate compound). In embodiments, the MHC protein binds to the peptide antigen with a Kd of greater than 100 μM (in the presence of the candidate compound). In embodiments, the MHC protein binds to the peptide antigen with a Kd of greater than 500 μM (in the presence of the candidate compound). In embodiments, the MHC protein binds to the peptide antigen with a Kd of greater than 1 mM (in the presence of the candidate compound).

In embodiments, the MHC protein contacting the peptide antigen and a candidate compound is folded, partially folded or unfolded. In embodiments, the MHC protein contacting the peptide antigen and a candidate compound is folded or unfolded. In embodiments, the MHC protein contacting the peptide antigen and a candidate compound is folded. In embodiments, the MHC protein contacting the peptide antigen and a candidate compound is partially folded. In embodiments, the MHC protein contacting the peptide antigen and a candidate compound is unfolded.

In embodiments, the MHC protein is an MHC class I protein or an MHC class II protein. In embodiments, the MHC protein is an MHC class I protein. In embodiments, the MHC protein is an MHC class II protein.

In embodiments, the MHC class I protein is the MHC class I heavy chain protein and light chain protein. In embodiments, the light chain protein is a β-microglobulin. In embodiments, the heavy chain protein is alpha chain. In embodiments, the alpha chain is alpha polypeptide chain. In embodiments, the alpha chain consists of three extracellular regions or domains, designated α1, α2, and α3. In embodiments, the α1 and α2 domains form a site for the binding of peptides derived from antigens. In embodiments, three loci encoding classical (major) MHC class I molecules in humans are designated HLA-A, HLA-B, and HLA-C. In embodiments, there are five non-classical (minor) MHC class I molecules in humans are designated HLA-E, HLA-F, HLA-G, HLA-K and HLA-L.

In embodiments, the MHC protein is HLA-A, HLA-B, HLA-C, HLA-E, HLA-F, HLA-G, HLA-K or HLA-L. In embodiments, the MHC protein is HLA-A, HLA-B or HLA-C. In embodiments, the MHC protein is HLA-A. In embodiments, the MHC protein is HLA-B. In embodiments, the MHC protein is HLA-C. In embodiments, the MHC protein is HLA-E. In embodiments, the MHC protein is HLA-F. In embodiments, the MHC protein is HLA-G. In embodiments, the MHC protein is HLA-K. In embodiments, the MHC protein is HLA-L.

In embodiments, the MHC protein is HLA-B. In embodiments, the MHC protein is HLA-B*57:01. In embodiments, the MHC protein is HLA-B*58:01.

In embodiments, a peptide antigen is a driver oncogene derived peptide. A driver oncogene derived peptide is a peptide antigen derived from a driver oncogene protein, and may also be referred to herein as a peptide cancer antigen. In embodiments, the driver oncogene derived peptide (peptide cancer antigen) includes an amino acid sequence that contains the mutation that changes the activity of the driver oncogene protein from which it is derived in a way that increases the likelihood that a cell having the driver oncogene mutation becomes cancerous. In embodiments, a peptide antigen is a common driver oncogene derived peptide. In embodiments, the peptide antigen includes an amino acid sequence encoded by a driver oncogene mutation. Thus, the peptide antigen may include an amino acid sequence that contains the mutation that changes the activity of the driver oncogene protein in a way that increases the likelihood that a cell having the driver oncogene mutation becomes cancerous. In embodiments, peptide antigen is altered driver oncogene derived peptide. In embodiments, driver oncogenic alterations, include mutation, truncation, gene fusion, and/or splice variants. In embodiments, common driver oncogenes are KRAS (G12D/V/C), BRAF (V600E), and PI3K (i.e., PIK3) (E545K/H1047R). In embodiments, common driver oncogene is KRAS (G12D/V/C). In embodiments, common driver oncogene is BRAF (V600E). In embodiments, common driver oncogene is PI3K (i.e., PIK3) (E545K/H1047R). Thus, in embodiments, the peptide antigen includes an amino acid sequence including KRAS (G12D/V/C), BRAF (V600E), or PI3K (E545K/H1047R).

In embodiments, the peptide antigen is 5 to 100 amino acids in length. In embodiments, the peptide antigen is 5 to 50 amino acids in length. In embodiments, the peptide antigen is 5 to 25 amino acids in length. In embodiments, the peptide antigen is 5 to 20 amino acids in length. In embodiments, the peptide antigen is 5 to 15 amino acids in length. In embodiments, the peptide antigen is 5 to 14 amino acids in length. In embodiments, the peptide antigen is 5 to 13 amino acids in length. In embodiments, the peptide antigen is 5 to 12 amino acids in length. In embodiments, the peptide antigen is 5 to 11 amino acids in length. In embodiments, the peptide antigen is 5 to 10 amino acids in length.

In embodiments, the peptide antigen is 6 to 20 amino acids in length. In embodiments, the peptide antigen is 6 to 15 amino acids in length. In embodiments, the peptide antigen is 6 to 14 amino acids in length. In embodiments, the peptide antigen is 6 to 13 amino acids in length. In embodiments, the peptide antigen is 6 to 12 amino acids in length. In embodiments, the peptide antigen is 6 to 11 amino acids in length. In embodiments, the peptide antigen is 6 to 10 amino acids in length.

In embodiments, the peptide antigen is 7 to 20 amino acids in length. In embodiments, the peptide antigen is 7 to 15 amino acids in length. In embodiments, the peptide antigen is 7 to 14 amino acids in length. In embodiments, the peptide antigen is 7 to 13 amino acids in length. In embodiments, the peptide antigen is 7 to 12 amino acids in length. In embodiments, the peptide antigen is 7 to 11 amino acids in length. In embodiments, the peptide antigen is 7 to 10 amino acids in length.

In embodiments, the peptide antigen is 8 to 20 amino acids in length. In embodiments, the peptide antigen is 8 to 15 amino acids in length. In embodiments, the peptide antigen is 8 to 14 amino acids in length. In embodiments, the peptide antigen is 8 to 13 amino acids in length. In embodiments, the peptide antigen is 8 to 12 amino acids in length. In embodiments, the peptide antigen is 8 to 11 amino acids in length. In embodiments, the peptide antigen is 8 to 10 amino acids in length.

In embodiments, the peptide antigen is 9 to 20 amino acids in length. In embodiments, the peptide antigen is 9 to 15 amino acids in length. In embodiments, the peptide antigen is 9 to 14 amino acids in length. In embodiments, the peptide antigen is 9 to 13 amino acids in length. In embodiments, the peptide antigen is 9 to 12 amino acids in length. In embodiments, the peptide antigen is 9 to 11 amino acids in length. In embodiments, the peptide antigen is 9 to 10 amino acids in length.

In embodiments, driver oncogene derived peptides are presented by the MHC protein. In embodiments, driver oncogene derived peptides are not sufficiently presented by the MHC protein for T-cell recognition. In embodiments, driver oncogene derived peptides are not presented by the MHC protein. In embodiments, driver oncogene derived peptides are intracellular antigens. In embodiments, an MHC-peptide antigen stabilizing compound increases the MHC presentation of driver oncogene derived peptides, bringing them into the visibility of T-cell surveillance. Thus, an MHC-peptide antigen stabilizing compound may increase the MHC presentation of peptide antigen (e.g. a driver oncogene derived peptide) relative to the absence of the MHC-peptide antigen stabilizing compound. In embodiments, an MHC-peptide antigen stabilizing compound induces the presentation of KRAS (G12D) derived peptides by HLA-B*57:01. In embodiments, an MHC-peptide antigen stabilizing compound induced the presentation of KRAS (G12V) derived peptides by HLA-B*57:01. In embodiments, where the MHC presentiation is increased or induced, the MHC-peptide antigen is stabilized byt the MHC-peptide antigen stabilizing compound relative to the absence of the MHC-peptide antigen stabilizing compound.

In embodiments, a candidate compound is a compound to be tested. In embodiments, a candidate compound is an MHC-peptide antigen stabilizing compound. In embodiments, provided herein is an MHC-peptide antigen stabilizing compound having molecular weight of less than 2000 g/mol. In embodiments, the MHC-peptide antigen stabilizing compound has molecular weight of less than 1500 g/mol. In embodiments, the MHC-peptide antigen stabilizing compound has molecular weight of less than 1000 g/mol. In embodiments, the MHC-peptide antigen stabilizing compound has molecular weight of less than 750 g/mol. In embodiments, the MHC-peptide antigen stabilizing compound has molecular weight of less than 500 g/mol. In embodiments, the MHC-peptide antigen stabilizing compound has molecular weight of less than 100 g/mol.

In embodiments, provided herein is an MHC-peptide antigen stabilizing compound having the structure of formula I or II, or a salt thereof:

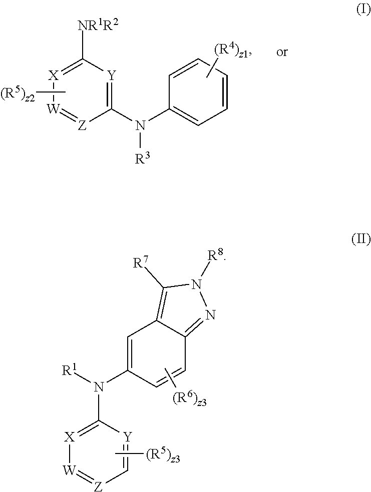

W, X, Y and Z are each independently C or N.

R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SOₙ₁R^{1A}, —SOᵥ₁NR^{1A}R^{1B}, —POₘ₁R^{1A}, —POᵣ₁NR^{1A}R^{1B} substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl.

R² is hydrogen,

halogen, —CCl₃, —CBr₃, —CF₃, —CI₃, —CH₂Cl, —CH₂Br, —CH₂F, —CH₂I, —CHCl₂,
—CHBr₂, —CHF₂, —CHI₂, —CN, —OH, —NH₂, —COOH, —CONH₂, —NO₂, —SH, —SOₙ₂R^{2A},
—SOᵥ₂NR^{2A}R^{2B}, —POₘ₂R^{2A}, —POᵣ₂NR^{2A}R^{2B}, —NHNH₂, —ONH₂,
—NHC(O)NHNH₂, —NHC(O)NH₂, —NHSO₂H,
—NHC(O)H, —NHC(O)OH, —NHOH, —OCCl₃, —OCF₃, —OCBr₃, —OCI₃, —OCHCl₂, —OCHBr₂, —OCHI₂,
—OCHF₂, —N₃, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl.

R³ is hydrogen, halogen, —CCl₃, —CBr₃, —CF₃, —CI₃, —CH₂Cl, —CH₂Br, —CH₂F, —CH₂I, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl.

R⁴ is hydrogen, oxo, halogen, —CCl₃, —CBr₃, —CF₃, —CI₃, —CH₂C₁, —CH₂Br, —CH₂F, —CH₂I, —CHCl₂, —CHBr₂, —CHF₂, —CHI₂, —CN, —OR^{4A}, —NR^{4A}R^{4B}, —COOR^{4A}, —CONR^{4A}R^{4B}, —NO₂, —SR^{4A}, —SOₙ₄R^{4A}, —SOᵥ₄NR^{4A}R^{4B}, —PO(OH)₂, —P_{Om4}R^{4A}, —POᵣ₄NR^{4A}R^{4B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl.

R⁵ is hydrogen, oxo, halogen, —CCl₃, —CBr₃, —CF₃, —CI₃, —CH₂C₁, —CH₂Br, —CH₂F, —CH₂I, —CHCl₂, —CHBr₂, —CHF₂, —CHI₂, —CN, —OR^{5A}, —NR^{5A}R^{5B}, —COOH, —CONH₂, —NO₂, —SH, —SOₙ₅R^{5A}, —PO(OH)₂, —POₘ₅R^{5A}, —POᵣ₅NR^{5A}R^{5B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl.

R⁶ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{6A}, —NH₂, —COOH, —CONH₂, —NO₂, —SH, —SOₙ₆R^{6A}, —SOᵥ₆NR^{6A}R^{6B}, —PO(OH)₂, —POₘ₆R^{6A}, —POᵣ₆NR^{6A}R^{6B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl.

R⁷ is hydrogen,

halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{7A}, —NH₂, —COOH, —CONH₂, —NO₂, —SH, —SOₙ₇R^{7A},
—SOᵥ₇NR^{7A}R^{7B}, —PO(OH)₂, —POₘ₇R^{7A}, —POᵣ₇NR^{7A}R^{7B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl.

R⁸ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SOₙ₈R^{8A}, —SOᵥ₈NR^{8A}R^{8B}, —PO(OH)₂, —POₘ₈R^{8A}, —POᵣ₈NR^{8A}R^{8B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl.

Each R^{1A}, R^{1B}, R^{2A}, R^{2B}, R^{4A}, R^{4B}, R^{5A}, R^{5B}, R^{6A}, R^{6B}, R^{7A}, R^{7B}, R^{8A}, and R^{8B} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NH—NH₂, —NHC═(O)NH₂, —NHSO₂H, —NHC═(O)H,

—NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{1A} and R^{1B} substituents bonded to the same nitrogen atom may optionally be joined to form a substituted or unsubstituted heterocycloalkyl or substituted or unsubstituted heteroaryl. In embodiments, R^{2A} and R^{2B} substituents bonded to the same nitrogen atom may optionally be joined to form a substituted or unsubstituted heterocycloalkyl or substituted or unsubstituted heteroaryl. In embodiments, R^{4A} and R^{4B} substituents bonded to the same nitrogen atom may optionally be joined to form a substituted or unsubstituted heterocycloalkyl or substituted or unsubstituted heteroaryl. In embodiments, R^{4A} and R^{4B} substituents bonded to the same nitrogen atom may optionally be joined to form a substituted or unsubstituted heterocycloalkyl or substituted or unsubstituted heteroaryl. In embodiments, R^{6A} and R^{6B} substituents bonded to the same nitrogen atom may optionally be joined to form a substituted or unsubstituted heterocycloalkyl or substituted or unsubstituted heteroaryl. In embodiments, R^{7A} and R^{7B} substituents bonded to the same nitrogen atom may optionally be joined to form a substituted or unsubstituted heterocycloalkyl or substituted or unsubstituted heteroaryl. In embodiments, R^{8A} and R^{8B} substituents bonded to the same nitrogen atom may optionally be joined to form a substituted or unsubstituted heterocycloalkyl or substituted or unsubstituted heteroaryl.

X is —Cl, —Br, —I or —F.

Each n1, n2, n4, n5, n6, n7, and n8 is independently an integer from 0 to 4.

Each v1, v2, v4, v5, v6, v7, and v8 is independently 1 or 2.

Each m1, m2, m4, m5, m6, m7, and m8 is independently an integer from 0 to 3.

Each r1, r2, r4, r5, r6, r7, and r8 is independently 1 or 2.

Each z1 and z3 is independently 0 to 5. In embodiments, z2 is 0 to 4. In embodiments, z4 is 0 to 3.

In embodiments, R¹ is hydrogen or unsubstituted alkyl; R³ is hydrogen or unsubstituted alkyl; R² is hydrogen, substituted or unsubstituted alkyl, substituted or unsubstituted aryl, substituted or unsubstituted heteroaryl, or substituted or unsubstituted cycloalkyl; R⁵ is hydrogen, oxo,

halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NR^{5A}R^{5B}, —COOH, —CONH₂, —NO₂, —SH, substituted or unsubstituted heteroalkyl, or substituted or unsubstituted alkyl; R⁴ is hydrogen, substituted or unsubstituted alkyl or —SO₂NR^{4A}R^{4B}; R⁶ is hydrogen, halogen, or substituted or unsubstituted alkyl; R⁷ is hydrogen or substituted or unsubstituted alkyl; R⁸ is hydrogen or substituted or unsubstituted alkyl; and each R^{4A}, R^{4B}, R^{5A} and R^{5B} is independently hydrogen or substituted or unsubstituted alkyl.

In embodiments, R¹ is hydrogen or methyl; R³ is hydrogen or methyl; R² is methyl, unsubstituted cycloalkyl, unsubstituted aryl or substituted heteroaryl; R⁵ is hydrogen, oxo, methyl, halogen, unsubstituted heteroalkyl or —NR^{5A}R^{5B}; R⁴ is hydrogen, methyl or —SO₂NR^{4A}R^{4B}; R⁶ is hydrogen or methyl; R⁷ is hydrogen or methyl; R⁸ is hydrogen or methyl; and each R^{4A}, R^{4B}, R^{5A} and R^{5B} is independently hydrogen or methyl.

In embodiments, R¹ is hydrogen or methyl; R³ is hydrogen or methyl; R² is methyl, cyclopropyl, phenyl, or substituted 2H-indazole; R⁵ is hydrogen, oxo, halogen, ethoxy or —NR^{5A}R^{5B}; R⁴ is hydrogen, methyl or —SO₂NR^{4A}R^{4B}; R⁶ is hydrogen or methyl; R⁷ is methyl; R⁸ is methyl; and each R^{4A}, R^{4B}, R^{5A} and R^{5B} is independently hydrogen or methyl.

In embodiments, W, X, Y and Z are each independently C or N. In embodiments, W is C or N. In embodiments, X is C or N. In embodiments, Y is C or N. In embodiments, Z is C or N. In embodiments, Y and Z are N. In embodiments, X and W are C. In embodiments, X and Y are N. In embodiments, W and Z are C. In embodiments, X, Y, W and Z are N. In embodiments, X, Y, W and Z are C. In embodiments, X is N. In embodiments, Y, W and Z are C. In embodiments, Y is N. In embodiments, W is N. In embodiments, Z is N. In embodiments, X and W are N. In embodiments, X and Z are N. In embodiments, Y and W are N.

In embodiments, Y and Z are N, and W and X are C. In embodiments, X and Y are N, and W and Z are C.

In embodiments, R¹ is hydrogen or unsubstituted alkyl (e.g., C₁-C₈ alkyl, C₁-C₆ alkyl, or C₁-C₄ alkyl). In embodiments, R¹ is hydrogen. In embodiments, R¹ is unsubstituted alkyl (e.g., C₁-C₈ alkyl, C₁-C₆ alkyl, or C₁-C₄ alkyl). In embodiments, R¹ is hydrogen, methyl, ethyl, propyl, butyl or pentyl. In embodiments, R¹ is hydrogen or methyl. In embodiments, R¹ is hydrogen. In embodiments, R¹ is methyl. In embodiments, R¹ is ethyl. In embodiments, R¹ is propyl. In embodiments, R¹ is butyl. In embodiments, R¹ is pentyl.

In embodiments, R³ is hydrogen or unsubstituted alkyl (e.g., C₁-C₈ alkyl, C₁-C₆ alkyl, or C₁-C₄ alkyl). In embodiments, R³ is hydrogen. In embodiments, R³ is unsubstituted alkyl (e.g., C₁-C₈ alkyl, C₁-C₆ alkyl, or C₁-C₄ alkyl). In embodiments, R³ is hydrogen, methyl, ethyl, propyl, butyl or pentyl. In embodiments, R³ is hydrogen or methyl. In embodiments, R³ is hydrogen. In embodiments, R³ is methyl. In embodiments, R³ is ethyl. In embodiments, R³ is propyl. In embodiments, R³ is butyl. In embodiments, R³ is pentyl.

In embodiments, R² is hydrogen, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R² is hydrogen, unsubstituted alkyl (e.g., C₁-C₈ alkyl, C₁-C₆ alkyl, or C₁-C₄ alkyl), unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, R² is hydrogen. In embodiments, R² is unsubstituted alkyl (e.g., C₁-C₈ alkyl, C₁-C₆ alkyl, or C₁-C₄ alkyl). In embodiments, R² is unsubstituted cycloalkyl (e.g., C₃-C₈ cycloalkyl, C₃-C₆ cycloalkyl, or C₅-C₆ cycloalkyl). In embodiments, R² is substituted (e.g. with a substituent group, a size-limited substituent group or a lower substituent group) aryl (e.g., C₆-C₁₀ aryl, C₁₀ aryl, or phenyl). In embodiments, R² is unsubstituted aryl (e.g., C₆-C₁₀ aryl, C₁₀ aryl, or phenyl). In embodiments, R² is substituted (e.g. with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, R² is unsubstituted heteroaryl (e.g., 5 to 10 membered heteroaryl, 5 to 9 membered heteroaryl, or 5 to 6 membered heteroaryl).

In embodiments, R² is methyl, ethyl, propyl, butyl, pentyl, hexyl, cyclopropyl, cyclobutyl, cyclopentyl, cyclohexyl, unsubstituted aryl (e.g., C₆-C₁₀ aryl, C₁₀ aryl, or phenyl), or substituted (e.g. with a substituent group, a size-limited substituent group or a lower substituent group) heteroaryl (e.g., 5 to 10 membered heteroaryl, 5 to 9 membered heteroaryl, or 5 to 6 membered heteroaryl).

In embodiments, R² is methyl, ethyl, propyl, cyclopropyl, cyclobutyl, cyclopentyl, cyclohexyl, phenyl, or substituted (e.g. with a substituent group, a size-limited substituent group or a lower substituent group) heteroaryl (e.g., 5 to 10 membered heteroaryl, 5 to 9 membered heteroaryl, or 5 to 6 membered heteroaryl).

In embodiments, R² is methyl, cyclopropyl, phenyl, 1H-indazole or 2H-indazole. In embodiments, R² is methyl. In embodiments, R² is cyclopropyl. In embodiments, R² is phenyl. In embodiments, R² is substituted (e.g. with a substituent group, a size-limited substituent group or a lower substituent group) 1H-indazole. In embodiments, R² is substituted (e.g. with a substituent group, a size-limited substituent group or a lower substituent group) 2H-indazole.

In embodiments, R² is:

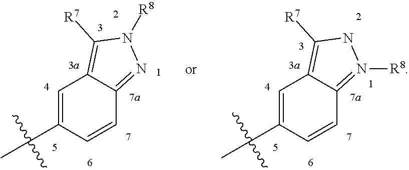

In embodiments, R² is

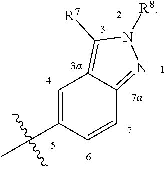

In embodiments, R² is

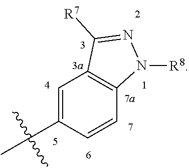

In embodiments, R² is

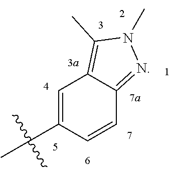 [SMILES: *c1ccc2nn(C)c(C)c2c1]

In embodiments, R² is

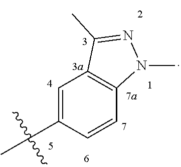 [SMILES: *c1ccc2c(c1)c(C)nn2C]

In embodiments, R⁷ is hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments R⁷ is hydrogen, methyl, ethyl, propyl, butyl, isopropyl, isobutyl or tert-butyl. In embodiments, R⁷ is hydrogen. In embodiments, R⁷ is methyl. In embodiments, R⁷ is ethyl. In embodiments, R⁷ is propyl. In embodiments, R⁷ is butyl. In embodiments, R⁷ is isobutyl. In embodiments, R⁷ is tert-butyl.

In embodiments, R⁸ is hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments R⁸ is hydrogen, methyl, ethyl, propyl, butyl, isopropyl, isobutyl or tert-butyl. In embodiments, R⁸ is hydrogen. In embodiments, R⁸ is methyl. In embodiments, R⁸ is ethyl. In embodiments, R⁸ is propyl. In embodiments, R⁸ is butyl. In embodiments, R⁸ is isobutyl. In embodiments, R⁸ is tert-butyl.

In embodiments, R⁴ is hydrogen, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), or —SO₂NR^{4A}R^{4B}. In embodiments, R⁴ is hydrogen, methyl, ethyl, propyl, butyl, isopropyl, isobutyl, tert-butyl, or —SO₂NR^{4A}R^{4B}. In embodiments, R⁴ is hydrogen. In embodiments, R⁴ is methyl. In embodiments, R⁴ is ethyl. In embodiments, R⁴ is propyl. In embodiments, R⁴ is butyl. In embodiments, R⁴ is isobutyl. In embodiments, R⁴ is tert-butyl.

In embodiments, R⁴ is —SO₂NR^{4A}R^{4B}, wherein each R^{4A} and R^{4B} is independently as described herein including embodiments. In embodiments, R⁴ is —SO₂NR^{4A}R^{4B}, wherein each R^{4A} and R^{4B} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R⁴ is —SO₂NR^{4A}R^{4B}, wherein each R^{4A} and R^{4B} is independently hydrogen. In embodiments, R⁴ is —SO₂NR^{4A}R^{4B}, wherein each R^{4A} and R^{4B} is independently substituted (e.g. with a substituent group, a size-limited substituent group or a lower substituent group) alkyl (e.g., C₁-C₈ alkyl, C₁-C₆ alkyl, or C₁-C₄ alkyl). In embodiments, R⁴ is —SO₂NR^{4A}R^{4B}, wherein each R^{4A} and R^{4B} is independently unsubstituted alkyl (e.g., C₁-C₈ alkyl, C₁-C₆ alkyl, or C₁-C₄ alkyl). In embodiments, R⁴ is —SO₂NR^{4A}R^{4B}, wherein each R^{4A} and R^{4B} is independently hydrogen or methyl. In embodiments, R⁴ is —SO₂NR^{4A}R^{4B}, wherein each R^{4A} and R^{4B} is independently hydrogen. In embodiments, R⁴ is —SO₂NR^{4A}R^{4B}, wherein each R^{4A} and R^{4B} is independently methyl.

In embodiments, R⁴ is —SO₂NH₂. In embodiments, R⁴ is —SO₂NHCH₃. In embodiments, R⁴ is —SO₂N(CH₃)₂.

In embodiments, R⁵ is hydrogen, oxo,

halogen, —CCl₃, —CBr₃, —CF₃, —C₁₃, —CH₂Cl, —CH₂Br, —CH₂F, —CH₂I, —CHCl₂, —CHBr₂, —CHF₂, —CHI₂, —CN, —OR^{5A}, —NR^{5A}R^{5B}, —COOH, —CONH₂, —NO₂, —SH, substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), or substituted (e.g. with a substituent group, a size-limited substituent group or a lower substituent group) or unsubstituted alkyl (e.g., C₁-C₈ alkyl, C₁-C₆ alkyl, or C₁-C₄ alkyl).

In embodiments, R⁵ is hydrogen, methyl, oxo,

halogen, —CCl₃, —CBr₃, —CF₃, —CI₃, —CH₂Cl, —CH₂Br,
—CH₂F, —CH₂I, —CHCl₂, —CHBr₂, —CHF₂, —CHI₂, —CN, —OH, —NR^{5A}R^{5B}, —COOH, —CONH₂, —NO₂, or —SH. In embodiments, R⁵ is hydrogen, methyl, halogen, oxo, —NR^{5A}R^{5B}, or unsubstituted heteroalkyl (e.g., 2 to 8 membered heteroalkyl, 2 to 6 membered heteroalkyl, or 2 to 4 membered heteroalkyl). In embodiments, R⁵ is hydrogen. In embodiments, R⁵ is methyl. In embodiments, R⁵ is halogen. In embodiments, R⁵ is Cl. In embodiments, R⁵ is Br. In embodiments, R⁵ is F. In embodiments, R⁵ is I. In embodiments, R⁵ is oxo. In embodiments, R⁵ is —NR^{5A}R^{5B}. In embodiments, R⁵ is unsubstituted alkoxy. In embodiments, R⁵ is methoxy, ethoxy, propoxy, butoxy or pentoxy. In embodiments, R⁵ is methoxy. In embodiments, R⁵ is ethoxy. In embodiments, R⁵ is propoxy. In embodiments, R⁵ is butoxy. In embodiments, R⁵ is pentoxy.

In embodiments, R⁵ is —NR^{5A}R^{5B}, wherein each R^{5A} and R^{5B} is independently as described herein including embodiments. In embodiments, R⁵ is —NR^{5A}R^{5B}, wherein each R^{5A} and R^{5B} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R⁵ is —NR^{5A}R^{5B}, wherein each R^{5A} and R^{5B} is independently hydrogen. In embodiments, R⁵ is —NR^{5A}R^{5B}, wherein each R^{5A} and R^{5B} is independently substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R⁵ is —NR^{5A}R^{5B}, wherein each R^{5A} and R^{5B} is independently substituted (e.g. with a substituent group, a size-limited substituent group or a lower substituent group) alkyl (e.g., C₁-C₈ alkyl, C₁-C₆ alkyl, or C₁-C₄ alkyl). In embodiments, R⁵ is —NR^{5A}R^{5B}, wherein each R^{5A} and R^{5B} is independently unsubstituted alkyl (e.g., C₁-C₈ alkyl, C₁-C₆ alkyl, or C₁-C₄ alkyl). In embodiments, R⁵ is —NR^{5A}R^{5B}, wherein each R^{5A} and R^{5B} is independently hydrogen or methyl. In embodiments, R⁵ is —NR^{5A}R^{5B}, wherein each R^{5A} and R^{5B} is independently hydrogen. In embodiments, R⁵ is —NR^{5A}R^{5B}, wherein each R^{5A} and R^{5B} is independently methyl.

In embodiments, R⁵ is —NH₂. In embodiments, R⁵ is —NHCH₃. In embodiments, R⁵ is —N(CH₃)₂.

In embodiments, R⁶ is hydrogen, halogen, or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R⁶ is hydrogen. In embodiments, R⁶ is halogen. In embodiments, R⁶ is F. In embodiments, R⁶ is Cl. In embodiments, R⁶ is Br. In embodiments, R⁶ is I. In embodiments, R⁶ is substituted (e.g. with a substituent group, a size-limited substituent group or a lower substituent group) alkyl (e.g., C₁-C₈ alkyl, C₁-C₆ alkyl, or C₁-C₄ alkyl). In embodiments, R⁶ is unsubstituted alkyl (e.g., C₁-C₈ alkyl, C₁-C₆ alkyl, or C₁-C₄ alkyl). In embodiments, R⁶ is methyl, ethyl, propyl, butyl, isopropyl, isobutyl or tert-butyl. In embodiments, R⁶ is methyl. In embodiments, R⁶ is ethyl. In embodiments, R⁶ is propyl. In embodiments, R⁶ is butyl. In embodiments, R⁶ is isopropyl. In embodiments, R⁶ is isobutyl. In embodiments, R⁶ is tert-butyl.

In embodiments, provided herein is an MHC-peptide antigen stabilizing compound having the structure of formula III, or a salt thereof.

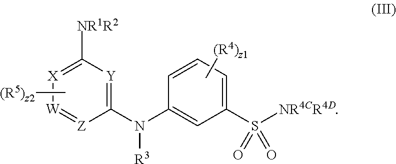

R¹, R², R⁴, R⁵, X, Y, W, Z, and z2 are as described herein including embodiments. R³ is hydrogen or methyl. z1 is 0 to 4. Each R^{4C} and R^{4D} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4C} and R^{4D} substituents bonded to the same nitrogen atom may optionally be joined to form a substituted or unsubstituted heterocycloalkyl or substituted or unsubstituted heteroaryl.

In embodiments, R³ is hydrogen or methyl. R³ is hydrogen. R³ is methyl.

In embodiments, z1 is 0. In embodiments, z1 is 1. In embodiments, z1 is 2. In embodiments, z1 is 3. In embodiments, z1 is 4.

In embodiments, each R^{4C} and R^{4D} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted aryl (e.g., C₆-C₁₀ aryl, C₁₀ aryl, or phenyl), substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), or substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). R^{4C} and R^{4D} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl).

In embodiments, each R^{4C} and R^{4D} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, each R^{4C} and R^{4D} is independently hydrogen or unsubstituted alkyl (e.g., C₁-C₈ alkyl, C₁-C₆ alkyl, or C₁-C₄ alkyl). In embodiments, each R^{4C} and R^{4D} is independently hydrogen. In embodiments, each R^{4C} and R^{4D} is independently unsubstituted alkyl (e.g., C₁-C₈ alkyl, C₁-C₆ alkyl, or C₁-C₄ alkyl). In embodiments, each R^{4C} and R^{4D} is independently hydrogen, methyl, ethyl, propyl, butyl, pentyl or hexyl. In embodiments, each R^{4C} and R^{4D} is independently hydrogen. In embodiments, each R^{4C} and R^{4D} is independently methyl. In embodiments, each R^{4C} and R^{4D} is independently ethyl. In embodiments, each R^{4C} and R^{4D} is independently propyl. In embodiments, each R^{4C} and R^{4D} is independently butyl. In embodiments, each R^{4C} and R^{4D} is independently pentyl. In embodiments, each R^{4C} and R^{4D} is independently hexyl.

In embodiments, R^{4C} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{4C} is independently hydrogen or unsubstituted alkyl (e.g., C₁-C₈ alkyl, C₁-C₆ alkyl, or C₁-C₄ alkyl). In embodiments, R^{4C} is independently hydrogen. In embodiments, R^{4C} is independently unsubstituted alkyl (e.g., C₁-C₈ alkyl, C₁-C₆ alkyl, or C₁-C₄ alkyl). In embodiments, R^{4C} is independently hydrogen, methyl, ethyl, propyl, butyl, pentyl or hexyl. In embodiments, R^{4C} is independently hydrogen. In embodiments, R^{4C} is independently methyl. In embodiments, R^{4C} is independently ethyl. In embodiments, R^{4C} is independently propyl. In embodiments, R^{4C} is independently butyl. In embodiments, R^{4C} is independently pentyl. In embodiments, R^{4C} is independently hexyl.

In embodiments, R^{4D} is independently hydrogen or substituted (e.g. with a substituent group, a size-limited substituent group or a lower substituent group) or unsubstituted alkyl (e.g., C₁-C₈ alkyl, C₁-C₆ alkyl, or C₁-C₄ alkyl). In embodiments, R^{4D} is independently hydrogen or unsubstituted alkyl (e.g., C₁-C₈ alkyl, C₁-C₆ alkyl, or C₁-C₄ alkyl). In embodiments, R^{4D} is independently hydrogen. In embodiments, R^{4D} is independently unsubstituted alkyl (e.g., C₁-C₈ alkyl, C₁-C₆ alkyl, or C₁-C₄ alkyl). In embodiments, R^{4D} is independently hydrogen, methyl, ethyl, propyl, butyl, pentyl or hexyl. In embodiments, R^{4D} is independently hydrogen. In embodiments, R^{4D} is independently methyl. In embodiments, R^{4D} is independently ethyl. In embodiments, R^{4D} is independently propyl. In embodiments, R^{4D} is independently butyl. In embodiments, R^{4D} is independently pentyl. In embodiments, R^{4D} is independently hexyl.

In embodiments, provided herein is an MHC-peptide antigen stabilizing compound having the structure of formula IV, or a salt thereof:

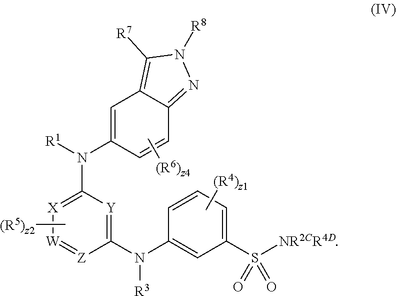

R¹, R³, R⁴, R⁵, R⁶, R⁷, R⁸, R^{4C}, R^{4D}, X, Y, W, Z, z1, z2 and z4 are as described herein including embodiments.

In embodiments, provided herein is an MHC-peptide antigen stabilizing compound having the structure of formula IV, or a salt thereof:

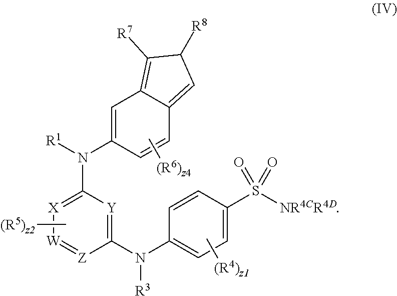

R¹, R³, R⁴, R⁵, R⁶, R⁷, R⁸, R^{4C}, R^{4D}, X Y, W, Z, z1, z2 and z4 are as described herein including embodiments.

In embodiments, provided herein is an MHC-peptide antigen stabilizing compound having the formula:

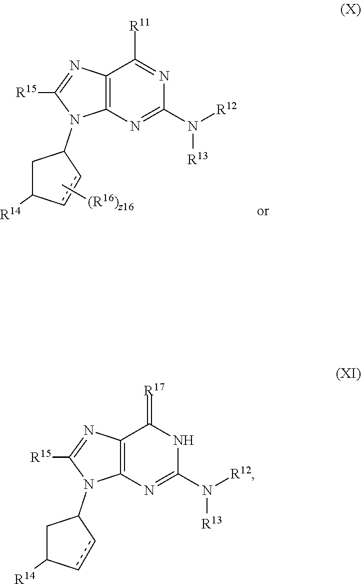

or salts thereof.

R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NHC(O)NR^{11A}R^{11B}, —N(O)ₘ₁₁, —NR^{11A}R^{11B}, —C(O)R^{11C}, —C(O)—OR^{11C}, —C(O)NR^{11A}R^{11B}, —OR^{11D}, —NR^{11A}CH₂C(O)R^{11C}, —NR^{11A}CH₂SO₂R^{11D}, —NR^{11A}SO₂R^{11D}, —NR^{11A}C(O)R^{11C}, —NR^{11A}C(O)OR^{11C}, —NR^{11A}OR^{11C}, —NR^{11A}OSO₂R^{11D}, —NR^{11A}OCH₂C(O)R^{11C}, —NR^{11A}CH₂P(O)R^{11C}R^{11D}, —PO^{q11}R^{11A}, —POᵣ₁₁R^{11C}R^{11D}, —POᵣ₁₁NR^{11A}R^{11B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹² is hydrogen,

halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹⁴ is —CH₂OR^{14A}, —C(O)OR^{14B}, or —CH₂OC(═NH)R^{14C}.

R¹⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹⁶ is independently hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹⁷ is ═O, ═S, or ═NR^{17A}.

each R^{11A}, R^{11B}, R^{11C}, and R^{11D} is independently

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —OSO₃H, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl.

R^{14A} and R^{14B} are independently hydrogen or unsubstituted C₁-C₅ alkyl.

R^{14C} is unsubstituted C₁-C₅ alkyl.

R^{17A} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —OSO₃H, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X¹¹ is —Cl, —Br, —I or —F.

n11 is an integer from 0 to 4.

v11 is 1 or 2.

m11 is an integer from 0 to 3.

each q11 and r11 is independently 1 or 2.

z16 is independently an integer from 0 to 8.

In embodiments, provided herein is an MHC-peptide antigen stabilizing compound having the formula:

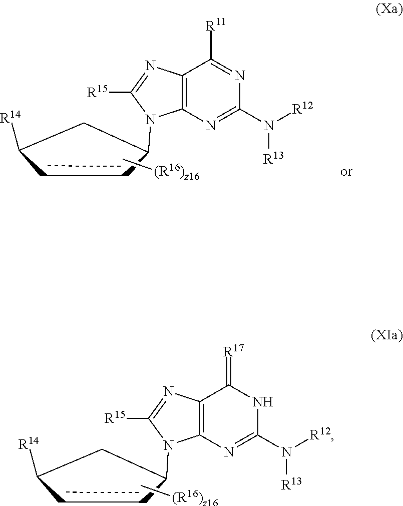

or salts thereof. R¹¹, R¹², R¹³, R¹⁴, R¹⁵, R¹⁶, R¹⁷, and z16 are as described herein including embodiments. In embodiments, the MHC-peptide antigen stabilizing compound has the formula:

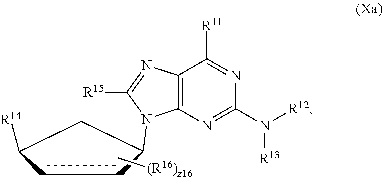

or salts thereof. R¹¹, R¹², R¹³, R¹⁴, R¹⁵, R¹⁶, and z16 are as described herein including embodiments. In embodiments, the MHC-peptide antigen stabilizing compound has the formula:

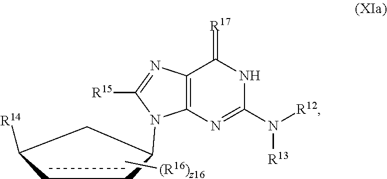

or salts thereof. R¹², R¹³, R¹⁴, R¹⁵, R¹⁶, R¹⁷, and z16 are as described herein including embodiments.

In embodiments, provided herein is an MHC-peptide antigen stabilizing compound having the formula:

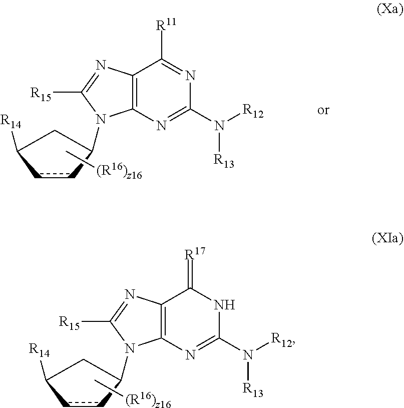

or salts thereof; R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOₙ₁₁R^{11D}, —SOᵥ₁₁NR^{11A}R^{11B}, —NHC(O)NR^{11A}R^{11B}, —N(O)ₘ₁₁, —NR^{11 A}R^{11B}, —C(O)R^{11C}, —C(O)—OR^{11C}, —C(O)NR^{11A}R^{11B}, —OR^{11D}, —NR^{11A}CH₂C(O)R^{11C}, —NR^{11A}CH₂SO₂R^{11D}, —NR^{11A}SO₂R^{11D}, —NR^{11A}C(O)R^{11C}, —NR^{11A}C(O)OR^{11C}, —NR^{11A}OR^{11C}, —NR^{11A}OSO₂R^{11D}, —NR^{11A}OCH₂C(O)R^{11C}, —NR^{11A}CH₂P(O)R^{11C}R^{11D}, —PO_{q11}R^{11A}, —POᵣ₁₁R^{11C}R^{11D}, —POᵣ₁₁NR^{11A}R^{11B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CH 12, —CN, —OH, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹³ is hydrogen,
halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁴ is —CH₂OR^{14A}, —C(O)OR^{14B}, or —CH₂OC(═NH)R^{14C}; R¹⁵ is hydrogen,
halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁶ is independently hydrogen,
halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R¹⁷ is ═O, ═S, or ═NR^{17A}; each R^{11A}, R^{11B}, R^{11C}, and R^{11D} is independently
hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —OSO₃H, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl; R^{14A} and R^{14B} are independently hydrogen or unsubstituted C₁-C₅ alkyl; R^{14C} is unsubstituted C₁-C₅ alkyl; R^{17A} is independently
hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —OSO₃H,
—NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X¹¹ is —Cl, —Br, —I or —F; n11 is independently an integer from 0 to 4; v11 is independently 1 or 2; m11 is independently an integer from 0 to 3; each q11 and r11 is independently 1 or 2; and z16 is independently an integer from 0 to 8.

In embodiments, R¹² is hydrogen.

In embodiments, R¹³ is hydrogen.

In embodiments, R¹⁵ is hydrogen.

In embodiments, R¹⁴ is —CH₂OR^{14A}.

In embodiments, R^{14A} is hydrogen. In embodiments, R^{14A} is unsubstituted C₁-C₅ alkyl. In embodiments, R^{14A} is unsubstituted methyl. In embodiments, R^{14A} is unsubstituted ethyl. In embodiments, R^{14A} is unsubstituted propyl.

In embodiments, R^{14B} is hydrogen. In embodiments, R^{14B} is unsubstituted C₁-C₅ alkyl. In embodiments, R^{14B} is unsubstituted methyl. In embodiments, R^{14B} is unsubstituted ethyl. In embodiments, R^{14B} is unsubstituted propyl.

In embodiments, R^{14C} is unsubstituted methyl. In embodiments, R^{14C} is unsubstituted ethyl. In embodiments, R^{14C} is unsubstituted propyl.

In embodiments, the MHC-peptide antigen stabilizing compound has the formula:

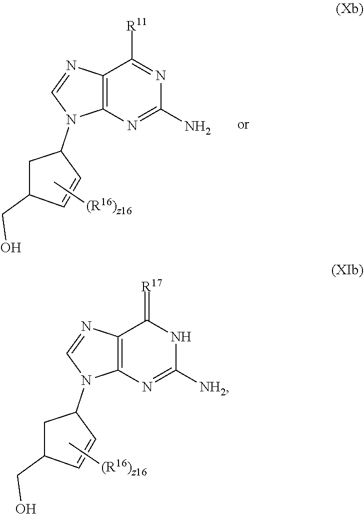

or salts thereof. R¹, R¹⁶, R¹⁷, and z16 are as described herein, including embodiments.

In embodiments, the MHC-peptide antigen stabilizing compound has the formula:

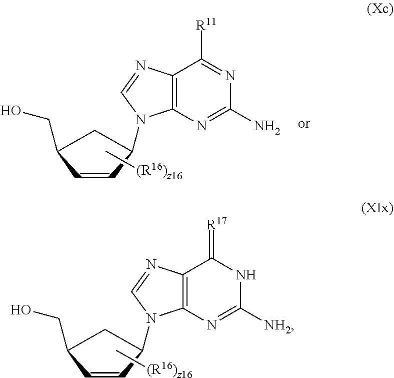

or salts thereof. R¹¹, R¹⁶, R¹⁷, and z16 are as described herein, including embodiments

In embodiments, the MHC-peptide antigen stabilizing compound has the formula:

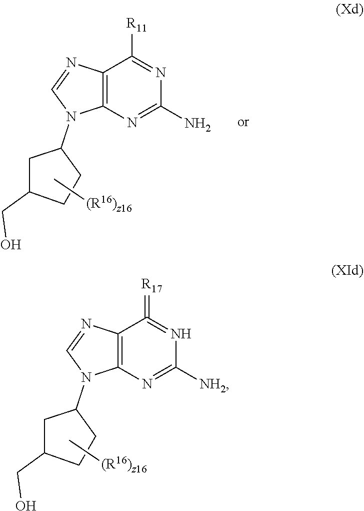

or salts thereof. R¹, R¹⁶, R¹⁷, and z16 are as described herein, including embodiments.

In embodiments, the MHC-peptide antigen stabilizing compound has the formula:

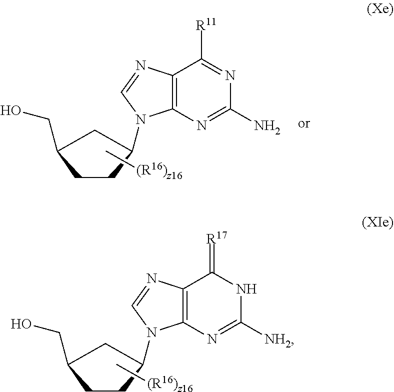

or salts thereof. R¹¹, R¹⁶, R¹⁷, and z16 are as described herein, including embodiments.

In embodiments, R¹⁶ is independently hydrogen or —OH.

In embodiments, z16 is 1 or 2.

In embodiments, z16 is 0.

In embodiments, R¹¹ is hydrogen, halogen, —CCl₃, —CBr₃, —CF₃, —CI₃, —CH₂C₁, —CH₂Br, —CH₂F, —CH₂I, —CHCl₂, —CHBr₂, —CHF₂, —CHI₂, —CN, —OH, —ONH₂, —NR^{11A}R^{11B}, —COOH, —COO(C₁-C₄ alkyl), —CONH₂, —NO₂, —SH, —SO₂OH, —SO₂NH₂, —PO(OH)₂, —OCCl₃, —OCF₃, —OCBr₃, —OCI₃, —OCHCl₂, —OCHBr₂, —OCHI₂, —OCHF₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is —NR^{11A}R^{11B}.

In embodiments, R¹¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R^{11A} and R^{11B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl.

In embodiments, R^{11A} and R^{11B} are independently hydrogen, substituted or unsubstituted C₁-C₄ alkyl, or substituted or unsubstituted C₃-C₆ cycloalkyl.

In embodiments, R^{11A} and R^{11B} substituents bonded to the same nitrogen atom are joined to form a substituted or unsubstituted 4 to 6 membered heterocycloalkyl or substituted or unsubstituted 5 to 6 membered heteroaryl.

In embodiments, R^{11A} and R^{11B} are independently hydrogen, —COCHCH₂, —CH₂COOH, —CH₂SO₂OH, —OSO₂OH, —CH₂P(O)(OH)₂, or —OCH₂COOH.

In embodiments, R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SOₙ₁R^{1A}, —SO^{v1}NR^{1A}R^{1B}, —POₘ₁R^{1A}, —POᵣ₁NR^{1A}R^{1B}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted aryl (e.g., C₆-C₁₀ aryl, C₁₀ aryl, or phenyl), substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), or substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R¹ is substituted with one or more substituent groups. In embodiments, R¹ is substituted with one or more size-limited substituent groups. In embodiments, R¹ is substituted with one or more lower substituent groups.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SOₙ₂R^{2A}, —SOᵥ₂NR^{2A}R^{2B}, —POₘ₂R^{2A}, —POᵣ₂NR^{2A}R^{2B}, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted aryl (e.g., C₆-C₁₀ aryl, C₁₀ aryl, or phenyl), substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), or substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R² is substituted with one or more substituent groups. In embodiments, R² is substituted with one or more size-limited substituent groups. In embodiments, R² is substituted with one or more lower substituent groups.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R³ is hydrogen, halogen, —CCl₃, —CBr₃, —CF₃, —CI₃, —CH₂Cl, —CH₂Br, —CH₂F, —CH₂I, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted aryl (e.g., C₆-C₁₀ aryl, C₁₀ aryl, or phenyl), substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), or substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R³ is substituted with one or more substituent groups. In embodiments, R³ is substituted with one or more size-limited substituent groups. In embodiments, R³ is substituted with one or more lower substituent groups.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, R⁴ is hydrogen, oxo,

halogen, —CCl₃, —CBr₃, —CF₃, —C₁₃, —CH₂Cl, —CH₂Br, —CH₂F, —CH₂I, —CHCl₂, —CHBr₂, —CHF₂, —CHI₂, —CN, —OR^{4A}, —NR^{4A}R^{4B}, —COOR^{4A}, —CONR^{4A}R^{4B}, —NO₂, —SR^{4A}, —SOₙ₄R^{4A}, —SOᵥ₄NR^{4A}R^{4B}, —PO(OH)₂, —POₘ₄R^{4A}, —POᵣ₄NR^{4A}R^{4B}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted aryl (e.g., C₆-C₁₀ aryl, C₁₀ aryl, or phenyl), substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), or substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R⁴ is substituted with one or more substituent groups. In embodiments, R⁴ is substituted with one or more size-limited substituent groups. In embodiments, R⁴ is substituted with one or more lower substituent groups.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, R⁵ is hydrogen, oxo,

halogen, —CCl₃, —CBr₃, —CF₃, —C₁₃, —CH₂Cl, —CH₂Br, —CH₂F, —CH₂I, —CHCl₂, —CHBr₂, —CHF₂, —CHI₂, —CN, —OR^{5A}, —NR^{5A}R^{5B}, —COOH, —CONH₂, —NO₂, —SH, —SOₙ₅R^{5A}, —PO(OH)₂, —POₘ₅R^{5A}, —POᵣ₅NR^{5A}R^{5B}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted aryl (e.g., C₆-C₁₀ aryl, C₁₀ aryl, or phenyl), substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), or substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R⁵ is substituted with one or more substituent groups. In embodiments, R⁵ is substituted with one or more size-limited substituent groups. In embodiments, R⁵ is substituted with one or more lower substituent groups.

In embodiments, a substituted R⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁶ is hydrogen, oxo,

halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{6A}, —NH₂, —COOH, —CONH₂, —NO₂, —SH, —SOₙ₆R^{6A}, —SOᵥ₆NR^{6A}R^{6B}, —PO(OH)₂, —POₘ₆R^{6A}, —POᵣ₆NR^{6A}R^{6B}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted aryl (e.g., C₆-C₁₀ aryl, C₁₀ aryl, or phenyl), substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), or substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R⁶ is substituted with one or more substituent groups. In embodiments, R⁶ is substituted with one or more size-limited substituent groups. In embodiments, R⁶ is substituted with one or more lower substituent groups.

In embodiments, a substituted R⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁶ is substituted with a plurality of groups selected from substituent groups, size-limited substituent groups, and lower substituent groups; each substituent group, size-limited substituent group, and/or lower substituent group may optionally be different. In embodiments, when R⁶ is substituted, it is substituted with at least one substituent group. In embodiments, when R⁶ is substituted, it is substituted with at least one size-limited substituent group. In embodiments, when R⁶ is substituted, it is substituted with at least one lower substituent group.

In embodiments, R⁷ is hydrogen, halogen, —CCl₃, —CBr₃, —CF₃, —C₁₃, —CH₂Cl, —CH₂Br, —CH₂F, —CH₂I, —CHCl₂, —CHBr₂, —CHF₂, —CHI₂, —CN, —OR^{7A}, —NH₂, —COOH, —CONH₂, —NO₂, —SH, —SOₙ₇R^{7A}, —SOᵥ₇NR^{7A}R^{7B}, —PO(OH)₂, —POₘ₇R^{7A}, —POᵣ₇NR^{7A}R^{7B}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted aryl (e.g., C₆-C₁₀ aryl, C₁₀ aryl, or phenyl), substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), or substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R⁷ is substituted with one or more substituent groups. In embodiments, R⁷ is substituted with one or more size-limited substituent groups. In embodiments, R⁷ is substituted with one or more lower substituent groups.

In embodiments, a substituted R⁷ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁷ is substituted with a plurality of groups selected from substituent groups, size-limited substituent groups, and lower substituent groups; each substituent group, size-limited substituent group, and/or lower substituent group may optionally be different. In embodiments, when R⁷ is substituted, it is substituted with at least one substituent group. In embodiments, when R⁷ is substituted, it is substituted with at least one size-limited substituent group. In embodiments, when R⁷ is substituted, it is substituted with at least one lower substituent group.

In embodiments, R⁸ is hydrogen, halogen, —CCl₃, —CBr₃, —CF₃, —C₁₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SOₙ₈R^{8A}, —SOᵥ₈NR^{8A}R^{8B}, —PO(OH)₂, —POₘ₈R^{8A}, —POᵣ₈NR^{8A}R^{8B}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted aryl (e.g., C₆-C₁₀ aryl, C₁₀ aryl, or phenyl), substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), or substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl). In embodiments, R⁸ is substituted with one or more substituent groups. In embodiments, R⁸ is substituted with one or more size-limited substituent groups. In embodiments, R⁸ is substituted with one or more lower substituent groups.

In embodiments, a substituted R⁸ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁸ is substituted with a plurality of groups selected from substituent groups, size-limited substituent groups, and lower substituent groups; each substituent group, size-limited substituent group, and/or lower substituent group may optionally be different. In embodiments, when R⁸ is substituted, it is substituted with at least one substituent group. In embodiments, when R⁸ is substituted, it is substituted with at least one size-limited substituent group. In embodiments, when R⁸ is substituted, it is substituted with at least one lower substituent group.

In embodiments, R^{1A}, R^{1B}, R^{2A}, R^{2B}, R^{4A}, R^{4B}, R^{5A}, R^{5B}, R^{6A}, R^{6B}, R^{7A}, R^{7B}, R^{8A}, and R^{8B} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{1A}, R^{1B}, R^{2A}, R^{2B}, R^{4A}, R^{4B}, R^{5A}, R^{5B}, R^{6A}, R^{6B}, R^{7A}, R^{7B}, R^{8A}, and R^{8B} is independently substituted with one or more substituent groups. In embodiments, each R^{1A}, R^{1B}, R^{2A}, R^{2B}, R^{4A}, R^{4B}, R^{5A}, R^{5B}, R^{6A}, R^{6B}, R^{7A}, R^{7B}, R^{8A}, and R^{8B} is independently substituted with one or more size-limited substituent groups. In embodiments, each R^{1A}, R^{1B}, R^{2A}, R^{2B}, R^{4A}, R^{4B}, R^{5A}, R^{5B}, R^{6A}, R^{6B}, R^{7A}, R^{7B}, R^{8A}, and R^{8B} is independently substituted with one or more lower substituent groups.

In embodiments, R^{1A} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{1A} is independently substituted with one or more substituent groups. In embodiments, each R^{1A} is independently substituted with one or more size-limited substituent groups. In embodiments, each R^{1A} is independently substituted with one or more lower substituent groups.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, R^{1B} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{1B} is independently substituted with one or more substituent groups. In embodiments, each R^{1B} is independently substituted with one or more size-limited substituent groups. In embodiments, each R^{1B} is independently substituted with one or more lower substituent groups.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, R^{2A} is independently hydrogen, —CX₃, —CHX₂, —CH₂X, —CN, —OH, —NH₂, —COOH,

—CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{2A} is independently substituted with one or more substituent groups. In embodiments, each R^{2A} is independently substituted with one or more size-limited substituent groups. In embodiments, each R^{2A} is independently substituted with one or more lower substituent groups.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, R^{2B} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{2B} is independently substituted with one or more substituent groups. In embodiments, each R^{2B} is independently substituted with one or more size-limited substituent groups. In embodiments, each R^{2B} is independently substituted with one or more lower substituent groups.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, R^{4A} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{4A} is independently substituted with one or more substituent groups. In embodiments, each R^{4A} is independently substituted with one or more size-limited substituent groups. In embodiments, each R^{4A} is independently substituted with one or more lower substituent groups.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, R^{4B} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{4B} is independently substituted with one or more substituent groups. In embodiments, each R^{4B} is independently substituted with one or more size-limited substituent groups. In embodiments, each R^{4B} is independently substituted with one or more lower substituent groups.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, R^{4C} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{4C} is independently substituted with one or more substituent groups. In embodiments, each R^{4C} is independently substituted with one or more size-limited substituent groups. In embodiments, each R^{4C} is independently substituted with one or more lower substituent groups.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, R^{4D} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{4D} is independently substituted with one or more substituent groups. In embodiments, each R^{4D} is independently substituted with one or more size-limited substituent groups. In embodiments, each R^{4D} is independently substituted with one or more lower substituent groups.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, R^{5A} is independently hydrogen, —CX₃, —CHX₂, —CH₂X, —CN, —OH, —NH₂, —COOH,

—CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{5A} is independently substituted with one or more substituent groups. In embodiments, each R^{5A} is independently substituted with one or more size-limited substituent groups. In embodiments, each R^{5A} is independently substituted with one or more lower substituent groups.

In embodiments, a substituted R^{5A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5A} is substituted with a plurality of groups selected from substituent groups, size-limited substituent groups, and lower substituent groups; each substituent group, size-limited substituent group, and/or lower substituent group may optionally be different. In embodiments, when R^{5A} is substituted, it is substituted with at least one substituent group. In embodiments, when R^{5A} is substituted, it is substituted with at least one size-limited substituent group. In embodiments, when R^{5A} is substituted, it is substituted with at least one lower substituent group.

In embodiments, R^{5B} is independently hydrogen, —CX₃, —CHX₂, —CH₂X, —CN, —OH, —NH₂, —COOH,

—CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{5B} is independently substituted with one or more substituent groups. In embodiments, each R^{5B} is independently substituted with one or more size-limited substituent groups. In embodiments, each R^{5B} is independently substituted with one or more lower substituent groups.

In embodiments, a substituted R^{5B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{5B} is substituted with a plurality of groups selected from substituent groups, size-limited substituent groups, and lower substituent groups; each substituent group, size-limited substituent group, and/or lower substituent group may optionally be different. In embodiments, when R^{5B} is substituted, it is substituted with at least one substituent group. In embodiments, when R^{5B} is substituted, it is substituted with at least one size-limited substituent group. In embodiments, when R^{5B} is substituted, it is substituted with at least one lower substituent group.

In embodiments, R^{6A} is independently hydrogen, —CX₃, —CHX₂, —CH₂X, —CN, —OH, —NH₂, —COOH,

—CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{6A} is independently substituted with one or more substituent groups. In embodiments, each R^{6A} is independently substituted with one or more size-limited substituent groups. In embodiments, each R^{6A} is independently substituted with one or more lower substituent groups.

In embodiments, a substituted R^{6A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6A} is substituted with a plurality of groups selected from substituent groups, size-limited substituent groups, and lower substituent groups; each substituent group, size-limited substituent group, and/or lower substituent group may optionally be different. In embodiments, when R^{6A} is substituted, it is substituted with at least one substituent group. In embodiments, when R^{6A} is substituted, it is substituted with at least one size-limited substituent group. In embodiments, when R^{6A} is substituted, it is substituted with at least one lower substituent group.

In embodiments, R^{6B} is independently hydrogen, —CX₃, —CHX₂, —CH₂X, —CN, —OH, —NH₂, —COOH,

—CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{6B} is independently substituted with one or more substituent groups. In embodiments, each R^{6B} is independently substituted with one or more size-limited substituent groups. In embodiments, each R^{6B} is independently substituted with one or more lower substituent groups.

In embodiments, a substituted R^{6B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{6B} is substituted with a plurality of groups selected from substituent groups, size-limited substituent groups, and lower substituent groups; each substituent group, size-limited substituent group, and/or lower substituent group may optionally be different. In embodiments, when R^{6B} is substituted, it is substituted with at least one substituent group. In embodiments, when R^{6B} is substituted, it is substituted with at least one size-limited substituent group. In embodiments, when R^{6B} is substituted, it is substituted with at least one lower substituent group.

In embodiments, R^{7A} is independently hydrogen, —CX₃, —CHX₂, —CH₂X, —CN, —OH, —NH₂, —COOH,

—CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{7A} is independently substituted with one or more substituent groups. In embodiments, each R^{7A} is independently substituted with one or more size-limited substituent groups. In embodiments, each R^{7A} is independently substituted with one or more lower substituent groups.

In embodiments, a substituted R^{7A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7A} is substituted with a plurality of groups selected from substituent groups, size-limited substituent groups, and lower substituent groups; each substituent group, size-limited substituent group, and/or lower substituent group may optionally be different. In embodiments, when R^{7A} is substituted, it is substituted with at least one substituent group. In embodiments, when R^{7A} is substituted, it is substituted with at least one size-limited substituent group. In embodiments, when R^{7A} is substituted, it is substituted with at least one lower substituent group.

In embodiments, R^{7B} is independently hydrogen, —CX₃, —CHX₂, —CH₂X, —CN, —OH, —NH₂, —COOH,

—CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{7B} is independently substituted with one or more substituent groups. In embodiments, each R^{7B} is independently substituted with one or more size-limited substituent groups. In embodiments, each R^{7B} is independently substituted with one or more lower substituent groups.

In embodiments, a substituted R^{7B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{7B} is substituted with a plurality of groups selected from substituent groups, size-limited substituent groups, and lower substituent groups; each substituent group, size-limited substituent group, and/or lower substituent group may optionally be different. In embodiments, when R^{7B} is substituted, it is substituted with at least one substituent group. In embodiments, when R^{7B} is substituted, it is substituted with at least one size-limited substituent group. In embodiments, when R^{7B} is substituted, it is substituted with at least one lower substituent group.

In embodiments, R^{8A} is independently hydrogen, —CX₃, —CHX₂, —CH₂X, —CN, —OH, —NH₂, —COOH,

—CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{8A} is independently substituted with one or more substituent groups. In embodiments, each R^{8A} is independently substituted with one or more size-limited substituent groups. In embodiments, each R^{8A} is independently substituted with one or more lower substituent groups.

In embodiments, a substituted R^{8A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8A} is substituted with a plurality of groups selected from substituent groups, size-limited substituent groups, and lower substituent groups; each substituent group, size-limited substituent group, and/or lower substituent group may optionally be different. In embodiments, when R^{8A} is substituted, it is substituted with at least one substituent group. In embodiments, when R^{8A} is substituted, it is substituted with at least one size-limited substituent group. In embodiments, when R^{8A} is substituted, it is substituted with at least one lower substituent group.

In embodiments, R^{8B} is independently hydrogen, —CX₃, —CHX₂, —CH₂X, —CN, —OH, —NH₂, —COOH,

—CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{8B} is independently substituted with one or more substituent groups. In embodiments, each R^{8B} is independently substituted with one or more size-limited substituent groups. In embodiments, each R^{8B} is independently substituted with one or more lower substituent groups.

In embodiments, a substituted R^{8B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{8B} is substituted with a plurality of groups selected from substituent groups, size-limited substituent groups, and lower substituent groups; each substituent group, size-limited substituent group, and/or lower substituent group may optionally be different. In embodiments, when R^{8B} is substituted, it is substituted with at least one substituent group. In embodiments, when R^{8B} is substituted, it is substituted with at least one size-limited substituent group. In embodiments, when R^{8B} is substituted, it is substituted with at least one lower substituent group.

In embodiments, R^{1A} and R^{1B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{1A} and R^{1B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{1A} and R^{1B} substituents bonded to the same nitrogen atom is substituted, it is substituted with at least one substituent group. In embodiments, when a heterocycloalkyl formed by the joining of R^{1A} and R^{1B} substituents bonded to the same nitrogen atom is substituted, it is substituted with at least one size-limited substituent group. In embodiments, when a heterocycloalkyl formed by the joining of R^{1A} and R^{1B} substituents bonded to the same nitrogen atom is substituted, it is substituted with at least one lower substituent group. In embodiments, when a heteroaryl formed by the joining of R^{1A} and R^{1B} substituents bonded to the same nitrogen atom is substituted, it is substituted with at least one substituent group. In embodiments, when a heteroaryl formed by the joining of R^{1A} and R^{1B} substituents bonded to the same nitrogen atom is substituted, it is substituted with at least one size-limited substituent group. In embodiments, when a heteroaryl formed by the joining of R^{1A} and R^{1B} substituents bonded to the same nitrogen atom is substituted, it is substituted with at least one lower substituent group.

In embodiments, R^{2A} and R^{2B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{2A} and R^{2B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{2A} and R^{2B} substituents bonded to the same nitrogen atom is substituted, it is substituted with at least one substituent group. In embodiments, when a heterocycloalkyl formed by the joining of R^{2A} and R^{2B} substituents bonded to the same nitrogen atom is substituted, it is substituted with at least one size-limited substituent group. In embodiments, when a heterocycloalkyl formed by the joining of R^{2A} and R^{2B} substituents bonded to the same nitrogen atom is substituted, it is substituted with at least one lower substituent group. In embodiments, when a heteroaryl formed by the joining of R^{2A} and R^{2B} substituents bonded to the same nitrogen atom is substituted, it is substituted with at least one substituent group. In embodiments, when a heteroaryl formed by the joining of R^{2A} and R^{2B} substituents bonded to the same nitrogen atom is substituted, it is substituted with at least one size-limited substituent group. In embodiments, when a heteroaryl formed by the joining of R^{2A} and R^{2B} substituents bonded to the same nitrogen atom is substituted, it is substituted with at least one lower substituent group.

In embodiments, R^{4A} and R^{4B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{4A} and R^{4B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{4A} and R^{4B} substituents bonded to the same nitrogen atom is substituted, it is substituted with at least one substituent group. In embodiments, when a heterocycloalkyl formed by the joining of R^{4A} and R^{4B} substituents bonded to the same nitrogen atom is substituted, it is substituted with at least one size-limited substituent group. In embodiments, when a heterocycloalkyl formed by the joining of R^{4A} and R^{4B} substituents bonded to the same nitrogen atom is substituted, it is substituted with at least one lower substituent group. In embodiments, when a heteroaryl formed by the joining of R^{4A} and R^{4B} substituents bonded to the same nitrogen atom is substituted, it is substituted with at least one substituent group. In embodiments, when a heteroaryl formed by the joining of R^{4A} and R^{4B} substituents bonded to the same nitrogen atom is substituted, it is substituted with at least one size-limited substituent group. In embodiments, when a heteroaryl formed by the joining of R^{4A} and R^{4B} substituents bonded to the same nitrogen atom is substituted, it is substituted with at least one lower substituent group.

In embodiments, R^{5A} and R^{5B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{5A} and R^{5B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{5A} and R^{5B} substituents bonded to the same nitrogen atom is substituted, it is substituted with at least one substituent group. In embodiments, when a heterocycloalkyl formed by the joining of R^{5A} and R^{5B} substituents bonded to the same nitrogen atom is substituted, it is substituted with at least one size-limited substituent group. In embodiments, when a heterocycloalkyl formed by the joining of R^{5A} and R^{5B} substituents bonded to the same nitrogen atom is substituted, it is substituted with at least one lower substituent group. In embodiments, when a heteroaryl formed by the joining of R^{5A} and R^{5B} substituents bonded to the same nitrogen atom is substituted, it is substituted with at least one substituent group. In embodiments, when a heteroaryl formed by the joining of R^{5A} and R^{5B} substituents bonded to the same nitrogen atom is substituted, it is substituted with at least one size-limited substituent group. In embodiments, when a heteroaryl formed by the joining of R^{5A} and R^{5B} substituents bonded to the same nitrogen atom is substituted, it is substituted with at least one lower substituent group.

In embodiments, R^{6A} and R^{6B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{6A} and R^{6B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{6A} and R^{6B} substituents bonded to the same nitrogen atom is substituted, it is substituted with at least one substituent group. In embodiments, when a heterocycloalkyl formed by the joining of R^{6A} and R^{6B} substituents bonded to the same nitrogen atom is substituted, it is substituted with at least one size-limited substituent group. In embodiments, when a heterocycloalkyl formed by the joining of R^{6A} and R^{6B} substituents bonded to the same nitrogen atom is substituted, it is substituted with at least one lower substituent group. In embodiments, when a heteroaryl formed by the joining of R^{6A} and R^{6B} substituents bonded to the same nitrogen atom is substituted, it is substituted with at least one substituent group. In embodiments, when a heteroaryl formed by the joining of R^{6A} and R^{6B} substituents bonded to the same nitrogen atom is substituted, it is substituted with at least one size-limited substituent group. In embodiments, when a heteroaryl formed by the joining of R^{6A} and R^{6B} substituents bonded to the same nitrogen atom is substituted, it is substituted with at least one lower substituent group.

In embodiments, R^{7A} and R^{7B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{7A} and R^{7B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{7A} and R^{7B} substituents bonded to the same nitrogen atom is substituted, it is substituted with at least one substituent group. In embodiments, when a heterocycloalkyl formed by the joining of R^{7A} and R^{7B} substituents bonded to the same nitrogen atom is substituted, it is substituted with at least one size-limited substituent group. In embodiments, when a heterocycloalkyl formed by the joining of R^{7A} and R^{7B} substituents bonded to the same nitrogen atom is substituted, it is substituted with at least one lower substituent group. In embodiments, when a heteroaryl formed by the joining of R^{7A} and R^{7B} substituents bonded to the same nitrogen atom is substituted, it is substituted with at least one substituent group. In embodiments, when a heteroaryl formed by the joining of R^{7A} and R^{7B} substituents bonded to the same nitrogen atom is substituted, it is substituted with at least one size-limited substituent group. In embodiments, when a heteroaryl formed by the joining of R^{7A} and R^{7B} substituents bonded to the same nitrogen atom is substituted, it is substituted with at least one lower substituent group.

In embodiments, R^{8A} and R^{8B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{8A} and R^{8B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{8A} and R^{8B} substituents bonded to the same nitrogen atom is substituted, it is substituted with at least one substituent group. In embodiments, when a heterocycloalkyl formed by the joining of R^{8A} and R^{8B} substituents bonded to the same nitrogen atom is substituted, it is substituted with at least one size-limited substituent group. In embodiments, when a heterocycloalkyl formed by the joining of R^{8A} and R^{8B} substituents bonded to the same nitrogen atom is substituted, it is substituted with at least one lower substituent group. In embodiments, when a heteroaryl formed by the joining of R^{8A} and R^{8B} substituents bonded to the same nitrogen atom is substituted, it is substituted with at least one substituent group. In embodiments, when a heteroaryl formed by the joining of R^{8A} and R^{8B} substituents bonded to the same nitrogen atom is substituted, it is substituted with at least one size-limited substituent group. In embodiments, when a heteroaryl formed by the joining of R^{8A} and R^{8B} substituents bonded to the same nitrogen atom is substituted, it is substituted with at least one lower substituent group.

In embodiments, R¹¹ is independently hydrogen, halogen, —CX¹¹₃, —CHX¹¹₂, —CH₂X¹¹, —OCX¹¹₃,

—OCH₂X¹¹, —OCHX¹¹₂, —CN, —SOₙ₁₁R^{11D}, —SOᵥ₁₁NR^{11A}R^{11B}, —NHC(O)NR^{11A}R^{11B}, —N(O)ₘ₁₁, —NR^{11A}R^{11B}, —C(O)R^{11C}, —C(O)—OR^{11C}, —C(O)NR^{11A}R^{11B}, —OR^{11D}, —NR^{11A}CH₂C(O)R^{11C}, —NR^{11A}CH₂SO₂R^{11D}, —NR^{11A}SO₂R^{11D}, —NR^{11A}C(O)R^{11C}, —NR^{11A}C(O)OR^{11C}, —NR^{11A}O^{11C}, —NR^{11A}OSO₂R^{11D}, —NR^{11A}OCH₂C(O)R^{11C}, —NR^{11A}CH₂P(O)R^{11C}R^{11D}, —PO_{q11}R^{11A}, —POᵣ₁₁R^{11C}R^{IID}, —POᵣ₁₁NR^{11A}R^{11B}, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R¹¹ is independently substituted with one or more substituent groups. In embodiments, each R¹¹ is independently substituted with one or more size-limited substituent groups. In embodiments, each R¹¹ is independently substituted with one or more lower substituent groups.

In embodiments, a substituted R¹¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹¹ is substituted with a plurality of groups selected from substituent groups, size-limited substituent groups, and lower substituent groups; each substituent group, size-limited substituent group, and/or lower substituent group may optionally be different. In embodiments, when R¹¹ is substituted, it is substituted with at least one substituent group. In embodiments, when R¹¹ is substituted, it is substituted with at least one size-limited substituent group. In embodiments, when R¹¹ is substituted, it is substituted with at least one lower substituent group.

In embodiments, R¹² is independently hydrogen,

halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R¹² is independently substituted with one or more substituent groups. In embodiments, each R¹² is independently substituted with one or more size-limited substituent groups. In embodiments, each R¹² is independently substituted with one or more lower substituent groups.

In embodiments, a substituted R¹² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹² is substituted with a plurality of groups selected from substituent groups, size-limited substituent groups, and lower substituent groups; each substituent group, size-limited substituent group, and/or lower substituent group may optionally be different. In embodiments, when R¹² is substituted, it is substituted with at least one substituent group. In embodiments, when R¹² is substituted, it is substituted with at least one size-limited substituent group. In embodiments, when R¹² is substituted, it is substituted with at least one lower substituent group.

In embodiments, R¹³ is independently hydrogen,

halogen, —CCl₃, —CBr₃, —CF₃, —CI₃, —CH₂Cl, —CH₂Br, —CH₂F, —CH₂I, —CHCl₂, —CHBr₂, —CHF₂, —CH 12, —CN, —OH, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R¹³ is independently substituted with one or more substituent groups. In embodiments, each R¹³ is independently substituted with one or more size-limited substituent groups. In embodiments, each R¹³ is independently substituted with one or more lower substituent groups.

In embodiments, a substituted R¹³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹³ is substituted with a plurality of groups selected from substituent groups, size-limited substituent groups, and lower substituent groups; each substituent group, size-limited substituent group, and/or lower substituent group may optionally be different. In embodiments, when R¹³ is substituted, it is substituted with at least one substituent group. In embodiments, when R¹³ is substituted, it is substituted with at least one size-limited substituent group. In embodiments, when R¹³ is substituted, it is substituted with at least one lower substituent group.

In embodiments, R¹⁵ is independently hydrogen,

halogen, —CCl₃, —CBr₃, —CF₃, —CI₃, —CH₂Cl, —CH₂Br, —CH₂F, —CH₂I, —CHCl₂, —CHBr₂, —CHF₂, —CH 12, —CN, —OH, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R¹⁵ is independently substituted with one or more substituent groups. In embodiments, each R¹⁵ is independently substituted with one or more size-limited substituent groups. In embodiments, each R¹⁵ is independently substituted with one or more lower substituent groups.

In embodiments, a substituted R¹⁵ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁵ is substituted with a plurality of groups selected from substituent groups, size-limited substituent groups, and lower substituent groups; each substituent group, size-limited substituent group, and/or lower substituent group may optionally be different. In embodiments, when R¹⁵ is substituted, it is substituted with at least one substituent group. In embodiments, when R¹⁵ is substituted, it is substituted with at least one size-limited substituent group. In embodiments, when R¹⁵ is substituted, it is substituted with at least one lower substituent group.

In embodiments, R¹⁶ is independently hydrogen,

halogen, —CCl₃, —CBr₃, —CF₃, —CI₃, —CH₂Cl, —CH₂Br, —CH₂F, —CH₂I, —CHCl₂, —CHBr₂, —CHF₂, —CH 12, —CN, —OH, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R¹⁶ is independently substituted with one or more substituent groups. In embodiments, each R¹⁶ is independently substituted with one or more size-limited substituent groups. In embodiments, each R¹⁶ is independently substituted with one or more lower substituent groups.

In embodiments, a substituted R¹⁶ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁶ is substituted with a plurality of groups selected from substituent groups, size-limited substituent groups, and lower substituent groups; each substituent group, size-limited substituent group, and/or lower substituent group may optionally be different. In embodiments, when R¹⁶ is substituted, it is substituted with at least one substituent group. In embodiments, when R¹⁶ is substituted, it is substituted with at least one size-limited substituent group. In embodiments, when R¹⁶ is substituted, it is substituted with at least one lower substituent group.

In embodiments, R^{11A} is independently

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, CN, —OH, —OSO₃H, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{11A} is independently substituted with one or more substituent groups. In embodiments, each R^{11A} is independently substituted with one or more size-limited substituent groups. In embodiments, each R^{11A} is independently substituted with one or more lower substituent groups.

In embodiments, a substituted R^{11A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11A} is substituted with a plurality of groups selected from substituent groups, size-limited substituent groups, and lower substituent groups; each substituent group, size-limited substituent group, and/or lower substituent group may optionally be different. In embodiments, when R^{11A} is substituted, it is substituted with at least one substituent group. In embodiments, when R^{11A} is substituted, it is substituted with at least one size-limited substituent group. In embodiments, when R^{11A} is substituted, it is substituted with at least one lower substituent group.

In embodiments, R^{11B} is independently

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, CN, —OH, —OSO₃H, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{11B} is independently substituted with one or more substituent groups. In embodiments, each R^{11B} is independently substituted with one or more size-limited substituent groups. In embodiments, each R^{11B} is independently substituted with one or more lower substituent groups.

In embodiments, a substituted R^{11B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11B} is substituted with a plurality of groups selected from substituent groups, size-limited substituent groups, and lower substituent groups; each substituent group, size-limited substituent group, and/or lower substituent group may optionally be different. In embodiments, when R^{11B} is substituted, it is substituted with at least one substituent group. In embodiments, when R^{11B} is substituted, it is substituted with at least one size-limited substituent group. In embodiments, when R^{11B} is substituted, it is substituted with at least one lower substituent group.

In embodiments, R^{11A} and R^{11B} substituents bonded to the same nitrogen atom may optionally be joined to form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, a substituted heterocycloalkyl or substituted heteroaryl formed by the joining of R^{11A} and R^{11B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{11A} and R^{11B} substituents bonded to the same nitrogen atom is substituted, it is substituted with at least one substituent group. In embodiments, when a heterocycloalkyl formed by the joining of R^{11A} and R^{11B} substituents bonded to the same nitrogen atom is substituted, it is substituted with at least one size-limited substituent group. In embodiments, when a heterocycloalkyl formed by the joining of R^{11A} and R^{11B} substituents bonded to the same nitrogen atom is substituted, it is substituted with at least one lower substituent group. In embodiments, when a heteroaryl formed by the joining of R^{11A} and R^{11B} substituents bonded to the same nitrogen atom is substituted, it is substituted with at least one substituent group. In embodiments, when a heteroaryl formed by the joining of R^{11A} and R^{11B} substituents bonded to the same nitrogen atom is substituted, it is substituted with at least one size-limited substituent group. In embodiments, when a heteroaryl formed by the joining of R^{11A} and R^{11B} substituents bonded to the same nitrogen atom is substituted, it is substituted with at least one lower substituent group.

In embodiments, R^{11C} is independently

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, CN, —OH, —OSO₃H, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{11C} is independently substituted with one or more substituent groups. In embodiments, each R^{11C} is independently substituted with one or more size-limited substituent groups. In embodiments, each R^{11C} is independently substituted with one or more lower substituent groups.

In embodiments, a substituted R^{11C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11C} is substituted with a plurality of groups selected from substituent groups, size-limited substituent groups, and lower substituent groups; each substituent group, size-limited substituent group, and/or lower substituent group may optionally be different. In embodiments, when R^{11C} is substituted, it is substituted with at least one substituent group. In embodiments, when R^{11C} is substituted, it is substituted with at least one size-limited substituent group. In embodiments, when R^{11C} is substituted, it is substituted with at least one lower substituent group.

In embodiments, R^{11D} is independently

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, CN, —OH, —OSO₃H, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{11D} is independently substituted with one or more substituent groups. In embodiments, each R^{11D} is independently substituted with one or more size-limited substituent groups. In embodiments, each R^{11D} is independently substituted with one or more lower substituent groups.

In embodiments, a substituted R^{11D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{11D} is substituted with a plurality of groups selected from substituent groups, size-limited substituent groups, and lower substituent groups; each substituent group, size-limited substituent group, and/or lower substituent group may optionally be different. In embodiments, when R^{11D} is substituted, it is substituted with at least one substituent group. In embodiments, when R^{11D} is substituted, it is substituted with at least one size-limited substituent group. In embodiments, when RID is substituted, it is substituted with at least one lower substituent group.

In embodiments, R^{17A} is independently

hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, CN, —OH, —OSO₃H, —NH₂, —COOH, —CONH₂, —NO₂, —SH, substituted (e.g. with a substituent group, a size-limited substituent group or a lower substituent group) or unsubstituted alkyl (e.g., C₁-C₈ alkyl, C₁-C₆ alkyl, or C₁-C₄ alkyl), substituted (e.g. with a substituent group, a size-limited substituent group or a lower substituent group) or unsubstituted heteroalkyl (e.g., 2 to 8 membered heteroalkyl, 2 to 6 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, each R^{17A} is independently substituted with one or more substituent groups. In embodiments, each R^{17A} is independently substituted with one or more size-limited substituent groups. In embodiments, each R^{17A} is independently substituted with one or more lower substituent groups.

In embodiments, a substituted R^{17A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{17A} is substituted with a plurality of groups selected from substituent groups, size-limited substituent groups, and lower substituent groups; each substituent group, size-limited substituent group, and/or lower substituent group may optionally be different. In embodiments, when R^{17A} is substituted, it is substituted with at least one substituent group. In embodiments, when R^{17A} is substituted, it is substituted with at least one size-limited substituent group. In embodiments, when R^{17A} is substituted, it is substituted with at least one lower substituent group.

In embodiments, X is —Cl, —Br, —I or —F. In embodiments, X is —Cl. In embodiments, X is —Br. In embodiments, X is —I. In embodiments, X is —F.

In embodiments, X¹¹ is —Cl, —Br, —I or —F. In embodiments, X¹¹ is —Cl. In embodiments, X¹¹ is —Br. In embodiments, X¹¹ is —I. In embodiments, X¹¹ is —F.

In embodiments, n1 is independently 0. In embodiments, n1 is independently 1. In embodiments, n1 is independently 2. In embodiments, n1 is independently 3. In embodiments, n1 is independently 4. In embodiments, n2 is independently 0. In embodiments, n2 is independently 1. In embodiments, n2 is independently 2. In embodiments, n2 is independently 3. In embodiments, n2 is independently 4. In embodiments, n4 is independently 0. In embodiments, n4 is independently 1. In embodiments, n4 is independently 2. In embodiments, n4 is independently 3. In embodiments, n4 is independently 4. In embodiments, n5 is independently 0. In embodiments, n5 is independently 1. In embodiments, n5 is independently 2. In embodiments, n5 is independently 3. In embodiments, n5 is independently 4. In embodiments, n6 is independently 0. In embodiments, n6 is independently 1. In embodiments, n6 is independently 2. In embodiments, n6 is independently 3. In embodiments, n6 is independently 4. In embodiments, n7 is independently 0. In embodiments, n7 is independently 1. In embodiments, n7 is independently 2. In embodiments, n7 is independently 3. In embodiments, n7 is independently 4. In embodiments, n8 is independently 0. In embodiments, n8 is independently 1. In embodiments, n8 is independently 2. In embodiments, n8 is independently 3. In embodiments, n8 is independently 4.

In embodiments, n11 is independently an integer from 0 to 4. In embodiments, n11 is independently 0. In embodiments, n11 is independently 1. In embodiments, n11 is independently 2. In embodiments, n11 is independently 3. In embodiments, n11 is independently 4.

In embodiments, v1 is 1. In embodiments, v1 is 2. In embodiments, v2 is 1. In embodiments, v2 is 2. In embodiments, v4 is 1. In embodiments, v4 is 2. In embodiments, v5 is 1. In embodiments, v5 is 2. In embodiments, v6 is 1. In embodiments, v6 is 2. In embodiments, v7 is 1. In embodiments, v7 is 2. In embodiments, v8 is 1. In embodiments, v8 is 2.

In embodiments, v11 is independently 1 or 2. In embodiments, v11 is 1. In embodiments, v11 is 2.

In embodiments, m1 is 0. In embodiments, m1 is 1. In embodiments, m1 is 2. In embodiments, m1 is 3. In embodiments, m2 is 0. In embodiments, m2 is 1. In embodiments, m2 is 2. In embodiments, m2 is 3. In embodiments, m4 is 0. In embodiments, m4 is 1. In embodiments, m4 is 2. In embodiments, m4 is 3. In embodiments, m5 is 0. In embodiments, m5 is 1. In embodiments, m5 is 2. In embodiments, m5 is 3. In embodiments, m6 is 0. In embodiments, m6 is 1. In embodiments, m6 is 2. In embodiments, m6 is 3. In embodiments, m7 is 0. In embodiments, m7 is 1. In embodiments, m7 is 2. In embodiments, m7 is 3. In embodiments, m8 is 0. In embodiments, m8 is 1. In embodiments, m8 is 2. In embodiments, m8 is 3.

In embodiments, m11 is independently an integer from 0 to 3. In embodiments, m11 is independently 0. In embodiments, m11 is independently 1. In embodiments, m11 is independently 2. In embodiments, m11 is independently 3.

In embodiments, r1 is 1. In embodiments, r1 is 2. In embodiments, r2 is 1. In embodiments, r2 is 2. In embodiments, r4 is 1. In embodiments, r4 is 2. In embodiments, r5 is 1. In embodiments, r5 is 2. In embodiments, r6 is 1. In embodiments, r6 is 2. In embodiments, r7 is 1. In embodiments, r7 is 2. In embodiments, r8 is 1. In embodiments, r8 is 2.

In embodiments, q11 is independently 1 or 2. In embodiments, q11 is independently 1. In embodiments, q11 is independently 2.

In embodiments, r11 is independently 1 or 2. In embodiments, r11 is independently 1. In embodiments, r11 is independently 2.

In embodiments, z1 is independently 0. In embodiments, z1 is independently 1. In embodiments, z1 is independently 2. In embodiments, z1 is independently 3. In embodiments, z1 is independently 4. In embodiments, z1 is independently 5. In embodiments, z3 is independently 0. In embodiments, z3 is independently 1. In embodiments, z3 is independently 2. In embodiments, z3 is independently 3. In embodiments, z3 is independently 4. In embodiments, z3 is independently 5.

In embodiments, z2 is independently 0. In embodiments, z2 is independently 1. In embodiments, z2 is independently 2. In embodiments, z2 is independently 3. In embodiments, z2 is independently 4. In embodiments, z4 is independently 0. In embodiments, z4 is independently 1. In embodiments, z4 is independently 2. In embodiments, z4 is independently 3.

In embodiments, z16 is independently an integer from 0 to 8. In embodiments, z16 is independently 0. In embodiments, z16 is independently 1. In embodiments, z16 is independently 2. In embodiments, z16 is independently 3. In embodiments, z16 is independently 4. In embodiments, z16 is independently 5. In embodiments, z16 is independently 6. In embodiments, z16 is independently 7. In embodiments, z16 is independently 8.

In embodiments, provided herein is an MHC-peptide antigen stabilizing compound having the structure:

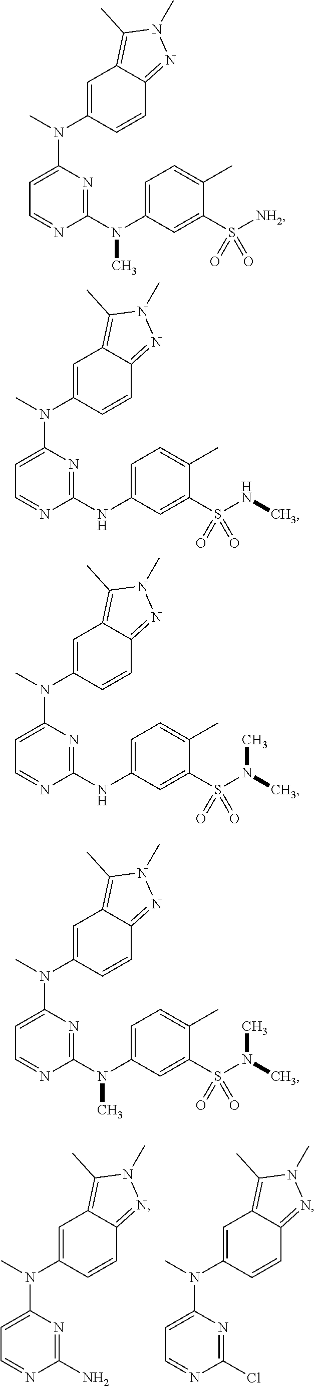

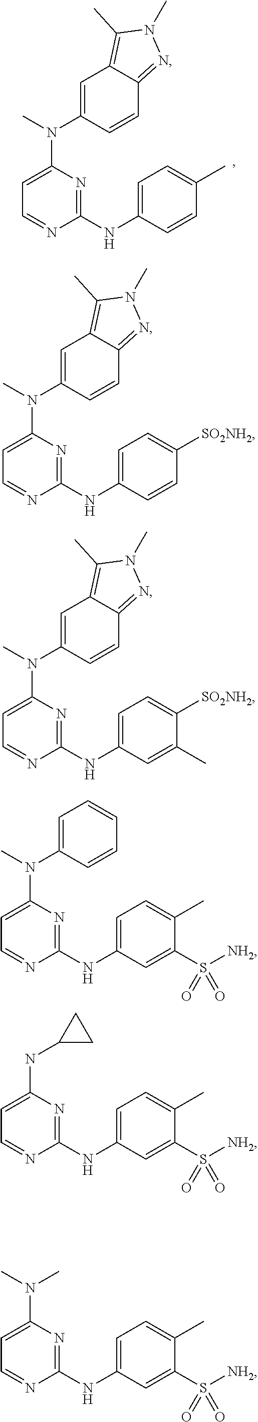

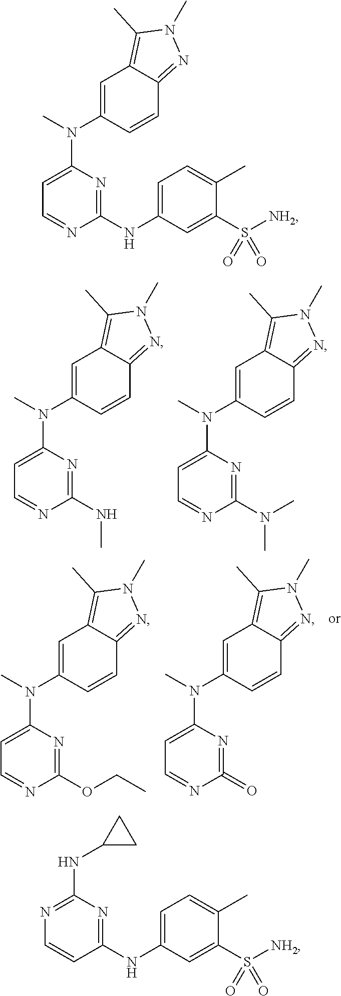

salt thereof.

In embodiments, the MHC-peptide antigen stabilizing compound is:

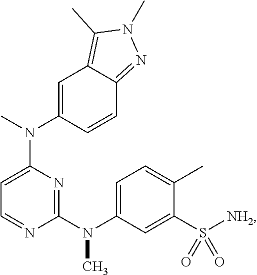

or a salt thereof. In embodiments, the MHC-peptide antigen stabilizing compound is:

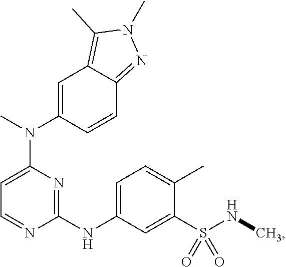

or a salt thereof. In embodiments, the MHC-peptide antigen stabilizing compound is:

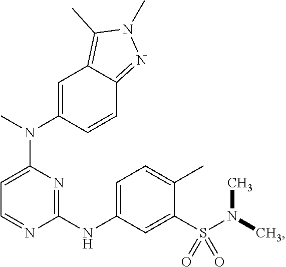

or a salt thereof. In embodiments, the MHC-peptide antigen stabilizing compound is

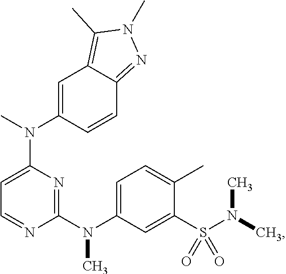

or a salt thereof. In embodiments, the MHC-peptide antigen stabilizing compound is:

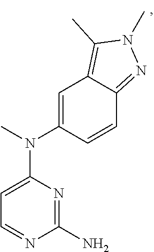 [SMILES: Cc1c2cc(N(C)c3ccnc(N)n3)ccc2nn1C]

or a salt thereof. In embodiments, the MHC-peptide antigen stabilizing compound is:

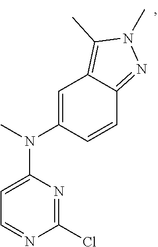 [SMILES: Cc1c2cc(N(C)c3ccnc(Cl)n3)ccc2nn1C]

or a salt thereof. In embodiments, the MHC-peptide antigen stabilizing compound is:

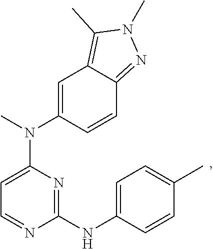 [SMILES: Cc1ccc(Nc2nccc(N(C)c3ccc4nn(C)c(C)c4c3)n2)cc1]

or a salt thereof. In embodiments, the MHC-peptide antigen stabilizing compound is:

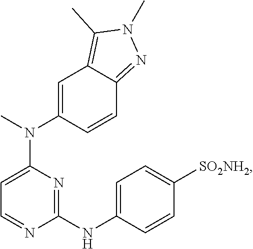 [SMILES: Cc1c2cc(N(C)c3ccnc(Nc4ccc(S(N)(=O)=O)cc4)n3)ccc2nn1C]

or a salt thereof. In embodiments, the MHC-peptide antigen stabilizing compound is:

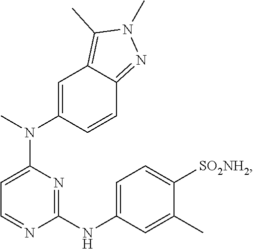 [SMILES: Cc1cc(Nc2nccc(N(C)c3ccc4nn(C)c(C)c4c3)n2)ccc1S(N)(=O)=O]

or a salt thereof. In embodiments, the MHC-peptide antigen stabilizing compound is:

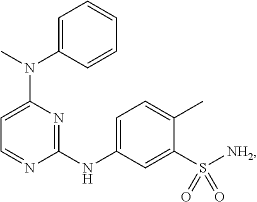 [SMILES: Cc1ccc(Nc2nccc(N(C)c3ccccc3)n2)cc1S(N)(=O)=O]

or a salt thereof. In embodiments, the MHC-peptide antigen stabilizing compound is:

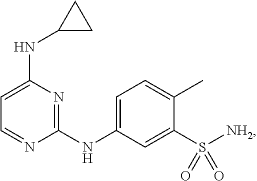 [SMILES: Cc1ccc(Nc2nccc(NC3CC3)n2)cc1S(N)(=O)=O]

or a salt thereof. In embodiments, the MHC-peptide antigen stabilizing compound is:

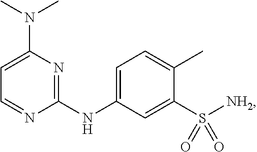 [SMILES: Cc1ccc(Nc2nccc(N(C)C)n2)cc1S(N)(=O)=O]

or a salt thereof. In embodiments, the MHC-peptide antigen stabilizing compound is:

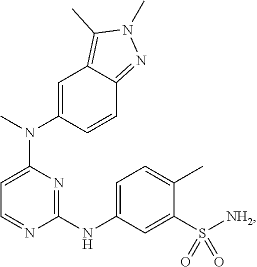 [SMILES: Cc1ccc(Nc2nccc(N(C)c3ccc4nn(C)c(C)c4c3)n2)cc1S(N)(=O)=O]

or a salt thereof. In embodiments, the MHC-peptide antigen stabilizing compound is:

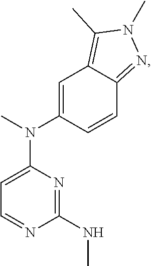 [SMILES: CNc1nccc(N(C)c2ccc3nn(C)c(C)c3c2)n1]

or a salt thereof. In embodiments, the MHC-peptide antigen stabilizing compound is:

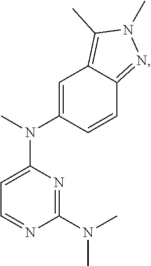 [SMILES: Cc1c2cc(N(C)c3ccnc(N(C)C)n3)ccc2nn1C]

or a salt thereof. In embodiments, the MHC-peptide antigen stabilizing compound is:

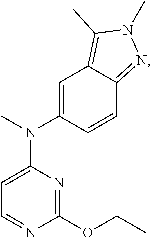 [SMILES: CCOc1nccc(N(C)c2ccc3nn(C)c(C)c3c2)n1]

or a salt thereof. In embodiments, the MHC-peptide antigen stabilizing compound is:

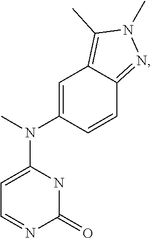 [SMILES: Cc1c2cc(N(C)c3ccnc(=O)[nH]3)ccc2nn1C]

or a salt thereof. In embodiments, the MHC-peptide antigen stabilizing compound is:

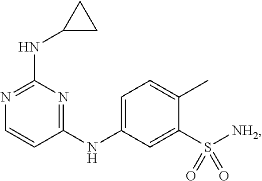 [SMILES: Cc1ccc(Nc2ccnc(NC3CC3)n2)cc1S(N)(=O)=O]

or a salt thereof.

In embodiments, provided herein is an MHC-peptide antigen stabilizing compound having the structure:

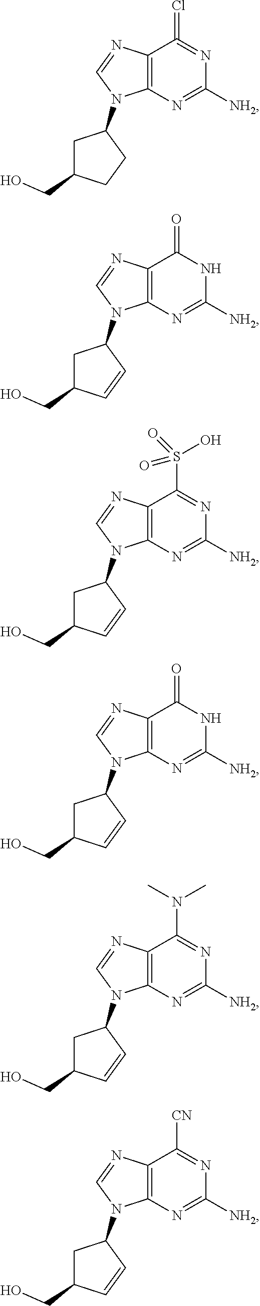

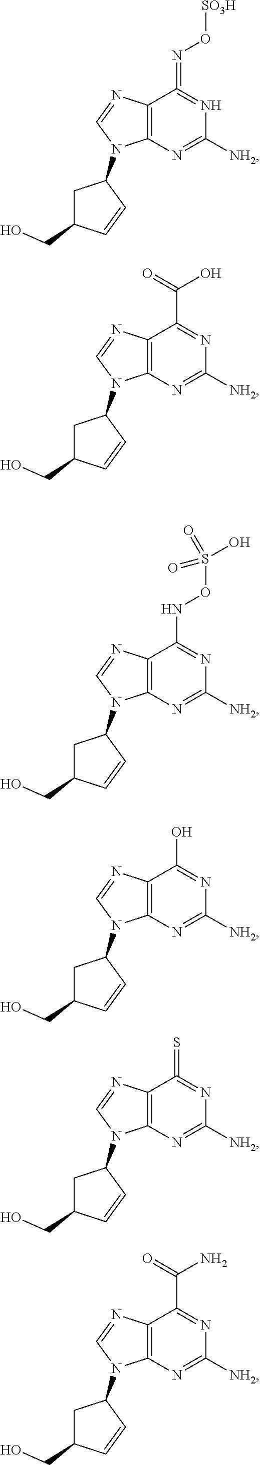

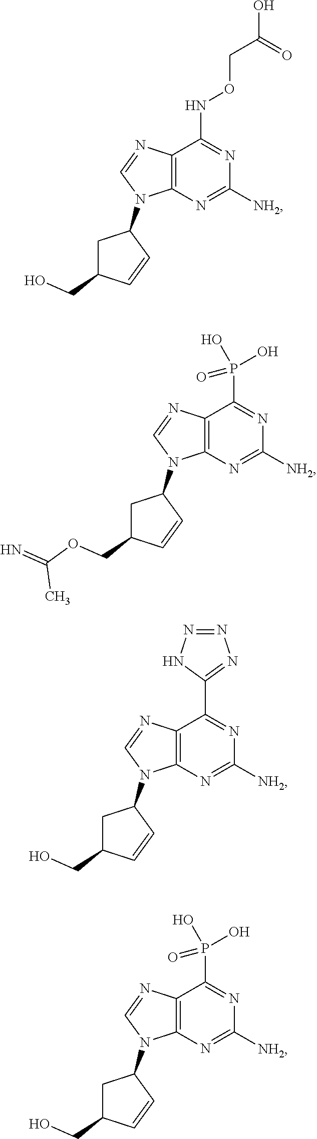 [SMILES: Nc1nc(NOCC(=O)O)c2ncn([C@H]3C=C[C@@H](CO)C3)c2n1 ; CC(=N)OC[C@@H]1C=C[C@H](n2cnc3c(P(=O)(O)O)nc(N)nc32)C1 ; Nc1nc(-c2nnn[nH]2)c2ncn([C@H]3C=C[C@@H](CO)C3)c2n1 ; Nc1nc(P(=O)(O)O)c2ncn([C@H]3C=C[C@@H](CO)C3)c2n1]

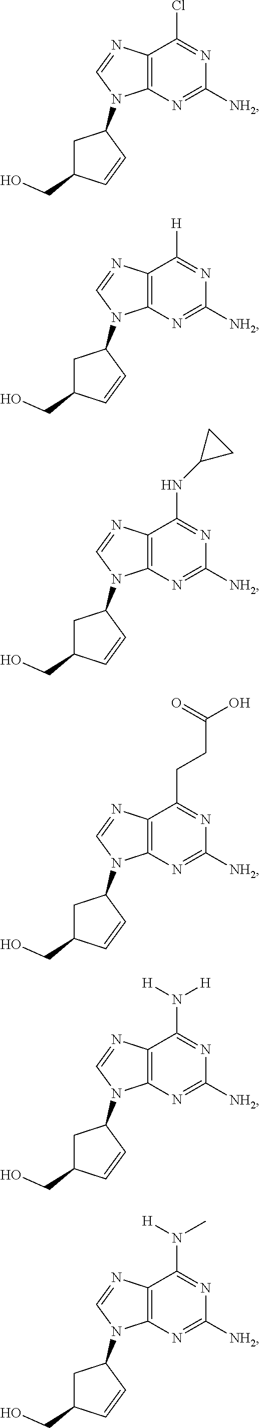 [SMILES: Nc1nc(Cl)c2ncn([C@H]3C=C[C@@H](CO)C3)c2n1 ; Nc1ncc2ncn([C@H]3C=C[C@@H](CO)C3)c2n1 ; Nc1nc(NC2CC2)c2ncn([C@H]3C=C[C@@H](CO)C3)c2n1 ; Nc1nc(CCC(=O)O)c2ncn([C@H]3C=C[C@@H](CO)C3)c2n1 ; Nc1nc(N)c2ncn([C@H]3C=C[C@@H](CO)C3)c2n1 ; CNc1nc(N)nc2c1ncn2[C@H]1C=C[C@@H](CO)C1]

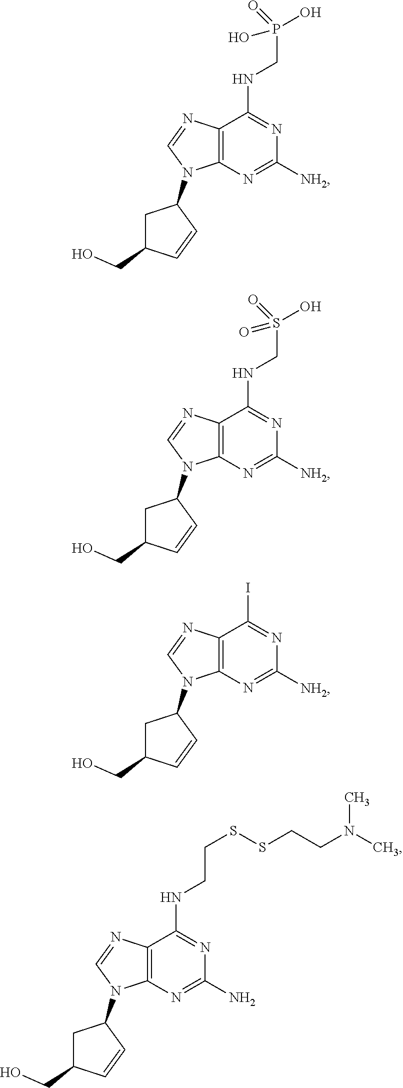 [SMILES: Nc1nc(NCP(=O)(O)O)c2ncn([C@H]3C=C[C@@H](CO)C3)c2n1 ; Nc1nc(NCS(=O)(=O)O)c2ncn([C@H]3C=C[C@@H](CO)C3)c2n1 ; Nc1nc(I)c2ncn([C@H]3C=C[C@@H](CO)C3)c2n1 ; CN(C)CCSSCCNc1nc(N)nc2c1ncn2[C@H]1C=C[C@@H](CO)C1]

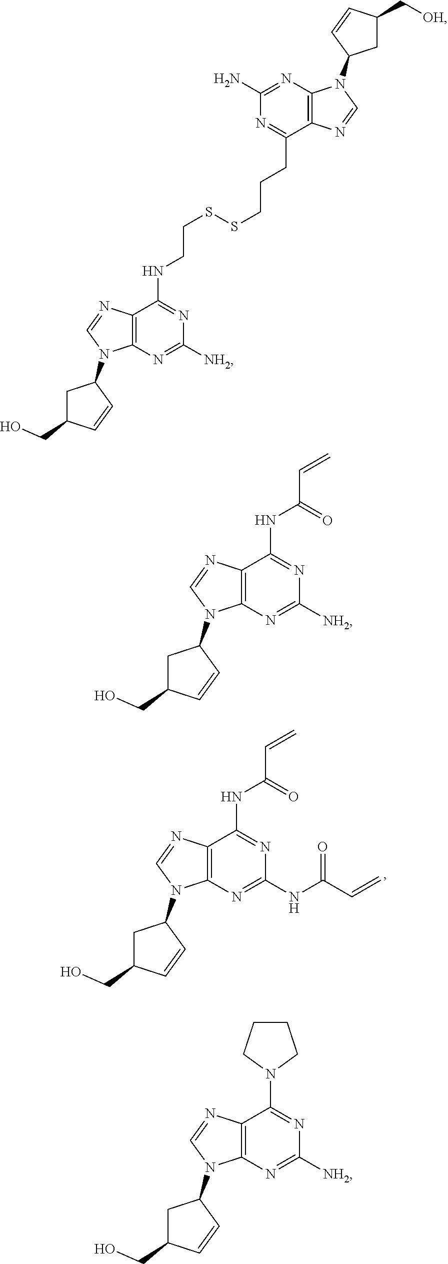 [SMILES: Nc1nc(CCCSSCCNc2nc(N)nc3c2ncn3[C@H]2C=C[C@@H](CO)C2)c2ncn([C@H]3C=C[C@@H](CO)C3)c2n1 ; C=CC(=O)Nc1nc(N)nc2c1ncn2[C@H]1C=C[C@@H](CO)C1 ; C=CC(=O)Nc1nc(NC(=O)C=C)c2ncn([C@H]3C=C[C@@H](CO)C3)c2n1 ; Nc1nc(N2CCCC2)c2ncn([C@H]3C=C[C@@H](CO)C3)c2n1]

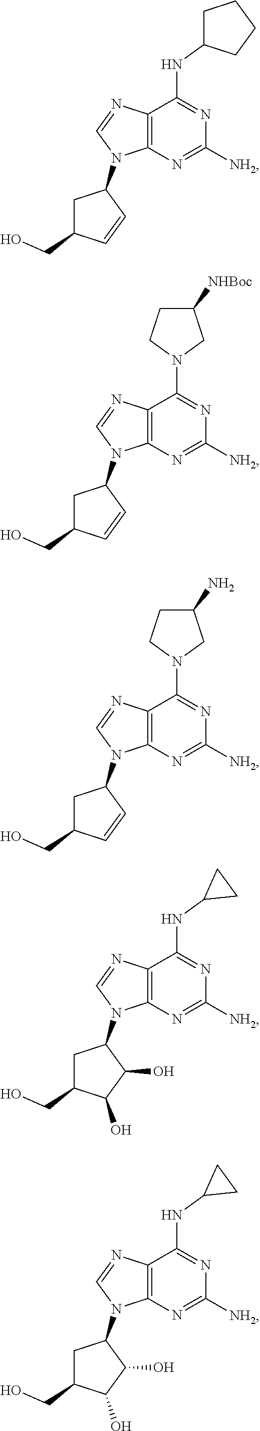 [SMILES: Nc1nc(NC2CCCC2)c2ncn([C@H]3C=C[C@@H](CO)C3)c2n1 ; CC(C)(C)OC(=O)N[C@@H]1CCN(c2nc(N)nc3c2ncn3[C@H]2C=C[C@@H](CO)C2)C1 ; Nc1nc(N2CC[C@@H](N)C2)c2ncn([C@H]3C=C[C@@H](CO)C3)c2n1 ; Nc1nc(NC2CC2)c2ncn([C@@H]3C[C@H](CO)[C@H](O)[C@@H]3O)c2n1 ; Nc1nc(NC2CC2)c2ncn([C@@H]3C[C@H](CO)[C@@H](O)[C@H]3O)c2n1]

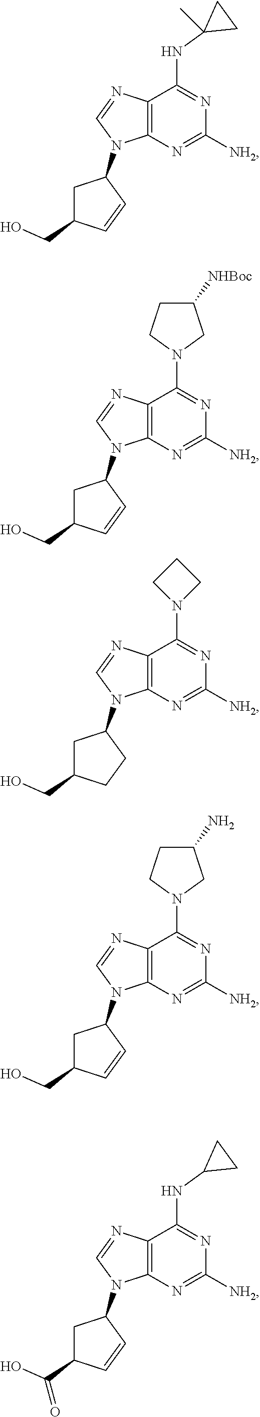 [SMILES: CC1(Nc2nc(N)nc3c2ncn3[C@H]2C=C[C@@H](CO)C2)CC1 ; CC(C)(C)OC(=O)N[C@H]1CCN(c2nc(N)nc3c2ncn3[C@H]2C=C[C@@H](CO)C2)C1 ; Nc1nc(N2CCC2)c2ncn([C@H]3CC[C@@H](CO)C3)c2n1 ; Nc1nc(N2CC[C@H](N)C2)c2ncn([C@H]3C=C[C@@H](CO)C3)c2n1 ; Nc1nc(NC2CC2)c2ncn([C@H]3C=C[C@@H](C(=O)O)C3)c2n1]

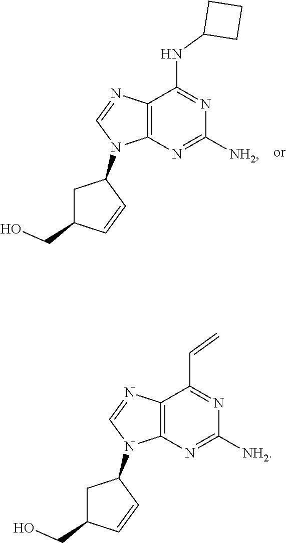 [SMILES: Nc1nc(NC2CCC2)c2ncn([C@H]3C=C[C@@H](CO)C3)c2n1 ; C=Cc1nc(N)nc2c1ncn2[C@H]1C=C[C@@H](CO)C1]

In embodiments, the MHC-peptide antigen stabilizing compound is:

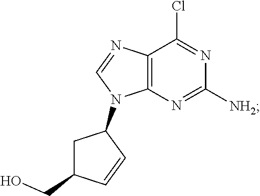 [SMILES: Nc1nc(Cl)c2ncn([C@H]3C=C[C@@H](CO)C3)c2n1]

or a salt thereof. In embodiments, the MHC-peptide antigen stabilizing compound is:

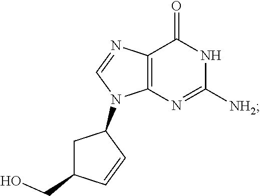 [SMILES: Nc1nc2c(ncn2[C@H]2C=C[C@@H](CO)C2)c(=O)[nH]1]

or a salt thereof. In embodiments, the MHC-peptide antigen stabilizing compound is:

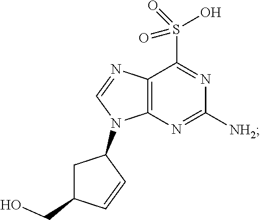 [SMILES: Nc1nc(S(=O)(=O)O)c2ncn([C@H]3C=C[C@@H](CO)C3)c2n1]

or a salt thereof. In embodiments, the MHC-peptide antigen stabilizing compound is:

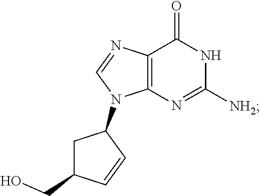 [SMILES: Nc1nc2c(ncn2[C@H]2C=C[C@@H](CO)C2)c(=O)[nH]1]

or a salt thereof. In embodiments, the MHC-peptide antigen stabilizing compound is:

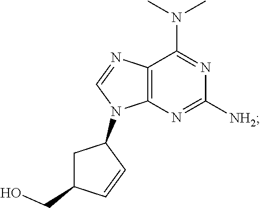 [SMILES: CN(C)c1nc(N)nc2c1ncn2[C@H]1C=C[C@@H](CO)C1]

or a salt thereof. In embodiments, the MHC-peptide antigen stabilizing compound is:

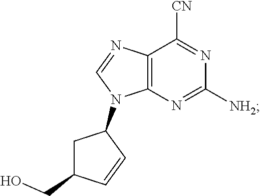 [SMILES: N#Cc1nc(N)nc2c1ncn2[C@H]1C=C[C@@H](CO)C1]

or a salt thereof. In embodiments, the MHC-peptide antigen stabilizing compound is:

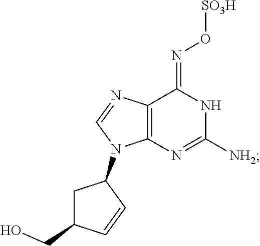 [SMILES: Nc1nc2c(ncn2[C@H]2C=C[C@@H](CO)C2)/c(=N/OS(=O)(=O)O)[nH]1]

or a salt thereof. In embodiments, the MHC-peptide antigen stabilizing compound is:

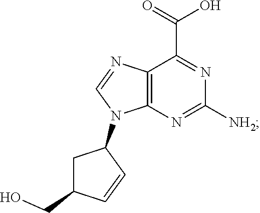 [SMILES: Nc1nc(C(=O)O)c2ncn([C@H]3C=C[C@@H](CO)C3)c2n1]

or a salt thereof. In embodiments, the MHC-peptide antigen stabilizing compound is:

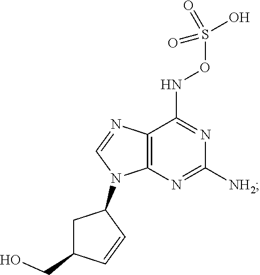 [SMILES: Nc1nc(NOS(=O)(=O)O)c2ncn([C@H]3C=C[C@@H](CO)C3)c2n1]

or a salt thereof. In embodiments, the MHC-peptide antigen stabilizing compound is:

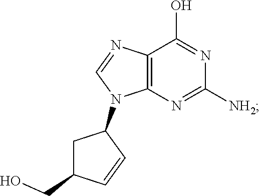 [SMILES: Nc1nc(O)c2ncn([C@H]3C=C[C@@H](CO)C3)c2n1]

or a salt thereof. In embodiments, the MHC-peptide antigen stabilizing compound is:

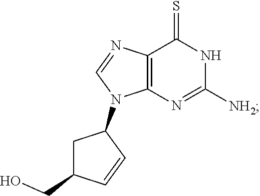 [SMILES: Nc1nc2c(ncn2[C@H]2C=C[C@@H](CO)C2)c(=S)[nH]1]

or a salt thereof. In embodiments, the MHC-peptide antigen stabilizing compound is:

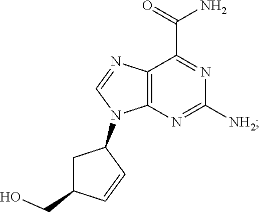 [SMILES: NC(=O)c1nc(N)nc2c1ncn2[C@H]1C=C[C@@H](CO)C1]

or a salt thereof. In embodiments, the MHC-peptide antigen stabilizing compound is:

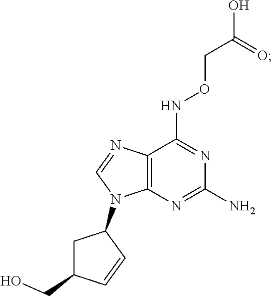 [SMILES: Nc1nc(NOCC(=O)O)c2ncn([C@H]3C=C[C@@H](CO)C3)c2n1]

or a salt thereof. In embodiments, the MHC-peptide antigen stabilizing compound is:

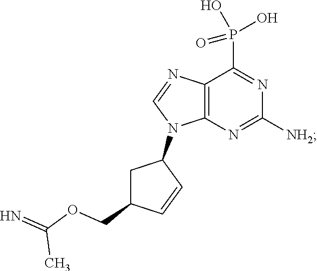 [SMILES: CC(=N)OC[C@@H]1C=C[C@H](n2cnc3c(P(=O)(O)O)nc(N)nc32)C1]

or a salt thereof. In embodiments, the MHC-peptide antigen stabilizing compound is:

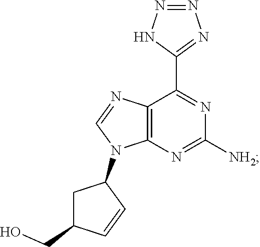 [SMILES: Nc1nc(-c2nnn[nH]2)c2ncn([C@H]3C=C[C@@H](CO)C3)c2n1]

or a salt thereof. In embodiments, the MHC-peptide antigen stabilizing compound is:

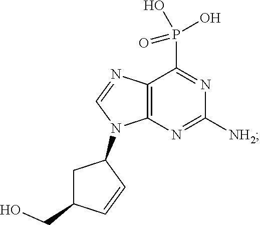 [SMILES: Nc1nc(P(=O)(O)O)c2ncn([C@H]3C=C[C@@H](CO)C3)c2n1]

a salt thereof. In embodiments, the MHC-peptide antigen stabilizing compound is:

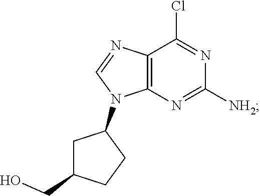 [SMILES: Nc1nc(Cl)c2ncn([C@H]3CC[C@@H](CO)C3)c2n1]

or a salt thereof. In embodiments, the MHC-peptide antigen stabilizing compound is:

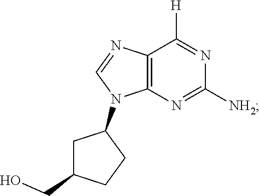 [SMILES: Nc1ncc2ncn([C@H]3CC[C@@H](CO)C3)c2n1]

or a salt thereof. In embodiments, the MHC-peptide antigen stabilizing compound is:

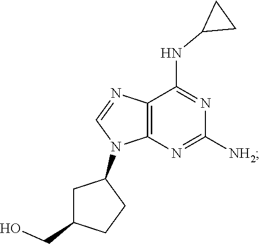 [SMILES: Nc1nc(NC2CC2)c2ncn([C@H]3CC[C@@H](CO)C3)c2n1]

or a salt thereof. In embodiments, the MHC-peptide antigen stabilizing compound is:

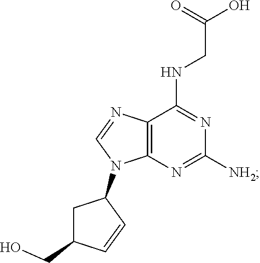 [SMILES: Nc1nc(NCC(=O)O)c2ncn([C@H]3C=C[C@@H](CO)C3)c2n1]

or a salt thereof. In embodiments, the MHC-peptide antigen stabilizing compound is:

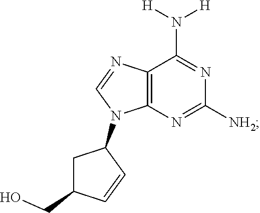 [SMILES: Nc1nc(N)c2ncn([C@H]3C=C[C@@H](CO)C3)c2n1]

or a salt thereof. In embodiments, the MHC-peptide antigen stabilizing compound is:

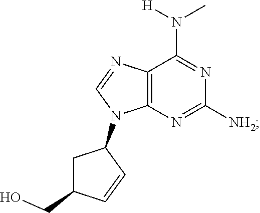 [SMILES: CNc1nc(N)nc2c1ncn2[C@H]1C=C[C@@H](CO)C1]

or a salt thereof. In embodiments, the MHC-peptide antigen stabilizing compound is:

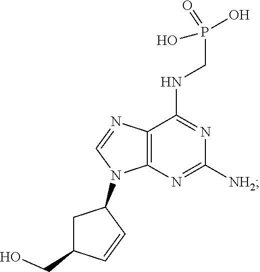 [SMILES: Nc1nc(NCP(=O)(O)O)c2ncn([C@H]3C=C[C@@H](CO)C3)c2n1]

or a salt thereof. In embodiments, the MHC-peptide antigen stabilizing compound is:

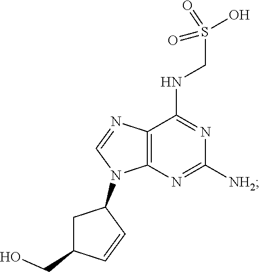 [SMILES: Nc1nc(NCS(=O)(=O)O)c2ncn([C@H]3C=C[C@@H](CO)C3)c2n1]

or a salt thereof. In embodiments, the MHC-peptide antigen stabilizing compound is:

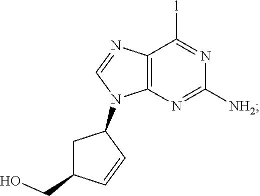

or a salt thereof. In embodiments, the MHC-peptide antigen stabilizing compound is:

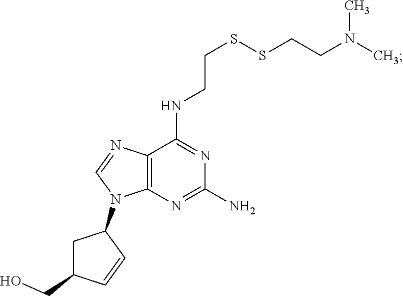 [SMILES: CN(C)CCSSCCNc1nc(N)nc2c1ncn2[C@H]1C=C[C@@H](CO)C1]

or a salt thereof. In embodiments, the MHC-peptide antigen stabilizing compound is:

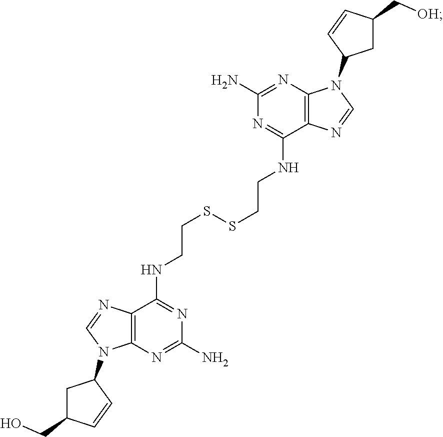

or a salt thereof. In embodiments, the MHC-peptide antigen stabilizing compound is:

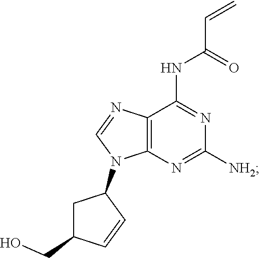 [SMILES: C=CC(=O)Nc1nc(N)nc2c1ncn2[C@H]1C=C[C@@H](CO)C1]

or a salt thereof. In embodiments, the MHC-peptide antigen stabilizing compound is:

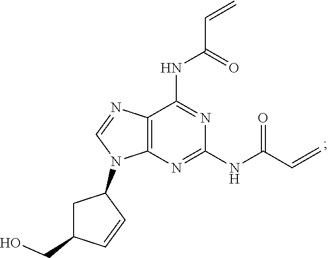 [SMILES: C=CC(=O)Nc1nc(NC(=O)C=C)c2ncn([C@H]3C=C[C@@H](CO)C3)c2n1]

or a salt thereof. In embodiments, the MHC-peptide antigen stabilizing compound is:

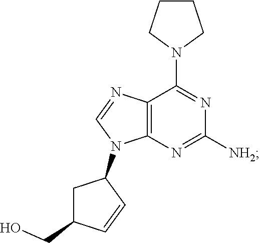 [SMILES: Nc1nc(N2CCCC2)c2ncn([C@H]3C=C[C@@H](CO)C3)c2n1]

or a salt thereof. In embodiments, the MHC-peptide antigen stabilizing compound is:

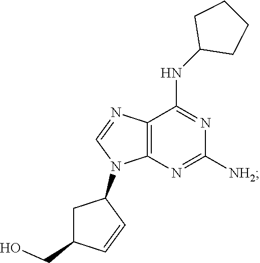 [SMILES: Nc1nc(NC2CCCC2)c2ncn([C@H]3C=C[C@@H](CO)C3)c2n1]

or a salt thereof. In embodiments, the MHC-peptide antigen stabilizing compound is:

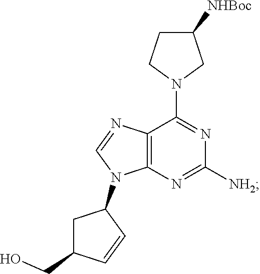 [SMILES: CC(C)(C)OC(=O)N[C@@H]1CCN(c2nc(N)nc3c2ncn3[C@H]2C=C[C@@H](CO)C2)C1]

or a salt thereof. In embodiments, the MHC-peptide antigen stabilizing compound is:

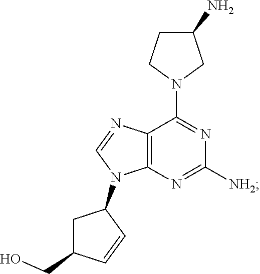 [SMILES: Nc1nc(N2CC[C@@H](N)C2)c2ncn([C@H]3C=C[C@@H](CO)C3)c2n1]

or a salt thereof. In embodiments, the MHC-peptide antigen stabilizing compound is:

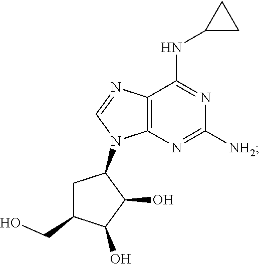 [SMILES: Nc1nc(NC2CC2)c2ncn([C@@H]3C[C@H](CO)[C@H](O)[C@@H]3O)c2n1]

or a salt thereof. In embodiments, the MHC-peptide antigen stabilizing compound is:

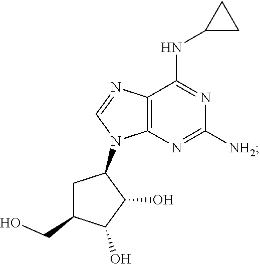 [SMILES: Nc1nc(NC2CC2)c2ncn([C@@H]3C[C@H](CO)[C@@H](O)[C@H]3O)c2n1]

or a salt thereof. In embodiments, the MHC-peptide antigen stabilizing compound is:

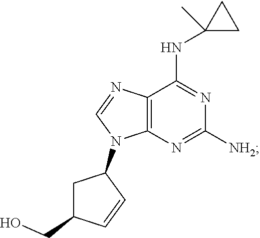 [SMILES: CC1(Nc2nc(N)nc3c2ncn3[C@H]2C=C[C@@H](CO)C2)CC1]

or a salt thereof. In embodiments, the MHC-peptide antigen stabilizing compound is:

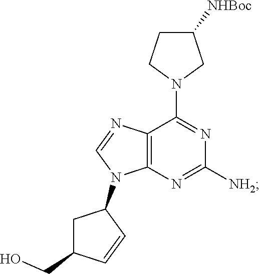 [SMILES: CC(C)(C)OC(=O)N[C@H]1CCN(c2nc(N)nc3c2ncn3[C@H]2C=C[C@@H](CO)C2)C1]

or a salt thereof. In embodiments, the MHC-peptide antigen stabilizing compound is:

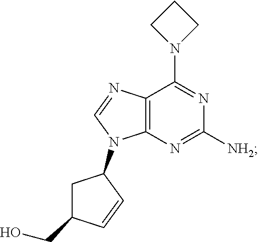 [SMILES: Nc1nc(N2CCC2)c2ncn([C@H]3C=C[C@@H](CO)C3)c2n1]

or a salt thereof. In embodiments, the MHC-peptide antigen stabilizing compound is:

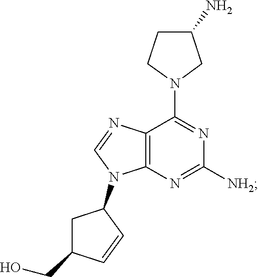 [SMILES: Nc1nc(N2CC[C@H](N)C2)c2ncn([C@H]3C=C[C@@H](CO)C3)c2n1]

or a salt thereof. In embodiments, the MHC-peptide antigen stabilizing compound is:

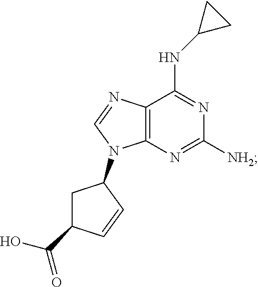 [SMILES: Nc1nc(NC2CC2)c2ncn([C@H]3C=C[C@@H](C(=O)O)C3)c2n1]

or a salt thereof. In embodiments, the MHC-peptide antigen stabilizing compound is:

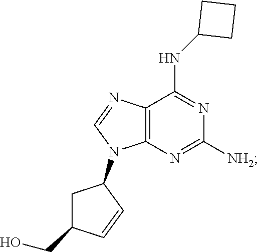 [SMILES: Nc1nc(NC2CCC2)c2ncn([C@H]3C=C[C@@H](CO)C3)c2n1]

or a salt thereof. In embodiments, the MHC-peptide antigen stabilizing compound is:

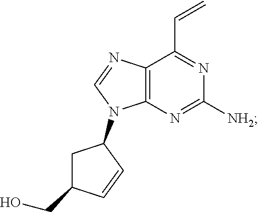 [SMILES: C=Cc1nc(N)nc2c1ncn2[C@H]1C=C[C@@H](CO)C1]

Genes commonly mutated in cancer that drive oncogenesis are listed in Table 1. Thus, Table 1 provides examples of driver oncogenes and driver oncogene proteins from which the peptide antigens may be derived (e.g. a driver oncogene derived peptide). In embodiments, the peptide antigen includes an amino acid sequence including an amino acid mutation found in a driver oncogene protein product of a driver oncogene mutation within a gene in Table 1.

TABLE 1
Genes Commonly Mutated in Cancer

| | | | | | | | | | |
|---|---|---|---|---|---|---|---|---|---|
| TP53 | KMT2A | SPTAN1 | KEL | ATF7IP | CUL3 | EEF1A1 | SF1 | ARAF | GNA13 |
| PIK3CA | EPHA3 | MTOR | KIT | ARID5B | STK11 | ESR1 | TXNIP | GNA11 | SRSF2 |
| SPTA1 | CTNNB1 | MECOM | MAP3K1 | ALB | SMC1A | FAM46D | RFC1 | IRF6 | BTG2 |
| KMT2D | CACNA1A | NRAS | RET | SIN3A | TBX3 | JAK2 | RPS6KA3 | CCND1 | PIM1 |
| KMT2C | NIPBL | BCOR | DNMT3A | SCAF4 | RBM10 | ACVR1B | FOXA2 | PTPDC1 | BCL2L11 |
| APOB | SETD2 | SETBP1 | CDK12 | SPOP | RUNX1 | SMC3 | PIK3R2 | PHF6 | FOXQ1 |
| PTEN | EP300 | TAF1 | BRCA1 | DIAPH2 | JAK1 | HIST1H1E | HLA-B | ACVR1 | NPM1 |
| BRAF | SMARCA4 | PIK3CG | CHD3 | EPHA2 | FOXA1 | RAD21 | CDKN1A | CYSLTR2 | GPS2 |
| DMD | MGA | PLXNB2 | GNAS | WHSC1 | PLCG1 | RNF111 | B2M | RAC1 | CBFB |
| KRAS | BRCA2 | ARHGAP35 | GABRA6 | JAK3 | FUBP1 | EEF2 | GNAQ | RPL22 | MYD88 |
| ARID1A | FLNA | ERBB3 | FGFR2 | KDM5C | ELF3 | TCF12 | AJUBA | PTMA | CDK4 |
| FAT1 | MED12 | KIF1A | CTCF | FGFR3 | EPAS1 | ZBTB7B | PTPN11 | HLA-A | LEMD2 |
| MACF1 | KMT2B | KEAP1 | DICER1 | CTNND1 | LATS2 | MSH6 | MAP2K1 | H3F3C | ATXN3 |
| APC | PIK3R1 | CDH1 | TET2 | SMARCA1 | DHX9 | PMS2 | ZCCHC12 | RARA | BCL2 |
| ATRX | NSD1 | PDGFRA | MET | TSC1 | TCF7L2 | TBL1XR1 | DAZAP1 | MAPK1 | CD79B |
| IDH1 | ARID2 | MUC6 | IL7R | TGFBR2 | AXIN2 | AKT1 | CHEK2 | RAF1 | CD70 |
| NF1 | CHD4 | ALK | ZBTB20 | PGR | NUP133 | RHOA | ZC3H12A | PPP6C | CDKN2C |
| HUWE1 | POLQ | NOTCH2 | INPPL1 | LZTR1 | PIK3CB | TNFAIP3 | BRD7 | PCBP1 | RQCD1 |
| PTPRD | PTPRC | STAG2 | ZMYM3 | AR | ZMYM2 | XPO1 | MYCN | RHOB | RIT1 |
| ZFHX3 | NCOR1 | GATA3 | ASXL2 | SOS1 | CUL1 | MSH3 | U2AF1 | RPL5 | KRT222 |
| ATM | TLR4 | POLE | ZFP36L1 | LATS1 | MAP2K4 | PSIP1 | NUP93 | PPM1D | H3F3A |
| CDKN2A | KDM6A | HGF | PTCH1 | GTF2I | NF2 | POLRMT | MSH2 | TGIF1 | RHEB |
| COL5A1 | USP9X | CHD8 | KANSL1 | HRAS | CSDE1 | RXRA | PAX5 | CDKN1B | TMSB4X |
| NOTCH1 | ATR | SMAD4 | RASA1 | DDX3X | GRIN2D | ZFP36L2 | SOX17 | IDH2 | MGMT |
| ERBB4 | CARD11 | ERBB2 | MAP3K4 | SOX9 | THRAP3 | CYLD | SMAD2 | MAX | RRAS2 |
| EGFR | MYH9 | VHL | RNF43 | PDS5B | ABL1 | IL6ST | EZH2 | SMARCB1 | UNCX |
| FBXW7 | NFE2L2 | SF3B1 | PPP2R1A | AXIN1 | ZNF750 | KLF5 | MEN1 | PRKAR1A | CEBPA |
| PBRM1 | CIC | BAP1 | DACH1 | WT1 | FGFR1 | MLH1 | IRF2 | EIF1AX | TCEB1 |
| RB1 | AMER1 | TSC2 | FLT3 | CNBD1 | HIST1H1C | CREB3L3 | MYC | EGR3 | CBWD3 |
| CREBBP | PLCB4 | ASXL1 | CASP8 | ACVR2A | ERCC2 | PMS1 | TRAF3 | ZNF133 | |

Both human MHC Class I alleles and Class II alleles are highly polymorphic, with 18,691 and 7,065 known sequence variants, which can be found at https://www.ebi.ac.uk/ipd/imgt/hla/stats.html. 69 most common MHC I protein alleles that occur in at least 100 of the human population are disclosed in Table 2 below. This list is available from the Immune Epitope Database and Analysis Resource, at https://iedb.org.

TABLE 2
Common MHC I Protein Alleles

HLA-A02:01
HLA-A02:06
HLA-A03:01
HLA-A11:01
HLA-A23:01
HLA-A24:02
HLA-A25:01
HLA-A26:01
HLA-A29:02
HLA-A30:01
HLA-A30:02
HLA-A31:01
HLA-A32:01
HLA-A33:03
HLA-A68:01
HLA-B13:02
HLA-B14:02
HLA-B15:01
HLA-B15:02
HLA-B15:25
HLA-B18:01
HLA-B27:02
HLA-B27:05
HLA-B35:01
HLA-B35:03
HLA-A68:02
HLA-A74:01
HLA-B07:02
HLA-B08:01
HLA-B13:01
HLA-B37:01
HLA-B38:01
HLA-B39:01
HLA-B40:01
HLA-B40:02
HLA-B44:02
HLA-B44:03
HLA-B46:01
HLA-B48:01
HLA-B49:01
HLA-B50:01
HLA-B51:01
HLA-B52:01
HLA-B53:01
HLA-B55:01
HLA-C02:02
HLA-C02:09
HLA-C03:02
HLA-C03:03
HLA-C03:04
HLA-C04:01
HLA-C05:01
HLA-C06:02
HLA-C07:01
HLA-C07:02
HLA-B56:01
HLA-B57:01
HLA-B58:01
HLA-B58:02
HLA-C01:02
HLA-C07:04
HLA-C08:01
HLA-C08:02
HLA-C12:02
HLA-C12:03
HLA-C14:02
HLA-C15:02
HLA-C16:01
HLA-C17:01

Additional MHC I and II protein alleles can be found for example in Tables 3 and 4 below.

TABLE 3
MHC I protein alleles covering >97% of human population

HLA-A*01:01.8
HLA-A*01:01.9
HLA-A*01:01.10
HLA-A*01:01.11
HLA-A*02:01.8
HLA-A*02:01.9
HLA-A*02:01.10
HLA-A*02:01.11
HLA-A*02:03.8
HLA-A*02:03.9
HLA-A*02:03.10
HLA-A*02:03.11
HLA-A*02:06.8
HLA-A*02:06.9
HLA-A*02:06.10
HLA-A*02:06.11
HLA-A*03:01.8
HLA-A*03:01.9
HLA-A*03:01.10
HLA-A*03:01.11
HLA-A*11:01.8
HLA-A*11:01.9
HLA-A*11:01.10
HLA-A*11:01.1
HLA-A*23:01.8
HLA-A*23:01.9
HLA-A*23:01.10
HLA-A*23:01.11
HLA-A*24:02.8
HLA-A*24:02.9
HLA-A*24:02.10
HLA-A*24:02.11
HLA-A*26:01.8
HLA-A*26:01.9
HLA-A*26:01.10
HLA-A*26:01.11
HLA-A*30:01.8
HLA-A*30:01.9
HLA-A*30:01.10
HLA-A*30:01.11
HLA-A*30:02.8
HLA-A*30:02.9
HLA-A*30:02.10
HLA-A*30:02.11
HLA-A*31:01.8
HLA-A*31:01.9
HLA-A*31:01.10
HLA-A*31:01.11
HLA-A*32:01.8
HLA-A*32:01.9
HLA-A*32:01.10
HLA-A*32:01.11
HLA-A*33:01.8
HLA-A*33:01.9
HLA-A*33:01.10
HLA-A*33:01.11
HLA-A*68:01.8
HLA-A*68:01.9
HLA-A*68:01.10
HLA-A*68:01.11
HLA-A*68:02.8
HLA-A*68:02.9
HLA-A*68:02.10
HLA-A*68:02.11
HLA-B*07:02.8
HLA-B*07:02.9
HLA-B*07:02.10
HLA-B*07:02.11
HLA-B*08:01.8
HLA-B*08:01.9
HLA-B*08:01.10
HLA-B*08:01.11
HLA-B*15:01.8
HLA-B*15:01.9
HLA-B*15:01.10
HLA-B*15:01.11
HLA-B*35:01.8
HLA-B*35:01.9
HLA-B*35:01.10
HLA-B*35:01.11
HLA-B*40:01.8
HLA-B*40:01.9
HLA-B*40:01.10
HLA-B*40:01.11
HLA-B*44:02.8
HLA-B*44:02.9
HLA-B*44:02.10
HLA-B*44:02.11
HLA-B*44:03.8
HLA-B*44:03.9
HLA-B*44:03.10
HLA-B*44:03.11
HLA-B*51:01.8
HLA-B*51:01.9
HLA-B*51:01.10
HLA-B*51:01.11
HLA-B*53:01.8
HLA-B*53:01.9
HLA-B*53:01.10
HLA-B*53:01.11
HLA-B*57:01.8
HLA-B*57:01.9
HLA-B*57:01.10
HLA-B*57:01.11
HLA-B*58:01.8
HLA-B*58:01.9
HLA-B*58:01.10
HLA-B*58:01.11

TABLE 4
MHC II protein alleles covering >99% of human population

HLA-DRB1*01:01
HLA-DRB1*03:01
HLA-DRB1*04:01
HLA-DRB1*04:05
HLA-DRB1*07:01
HLA-DRB1*08:02
HLA-DRB1*09:01
HLA-DRB1*11:01
HLA-DRB1*12:01
HLA-DRB1*13:02
HLA-DRB1*15:01
HLA-DRB3*01:01
HLA-DRB3*02:02
HLA-DRB4*01:01
HLA-DRB5*01:01
HLA-DQA1*05:01/DQB1*02:01
HLA-DQA1*05:01/DQB1*03:01
HLA-DQA1*03:01/DQB1*03:02
HLA-DQA1*04:01/DQB1*04:02
HLA-DQA1*01:01/DQB1*05:01
HLA-DQA1*01:02/DQB1*06:02
HLA-DPA1*02:01/DPB1*01:01
HLA-DPA1*01:03/DPB1*02:01
HLA-DPA1*01/DPB1*04:01
HLA-DPA1*03:01/DPB1*04:02
HLA-DPA1*02:01/DPB1*05:01
HLA-DPA1*02:01/DPB1*14:01

In another aspect, provided herein is a method of treating cancer in a subject in need thereof which includes detecting an MHC allele of an MHC protein of the subject, detecting a driver oncogene mutation in the subject, and then administering an effective amount of a MHC-peptide antigen stabilizing compound.

In embodiments, the MHC-peptide antigen stabilizing compound is identified in vitro by contacting the MHC protein with a peptide cancer antigen and a MHC-peptide antigen stabilizing compound, thus forming an MHC-peptide-compound complex, and then detecting the increased stability of the MHC-peptide-compound complex relative to the stability of an MHC-peptide complex which does not include the MHC-peptide antigen stabilizing compound.

In embodiments, the MHC-peptide antigen stabilizing compound has molecular weight of at least 10 g/mol. In embodiments, the MHC-peptide antigen stabilizing compound has molecular weight of at least 50 g/mol. In embodiments, the MHC-peptide antigen stabilizing compound has molecular weight of at least 100 g/mol. In embodiments, the MHC-peptide antigen stabilizing compound has molecular weight of at least 200 g/mol. In embodiments, the MHC-peptide antigen stabilizing compound has molecular weight of at least 300 g/mol. In embodiments, the MHC-peptide antigen stabilizing compound has molecular weight of at least 4000 g/mol. In embodiments, the MHC-peptide antigen stabilizing compound has molecular weight of at least 500 g/mol. In embodiments, the MHC-peptide antigen stabilizing compound has molecular weight of at least 600 g/mol. In embodiments, the MHC-peptide antigen stabilizing compound has molecular weight of at least 700 g/mol. In embodiments, the MHC-peptide antigen stabilizing compound has molecular weight of at least 800 g/mol. In embodiments, the MHC-peptide antigen stabilizing compound has molecular weight of at least 900 g/mol. In embodiments, the MHC-peptide antigen stabilizing compound has molecular weight of at least 1000 g/mol. In embodiments, the MHC-peptide antigen stabilizing compound has molecular weight of at least 1500 g/mol. In embodiments, the MHC-peptide antigen stabilizing compound has molecular weight of at least 2000 g/mol.

In embodiments, the MHC protein binds to the peptide cancer antigen with a Kd of greater than 1 micromolar. In embodiments, the MHC protein binds to the peptide cancer antigen with a Kd of greater than 10 nM, 100 nM, 500 nM, 1 μM, 10 μM, 50 μM, 100 μM, 500 μM, or 1 mM. The Kd may be a specific value in a range from 10 nM to 100 nM, 100 nM to 500 nM, 500 nM to 1 μM, 1 μM to 10 μM, 10 μM to 50 μM, 50 μM to 100 μM, 100 μM to 500 μM, or 500 μM to 1 mM. The specific value may be selected from any 1 nM increment in the selected range. The Kd may be selected from a sub-range within any of the aforementioned Kd ranges. The low endpoint of a sub-range may be the low end of the range or any value selected from 1 nM increments above the low end of the range up to 1 nM less that the high end of the range. The high endpoint of a sub-range may be the high end of the range or any value selected from 1 nM below the high end of the range to 1 nM greater than the low end of the range.

In embodiments, the MHC protein binds to the peptide cancer antigen with a Kd of greater than 10 nM. In embodiments, the MHC protein binds to the peptide cancer antigen with a Kd of greater than 100 nM. In embodiments, the MHC protein binds to the peptide cancer antigen with a Kd of greater than 500 nM. In embodiments, the MHC protein binds to the peptide cancer antigen with a Kd of greater than 1 μM. In embodiments, the MHC protein binds to the peptide cancer antigen with a Kd of greater than 10 μM. In embodiments, the MHC protein binds to the peptide cancer antigen with a Kd of greater than 50 μM. In embodiments, the MHC protein binds to the peptide cancer antigen with a Kd of greater than 100 μM. In embodiments, the MHC protein binds to the peptide cancer antigen with a Kd of greater than 500 μM. In embodiments, the MHC protein binds to the peptide cancer antigen with a Kd of greater than 1 mM.

In embodiments, the MHC protein contacting the peptide cancer antigen and a candidate compound is folded or unfolded. In embodiments, the MHC protein contacting the peptide cancer antigen and a candidate compound is folded. In embodiments, the MHC protein contacting the peptide cancer antigen and a candidate compound is unfolded.

In embodiments, the MHC protein is an MHC class I protein or an MHC class II protein. In embodiments, the MHC protein is an MHC class I protein. In embodiments, the MHC protein is an MHC class II protein.

In another aspect, provided herein is a method of identifying a modified peptide-MHC protein allele binding pair which includes (a) contacting a plurality of different modified peptides with a plurality of different MHC protein alleles, and (b) detecting or computationally predicting binding of a first modified peptide to a first MHC protein allele, thereby identifying a modified peptide-MHC protein allele binding pair.

In embodiments, the first modified peptide is modified with a tryptophan. In embodiments, a plurality of different modified peptides are modified with a tryptophan. In embodiments, a peptide modified with a tryptophan is substituted with a tryptophan. In embodiments, the natural sequence of the peptide is modified by replacing one amino acid with a tryptophan. In embodiments the chemical structure of the tryptophan is similar to the structure of the MHC-peptide antigen stabilizing compound.

In embodiments, the plurality of different modified peptides are modified with a tryptophan on any one of the residues. In embodiments, the plurality of different modified peptides are modified with a tryptophan on the first residue. In embodiments, the plurality of different modified peptides are modified with a tryptophan on the last residue. In embodiments, the natural sequence of the peptide is modified by replacing the first amino acid with a tryptophan. In embodiments, the natural sequence of the peptide is modified by replacing the last amino acid with a tryptophan.

In embodiments, the plurality of different modified peptides are derived from a driver oncogene protein. In embodiments, the plurality of different modified peptides are derived from a common driver oncogene protein. In embodiments, driver oncogenic alterations, can include mutation, truncation, gene fusion, and/or splice variants. In embodiments, driver oncogenic alterations, can include mutation. In embodiments, driver oncogenic alterations, can include truncation. In embodiments, driver oncogenic alterations, can include gene fusion. In embodiments, driver oncogenic alterations, can include splice variants.

In embodiments, the plurality of different modified peptides are derived from a K-Ras protein or a mutant K-Ras protein. In embodiments, the plurality of different modified peptides are derived from a K-Ras protein. In embodiments, the plurality of different modified peptides are derived from a mutant K-Ras protein. In embodiments, the plurality of different modified peptides are derived from a recurrent mutant K-Ras protein. In embodiments, the peptide antigen includes a K-Ras amino acid sequence including an amino acid mutation found in a K-Ras driver oncogene protein product of a K-Ras driver oncogene mutation.

In embodiments, the plurality of different modified peptides are derived from a N-Ras protein or a mutant N-Ras protein. In embodiments, the plurality of different modified peptides are derived from a N-Ras protein. In embodiments, the plurality of different modified peptides are derived from a mutant N-Ras protein. In embodiments, the plurality of different modified peptides are derived from a recurrent mutant N-Ras protein. In embodiments, the peptide antigen includes an N-Ras amino acid sequence including an amino acid mutation found in a N-Ras driver oncogene protein product of a N-Ras driver oncogene mutation.

In embodiments, the plurality of different modified peptides are derived from a H-Ras protein or a mutant H-Ras protein. In embodiments, the plurality of different modified peptides are derived from a H-Ras protein. In embodiments, the plurality of different modified peptides are derived from a mutant H-Ras protein. In embodiments, the plurality of different modified peptides are derived from a recurrent mutant H-Ras protein. In embodiments, the peptide antigen includes an H-Ras amino acid sequence including an amino acid mutation found in a H-Ras driver oncogene protein product of a H-Ras driver oncogene mutation.

In embodiments, the plurality of different modified peptides are derived from a B-Raf protein or a mutant B-Raf protein. In embodiments, the plurality of different modified peptides are derived from a B-Raf protein. In embodiments, the plurality of different modified peptides are derived from a mutant B-Raf protein. In embodiments, the plurality of different modified peptides are derived from a recurrent mutant B-Raf protein. In embodiments, the peptide antigen includes a B-Raf amino acid sequence including an amino acid mutation found in a B-Raf driver oncogene protein product of a B-Raf driver oncogene mutation.

In embodiments, the plurality of different modified peptides are derived from a PI3K protein or a mutant PI3K protein. In embodiments, the plurality of different modified peptides are derived from a PI3K protein. In embodiments, the plurality of different modified peptides are derived from a mutant PI3K protein. In embodiments, the peptide antigen includes a PI3K amino acid sequence including an amino acid mutation found in a PI3K driver oncogene protein product of a PI3K driver oncogene mutation.

In embodiments, the plurality of different modified peptides are derived from a p53 protein or a mutant p53 protein. In embodiments, the plurality of different modified peptides are derived from a p53 protein. In embodiments, the plurality of different modified peptides are derived from a mutant p53 protein. In embodiments, the plurality of different modified peptides are derived from a recurrent mutant p53 protein. In embodiments, the peptide antigen includes a p53 amino acid sequence including an amino acid mutation found in a p53 driver oncogene protein product of a p53 driver oncogene mutation.

In embodiments, the mutant K-Ras protein is G12D, G12V, G12C, G12R, G12S, G12A, G13D, G13C, A59E, A59G, A59T, Q61K, Q61L, Q61R, Q61H, K117N, A146P, A146T, or A146V. In embodiments, the mutant K-Ras protein is G12D. In embodiments, the mutant K-Ras protein is G12V. In embodiments, the mutant K-Ras protein is G12C. In embodiments, the peptide antigen includes a K-Ras amino acid sequence including one for more of the following amino acid mutations found in a K-Ras driver oncogene protein: G12D, G12V, G12C, G12R, G12S, G12A, G13D, G13C, A59E, A59G, A59T, Q61K, Q61L, Q61R, Q61H, K117N, A146P, A146T, or A146V. In embodiments, the peptide antigen includes an amino acid sequence including a G12D K-Ras mutation. In embodiments, the peptide antigen includes an amino acid sequence including a G12V K-Ras mutation. In embodiments, the peptide antigen includes an amino acid sequence including a G12C K-Ras mutation.

In embodiments, the mutant N-Ras protein is G12C, G12S, G12D, G12A, G12V, G13D, G13C, G13V, G13R, A59T, Q61K, Q61L, Q61R, Q61H, K117N, A156P, A146T, or A146V. In embodiments, the peptide antigen includes an N-Ras amino acid sequence including one for more of the following amino acid mutations found in an N-Ras driver oncogene protein: G12C, G12S, G12D, G12A, G12V, G13D, G13C, G13V, G13R, A59T, Q61K, Q61L, Q61R, Q61H, K117N, A156P, A146T, or A146V.

In embodiments, the mutant H-Ras protein is G12C, G12S, G12D, G12A, G12V, G13D, G13C, G13V, G13R, A59T, Q61K, Q61L, Q61R, Q61H, K117N, A156P, A146T, or A146V. In embodiments, the peptide antigen includes an H-Ras amino acid sequence including one for more of the following amino acid mutations found in an H-Ras driver oncogene protein: G12C, G12S, G12D, G12A, G12V, G13D, G13C, G13V, G13R, A59T, Q61K, Q61L, Q61R, Q61H, K117N, A156P, A146T, or A146V.

In embodiments, the mutant B-Raf protein is V600E. In embodiments, the peptide antigen includes a B-Raf amino acid sequence including a V600E mutation. In embodiments, the mutant PI3K protein is E545K or H1047R. In embodiments, the peptide antigen includes a PI3K amino acid sequence including an E545K or H1047R mutation. In embodiments, the mutant PI3K protein is E545K. In embodiments, the peptide antigen includes a PI3K amino acid sequence including an E545K mutation. In embodiments, the mutant PI3K protein is H1047R. In embodiments, the peptide antigen includes a PI3K amino acid sequence including a H1047R mutation.

In embodiments, the mutant EGFR protein contains one or more inframe deletions in exon 19. In embodiments, the mutant EGFR protein contains one or more insertions in exon 20. In embodiments, the mutant EGFR protein contains one or more exon 20 insertions. In embodiments, the mutant EGFR protein contains one or more mutations at residues 761, 762, 763, 764, 765, 766, 767, 768, 769, 770, 771, 772, 773, 774, and 775. In embodiments, the mutant EGFR protein contains one or more insertions at residues 761, 762, 763, 764, 765, 766, 767, 768, 769, 770, 771, 772, 773, 774, and 775. In embodiments, the mutant EGFR protein contains one or more insertions at residue 761. In embodiments, the mutant EGFR protein contains one or more insertions at residue 762. In embodiments, the mutant EGFR protein contains one or more insertions at residue 763. In embodiments, the mutant EGFR protein contains one or more insertions at residue 764. In embodiments, the mutant EGFR protein contains one or more insertions at residue 765. In embodiments, the mutant EGFR protein contains one or more insertions at residue 766. In embodiments, the mutant EGFR protein contains one or more insertions at residue 767. In embodiments, the mutant EGFR protein contains one or more insertions at residue 768. In embodiments, the mutant EGFR protein contains one or more insertions at residue 769. In embodiments, the mutant EGFR protein contains one or more insertions at residue 770. In embodiments, the mutant EGFR protein contains one or more insertions at residue 771. In embodiments, the mutant EGFR protein contains one or more insertions at residue 772. In embodiments, the mutant EGFR protein contains one or more insertions at residue 773. In embodiments, the mutant EGFR protein contains one or more insertions at residue 774. In embodiments, the mutant EGFR protein contains one or more insertions at residue 775. In embodiments, the mutant EGFR protein contains one or more deletions at residues 761, 762, 763, 764, 765, 766, 767, 768, 769, 770, 771, 772, 773, 774, and 775. In embodiments, the mutant EGFR protein is L858R. In embodiments, the mutant EGFR protein is reported in “Lancet Oncol. 2012, 13, e23-31”, which is incorporated by reference, herein, in its entirety. Thus, in embodiments, the peptide antigen includes an EGFR amino acid sequence including one for more of the following amino acid mutations recited above.

In embodiments, the mutant p53 protein is R175H, R175G, R175L, R175C, Y220C, G245S, G245D, G245V, G245R, R248Q, R248W, R248L, R273H, R273C, R273L, R282W, or R282G. In embodiments, the peptide antigen includes an p53 amino acid sequence including one for more of the following amino acid mutations found in an p53 driver oncogene protein: R175H, R175G, R175L, R175C, Y220C, G245S, G245D, G245V, G245R, R248Q, R248W, R248L, R273H, R273C, R273L, R282W, or R282G.

In another aspect, provided herein is a method of vaccinating a subject for cancer, which includes administering a peptide cancer antigen, and a compound that stabilizes binding of an MHC protein to the peptide cancer antigen.

In another aspect, provided herein is a method of vaccinating a subject for cancer, which includes administering peptide-compound conjugate, wherein the peptide-compound conjugate includes a peptide cancer antigen that is linked to a compound via a chemical bond.

In embodiments, the MHC-peptide cancer antigen stabilizing compound used for vaccinating a subject for cancer is identified by contacting an MHC protein with a peptide antigen and a candidate compound thereby forming an MHC-peptide-compound complex, and then detecting the increased stability of the MHC-peptide-compound complex relative to the stability of an MHC-peptide complex which does not include the candidate compound.

In embodiments, a vaccine is administered in a single formulation.

### III. Compositions

In another aspect, provided herein is a composition including an MHC protein, a peptide antigen, and a compound; the MHC protein, the peptide antigen, and the compound are bound to form an MHC-peptide-compound complex. In embodiments, the compound stabilizes the binding of the MHC protein to the peptide antigen relative to the absence of the compound.

In embodiments, the MHC protein is covalently bound to the peptide antigen. In embodiments, the MHC protein is covalently bound to the peptide antigen through a disulfide bond. In embodiments, the MHC protein is covalently bound to the peptide antigen through a thioether. In embodiments, the MHC protein is covalently bound to the peptide antigen through a thioester. In embodiments, the MHC protein is covalently bound to the peptide antigen through a sulfonamide. In embodiments, the MHC protein is covalently bound to the peptide antigen through an amide. In embodiments, the MHC protein is covalently bound to the peptide antigen through an imine. In embodiments, the MHC protein is covalently bound to the peptide antigen through a Ser-O—B linker. In embodiments, the MHC protein is covalently bound to the peptide antigen through a Ser-O—B linker, wherein the Ser-O—B linker is formed between a serine residue and a benzoxaborole moiety.

In embodiments, the MHC protein is covalently bound to the peptide antigen through a disulfide bond. In embodiments, a cysteine amino acid within the MHC protein forms a part of the disulfide bond. In embodiments, a homocysteine amino acid within the MHC protein forms a part of the disulfide bond.

In embodiments, the compound is covalently bound to the peptide antigen. In embodiments, the compound is covalently bound to the peptide antigen through a reaction between an electrophilic moiety on the compound and a nucleophilic moiety on the peptide antigen.

In embodiments, the nucleophilic moiety is a cysteine sulfhydryl group. In embodiments, the nucleophilic moiety is a homocysteine sulfhydryl group.

In embodiments, the nucleophilic group is a lysine amine group. In embodiments, the nucleophilic group is an ornithine amine group.

In embodiments, the nucleophilic group is a serine hydroxyl group. In embodiments, the nucleophilic group is a threonine hydroxyl group.

In another aspect, provided herein is a composition including an MHC protein which is covalently bound to a peptide antigen. In embodiments, the MHC protein is covalently bound to the peptide antigen through a disulfide bond. In embodiments, a cysteine amino acid within the MHC protein forms a part of the disulfide bond. In embodiments, a homocysteine amino acid within the MHC protein forms a part of the disulfide bond.

In another aspect, provided herein is a composition including a peptide antigen, which is covalently bound to a compound. In embodiments, the compound is covalently bound to the peptide antigen through a reaction between an electrophilic moiety on the compound and a nucleophilic moiety on the peptide antigen. In embodiments, the nucleophilic moiety is a cysteine sulfhydryl group. In embodiments, the nucleophilic moiety is a homocysteine sulfhydryl group. In embodiments, the nucleophilic group is a lysine amine group. In embodiments, the nucleophilic group is an ornithine amine group. In embodiments, the nucleophilic group is a serine hydroxyl group. In embodiments, the nucleophilic group is a threonine hydroxyl group.

In embodiments, the electrophilic moiety is:

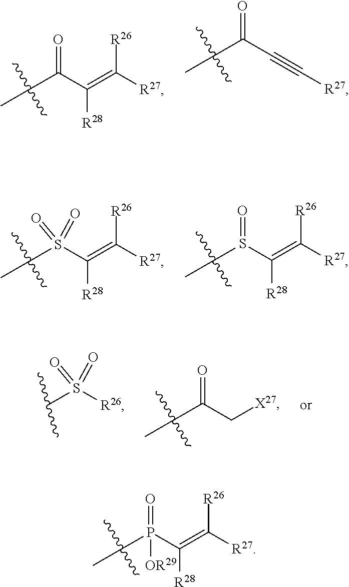

R²⁶ is independently hydrogen, halogen, —CX²⁶₃, —CHX²⁶₂, —CH₂X²⁶, —CN, —SOₙ₂₆R^{26A}, SOᵥ₂₆NR^{26A}R^{26B}, —NHNR^{26A}R^{26B}, —ONR^{26A}R^{26B}, —NHC(O)NHNR^{26A}R^{26B}, —NHC(O)NR^{26A}R^{26B}, —N(O)ₘ₂₆, —NR^{26A}R^{26B}, —C(O)R^{26A}, —C(O)—OR^{26A}, —C(O)NR^{26A}R^{26B}, —OR^{26A}, —NR^{26A}SO₂R^{26B}, —NR^{26A}C(O)R^{26B}, —NR^{26A}C(O)OR^{26B}, —NR^{26A}OR^{26B}, —OCX²⁶₃, —OCHX²⁶₂, —OCH₂X²⁶, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R²⁷ is independently hydrogen,

halogen, —CX²⁷₃, —CHX²⁷₂, —CH₂X²⁷, —CN, —SOₙ₂₇R^{27A}, —SOᵥ₂₇NR^{27A}R^{27B}, —NHNR^{27A}R^{27B}, —ONR^{27A}R^{27B}, —NHC(O)NHNR^{27A}R^{27B},
—NHC(O)NR^{27A}R^{27B}, —N(O)ₘ₂₇, —NR^{27A}R^{27B}, —C(O)R^{27A}, —C(O)—OR^{27A}, —C(O)NR^{27A}R^{27B}, —OR^{27A}, —NR^{27A}SO₂R^{27B}, —NR^{27A}C(O)R^{27B}, —NR^{27A}C(O)OR^{27B}, —NR^{27A}OR^{27B}, —OCX²⁷₃, —OCHX²⁷₂, —OCH ₂X²⁷, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R²⁸ is independently hydrogen,

halogen, —CX²⁸₃, —CHX²⁸₂, —CH₂X²⁸, —CN, —SOₙ₂₈R^{28A}, —SOᵥ₂₈NR^{28A}R^{28B}, —NHNR^{28A}R^{28B}, —ONR^{28A}R^{28B}, —NHC(O)NHNR^{28A}R^{28B},
—NHC(O)NR^{28A}R^{28B}, —N(O)ₘ₂₈, —NR^{28A}R^{28B}, —C(O)R^{28A}, —C(O)—OR^{28A}, —C(O)NR^{28A}R^{28B}, —OR^{28A}, —NR^{28A}SO₂R^{28B}, —NR^{28A}C(O)R^{28B}, —NR^{28A}C(O)OR^{28B}, —NR^{28A}OR^{28B}, —OCX²⁸₃, —OCHX²⁸₂, —OCH ₂X²⁸, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R²⁹ is independently hydrogen,

halogen, —CX²⁹₃, —CHX²⁹₂, —CH₂X²⁹, —CN, —SOₙ₂₉R^{29A}, —SOᵥ₂₉NR^{29A}R^{29B}, —NHNR^{29A}R^{29B}, —ONR^{29A}R^{29B}, —NHC(O)NHNR^{29A}R^{29B},
—NHC(O)NR^{29A}R^{29B}, —N(O)ₘ₂₉, —NR^{29A}R^{29B}, —C(O)R^{29A}, —C(O)—OR^{29A}, —C(O)NR^{29A}R^{29B}, —OR^{29A}, —NR^{29A}SO₂R^{29B}, —NR^{29A}C(O)R^{29B}, —NR^{29A}C(O)OR^{29B}, —NR^{29A}OR^{29B}, —OCX²⁹₃, —OCHX²⁹₂, —OCH ₂X²⁹, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl.

R^{26A}, R^{26B}, R^{27A}, R^{27B}, R^{28A}, R^{28B}, R^{29A}, and R^{29B} are independently hydrogen, —CX₃, —CHX₂, —CH₂X, —CN, —OH, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{26A} and R^{26B} substituents bonded to the same nitrogen atom may optionally be joined to form a substituted or unsubstituted heterocycloalkyl or substituted or unsubstituted heteroaryl; R^{27A} and R^{27B} substituents bonded to the same nitrogen atom may optionally be joined to form a substituted or unsubstituted heterocycloalkyl or substituted or unsubstituted heteroaryl; R^{28A} and R^{28B} substituents bonded to the same nitrogen atom may optionally be joined to form a substituted or unsubstituted heterocycloalkyl or substituted or unsubstituted heteroaryl; and R^{29A} and R^{29B} substituents bonded to the same nitrogen atom may optionally be joined to form a substituted or unsubstituted heterocycloalkyl or substituted or unsubstituted heteroaryl.

X²⁶, X²⁷, X²⁸, and X²⁹ are independently —F, —Cl, —Br, or —I.

n26, n27, n28, and n29 are independently an integer from 0 to 4.

m26, m27, m28, m29, v26, v27, v28, and v29 are independently 1 or 2.

In embodiments, the electrophilic moiety is:

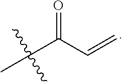 [SMILES: *C(=O)C=C]

In embodiments, the electrophilic moiety is:

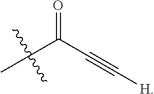

In embodiments, the electrophilic moiety is:

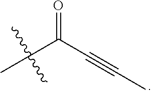 [SMILES: *C(=O)C#CC]

In embodiments, the electrophilic moiety is:

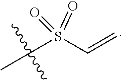 [SMILES: *S(=O)(=O)C=C]

In embodiments, the electrophilic moiety is:

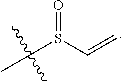 [SMILES: *S(=O)C=C]

In embodiments, the electrophilic moiety is:

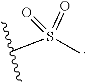 [SMILES: *S(C)(=O)=O]

In embodiments, the electrophilic moiety is:

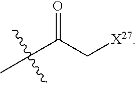

In embodiments, the electrophilic moiety is:

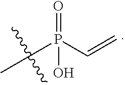 [SMILES: *P(=O)(O)C=C]

In embodiments, the electrophilic moiety is:

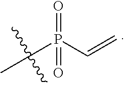

In embodiments, when the compound is covalently bound, the compound is bound through an irreversible covalent bond.

In embodiments, the compositions provided herein are used for a therapeutic purpose. In some embodiments, a therapeutic purpose encompasses a prophylactic purpose (a purpose of preventing a disease or condition from occurring) and a treatment purpose (a purpose of treating an existing disease or condition).

In embodiments, a composition can be a vaccine or a composition thereof, i.e. a composition that contains the vaccine and optionally a pharmaceutically acceptable carrier. The vaccine or vaccine composition can be used to prevent and/or treat a disease or condition. In embodiments, the pharmaceutical composition can further contain a pharmaceutically acceptable excipient and/or a pharmaceutically acceptable carrier.

In embodiments, pharmaceutical compositions can have a peptide cancer antigen and a compound that stabilizes binding of an MHC protein to the peptide cancer antigen, as an active ingredient and further contain pharmaceutically acceptable excipients or additives depending on the route of administration. Examples of such excipients or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used can be chosen from, but not limited to, the above or combinations thereof, as appropriate, depending on the dosage form of the present disclosure.

Formulation of the pharmaceutical compositions of the present disclosure can vary according to the route of administration selected (e.g., solution, emulsion). Routes of administration can be, for example, intramuscular, subcutaneous, intravenous, intralymphatic, subcutaneous, intramuscular, intraocular, topical skin, topical conjunctival, oral, intravessical (bladder), intraanal and intravaginal.

“Administration,” “administering” may mean oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

In embodiments, the formulation is a controlled release formulation. In embodiments, the composition includes a controlled release formulation. The term “controlled release formulation” includes sustained release and time-release formulations. Controlled release formulations are well-known in the art. These include excipients that allow for sustained, periodic, pulse, or delayed release of the composition. Controlled release formulations include, without limitation, embedding of the composition into a matrix; enteric coatings; micro-encapsulation; gels and hydrogels; implants; and any other formulation that allows for controlled release of a composition.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### IV. Compounds

In another aspect, provided herein is a compound having the formula:

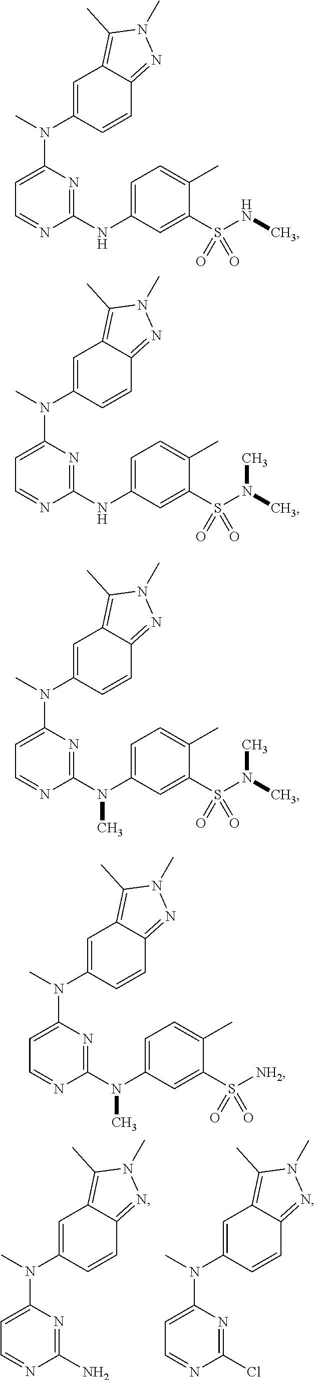

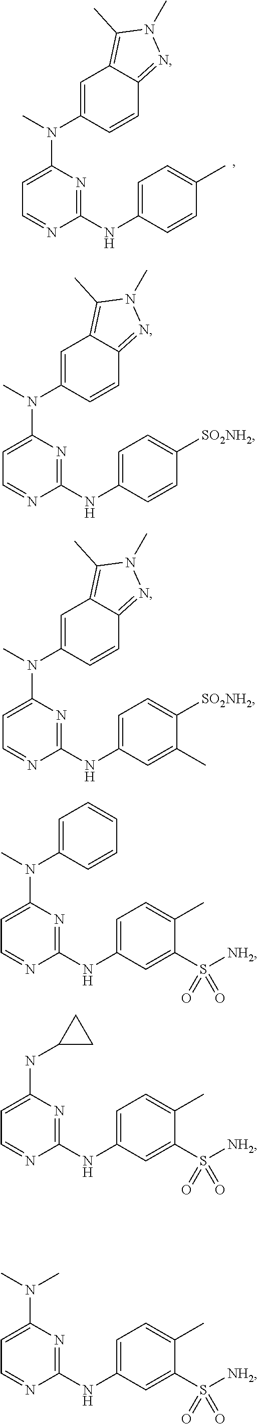

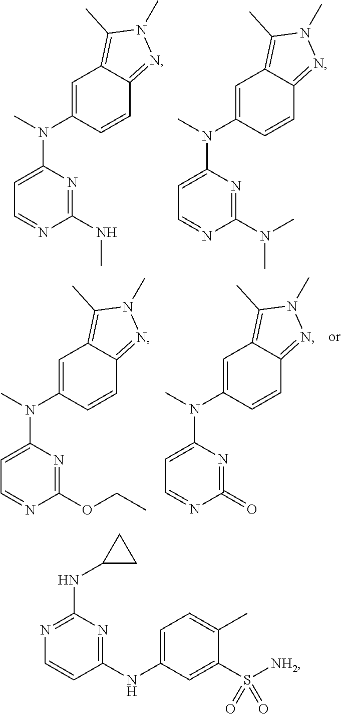

or a salt thereof.

In embodiments, the compound is:

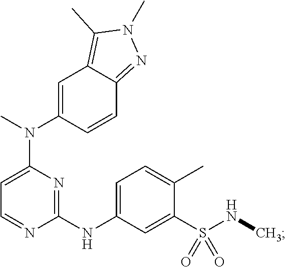

or a salt thereof. In embodiments, the compound is:

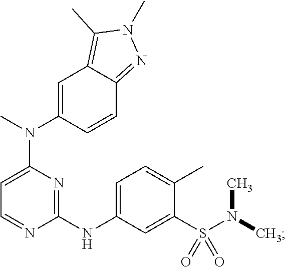

or a salt thereof. In embodiments, the compound is:

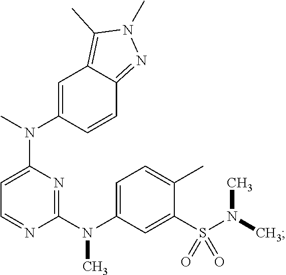

or a salt thereof. In embodiments, the compound is:

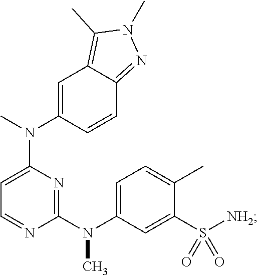

or a salt thereof. In embodiments, the compound is:

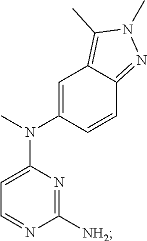 [SMILES: Cc1c2cc(N(C)c3ccnc(N)n3)ccc2nn1C]

or a salt thereof. In embodiments the compound is:

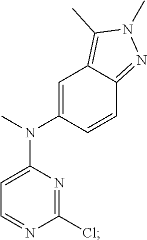 [SMILES: Cc1c2cc(N(C)c3ccnc(Cl)n3)ccc2nn1C]

or a salt thereof. In embodiments, the compound is:

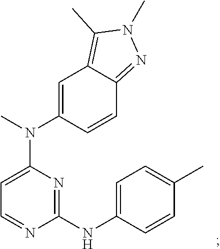 [SMILES: Cc1ccc(Nc2nccc(N(C)c3ccc4nn(C)c(C)c4c3)n2)cc1]

or a salt thereof. In embodiments, the compound is:

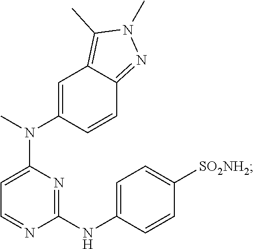 [SMILES: Cc1c2cc(N(C)c3ccnc(Nc4ccc(S(N)(=O)=O)cc4)n3)ccc2nn1C]

or a salt thereof. In embodiments, the compound is:

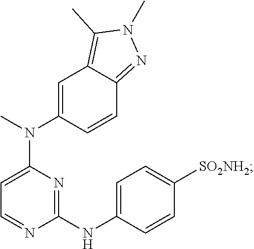 [SMILES: Cc1c2cc(N(C)c3ccnc(Nc4ccc(S(N)(=O)=O)cc4)n3)ccc2nn1C]

or a salt thereof. In embodiments, the compound is:

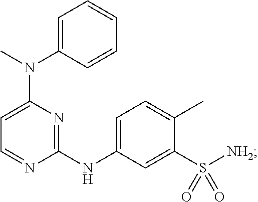 [SMILES: Cc1ccc(Nc2nccc(N(C)c3ccccc3)n2)cc1S(N)(=O)=O]

or a salt thereof. In embodiments, the compound is:

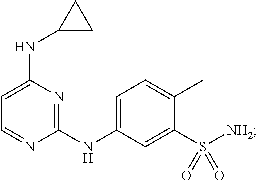 [SMILES: Cc1ccc(Nc2nccc(NC3CC3)n2)cc1S(N)(=O)=O]

or a salt thereof. In embodiments, the compound is:

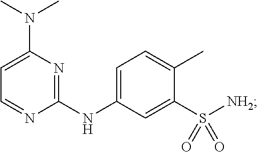 [SMILES: Cc1ccc(Nc2nccc(N(C)C)n2)cc1S(N)(=O)=O]

or a salt thereof. In embodiments, the compound is:

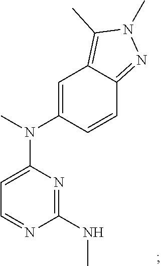 [SMILES: CNc1nccc(N(C)c2ccc3nn(C)c(C)c3c2)n1]

or a salt thereof. In embodiments, the compound is:

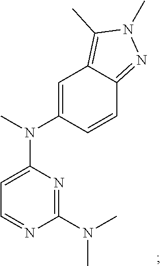 [SMILES: Cc1c2cc(N(C)c3ccnc(N(C)C)n3)ccc2nn1C]

or a salt thereof. In embodiments, the compound is:

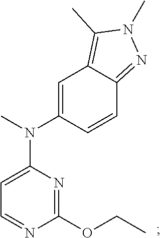 [SMILES: CCOc1nccc(N(C)c2ccc3nn(C)c(C)c3c2)n1]

or a salt thereof. In embodiments, the compound is:

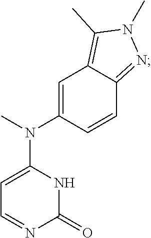 [SMILES: Cc1c2cc(N(C)c3ccnc(=O)[nH]3)ccc2nn1C]

or a salt thereof. In embodiments, the compound is:

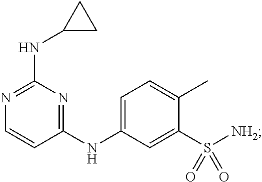 [SMILES: Cc1ccc(Nc2ccnc(NC3CC3)n2)cc1S(N)(=O)=O]

or a salt thereof.

In another aspect, provided herein is a compound having the formula:

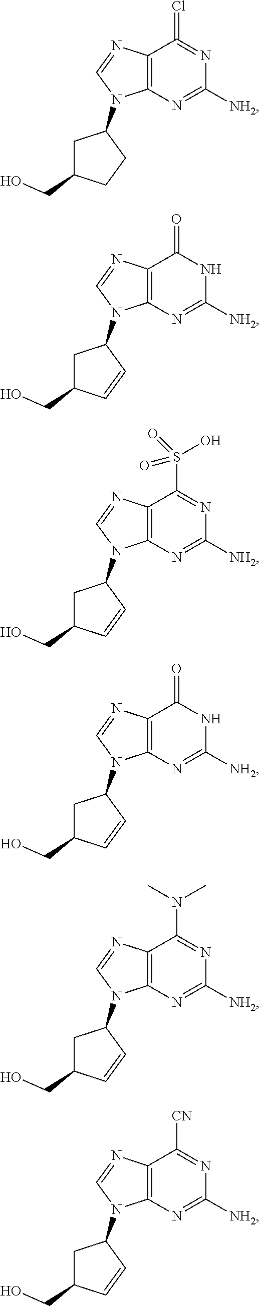

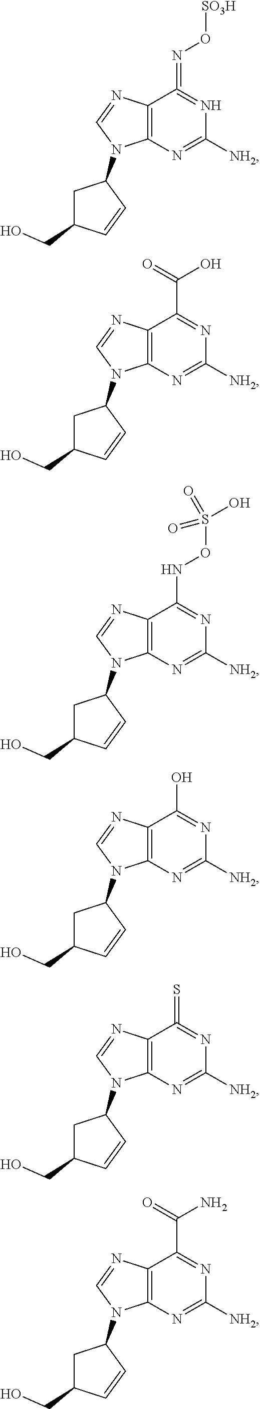

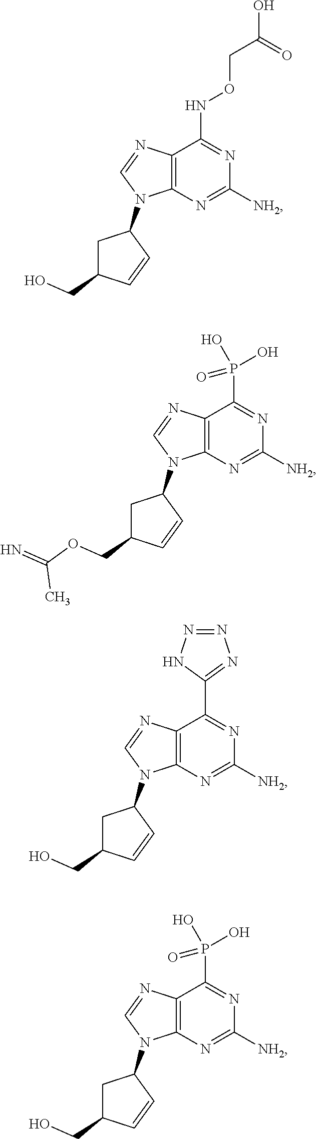 [SMILES: Nc1nc(NOCC(=O)O)c2ncn([C@H]3C=C[C@@H](CO)C3)c2n1 ; CC(=N)OC[C@@H]1C=C[C@H](n2cnc3c(P(=O)(O)O)nc(N)nc32)C1 ; Nc1nc(-c2nnn[nH]2)c2ncn([C@H]3C=C[C@@H](CO)C3)c2n1 ; Nc1nc(P(=O)(O)O)c2ncn([C@H]3C=C[C@@H](CO)C3)c2n1]

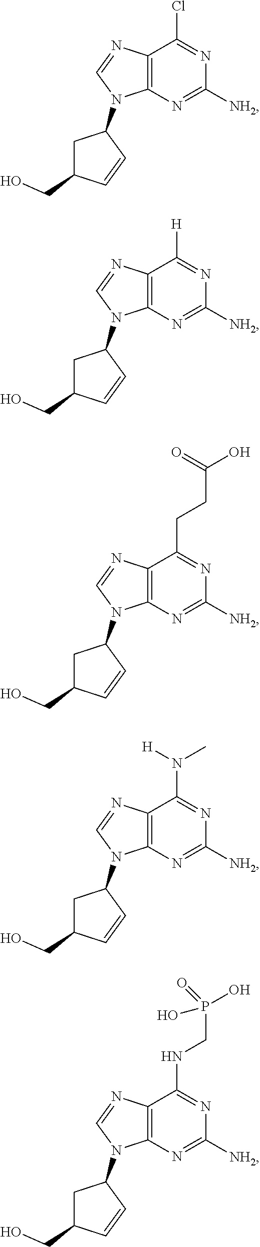 [SMILES: Nc1nc(Cl)c2ncn([C@H]3C=C[C@@H](CO)C3)c2n1 ; Nc1ncc2ncn([C@H]3C=C[C@@H](CO)C3)c2n1 ; Nc1nc(CCC(=O)O)c2ncn([C@H]3C=C[C@@H](CO)C3)c2n1 ; CNc1nc(N)nc2c1ncn2[C@H]1C=C[C@@H](CO)C1 ; Nc1nc(NCP(=O)(O)O)c2ncn([C@H]3C=C[C@@H](CO)C3)c2n1]

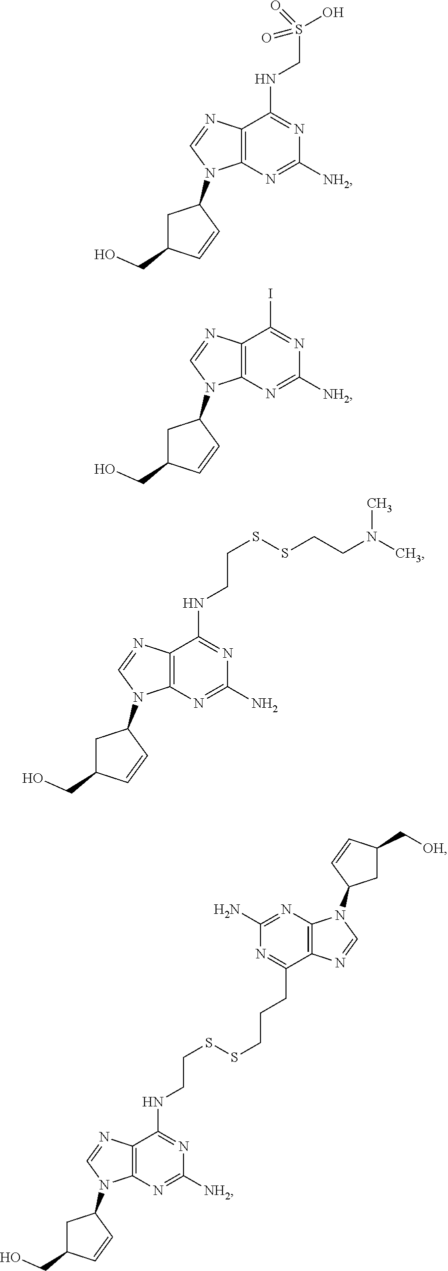 [SMILES: Nc1nc(NCS(=O)(=O)O)c2ncn([C@H]3C=C[C@@H](CO)C3)c2n1 ; Nc1nc(I)c2ncn([C@H]3C=C[C@@H](CO)C3)c2n1 ; CN(C)CCSSCCNc1nc(N)nc2c1ncn2[C@H]1C=C[C@@H](CO)C1 ; Nc1nc(CCCSSCCNc2nc(N)nc3c2ncn3[C@H]2C=C[C@@H](CO)C2)c2ncn([C@H]3C=C[C@@H](CO)C3)c2n1]

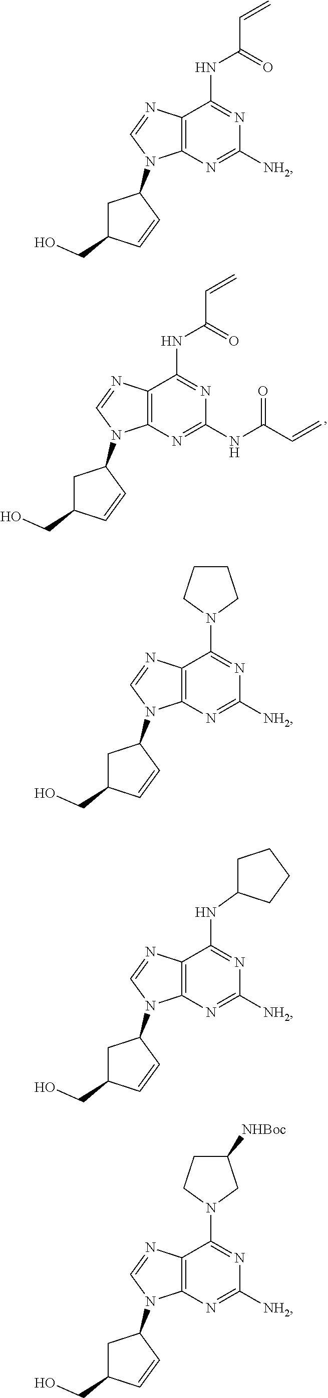 [SMILES: C=CC(=O)Nc1nc(N)nc2c1ncn2[C@H]1C=C[C@@H](CO)C1 ; C=CC(=O)Nc1nc(NC(=O)C=C)c2ncn([C@H]3C=C[C@@H](CO)C3)c2n1 ; Nc1nc(N2CCCC2)c2ncn([C@H]3C=C[C@@H](CO)C3)c2n1 ; Nc1nc(NC2CCCC2)c2ncn([C@H]3C=C[C@@H](CO)C3)c2n1 ; CC(C)(C)OC(=O)N[C@@H]1CCN(c2nc(N)nc3c2ncn3[C@H]2C=C[C@@H](CO)C2)C1]

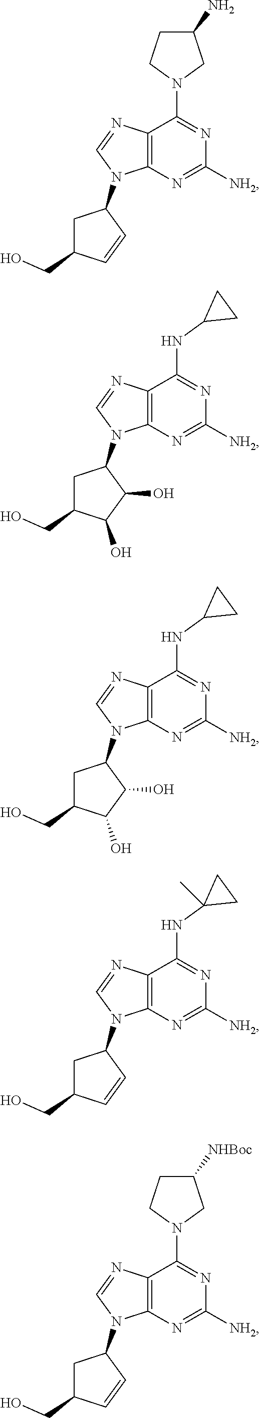 [SMILES: Nc1nc(N2CC[C@@H](N)C2)c2ncn([C@H]3C=C[C@@H](CO)C3)c2n1 ; Nc1nc(NC2CC2)c2ncn([C@@H]3C[C@H](CO)[C@H](O)[C@@H]3O)c2n1 ; Nc1nc(NC2CC2)c2ncn([C@@H]3C[C@H](CO)[C@@H](O)[C@H]3O)c2n1 ; CC1(Nc2nc(N)nc3c2ncn3[C@H]2C=C[C@@H](CO)C2)CC1 ; CC(C)(C)OC(=O)N[C@H]1CCN(c2nc(N)nc3c2ncn3[C@H]2C=C[C@@H](CO)C2)C1]

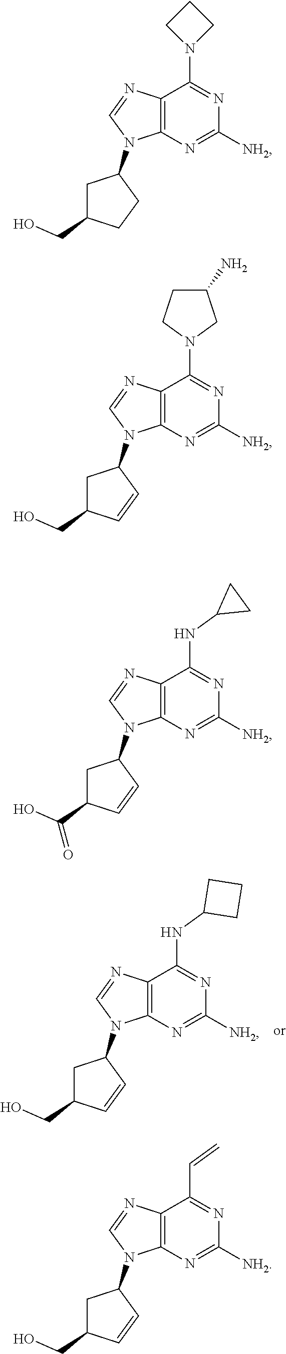 [SMILES: Nc1nc(N2CCC2)c2ncn([C@H]3CC[C@@H](CO)C3)c2n1 ; Nc1nc(N2CC[C@H](N)C2)c2ncn([C@H]3C=C[C@@H](CO)C3)c2n1 ; Nc1nc(NC2CC2)c2ncn([C@H]3C=C[C@@H](C(=O)O)C3)c2n1 ; Nc1nc(NC2CCC2)c2ncn([C@H]3C=C[C@@H](CO)C3)c2n1 ; C=Cc1nc(N)nc2c1ncn2[C@H]1C=C[C@@H](CO)C1]

or a salt thereof.

In embodiments, the compound is:

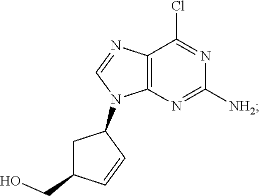 [SMILES: Nc1nc(Cl)c2ncn([C@H]3C=C[C@@H](CO)C3)c2n1]

or a salt thereof. In embodiments, the compound is:

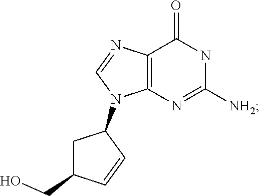 [SMILES: Nc1nc2c(ncn2[C@H]2C=C[C@@H](CO)C2)c(=O)[nH]1]

or a salt thereof. In embodiments, the compound is:

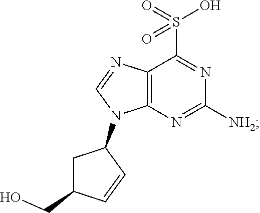 [SMILES: Nc1nc(S(=O)(=O)O)c2ncn([C@H]3C=C[C@@H](CO)C3)c2n1]

or a salt thereof. In embodiments, the compound is:

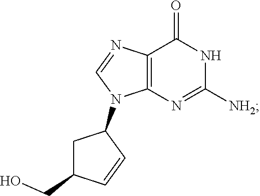 [SMILES: Nc1nc2c(ncn2[C@H]2C=C[C@@H](CO)C2)c(=O)[nH]1]

or a salt thereof. In embodiments, the compound is:

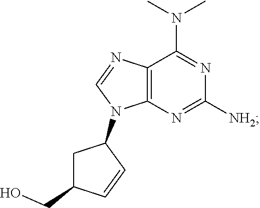 [SMILES: CN(C)c1nc(N)nc2c1ncn2[C@H]1C=C[C@@H](CO)C1]

or a salt thereof. In embodiments, the compound is:

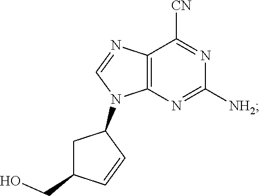 [SMILES: N#Cc1nc(N)nc2c1ncn2[C@H]1C=C[C@@H](CO)C1]

or a salt thereof. In embodiments, the compound is:

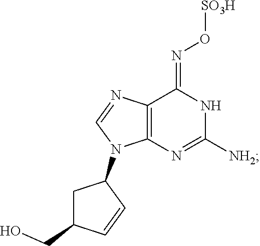 [SMILES: Nc1nc2c(ncn2[C@H]2C=C[C@@H](CO)C2)/c(=N/OS(=O)(=O)O)[nH]1]

or a salt thereof. In embodiments, the compound is:

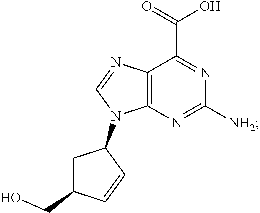 [SMILES: Nc1nc(C(=O)O)c2ncn([C@H]3C=C[C@@H](CO)C3)c2n1]

or a salt thereof. In embodiments, the compound is:

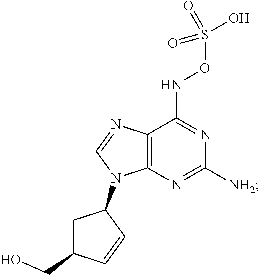 [SMILES: Nc1nc(NOS(=O)(=O)O)c2ncn([C@H]3C=C[C@@H](CO)C3)c2n1]

or a salt thereof. In embodiments, the compound is:

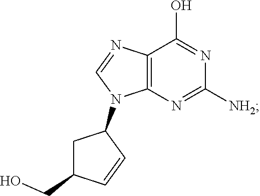 [SMILES: Nc1nc(O)c2ncn([C@H]3C=C[C@@H](CO)C3)c2n1]

or a salt thereof. In embodiments, the compound is:

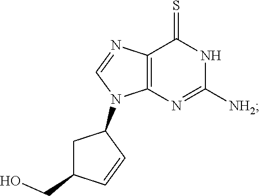 [SMILES: Nc1nc2c(ncn2[C@H]2C=C[C@@H](CO)C2)c(=S)[nH]1]

or a salt thereof. In embodiments, the compound is:

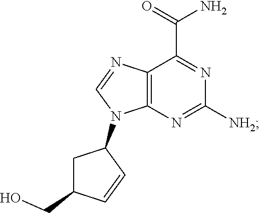 [SMILES: NC(=O)c1nc(N)nc2c1ncn2[C@H]1C=C[C@@H](CO)C1]

or a salt thereof. In embodiments, the compound is:

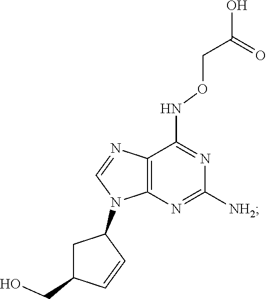 [SMILES: Nc1nc(NOCC(=O)O)c2ncn([C@H]3C=C[C@@H](CO)C3)c2n1]

or a salt thereof. In embodiments, the compound is:

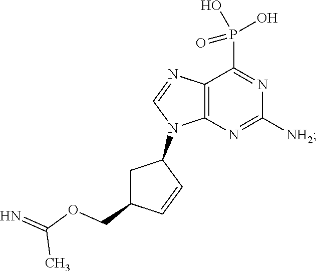 [SMILES: CC(=N)OC[C@@H]1C=C[C@H](n2cnc3c(P(=O)(O)O)nc(N)nc32)C1]

or a salt thereof. In embodiments, the compound is:

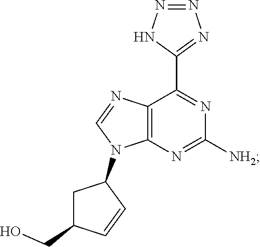 [SMILES: Nc1nc(-c2nnn[nH]2)c2ncn([C@H]3C=C[C@@H](CO)C3)c2n1]

or a salt thereof. In embodiments, the compound is:

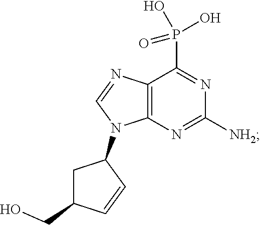 [SMILES: Nc1nc(P(=O)(O)O)c2ncn([C@H]3C=C[C@@H](CO)C3)c2n1]

or a salt thereof. In embodiments, the compound is:

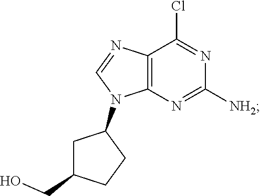 [SMILES: Nc1nc(Cl)c2ncn([C@H]3CC[C@@H](CO)C3)c2n1]

or a salt thereof. In embodiments, the compound is:

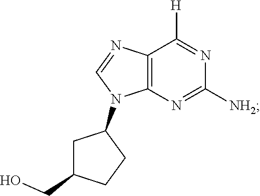 [SMILES: Nc1ncc2ncn([C@H]3CC[C@@H](CO)C3)c2n1]

or a salt thereof. In embodiments, the compound is:

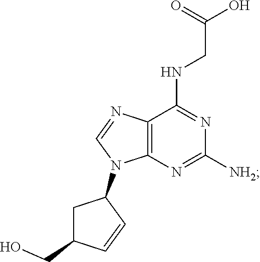 [SMILES: Nc1nc(NCC(=O)O)c2ncn([C@H]3C=C[C@@H](CO)C3)c2n1]

or a salt thereof. In embodiments, the compound is:

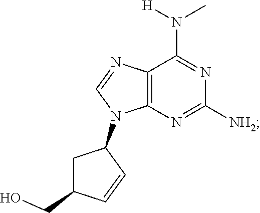 [SMILES: CNc1nc(N)nc2c1ncn2[C@H]1C=C[C@@H](CO)C1]

or a salt thereof. In embodiments, the compound is:

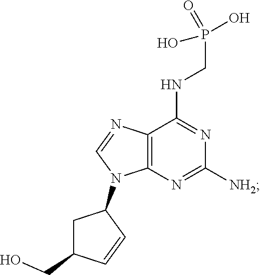 [SMILES: Nc1nc(NCP(=O)(O)O)c2ncn([C@H]3C=C[C@@H](CO)C3)c2n1]

or a salt thereof. In embodiments, the compound is:

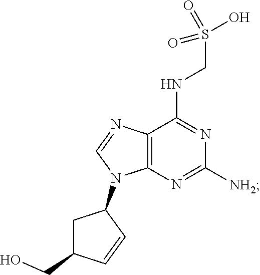 [SMILES: Nc1nc(NCS(=O)(=O)O)c2ncn([C@H]3C=C[C@@H](CO)C3)c2n1]

or a salt thereof. In embodiments, the compound is:

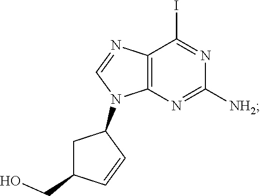 [SMILES: Nc1nc(I)c2ncn([C@H]3C=C[C@@H](CO)C3)c2n1]

or a salt thereof. In embodiments, the compound is:

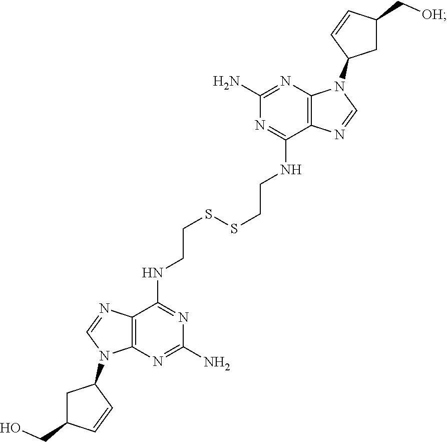 [SMILES: Nc1nc(NCCSSCCNc2nc(N)nc3c2ncn3[C@H]2C=C[C@@H](CO)C2)c2ncn([C@H]3C=C[C@@H](CO)C3)c2n1]

or a salt thereof. In embodiments, the compound is:

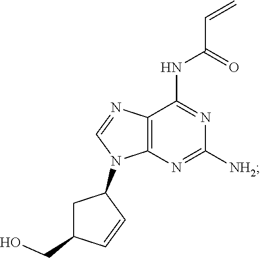 [SMILES: C=CC(=O)Nc1nc(N)nc2c1ncn2[C@H]1C=C[C@@H](CO)C1]

or a salt thereof. In embodiments, the compound is:

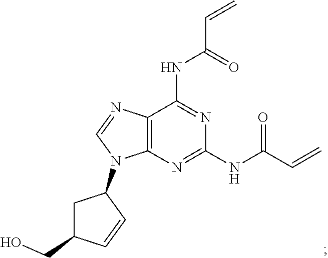 [SMILES: C=CC(=O)Nc1nc(NC(=O)C=C)c2ncn([C@H]3C=C[C@@H](CO)C3)c2n1]

or a salt thereof. In embodiments, the compound is:

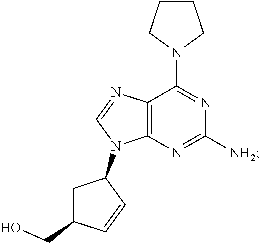 [SMILES: Nc1nc(N2CCCC2)c2ncn([C@H]3C=C[C@@H](CO)C3)c2n1]

or a salt thereof. In embodiments, the compound is:

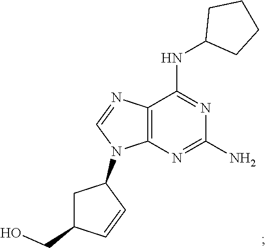 [SMILES: Nc1nc(NC2CCCC2)c2ncn([C@H]3C=C[C@@H](CO)C3)c2n1]

or a salt thereof. In embodiments, the compound is:

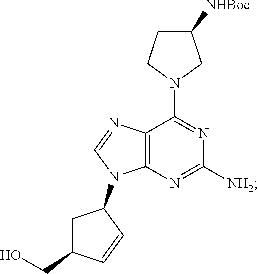 [SMILES: CC(C)(C)OC(=O)N[C@@H]1CCN(c2nc(N)nc3c2ncn3[C@H]2C=C[C@@H](CO)C2)C1]

or a salt thereof. In embodiments, the compound is:

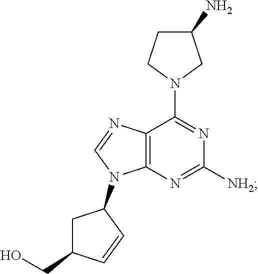 [SMILES: Nc1nc(N2CC[C@@H](N)C2)c2ncn([C@H]3C=C[C@@H](CO)C3)c2n1]

or a salt thereof. In embodiments, the compound is:

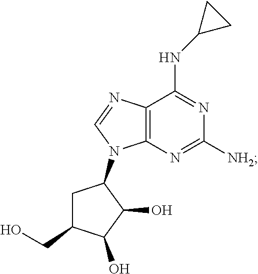 [SMILES: Nc1nc(NC2CC2)c2ncn([C@@H]3C[C@H](CO)[C@H](O)[C@@H]3O)c2n1]

or a salt thereof. In embodiments, the compound is:

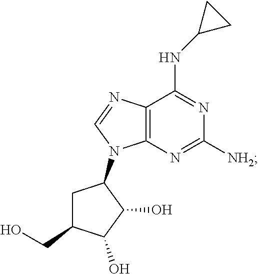 [SMILES: Nc1nc(NC2CC2)c2ncn([C@@H]3C[C@H](CO)[C@@H](O)[C@H]3O)c2n1]

or a salt thereof. In embodiments, the compound is:

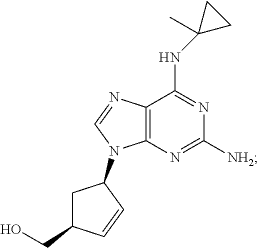 [SMILES: CC1(Nc2nc(N)nc3c2ncn3[C@H]2C=C[C@@H](CO)C2)CC1]

or a salt thereof. In embodiments, the compound is:

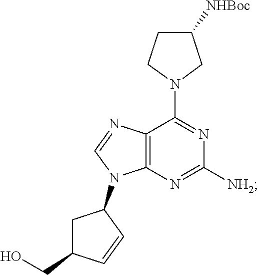 [SMILES: CC(C)(C)OC(=O)N[C@H]1CCN(c2nc(N)nc3c2ncn3[C@H]2C=C[C@@H](CO)C2)C1]

or a salt thereof. In embodiments, the compound is:

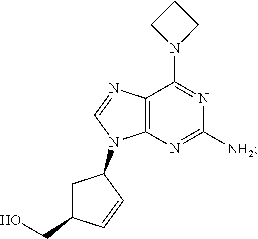 [SMILES: Nc1nc(N2CCC2)c2ncn([C@H]3C=C[C@@H](CO)C3)c2n1]

or a salt thereof. In embodiments, the compound is:

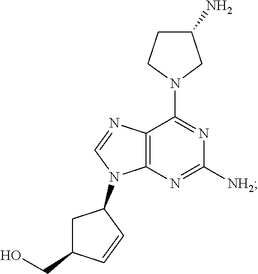 [SMILES: Nc1nc(N2CC[C@H](N)C2)c2ncn([C@H]3C=C[C@@H](CO)C3)c2n1]

or a salt thereof. In embodiments, the compound is:

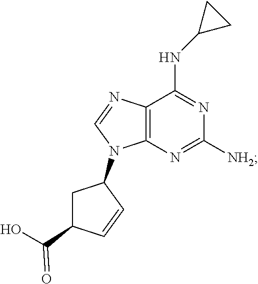 [SMILES: Nc1nc(NC2CC2)c2ncn([C@H]3C=C[C@@H](C(=O)O)C3)c2n1]

or a salt thereof. In embodiments, the compound is:

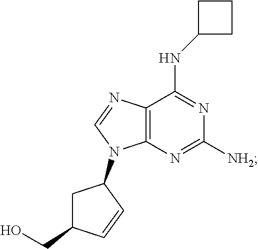 [SMILES: Nc1nc(NC2CCC2)c2ncn([C@H]3C=C[C@@H](CO)C3)c2n1]

or a salt thereof. In embodiments, the compound is:

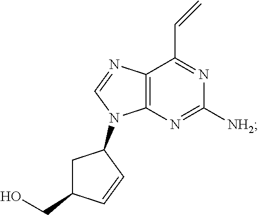 [SMILES: C=Cc1nc(N)nc2c1ncn2[C@H]1C=C[C@@H](CO)C1]

or a salt thereof.

### V. EMBODIMENTS

Embodiment P1. A method of identifying a candidate compound that stabilizes binding of an MHC protein to a peptide antigen, the method comprising:
a. contacting an MHC protein with a peptide antigen and a candidate compound thereby forming an MHC-peptide-compound complex;
b. detecting an increased stability of said MHC-peptide-compound complex relative to the stability of an MHC-peptide complex, wherein said MHC-peptide complex comprises said MHC protein and said peptide antigen in the absence of said candidate compound, thereby identifying said candidate compound as stabilizing binding of said MHC protein to said peptide antigen.

Embodiment P2. The method of embodiment P1, wherein said MHC protein binds to said peptide antigen with a Kd of greater than 1 micromolar.

Embodiment P3. The method of embodiment P1 or P2, wherein the MHC protein in step a is unfolded.

Embodiment P4. The method of any one of embodiments P1-P3, wherein the MHC protein is an MHC class I protein or an MHC class II protein.

Embodiment P5. The method of any one of embodiments P1-P4, wherein the MHC protein is the MHC class I heavy chain protein.

Embodiment P6. The method of any one of embodiments P1-P5, wherein the MHC protein is HLA-B*57:01.

Embodiment P7. A method of treating cancer in a subject in need thereof, the method comprising:
(a) detecting an MHC allele of an MHC protein of said subject;
(b) detecting a driver oncogene mutation in said subject;
(c) administering an effective amount of a MHC-peptide antigen stabilizing compound.

Embodiment P8. The method of embodiment P7, wherein said MHC-peptide antigen stabilizing compound was identified by a method comprising:
(i) contacting said MHC protein in vitro with a peptide cancer antigen and said MHC-peptide antigen stabilizing compound thereby forming an MHC-peptide-compound complex;
(ii) detecting an increased stability of said MHC-peptide-compound complex relative to the stability of an MHC-peptide complex, wherein said MHC-peptide complex comprises said MHC protein and said peptide cancer antigen in the absence of said MHC-peptide antigen stabilizing compound.

Embodiment P9. The method of any one of embodiments P1-P8, wherein said MHC-peptide antigen stabilizing compound has a molecular weight of less than 750 g/mol.

Embodiment P10. A method of identifying a modified peptide-MHC protein allele binding pair, the method comprising:
a. contacting a plurality of different modified peptides with a plurality of different MHC protein alleles;
b. detecting or computationally predicting binding of a first modified peptide to a first MHC protein allele, thereby identifying a modified peptide-MHC protein allele binding pair.

Embodiment P11. The method of embodiment P10, wherein the first modified peptide is modified with a tryptophan.

Embodiment P12. The method of embodiment P10 or P11, wherein the plurality of different modified peptides are modified with a tryptophan on the last residue.

Embodiment P13. The method of any one of embodiments P1-P12, wherein the plurality of different modified peptides are derived from a driver oncogene protein.

Embodiment P14. The method of any one of embodiments P1-P13, wherein the plurality of different modified peptides are derived from a K-Ras protein.

Embodiment P15. The method of any one of embodiments P1-P14, wherein the plurality of different modified peptides are derived from a mutant K-Ras protein.

Embodiment P16. The method of embodiment 15, wherein the mutant K-Ras protein is KRAS p.G12V.

Embodiment P17. The method of any of embodiments P1-P9, wherein the MHC-peptide antigen stabilizing compound has the formula:

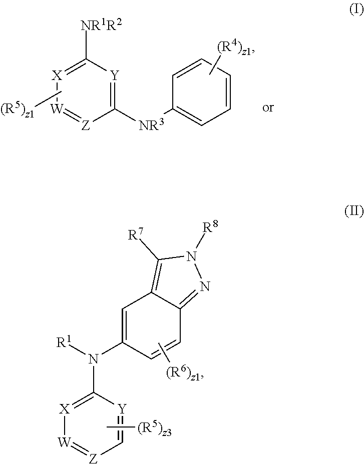

or salts thereof; wherein
W, X, Y and Z are each independently C or N;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂,
—NO₂, —SH, —SOₙ₁R^{1A}, —SOᵥ₁NR^{1A}R^{1B}, —POₘ₁R^{1A}, —POᵣ₁NR^{1A}R^{1B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R² is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SOₙ₂R^{2A}, —SOᵥ₂NR^{2A}R^{2B}, —POₘ₂R^{2A}, —POᵣ₂NR^{2A}R^{2B}, —NHNH₂, —ONH₂, —NHC(O)NHNH₂, —NHC(O)NH₂, —NHSO₂H, —NHC(O)H, —NHC(O)OH, —NHOH,
—OCCl₃, —OCF₃, —OCBr₃, —OCI₃, —OCHCl₂, —OCHBr₂, —OCHI₂, —OCHF₂, —N₃, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR, —NH₂, —COOH, —CONH₂, —NO₂, —SH, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁴ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{4A}, —NR^{4A}R^{4B}, —COOR^{4A}, —CONR^{4A}R^{4B}, —NO₂, —SR^{4A}, —SOₙ₄R^{4A}, —SOᵥ₄NR^{4A}R^{4B}, —PO(OH)₂, —POₘ₄R^{4A},
—POᵣ₄NR^{4A}R^{4B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;

R⁵ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{5A}, —NR^{5A}R^{5B}, —COOH, —CONH₂, —NO₂, —SH, —SOₙ₅R^{5A}, —SOᵥ₅NR^{5A}R^{5B}, —PO(OH)₂, —POₘ₅R^{5A},

—POᵣ₅NR^{5A}R^{5B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁶ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{6A}, —NH₂, —COOH, —CONH₂,
—NO₂, —SH, —SOₙ₆R^{6A}, —SOᵥ₆NR^{6A}R^{6B}, —PO(OH)₂, —POₘ₆R^{6A}, —POᵣ₆NR^{6A}R^{6B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁷ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{7A}, —NH₂, —COOH, —CONH₂, —NO₂, —SH, —SOₙ₇R^{7A}, —SOᵥ₇NR^{7A}R^{7B}, —PO(OH)₂, —POₘ₇R^{7A}, —POᵣ₇NR^{7A}R^{7B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁸ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SOₙ₈R^{8A}, —SOᵥ₈NR^{8A}R^{8B}, —PO(OH)₂, —POₘ₈R^{8A}, —POᵣ₈NR^{8A}R^{8B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
each R^{1A}, R^{1B}, R^{2A}, R^{2B}, R^{4A}, R^{4B}, R^{5A}, R^{5B}, R^{6A}, R^{6B}, R^{7A}, R^{7B}, R^{8A}, and R^{8B} is independently hydrogen, —CX₃, —CHX₂, —CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl;

### X is —Cl, —Br, —I or —F;

each n1, n2, n4, n5, n6, n7, and n8 is independently an integer from 0 to 4;
each v1, v2, v4, v5, v6, v7, and v8 is independently 1 or 2;
each m1, m2, m4, m5, m6, m7, and m8 is independently an integer from 0 to 3;
each r1, r2, r4, r5, r6, r7, and r8 is independently 1 or 2;
each z1 and z3 is independently 0 to 5;
z2 is 0 to 4; and
z4 is 0 to 3.

Embodiment P18. The method of any one of embodiments P1-P9 or P17, wherein R¹ is hydrogen or unsubstituted alkyl;

R³ is hydrogen or unsubstituted alkyl;
R² is hydrogen, substituted or unsubstituted alkyl, substituted or unsubstituted aryl, substituted or unsubstituted heteroaryl, or substituted or unsubstituted cycloalkyl;
R⁵ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NR^{5A}R^{5B}, —COOH, —CONH₂,
—NO₂, —SH, substituted or unsubstituted heteroalkyl, or substituted or unsubstituted alkyl;
R⁴ is hydrogen, substituted or unsubstituted alkyl or —SO₂NR^{4A}R^{4B};
R⁶ is hydrogen, halogen, or substituted or unsubstituted alkyl;
R⁷ is hydrogen or substituted or unsubstituted alkyl;
R⁸ is hydrogen or substituted or unsubstituted alkyl; and
each R^{4A}, R^{4B}, R^{5A} and R^{5B} is independently hydrogen or substituted or unsubstituted alkyl.

Embodiment P19. The method of any one of embodiments P1-P9 or P17-P18, wherein R¹ is hydrogen or methyl;

R³ is hydrogen or methyl;
R² is methyl, unsubstituted cycloalkyl, unsubstituted aryl or substituted heteroaryl;
R⁵ is hydrogen, oxo, methyl, halogen, unsubstituted heteroalkyl or —NR^{5A}R^{5B};
R⁴ is hydrogen, methyl or —SO₂NR^{4A}R^{4B};
R⁶ is hydrogen or methyl;
R⁷ is hydrogen or methyl;
R⁸ is hydrogen or methyl; and
each R^{4A}, R^{4B}, R^{5A} and R^{5B} is independently hydrogen or methyl.

Embodiment P20. The method of any one of embodiments P1-P9 or P17-P19, wherein R¹ is hydrogen or methyl;

R³ is hydrogen or methyl;
R² is methyl, cyclopropyl, phenyl, or substituted 2H-indazole;
R⁵ is hydrogen, oxo, halogen, ethoxy or —NR^{5A}R^{5B};
R⁴ is hydrogen, methyl or —SO₂NR^{4A}R^{4B}.
R⁶ is hydrogen or methyl;
R⁷ is methyl;
R⁸ is methyl; and
each R^{4A}, R^{4B}, R^{5A} and R^{5B} is independently hydrogen or methyl.

Embodiment P21. The method of any one of embodiments P1-P9 or P17-P20, wherein Y and Z are N; and W and X are C.

Embodiment P22. The method of any one of embodiments P1-P9 or P17-P20, wherein X and Y are N; and W and Z are C.

Embodiment P23. The method of any one of embodiments P1-P9 or P17-P22, wherein the MHC-peptide antigen stabilizing compound has the formula:

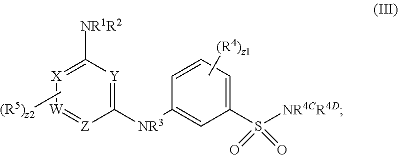

wherein
each R^{4C} and R^{4D} is independently hydrogen, —CX₃, —CHX₂,
—CH₂X, —C(O)OH, —C(O)NH₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4C} and R^{4D} substituents bonded to the same nitrogen atom may optionally be joined to form a substituted or unsubstituted heterocycloalkyl or substituted or unsubstituted heteroaryl;
R³ is hydrogen or methyl; and
z1 is 0 to 4.

Embodiment P24. The method of embodiment 23, wherein the MHC-peptide antigen stabilizing compound has the formula:

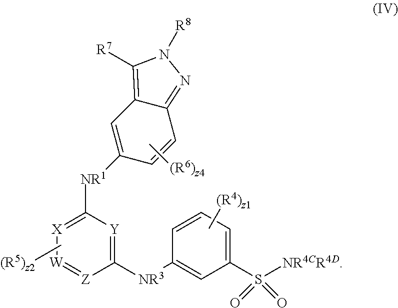

Embodiment P25. The method of any one of embodiments P1-P9 and P17-P24, wherein the MHC-peptide antigen stabilizing compound is selected from a group consisting of:

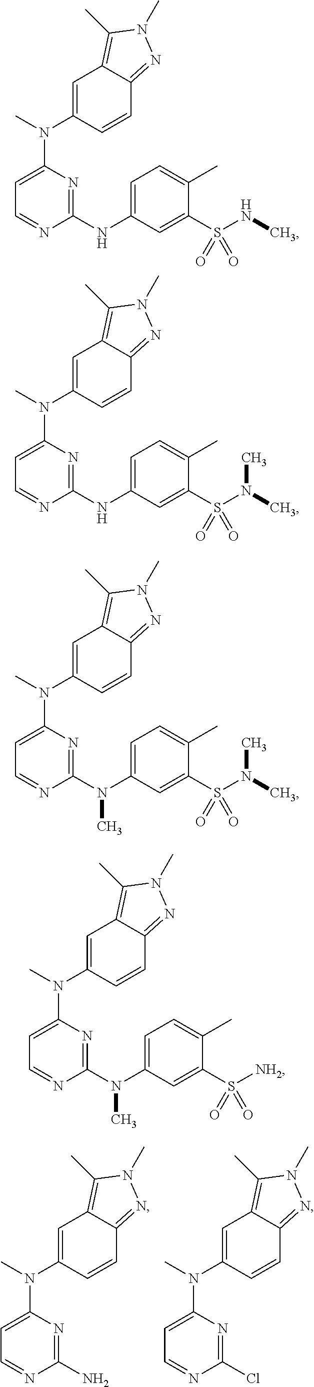

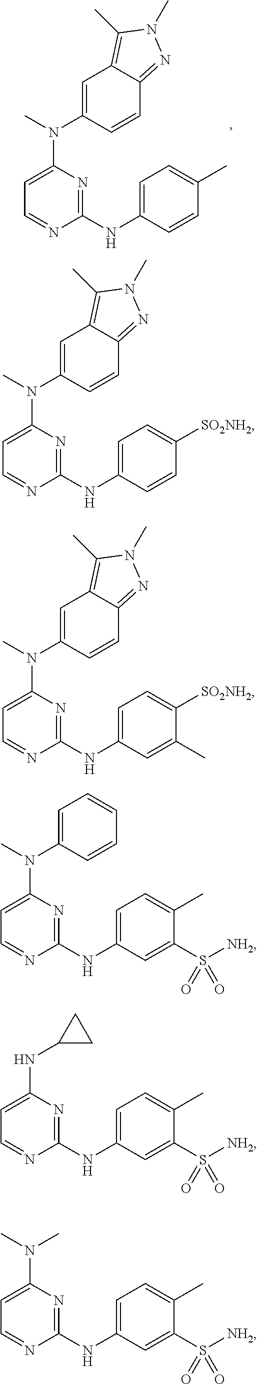

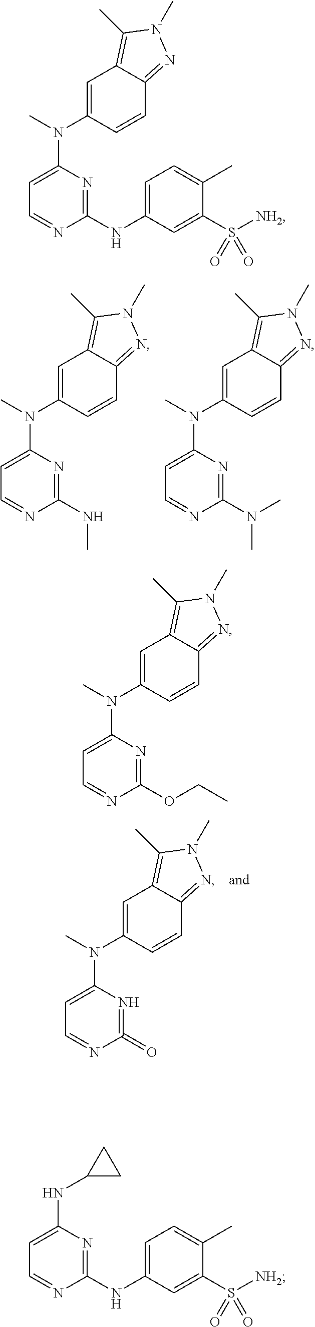

or a salt thereof.

Embodiment P26. A method of vaccinating a subject for cancer, the method comprising administering:
a. a peptide cancer antigen; and
b. a compound that stabilizes binding of an MHC protein to said peptide cancer antigen.

Embodiment P27. The method of embodiment 26, wherein said MHC-peptide cancer antigen stabilizing compound is identified by the method of any one of embodiments P1-P6.

Embodiment P28. The method of embodiment P26 or P27, wherein a vaccine is administered in a single formulation.

Embodiment P29. A compound having the formula:

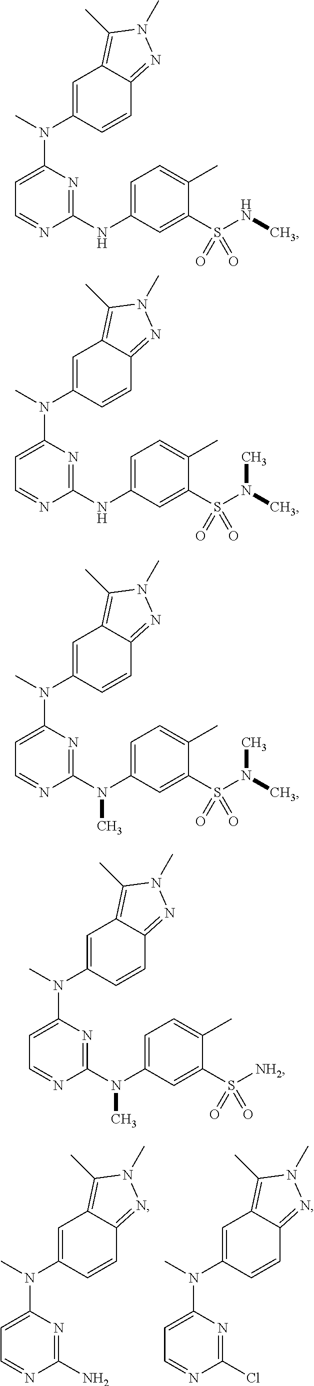

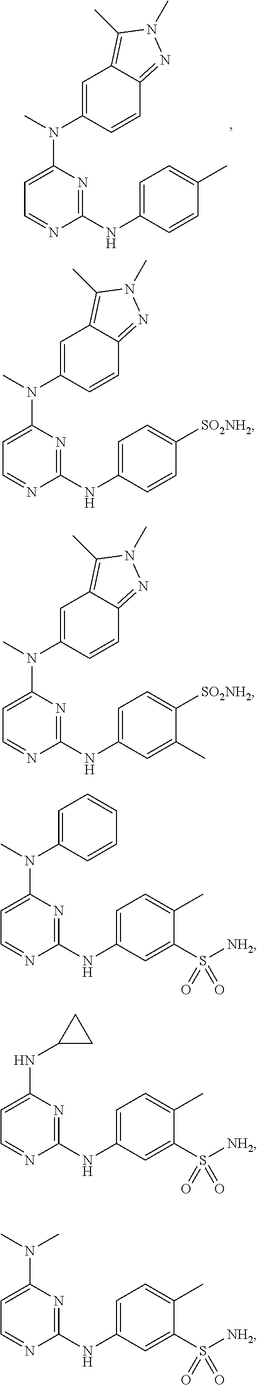

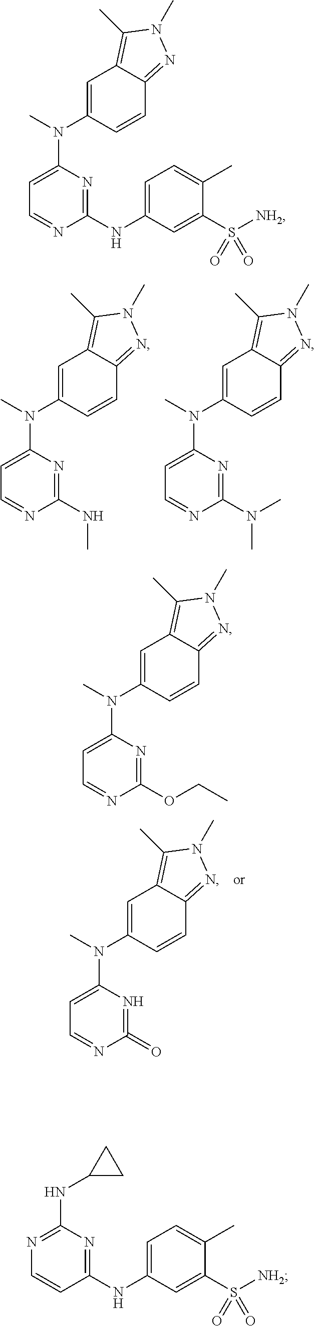

or a salt thereof.

### VI. ADDITIONAL EMBODIMENTS

Embodiment 1. A method of identifying a candidate compound that stabilizes binding of an MHC protein to a peptide antigen, the method comprising.
a. contacting an MHC protein with a peptide antigen and a candidate compound thereby forming an MHC-peptide-compound complex;
b. detecting an increased stability of said MHC-peptide-compound complex relative to the stability of an MHC-peptide complex, wherein said MHC-peptide complex comprises said MHC protein and said peptide antigen in the absence of said candidate compound, thereby identifying said candidate compound as stabilizing binding of said MHC protein to said peptide antigen.

Embodiment 2. The method of embodiment 1, wherein said MHC protein binds to said peptide antigen with a Kd of greater than 1 micromolar.

Embodiment 3. The method of embodiment 1 or 2, wherein the MHC protein in step a is unfolded.

Embodiment 4. The method of any one of embodiments 1 to 3, wherein the MHC protein is an MHC class I protein or an MHC class II protein.

Embodiment 5. The method of any one of embodiments 1 to 4, wherein the MHC protein is the MHC class I heavy chain protein.

Embodiment 6. The method of any one of embodiments 1 to 5, wherein the MHC protein is HLA-B*57:01.

Embodiment 7. A method of treating cancer in a subject in need thereof, the method comprising:
(a) detecting an MHC allele of an MHC protein of said subject;
(b) detecting a driver oncogene mutation in said subject;
(c) administering an effective amount of a MHC-peptide antigen stabilizing compound.

Embodiment 8. The method of embodiment 7, wherein said MHC-peptide antigen stabilizing compound was identified by a method comprising:
(i) contacting said MHC protein in vitro with a peptide cancer antigen and said MHC-peptide antigen stabilizing compound thereby forming an MHC-peptide-compound complex;
(ii) detecting an increased stability of said MHC-peptide-compound complex relative to the stability of an MHC-peptide complex, wherein said MHC-peptide complex comprises said MHC protein and said peptide cancer antigen in the absence of said MHC-peptide antigen stabilizing compound.

Embodiment 9. The method of any one of embodiments 7 to 8, wherein said MHC-peptide antigen stabilizing compound has a molecular weight of less than 750 g/mol.

Embodiment 10. A method of identifying a modified peptide-MHC protein allele binding pair, the method comprising:
a. contacting a plurality of different modified peptides with a plurality of different MHC protein alleles;
b. detecting or computationally predicting binding of a first modified peptide to a first MHC protein allele, thereby identifying a modified peptide-MHC protein allele binding pair.

Embodiment 11. The method of embodiment 10, wherein the first modified peptide is modified with a tryptophan.

Embodiment 12. The method of embodiment 10 or 11, wherein the plurality of different modified peptides are modified with a tryptophan on the last residue.

Embodiment 13. The method of any one of embodiments 10 to 12, wherein the plurality of different modified peptides are derived from a driver oncogene protein.

Embodiment 14. The method of any one of embodiments 10 to 13, wherein the plurality of different modified peptides are derived from a K-Ras protein.

Embodiment 15. The method of any one of embodiments 10 to 14, wherein the plurality of different modified peptides are derived from a mutant K-Ras protein.

Embodiment 16. The method of embodiment 15, wherein the mutant K-Ras protein is KRAS p.G12V.

Embodiment 17. The method of any one of embodiments 1 to 9, wherein the MHC-peptide antigen stabilizing compound has the formula:

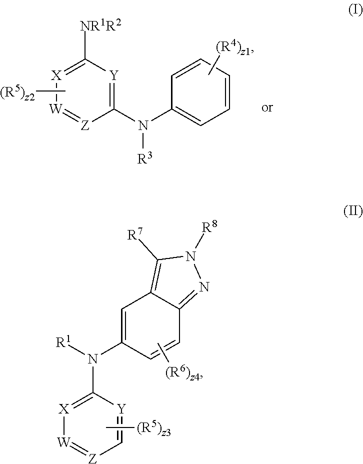

thereof;
wherein,
W, X, Y and Z are each independently C or N;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂,
NO2, —SH, —SOₙ₁R^{1A}, —SOᵥ₁N^{R1A}R^{1B}, —POₘ₁R^{1A}, —POᵣ₁NR^{1A}R^{1B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R² is hydrogen,
   halogen, —CCl₃, —CBr₃, —CF₃, —CI₃, —CH₂Cl, —CH₂Br, —CH₂F, —CH₂I,
CHCl₂, —CHBr₂, —CHF₂, —CHI₂, —CN, —OH, —NH₂, —COOH, —CONH₂, —NO2, —SH,
SOₙ₂R^{2A}, —SOᵥ₂NR^{2A}R^{2B}, —POₘ₂R^{2A}, —POᵣ₂NR^{2A}R^{2B}, —NHNH₂, —ONH₂, —NHC(O)NHNH₂, —NHC(O)NH₂, —NHSO₂H, —NHC(O)H, —NHC(O)OH, —NHOH,
—OCCl₃, —OCF₃, —OCBr₃, —OCI₃, —OCHCl₂, —OCHBr₂, —OCHI₂, —OCHF₂, —N₃, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR, —NH₂, —COOH, —CONH₂,
—NO₂, —SH, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁴ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{4A}, —NR^{4A}R^{4B}, —COOR^{4A},
—CONR^{4A}R^{4B}, —NO₂, —SR^{4A}, —SOₙ₄R^{4A}, —SOᵥ₄NR^{4A}R^{4B}, —PO(OH)₂, —POₘ₄R^{4A},
—POᵣ₄NR^{4A}R^{4B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁵ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{5A}, —NR^{5A}R^{5B}, —COOH,
—CONH₂, —NO₂, —SH, —SOₙ₅R^{5A}, —SOᵥ₅NR^{5A}R^{5B}, —PO(OH)₂, —POₘ₅R^{5A},
—POᵣ₅NR^{5A}R^{5B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁶ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{6A}, —NH₂, —COOH, —CONH₂,
—NO₂, —SH, —SO₁₆R^{6A}, —SOᵥ₆NR^{6A}R^{6B}, —PO(OH)₂, —POₘ₆R^{6A}, —POᵣ₆NR^{6A}R^{6B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁷ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{7A}, —NH₂, —COOH, —CONH₂,
—NO₂, —SH, —SOₙ₇R^{7A}, —SOᵥ₇NR^{7A}R^{7B}, —PO(OH)₂, —POₘ₇R^{7A}, —POᵣ₇NR^{7A}R^{7B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁸ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂,
—NO₂, —SH, —SOₙ₈R^{8A}, —SOᵥ₈NR^{8A}R^{8B}, —PO(OH)₂, —POₘ₈R^{8A}, —POᵣ₈NR^{8A}R^{8B} substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
each R^{1A}, R^{1B}, R^{2A}, R^{2B}, R^{4A}, R^{4B}, R^{5A}, R^{5B}, R^{6A}, R^{6B}, R^{7A}, R^{7B}, R^{8A}, and R^{8B} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl;
X is independently —Cl, —Br, —I or —F;
each n1, n2, n4, n5, n6, n7, and n8 is independently an integer from 0 to 4;
each v1, v2, v4, v5, v6, v7, and v8 is independently 1 or 2;
each m1, m2, m4, m5, m6, m7, and m8 is independently an integer from 0 to 3;
each r1, r2, r4, r5, r6, r7, and r8 is independently 1 or 2;
each z1 and z3 is independently an integer from 0 to 5;
z2 is an integer from 0 to 4; and
z4 is an integer from 0 to 3.

Embodiment 18. The method of embodiment 17, wherein R¹ is hydrogen or unsubstituted alkyl;
R³ is hydrogen or unsubstituted alkyl;
R² is hydrogen, substituted or unsubstituted alkyl, substituted or unsubstituted aryl, substituted or unsubstituted heteroaryl, or substituted or unsubstituted cycloalkyl;
R⁵ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NR^{5A}R^{5B}, —COOH, —CONH₂,
—NO₂, —SH, substituted or unsubstituted heteroalkyl, or substituted or unsubstituted alkyl;
R⁴ is hydrogen, substituted or unsubstituted alkyl or —SO₂NR^{4A}R^{4B};
R⁶ is hydrogen, halogen, or substituted or unsubstituted alkyl;
R⁷ is hydrogen or substituted or unsubstituted alkyl;
R⁸ is hydrogen or substituted or unsubstituted alkyl; and
each R^{4A}, R^{4B}, R^{5A} and R^{5B} is independently hydrogen or substituted or unsubstituted alkyl.

Embodiment 19. The method of any one of embodiments 17 to 18, wherein R¹ is hydrogen or methyl;
R³ is hydrogen or methyl;
R² is methyl, unsubstituted cycloalkyl, unsubstituted aryl or substituted heteroaryl;
R⁵ is hydrogen, oxo, methyl, halogen, unsubstituted heteroalkyl or —NR^{5A}R^{5B};
R⁴ is hydrogen, methyl or —SO₂NR^{4A}R^{4B};
R⁶ is hydrogen or methyl;
R⁷ is hydrogen or methyl;
R⁸ is hydrogen or methyl; and
each R^{4A}, R^{4B}, R^{5A} and R^{5B} is independently hydrogen or methyl.

Embodiment 20. The method of any one of embodiments 17 to 19, wherein R¹ is hydrogen or methyl;
R³ is hydrogen or methyl;
R² is methyl, cyclopropyl, phenyl, or substituted 2H-indazole;
R⁵ is hydrogen, oxo, halogen, ethoxy or —NR^{5A}R^{5B};
R⁴ is hydrogen, methyl or —SO₂NR^{4A}R^{4B};
R⁶ is hydrogen or methyl;
R⁷ is methyl;
R⁸ is methyl; and
each R^{4A}, R^{4B}, R^{5A} and R^{5B} is independently hydrogen or methyl.

Embodiment 21. The method of any one of embodiments 17 to 20, wherein Y and Z are N; and
W and X are C.

Embodiment 22. The method of any one of embodiments 17 to 20, wherein X and Y are N; and
W and Z are C.

Embodiment 23. The method of any one of embodiments 17 to 22, wherein the MHC-peptide antigen stabilizing compound has the formula:

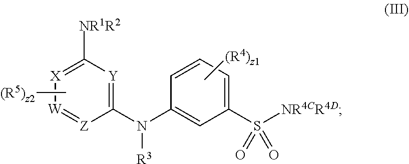

wherein
each R^{4C} and R^{4D} is independently hydrogen, —CX₃, —CHX₂, —CH₂X, —CN, —OH, —NH₂, —COOH, —CONH₂, —NO₂, —SH, —SO₃H,
—SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4C} and R^{4D} substituents bonded to the same nitrogen atom may optionally be joined to form a substituted or unsubstituted heterocycloalkyl or substituted or unsubstituted heteroaryl;
R³ is hydrogen or methyl; and
z1 is an integer from 0 to 4.

Embodiment 24. The method of embodiment 23, wherein the MHC-peptide antigen stabilizing compound has the formula:

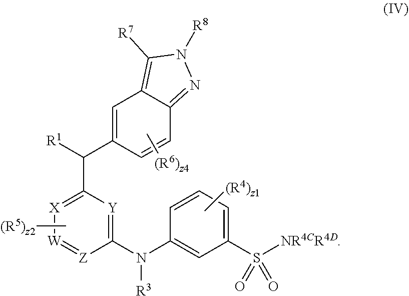

Embodiment 25. The method of any one of embodiments 17 to 24, wherein the MHC-peptide antigen stabilizing compound is selected from a group consisting of:

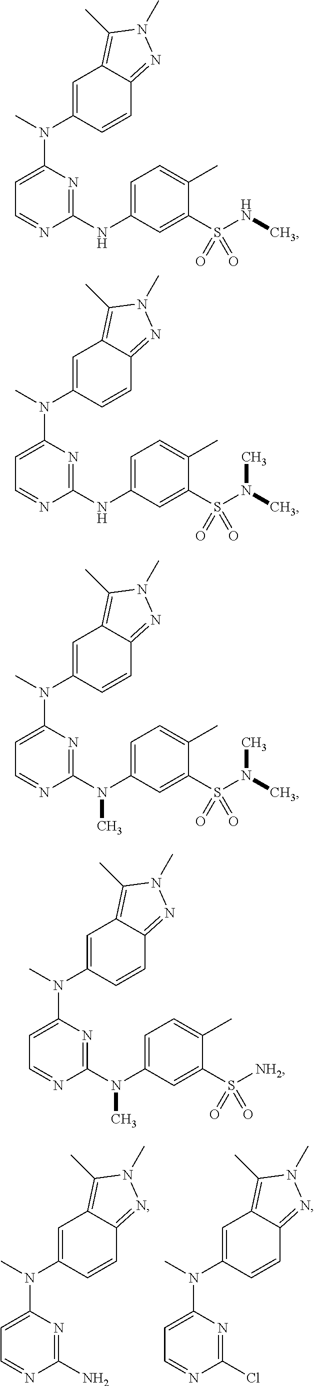

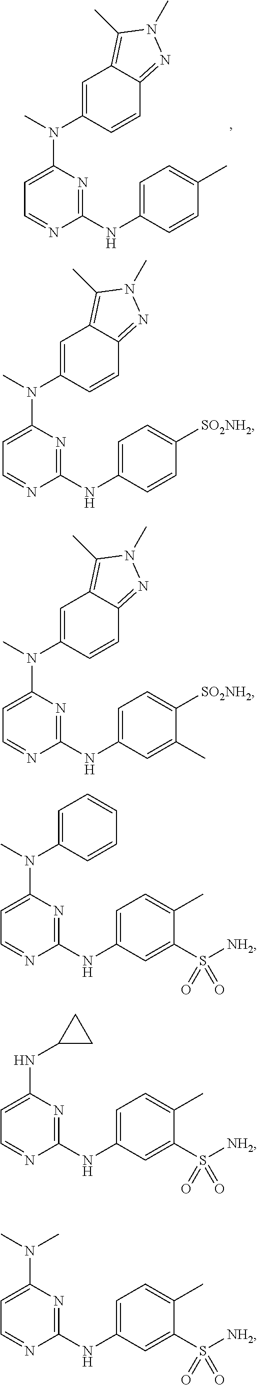

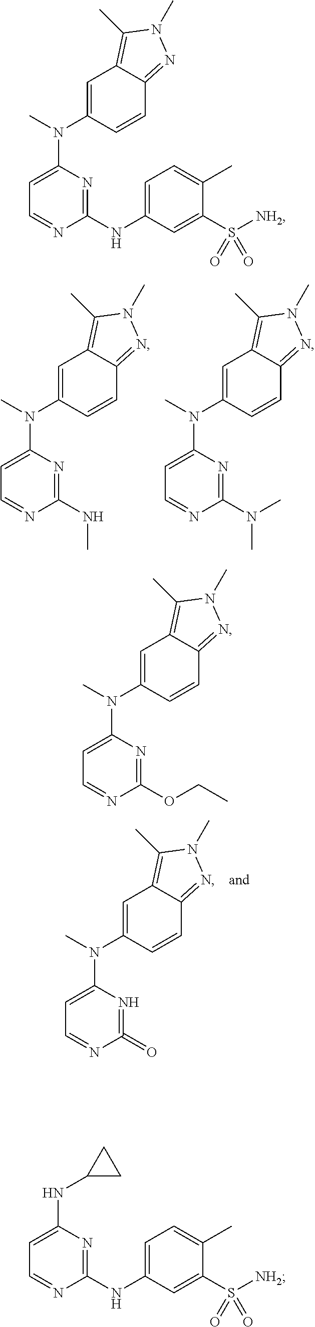

or a salt thereof.

Embodiment 26. The method of any one of embodiments 1 to 9, wherein the MHC-peptide antigen stabilizing compound has the formula:

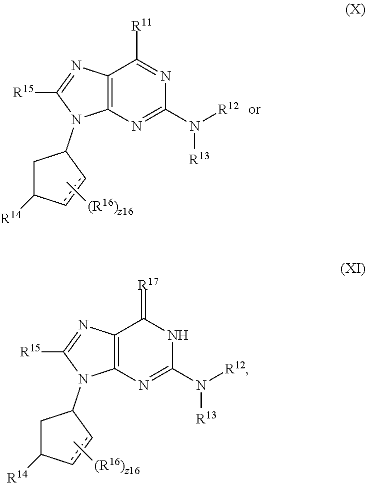

or salts thereof;
wherein,
R¹¹ is hydrogen, halogen, —CX¹¹₃, —CHX¹¹₂, —CH₂X¹¹, —OCX¹¹₃, —OCH₂X¹¹, —OCHX¹¹₂, —CN, —SOⁿ¹¹R^{11D}, —SOᵥ₁₁NR^{11A}R^{11B}, —NHC(O)NR^{11A}R^{11B}, —N(O)ₘ₁₁, —NR^{11A}R^{11B}, —C(O)R^{11C}, —C(O)—OR^{11C}, —C(O)NR^{11A}R^{11B}, —OR^{11D}, —NR^{11A}CH₂C(O)R^{11C}, —NR^{11A}CH₂SO₂R^{11D}, —NR^{11A}SO₂R^{11D}, —NR^{11A}C(O)R^{11C}, —NR^{11A}C(O)OR^{11C}, —NR^{11A}OR^{11C}, —NR^{11A}OSO₂R^{11D}, —NR^{11A}OCH₂C(O)R^{11C}, —NR^{11A}CH₂P(O)R^{11C}R^{11D}, —PO_{q11}R^{11A}, —POᵣ₁₁R^{11C}R^{11D}, —POᵣ₁₁NR^{11A}R^{11B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen,
   halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁴ is —CH₂OR^{14A}, —C(O)OR¹⁴B, or —CH₂OC(═NH)R^{14C};
R¹⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁶ is independently hydrogen,
   halogen, —CCl₃, —CBr₃, —CF₃, —CI₃, —CH₂Cl, —CH₂Br,
   —CH₂F, —CH₂I, —CHCl₂, —CHBr₂, —CHF₂, —CHI₂, —CN, —OH, —NH₂, —COOH, —CONH₂, —NO₂, —SH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁷ is ═O, ═S, or ═NR^{17A};
each R^{11A}, R^{11B}, R^{11C} and R^{11D} is independently
   hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —OSO₃H, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl;
R^{14A} and R^{14B} are independently hydrogen or unsubstituted C₁-C₅ alkyl;
R^{14C} is unsubstituted C₁-C₅ alkyl;
R^{17A} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —OSO₃H, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹¹ is —Cl, —Br, —I or —F;
n11 is independently an integer from 0 to 4;
v11 is independently 1 or 2;
m11 is independently an integer from 0 to 3;
each q11 and r11 is independently 1 or 2; and
z16 is independently an integer from 0 to 8.

Embodiment 27. The method of embodiment 26, wherein R¹² is hydrogen.

Embodiment 28. The method of any one of embodiments 26 to 27, wherein R¹³ is hydrogen.

Embodiment 29. The method of any one of embodiments 26 to 28, wherein R¹⁵ is hydrogen.

Embodiment 30. The method of any one of embodiments 26 to 29, wherein R¹⁴ is —CH₂OR^{14A}.

Embodiment 31. The method of embodiment 30, wherein R^{14A} is hydrogen.

Embodiment 32. The method of any one of embodiments 26 to 31, wherein the MHC-peptide antigen stabilizing compound has the formula:

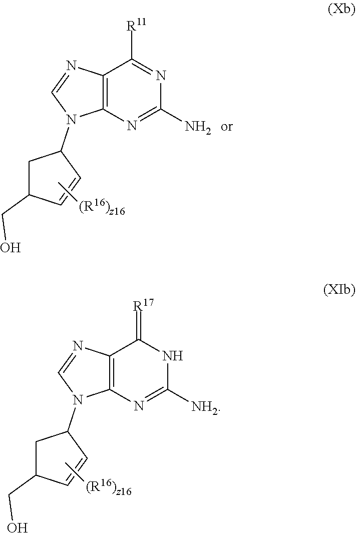

Embodiment 33. The method of any one of embodiments 26 to 31, wherein the MHC-peptide antigen stabilizing compound has the formula:

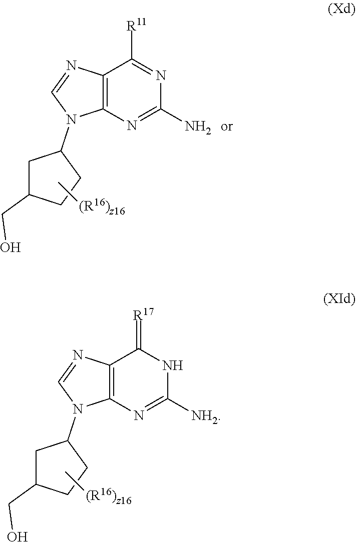

Embodiment 34. The method of one of embodiments 32 to 33, wherein R¹⁶ is independently hydrogen or —OH.

Embodiment 35. The method of one of embodiments 32 to 34, wherein z16 is 1 or 2.

Embodiment 36. The method of one of embodiments 32 to 34, wherein z16 is 0.

Embodiment 37. The method of one of embodiments 26 to 36, wherein R¹¹ is hydrogen, halogen, —CCl₃, —CBr₃, —CF₃, —CI₃, —CH₂Cl, —CH₂Br,

—CH₂F, —CH₂I, —CHCl₂, —CHBr₂, —CHF₂, —CHI₂, —CN, —OH, —
ONH₂, —NR^{11A}R11B, —COOH, —COO(C₁-C₄ alkyl), —CONH₂, —NO₂, —SH, —SO₂OH, —SO₂NH₂, —PO(OH)₂, —OCCl₃, —OCF₃, —OCBr₃, —OCI₃, —OCHCl₂, —OCHBr₂, —OCHI₂, —OCHF₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 38. The method of one of embodiments 26 to 36, wherein R¹¹ is —NR^{11A}R^{11B}.

Embodiment 39. The method of one of embodiments 26 to 36, wherein R¹¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 40. The method of embodiment 38, wherein R^{11A} and R^{11B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl.

Embodiment 41. The method of embodiment 38, wherein R^{11A} and R^{11B} are independently hydrogen, substituted or unsubstituted C₁-C₄ alkyl, or substituted or unsubstituted C₃-C₆ cycloalkyl.

Embodiment 42. The method of embodiment 38, wherein R^{11A} and R^{11B} substituents bonded to the same nitrogen atom are joined to form a substituted or unsubstituted 4 to 6 membered heterocycloalkyl or substituted or unsubstituted 5 to 6 membered heteroaryl.

Embodiment 43. The method of embodiment 38, wherein R^{11A} and R^{11B} are independently hydrogen, —COCHCH₂, —CH₂COOH, —CH₂SO₂OH, —OSO₂OH, —CH₂P(O)(OH)₂, or —OCH₂COOH.

Embodiment 44. The method of any one of embodiments 26 to 43, wherein the MHC-peptide antigen stabilizing compound is selected from a group consisting of:

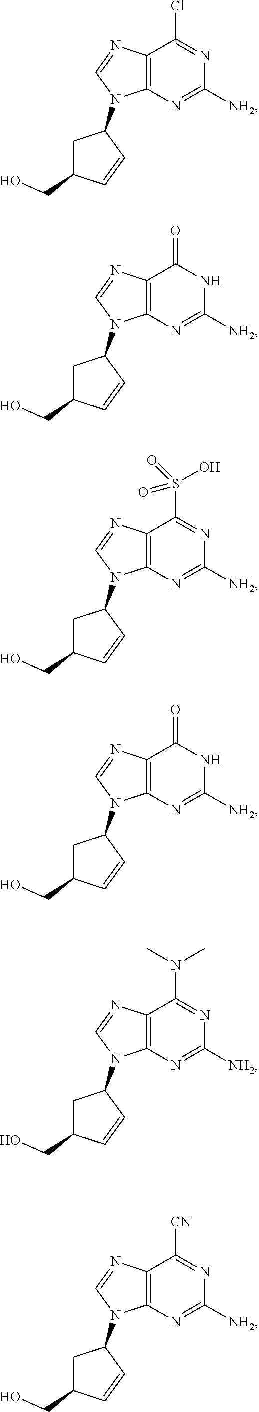 [SMILES: Nc1nc(Cl)c2ncn([C@H]3C=C[C@@H](CO)C3)c2n1 ; Nc1nc2c(ncn2[C@H]2C=C[C@@H](CO)C2)c(=O)[nH]1 ; Nc1nc(S(=O)(=O)O)c2ncn([C@H]3C=C[C@@H](CO)C3)c2n1 ; Nc1nc2c(ncn2[C@H]2C=C[C@@H](CO)C2)c(=O)[nH]1 ; CN(C)c1nc(N)nc2c1ncn2[C@H]1C=C[C@@H](CO)C1 ; N#Cc1nc(N)nc2c1ncn2[C@H]1C=C[C@@H](CO)C1]

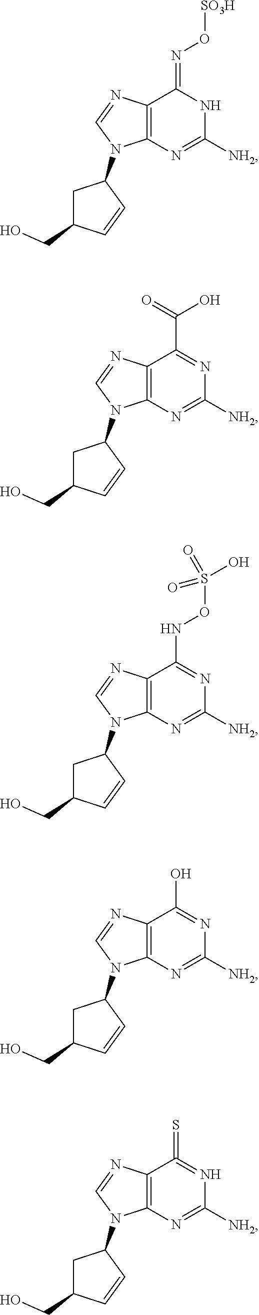

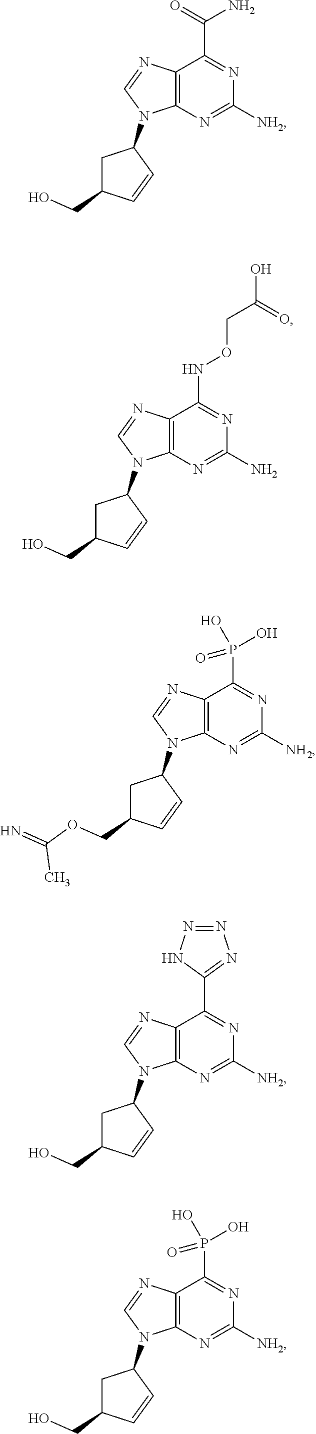 [SMILES: NC(=O)c1nc(N)nc2c1ncn2[C@H]1C=C[C@@H](CO)C1 ; Nc1nc(NOCC(=O)O)c2ncn([C@H]3C=C[C@@H](CO)C3)c2n1 ; CC(=N)OC[C@@H]1C=C[C@H](n2cnc3c(P(=O)(O)O)nc(N)nc32)C1 ; Nc1nc(-c2nnn[nH]2)c2ncn([C@H]3C=C[C@@H](CO)C3)c2n1 ; Nc1nc(P(=O)(O)O)c2ncn([C@H]3C=C[C@@H](CO)C3)c2n1]

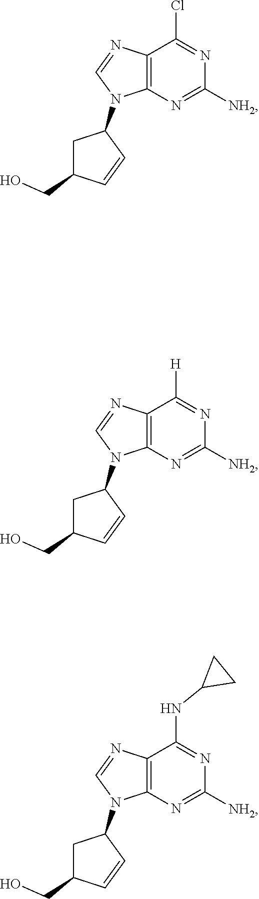 [SMILES: Nc1nc(Cl)c2ncn([C@H]3C=C[C@@H](CO)C3)c2n1 ; Nc1ncc2ncn([C@H]3C=C[C@@H](CO)C3)c2n1 ; Nc1nc(NC2CC2)c2ncn([C@H]3C=C[C@@H](CO)C3)c2n1]

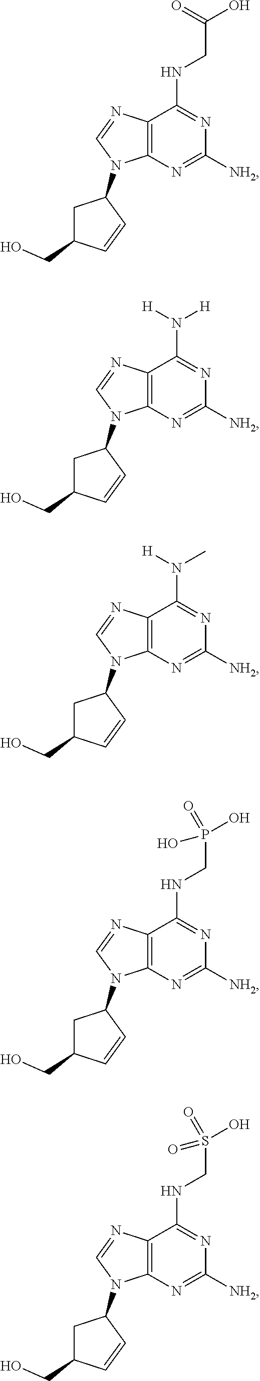 [SMILES: Nc1nc(NCC(=O)O)c2ncn([C@H]3C=C[C@@H](CO)C3)c2n1 ; Nc1nc(N)c2ncn([C@H]3C=C[C@@H](CO)C3)c2n1 ; CNc1nc(N)nc2c1ncn2[C@H]1C=C[C@@H](CO)C1 ; Nc1nc(NCP(=O)(O)O)c2ncn([C@H]3C=C[C@@H](CO)C3)c2n1 ; Nc1nc(NCS(=O)(=O)O)c2ncn([C@H]3C=C[C@@H](CO)C3)c2n1]

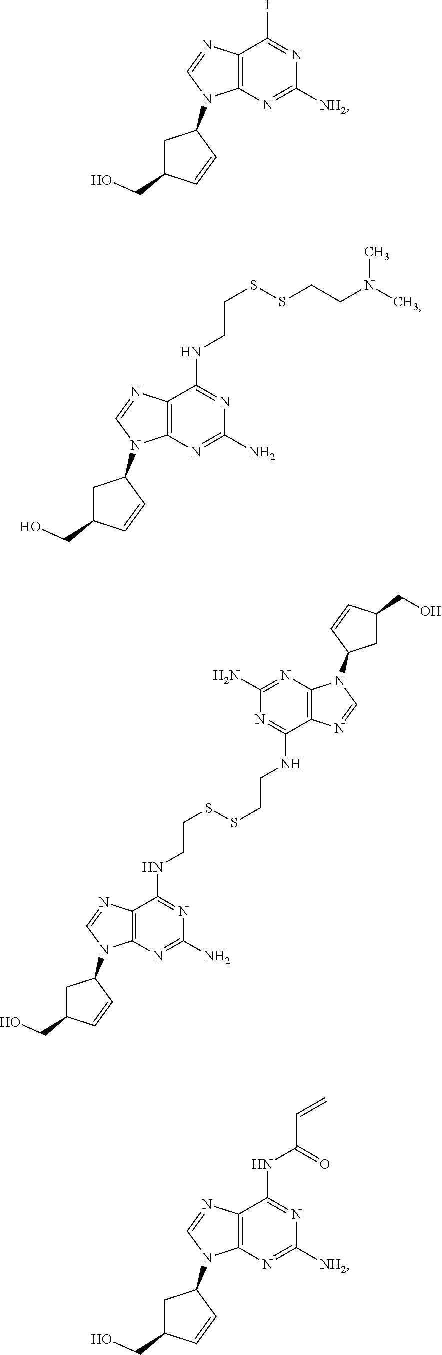 [SMILES: Nc1nc(I)c2ncn([C@H]3C=C[C@@H](CO)C3)c2n1 ; CN(C)CCSSCCNc1nc(N)nc2c1ncn2[C@H]1C=C[C@@H](CO)C1 ; Nc1nc(NCCSSCCNc2nc(N)nc3c2ncn3[C@H]2C=C[C@@H](CO)C2)c2ncn([C@H]3C=C[C@@H](CO)C3)c2n1 ; C=CC(=O)Nc1nc(N)nc2c1ncn2[C@H]1C=C[C@@H](CO)C1]

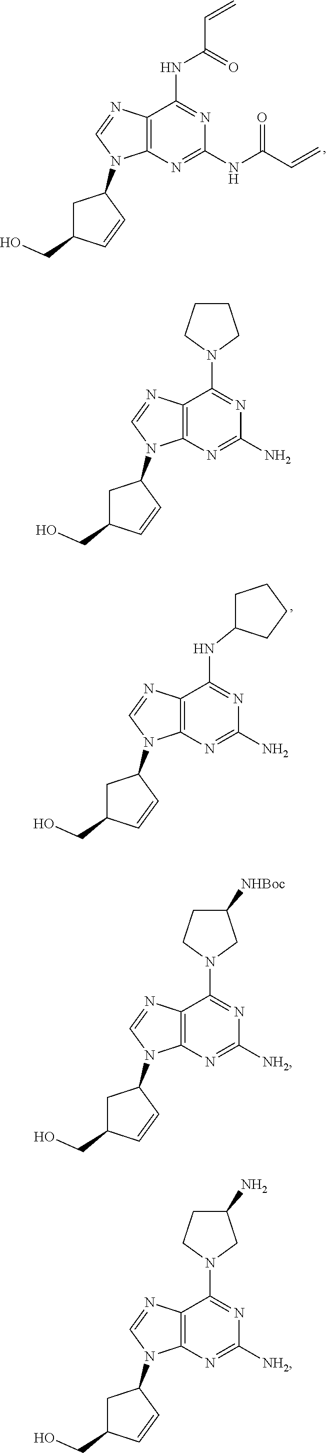 [SMILES: C=CC(=O)Nc1nc(NC(=O)C=C)c2ncn([C@H]3C=C[C@@H](CO)C3)c2n1 ; Nc1nc(N2CCCC2)c2ncn([C@H]3C=C[C@@H](CO)C3)c2n1 ; Nc1nc(NC2CCCC2)c2ncn([C@H]3C=C[C@@H](CO)C3)c2n1 ; CC(C)(C)OC(=O)N[C@@H]1CCN(c2nc(N)nc3c2ncn3[C@H]2C=C[C@@H](CO)C2)C1 ; Nc1nc(N2CC[C@@H](N)C2)c2ncn([C@H]3C=C[C@@H](CO)C3)c2n1]

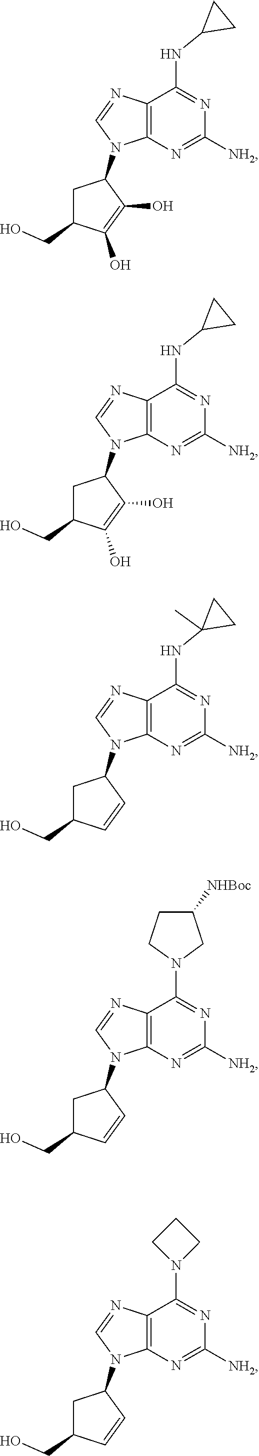

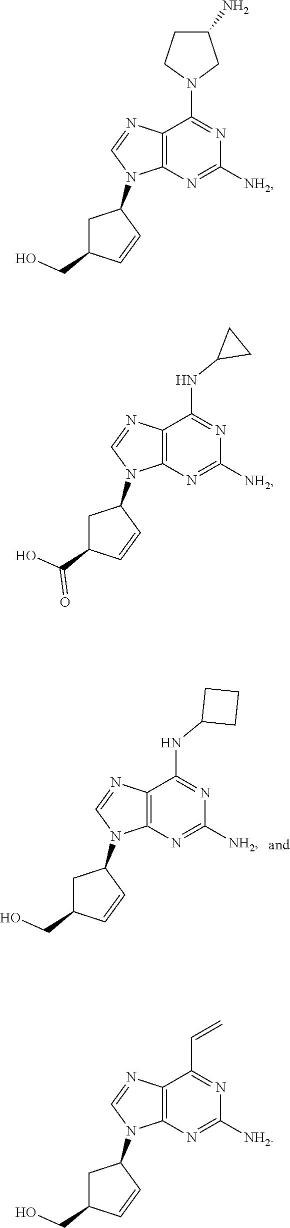 [SMILES: Nc1nc(N2CC[C@H](N)C2)c2ncn([C@H]3C=C[C@@H](CO)C3)c2n1 ; Nc1nc(NC2CC2)c2ncn([C@H]3C=C[C@@H](C(=O)O)C3)c2n1 ; Nc1nc(NC2CCC2)c2ncn([C@H]3C=C[C@@H](CO)C3)c2n1 ; C=Cc1nc(N)nc2c1ncn2[C@H]1C=C[C@@H](CO)C1]

Embodiment 45. A method of vaccinating a subject for cancer, the method comprising administering:
a. a peptide cancer antigen; and
b. a compound that stabilizes binding of an MHC protein to said peptide cancer antigen.

Embodiment 46. A method of vaccinating a subject for cancer, the method comprising administering a peptide-compound conjugate, wherein the peptide-compound conjugate comprises a peptide cancer antigen that is linked to a compound via a chemical bond.

Embodiment 47. The method of embodiment 45, wherein said MHC-peptide cancer antigen stabilizing compound is identified by the method of any one of embodiments 1 to 6.

Embodiment 48. The method of embodiment 45, wherein a vaccine is administered in a single formulation.

Embodiment 49. A composition comprising an MHC protein, a peptide antigen, and a compound; wherein, the MHC protein, the peptide antigen, and the compound are bound to form an MHC-peptide-compound complex and wherein the compound stabilizes the binding of the MHC protein to the peptide antigen relative to the absence of the compound.

Embodiment 50. The composition of embodiment 49, wherein the MHC protein is covalently bound to the peptide antigen.

Embodiment 51. The composition of embodiment 49, wherein the MHC protein is covalently bound to the peptide antigen through a disulfide bond.

Embodiment 52. The composition of embodiment 51, wherein a cysteine amino acid within the MHC protein forms a part of said disulfide bond.

Embodiment 53. The composition of embodiment 49, wherein the compound is covalently bound to the peptide antigen.

Embodiment 54. The composition of embodiment 53, wherein the compound is covalently bound to the peptide antigen through a reaction between an electrophilic moiety on the compound and a nucleophilic moiety on the peptide antigen.

Embodiment 55. The composition of embodiment 54, wherein the nucleophilic moiety is a cysteine sulfhydryl group.

Embodiment 56. The composition of embodiment 54, wherein the nucleophilic group is a lysine amine group.

Embodiment 57. A composition comprising an MHC protein which is covalently bound to a peptide antigen.

Embodiment 58. The composition of embodiment 57, wherein the MHC protein is covalently bound to the peptide antigen through a disulfide bond.

Embodiment 59. The composition of embodiment 58, wherein a cysteine amino acid within the MHC protein forms a part of said disulfide bond.

Embodiment 60. A composition comprising a peptide antigen, which is covalently bound to a compound.

Embodiment 61. The composition of embodiment 60, wherein the compound is covalently bound to the peptide antigen through a reaction between an electrophilic moiety on the compound and a nucleophilic moiety on the peptide antigen.

Embodiment 62. The composition of embodiment 61, wherein the nucleophilic moiety is a cysteine sulfhydryl group.

Embodiment 63. The composition of embodiment 61, wherein the nucleophilic group is a lysine amine group.

Embodiment 64. A compound having the formula:

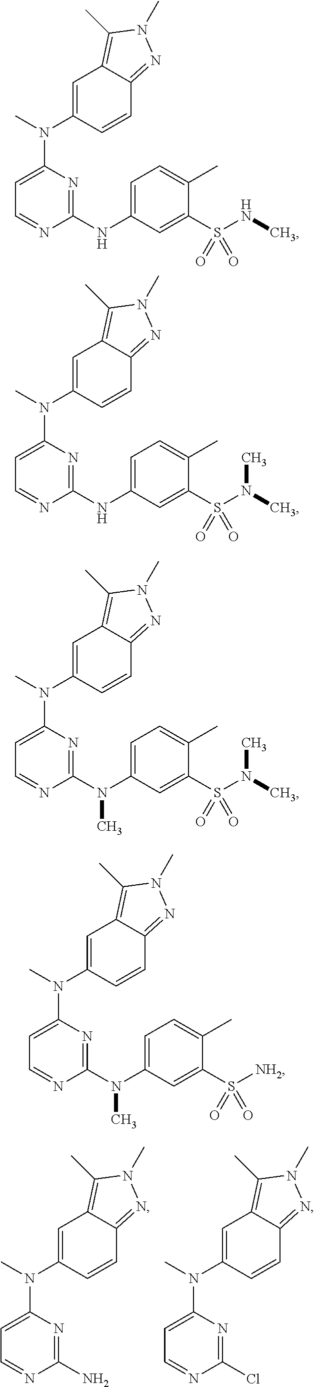

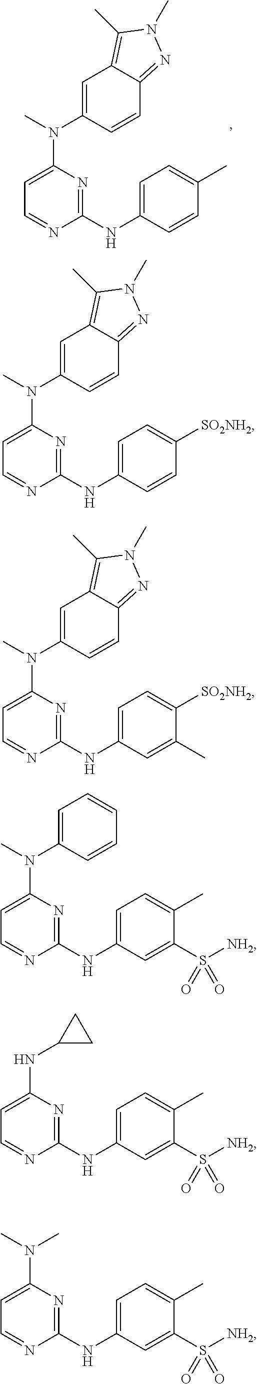

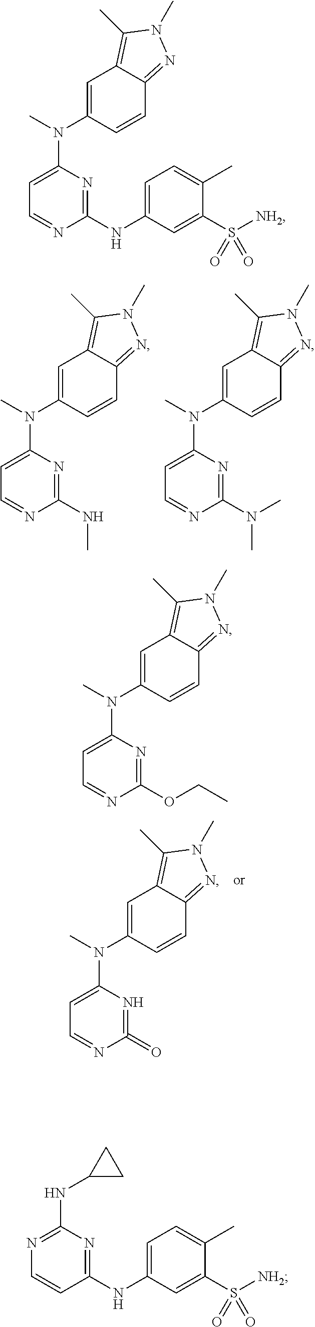

or a salt thereof.

Embodiment 65. A compound having the formula:

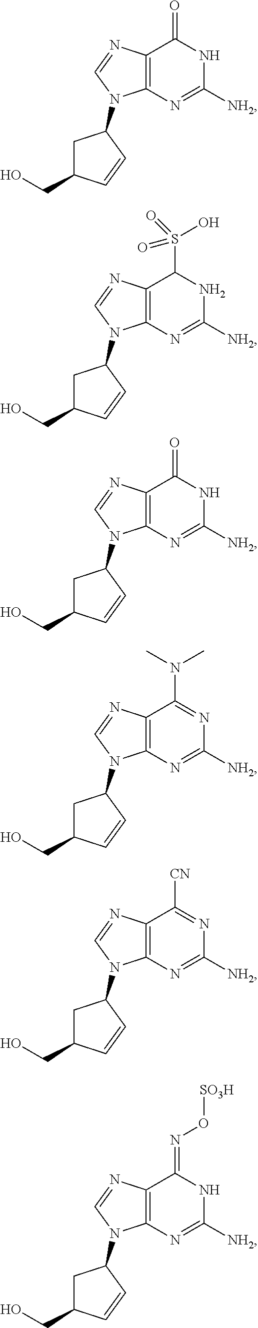 [SMILES: Nc1nc2c(ncn2[C@H]2C=C[C@@H](CO)C2)c(=O)[nH]1 ; NC1=Nc2c(ncn2[C@H]2C=C[C@@H](CO)C2)C(S(=O)(=O)O)N1 ; Nc1nc2c(ncn2[C@H]2C=C[C@@H](CO)C2)c(=O)[nH]1 ; CN(C)c1nc(N)nc2c1ncn2[C@H]1C=C[C@@H](CO)C1 ; N#Cc1nc(N)nc2c1ncn2[C@H]1C=C[C@@H](CO)C1 ; Nc1nc2c(ncn2[C@H]2C=C[C@@H](CO)C2)/c(=N/OS(=O)(=O)O)[nH]1]

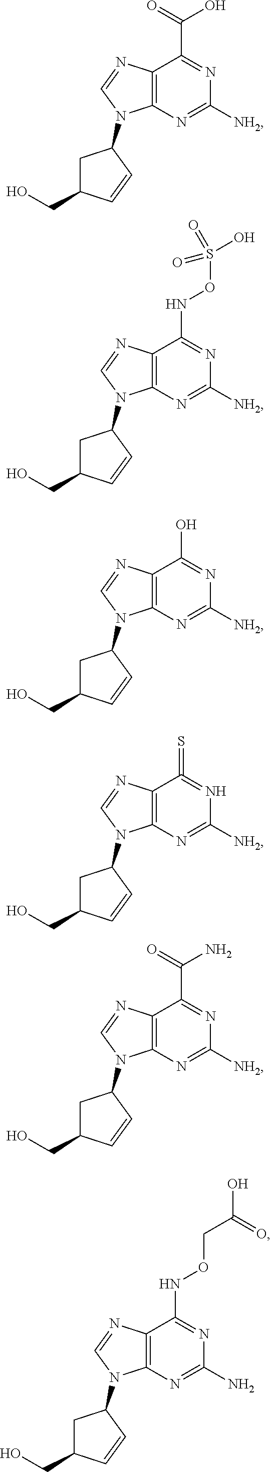

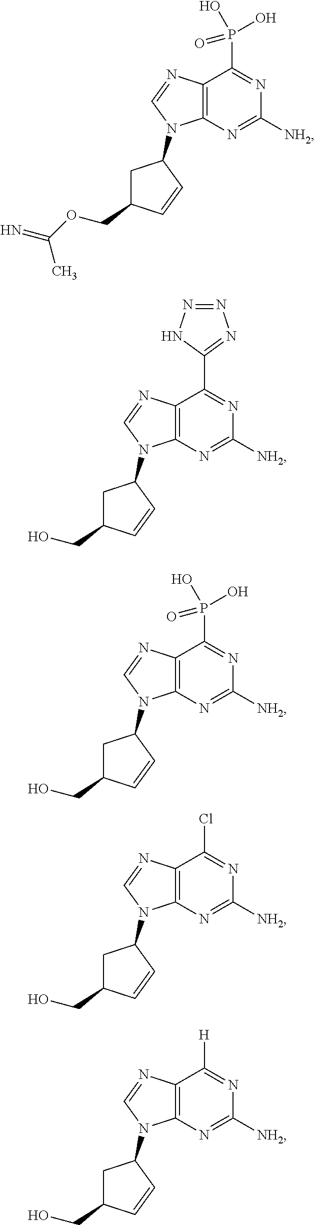 [SMILES: CC(=N)OC[C@@H]1C=C[C@H](n2cnc3c(P(=O)(O)O)nc(N)nc32)C1 ; Nc1nc(-c2nnn[nH]2)c2ncn([C@H]3C=C[C@@H](CO)C3)c2n1 ; Nc1nc(P(=O)(O)O)c2ncn([C@H]3C=C[C@@H](CO)C3)c2n1 ; Nc1nc(Cl)c2ncn([C@H]3C=C[C@@H](CO)C3)c2n1 ; Nc1ncc2ncn([C@H]3C=C[C@@H](CO)C3)c2n1]

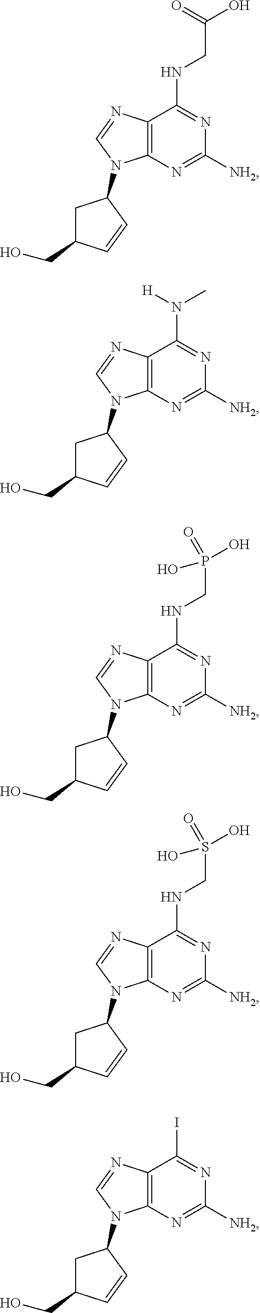

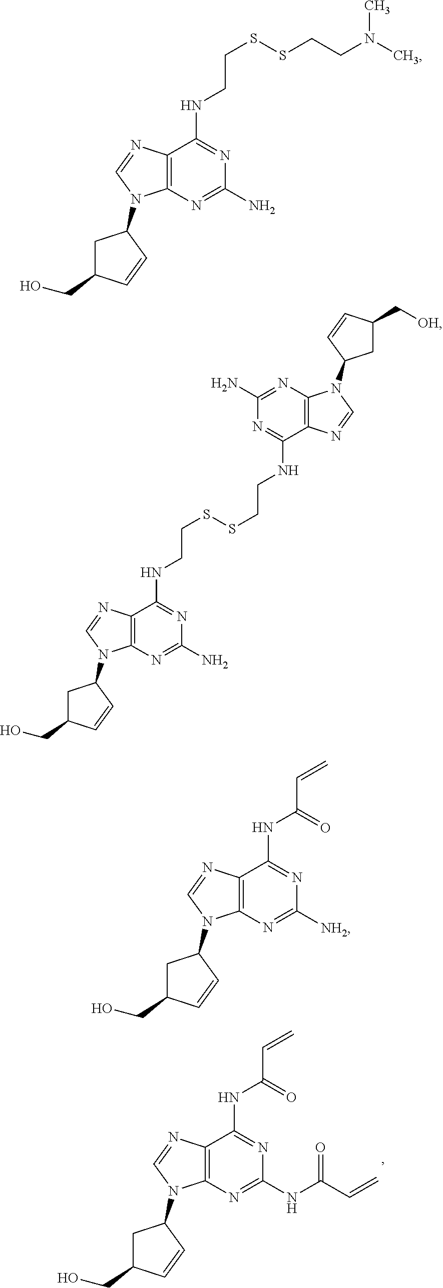 [SMILES: CN(C)CCSSCCNc1nc(N)nc2c1ncn2[C@H]1C=C[C@@H](CO)C1 ; Nc1nc(NCCSSCCNc2nc(N)nc3c2ncn3[C@H]2C=C[C@@H](CO)C2)c2ncn([C@H]3C=C[C@@H](CO)C3)c2n1 ; C=CC(=O)Nc1nc(N)nc2c1ncn2[C@H]1C=C[C@@H](CO)C1 ; C=CC(=O)Nc1nc(NC(=O)C=C)c2ncn([C@H]3C=C[C@@H](CO)C3)c2n1]

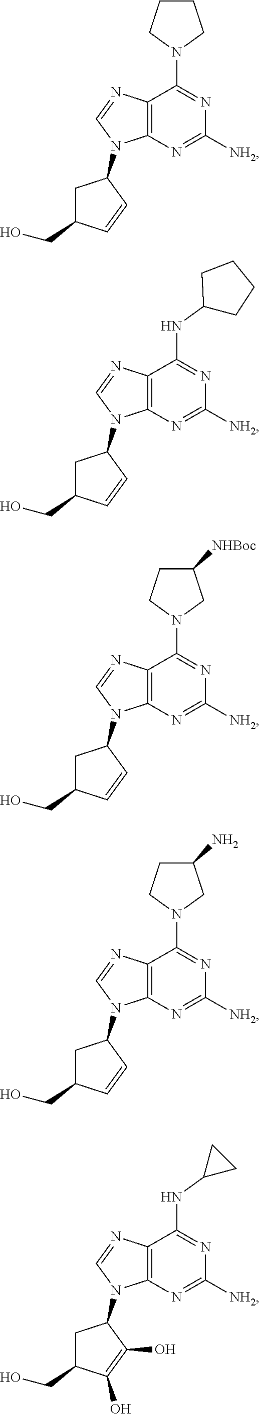

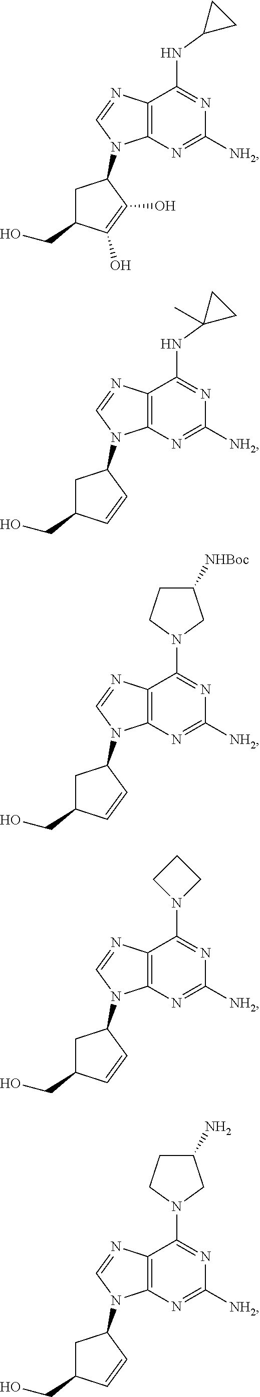

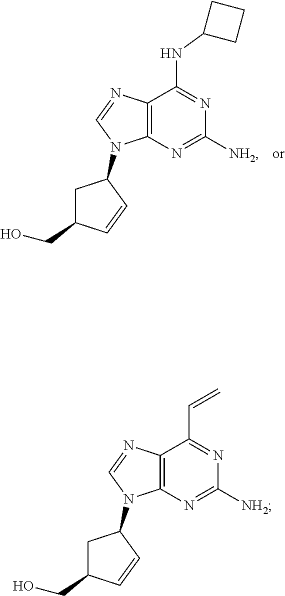 [SMILES: Nc1nc(NC2CCC2)c2ncn([C@H]3C=C[C@@H](CO)C3)c2n1 ; C=Cc1nc(N)nc2c1ncn2[C@H]1C=C[C@@H](CO)C1]

or a salt thereof.

### EXAMPLES

### Example 1: Screening for Compounds Inducing K-Ras Peptide Presentation

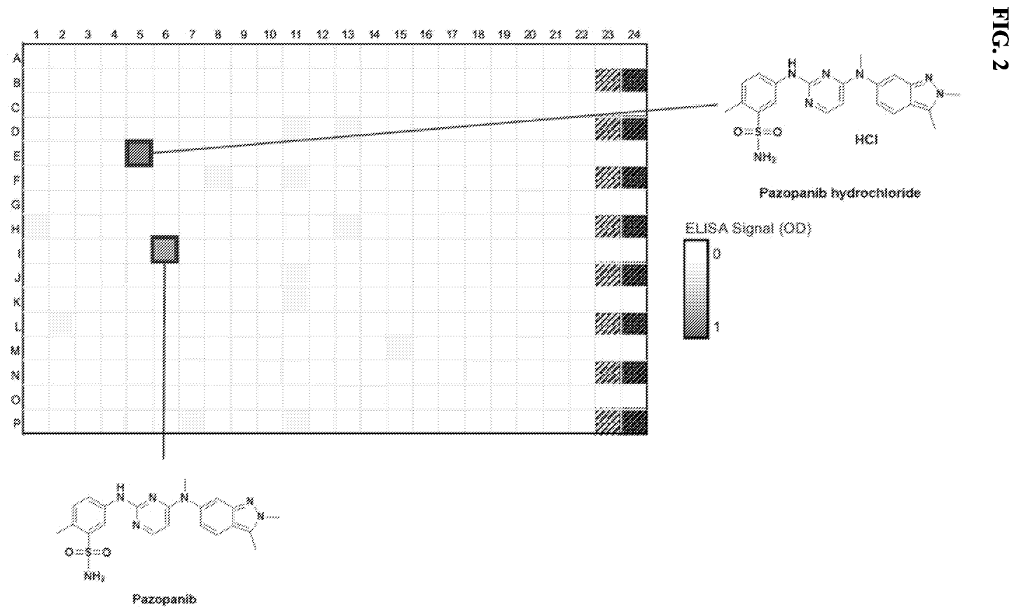

A Kinase inhibitor library was screened for compounds that induce K-Ras peptide presentation (FIG. 2). A Selleckchem kinase inhibitor library (368 compounds) were screened using the following conditions: 1 μM heavy chain, 2 μM 02-microglobulin, 100 μM compound, and 40 μM G12V 8-16 peptide. For a positive control, G12V 8-16 W16 was used, without additional compound. For a negative control, no compound was added. Shaded wells indicate positive and negative controls.

Refolding reactions were set up in 96-well plates on ice by the successive addition of the following:
1. Refolding Buffer (400 mM Arg-HCl, 100 mM Tris 8.0, 5 mM glutathione (GSH), 0.5 mM glutathione disulfide (GSSG), 1× cOmplete protease inhibitor cocktail);
2. Beta-2-microglobulin (final concentration 2 μM).
3. Peptide (final concentration 40 μM).
4. Denatured heavy chain (final concentration 1 μM).

| | | | |
|---|---|---|---|
| Component | Stock Conc | Final Conc | Volume |

| | | | | | | |
|---|---|---|---|---|---|---|
| Refolding Buffer | | | | | 98 | μL |
| Beta-2m | 302 | μM | 2 | μM | 0.67 | μL |
| Peptide | 10 | mM | 40 | μM | 0.40 | μL |
| Heavy chain | 100 | μM | 1 | μM | 1.0 | μL |

The contents were thoroughly mixed and then incubated at 10° C. for 18 h, with constant shaking at 100 rpm. Meanwhile, 96-well ELISA plate (Coming 3361) was treated with W6/32 (5 μg/mL) at 4° C. overnight. The plate was washed with PBS (2×200 μL), then blocked with 375 μL 3% BSA/PBS at 23° C. for 1 h. After washing the plate with PBST (3×200 μL), 90 μL 1% BSA/PBS was added to each well.

Detection of refolding was performed as follows:
1. Add 10 μL of the refolding mixture to each well of the ELISA plate.
2. Incubate the plate with shaking at 23° C. for 1 h.
3. Wash plate with 1% BSA/PBS (3×200 μL).
4. Add 50 μL BBM.1-HRP (1 μg/mL in 1% BSA/PBS) and incubate the plate with shaking at 23° C. for 1 h.
5. Wash plate with PBST (3×200 μL).
6. Wash plate with PBS (1×100 μL).
7. Add 50 μL TMB Ultra and incubate the plate with shaking for 5 min.
8. Stop the reaction by addition of 20 μL 2.0 M sulfuric acid.
Read absorbance at 450 nm within 10 min.

### Example 2: Presentation of Wild-Type and Mutant K-Ras Peptides with Pazopanib

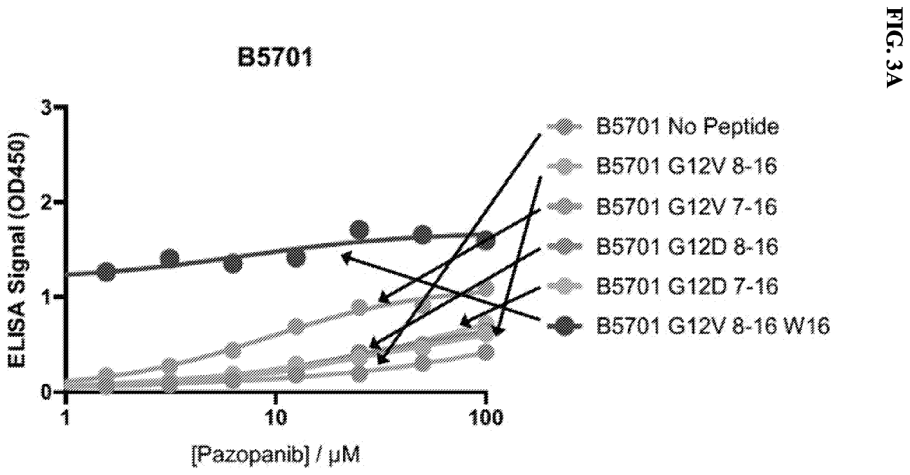

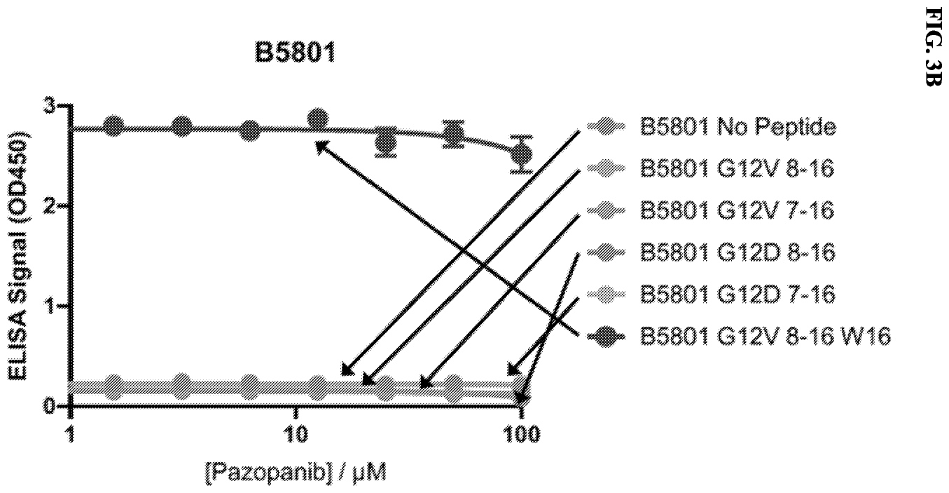

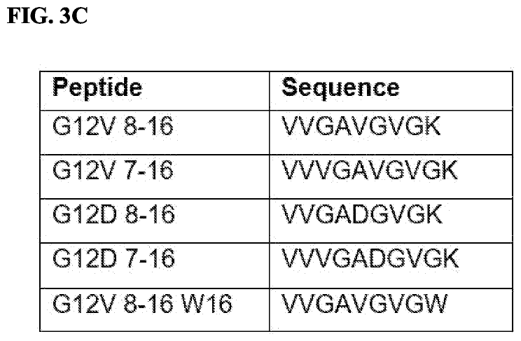

Pazopanib stabilizes the presentation of mutant K-Ras peptides by HLA-B*57:01 and HLA-B*58:01 (FIG. 3A-3B). The refolding conditions are as follows: 1 μM heavy chain, 2 μM β2-microglobulin, 40 μM peptide, and 1% DMSO, for 16 hours.

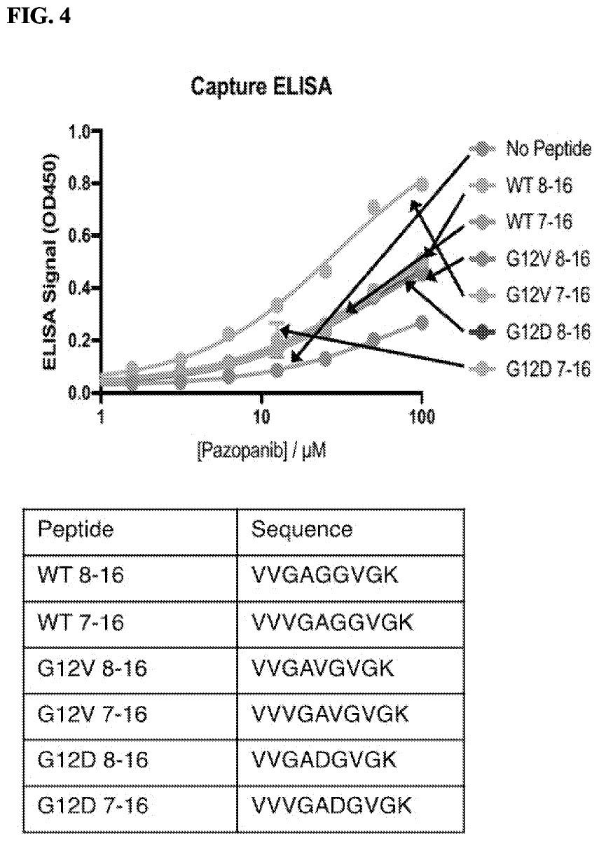

Pazopanib also stabilizes the presentation of wild-type K-Ras peptides by HLA-B*57:01 (FIG. 4). The refolding conditions are as follows: 1 μM heavy chain, 2 μM 02-microglobulin, 40 μM peptide, and 1% DMSO, for 16 hours.

### Example 3: Presentation of Mutant K-Ras Peptides with Pazopanib and Methylated Analogs of Pazopanib Over Time

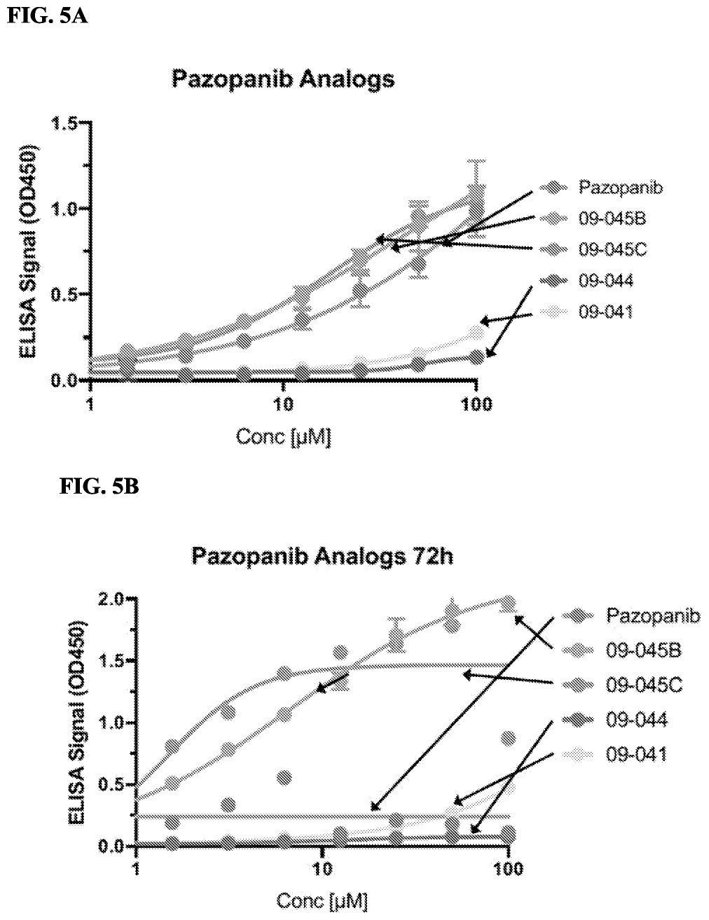

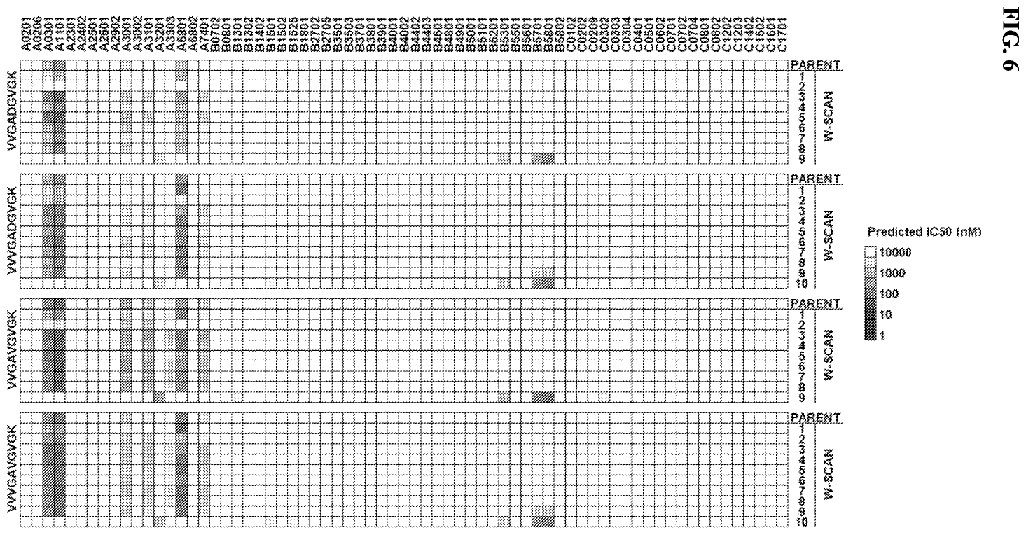

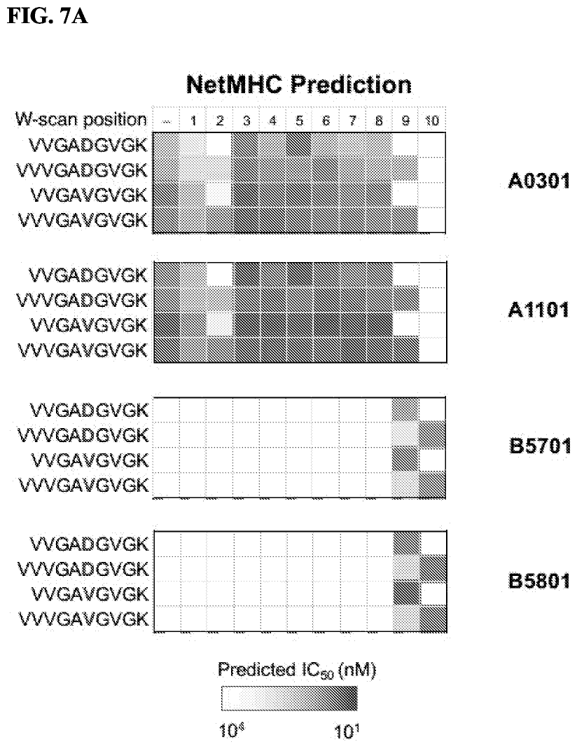

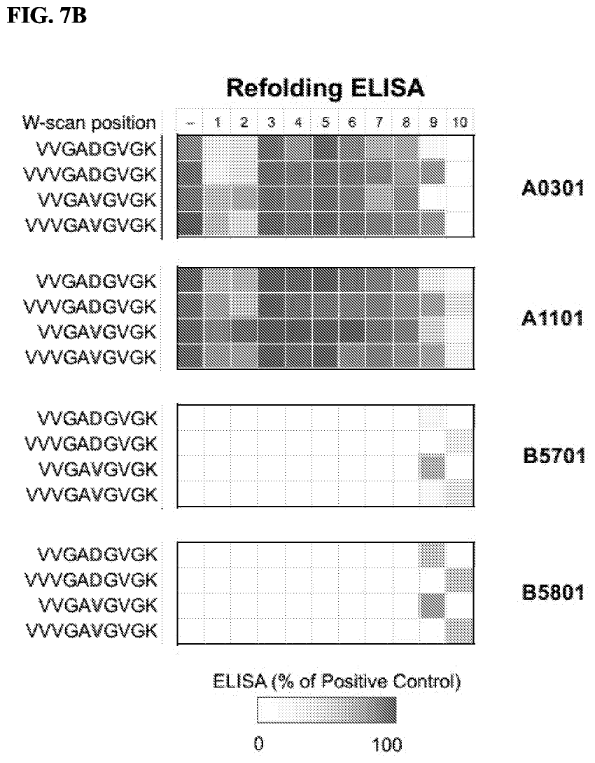

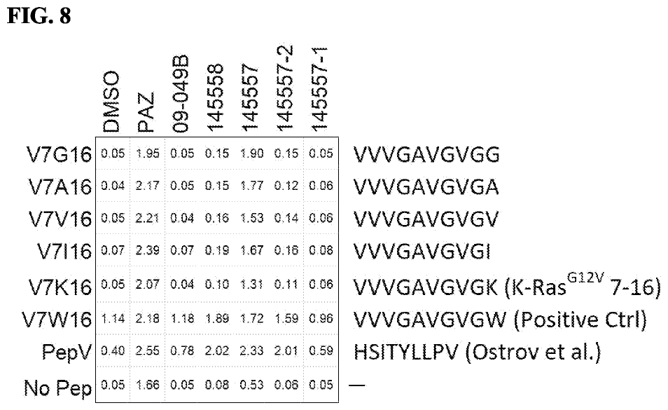

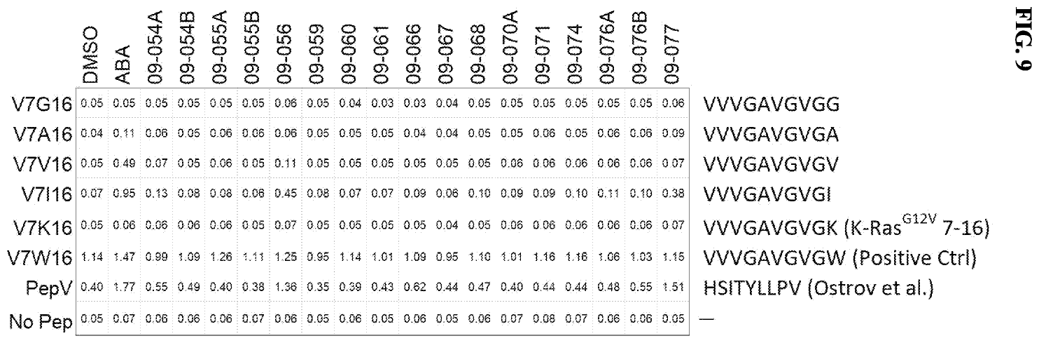

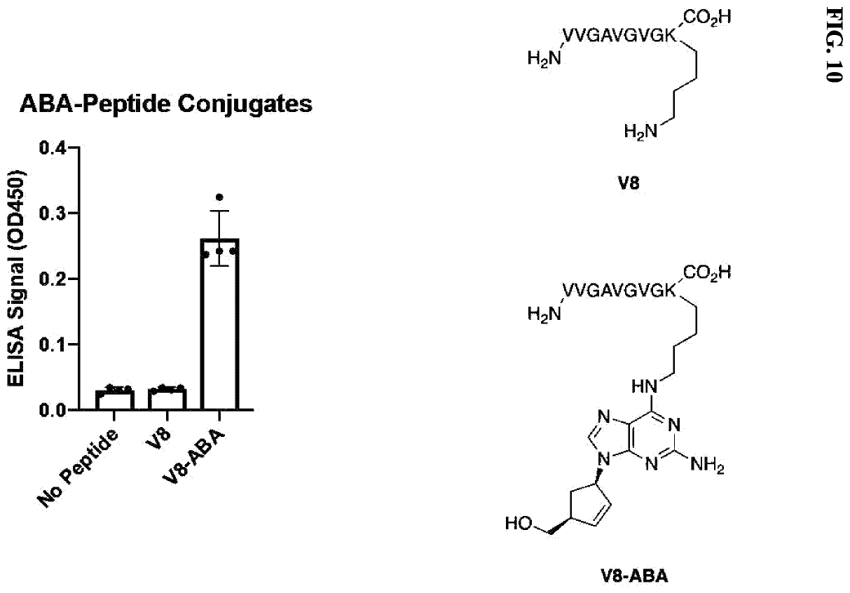

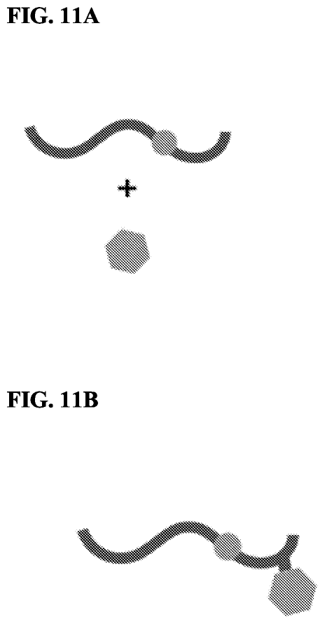

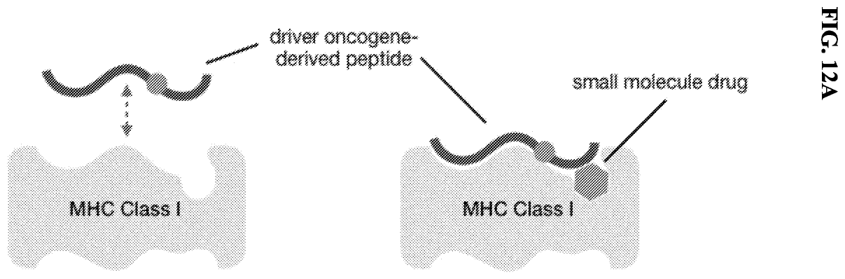

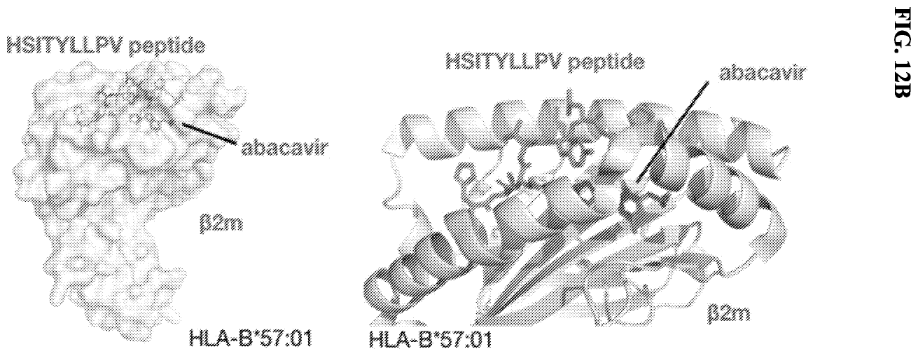

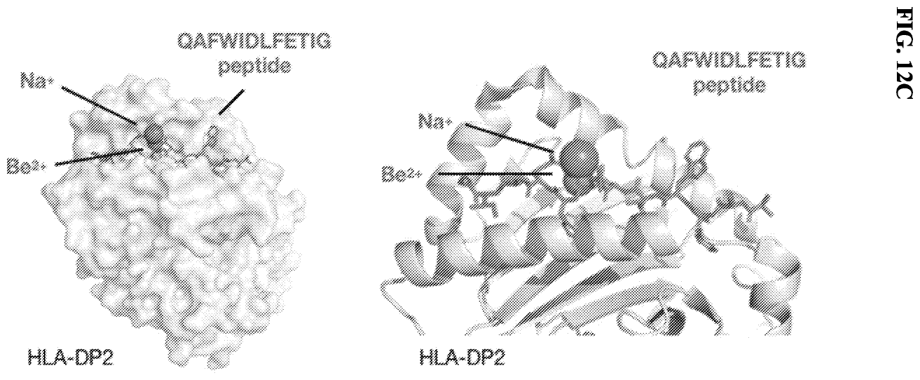

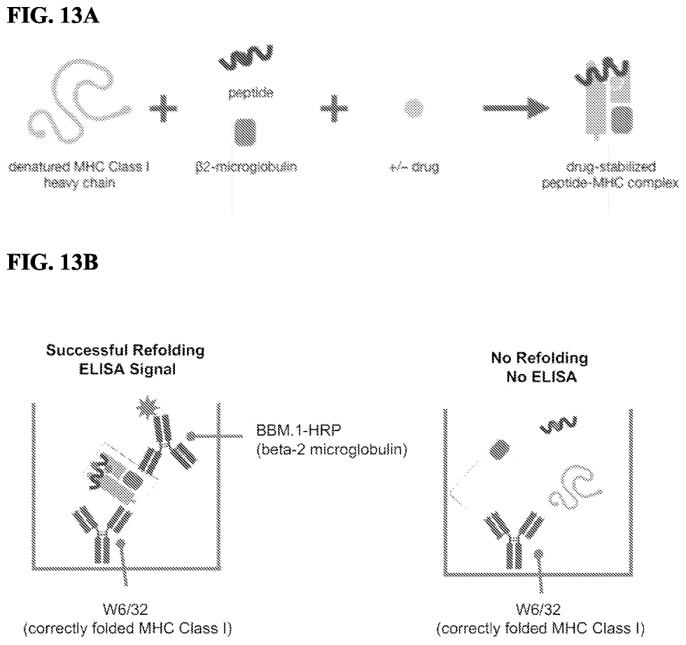

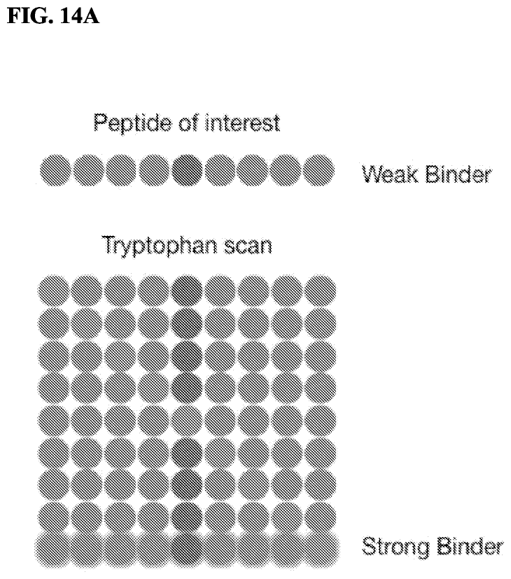

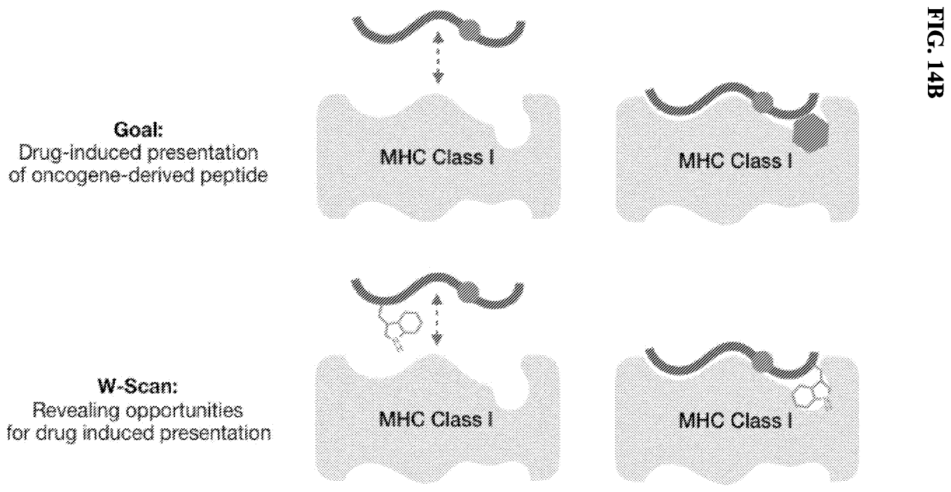

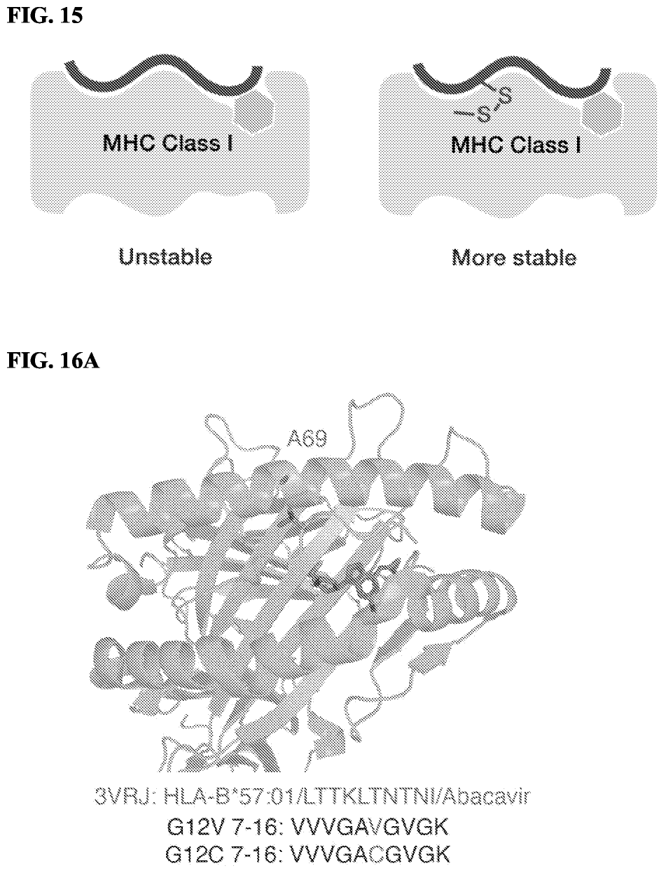

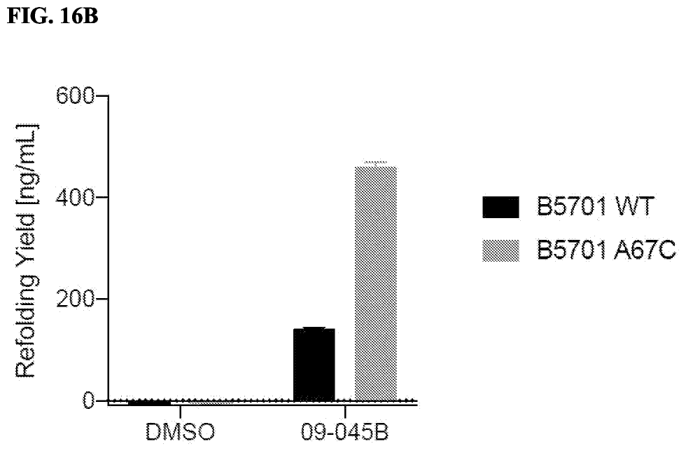

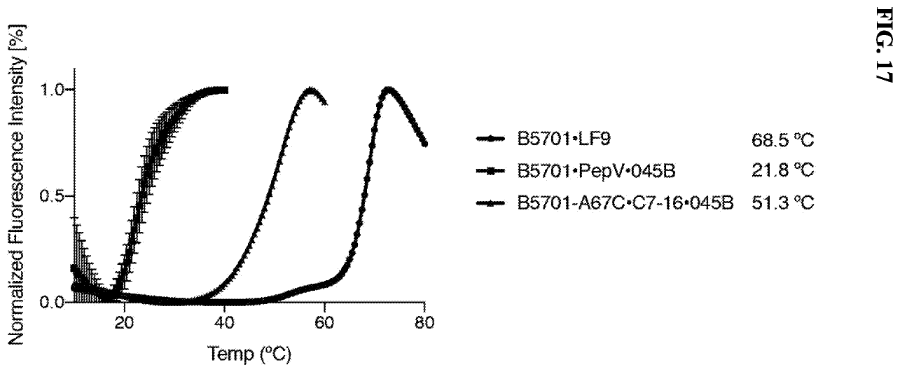

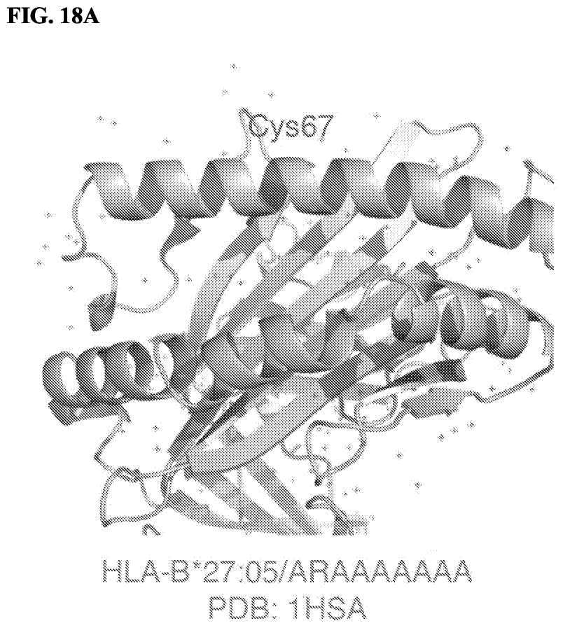

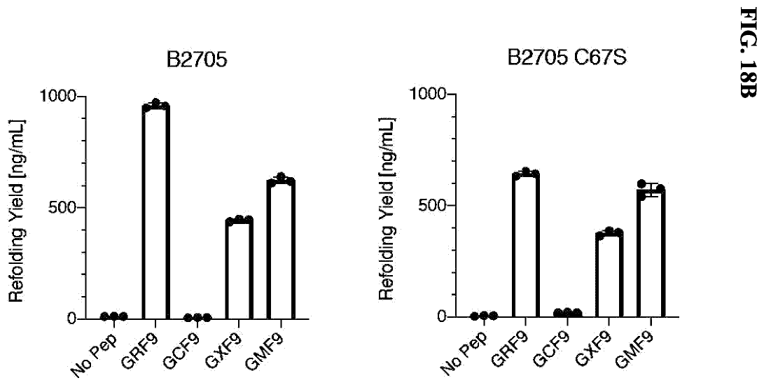

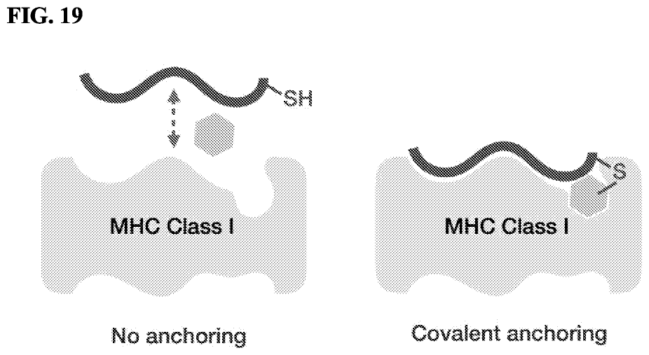

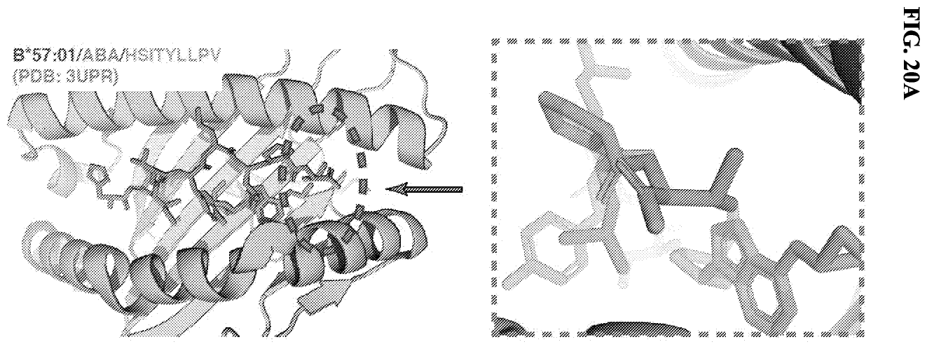

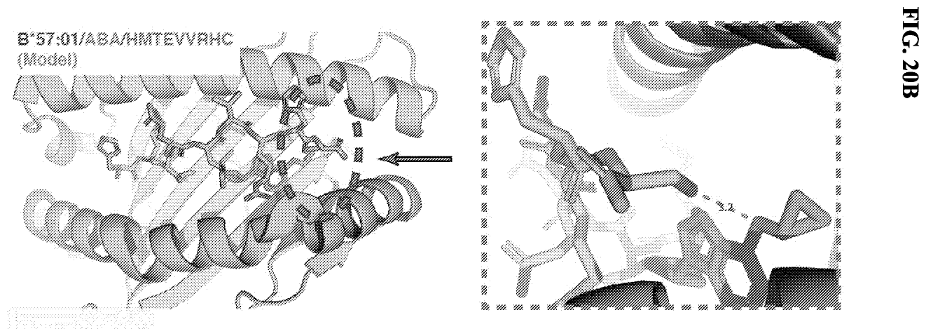

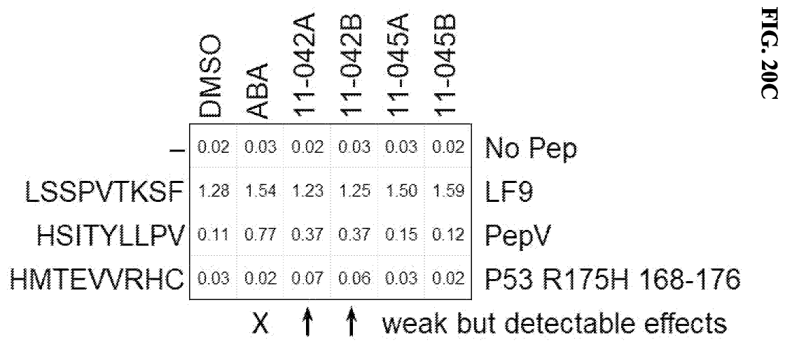

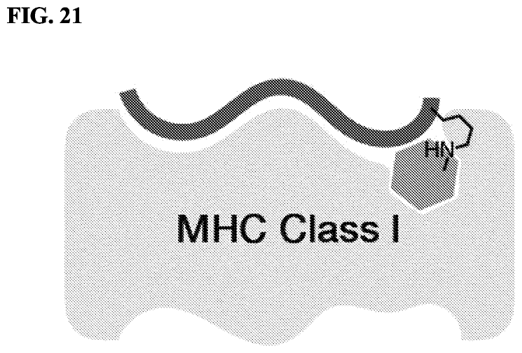

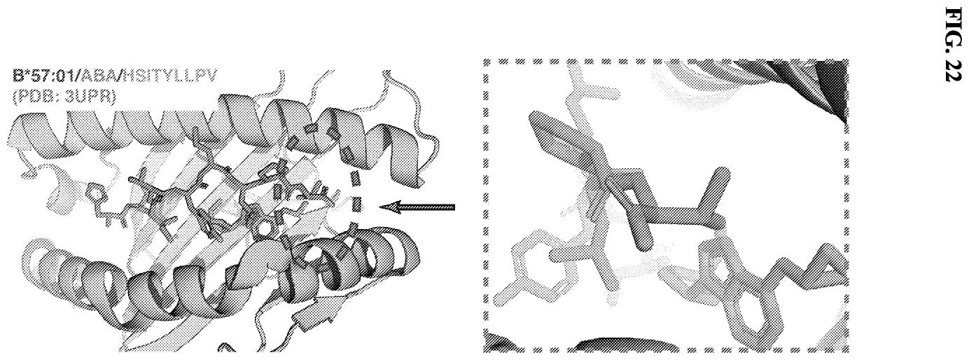

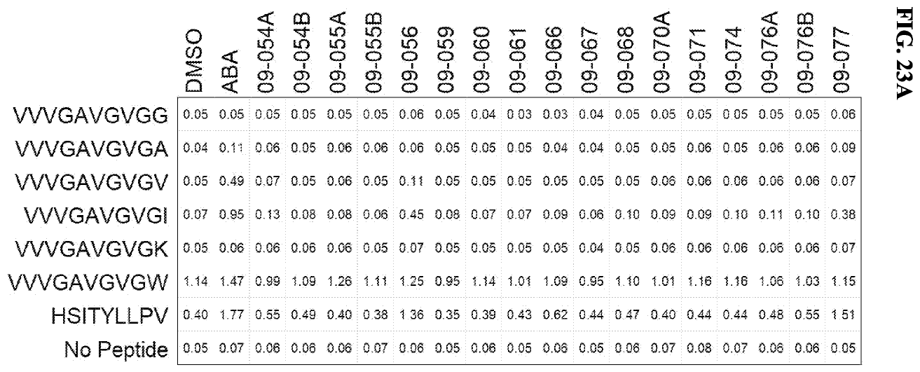

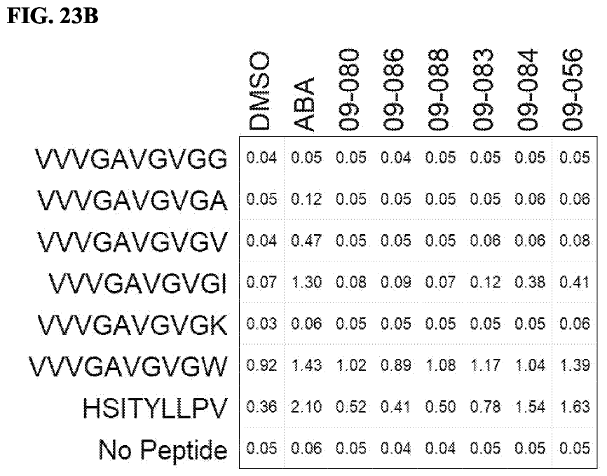

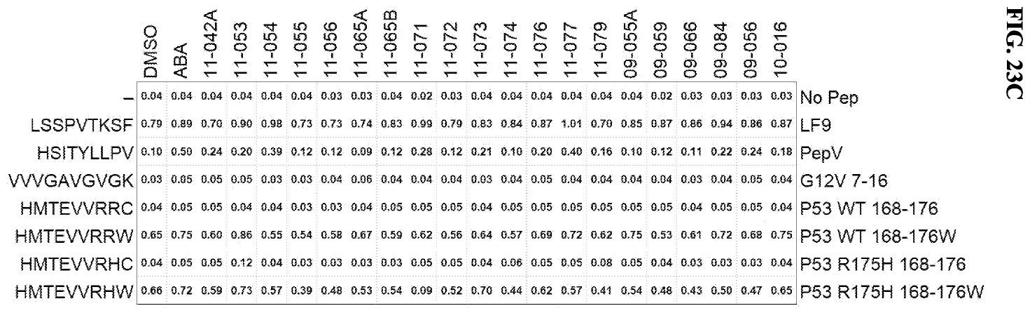

Methylated analogs of pazopanib maintain the ability to stabilize the presentation (FIG. 5A-5B). MHC-peptide complex formation decreases after 72 h with pazopanib, possibly due to instability of the complex. Methylated compound 09-045B may be less susceptible to this instability. The refolding conditions are as follows: 1 μM heavy chain, 2 μM 02-microglobulin, 40 μM G12V 7-16, and 1% DMSO, at 10° C. for 16 hours or 72 hours.

### Example 4: Pazopanib Analogs

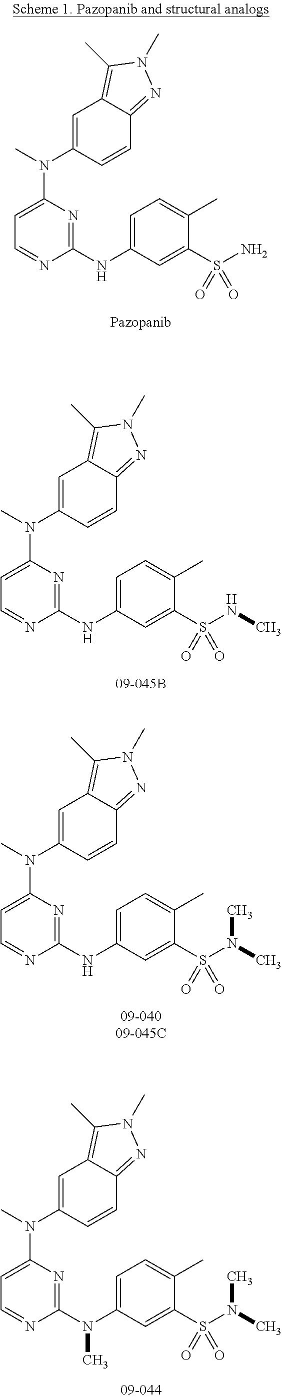

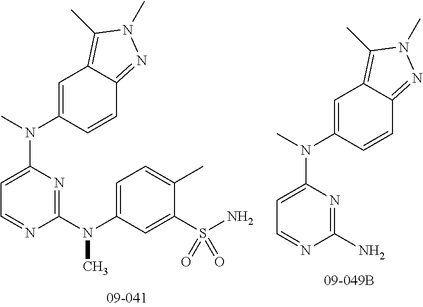

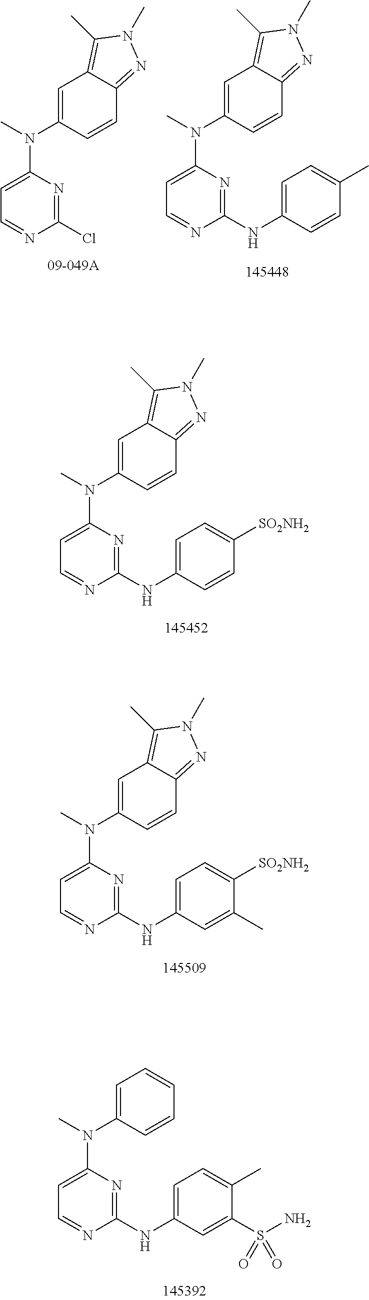 [SMILES: Cc1c2cc(N(C)c3ccnc(Cl)n3)ccc2nn1C ; Cc1ccc(Nc2nccc(N(C)c3ccc4nn(C)c(C)c4c3)n2)cc1 ; Cc1c2cc(N(C)c3ccnc(Nc4ccc(S(N)(=O)=O)cc4)n3)ccc2nn1C ; Cc1cc(Nc2nccc(N(C)c3ccc4nn(C)c(C)c4c3)n2)ccc1S(N)(=O)=O ; Cc1ccc(Nc2nccc(N(C)c3ccccc3)n2)cc1S(N)(=O)=O]

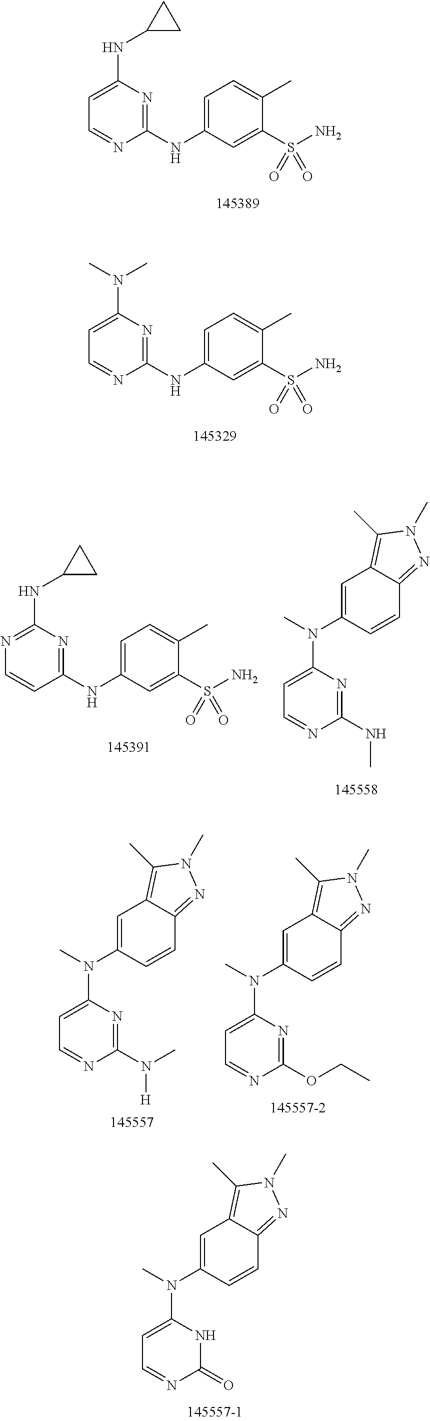

### Example 5: Abacavir Analogs

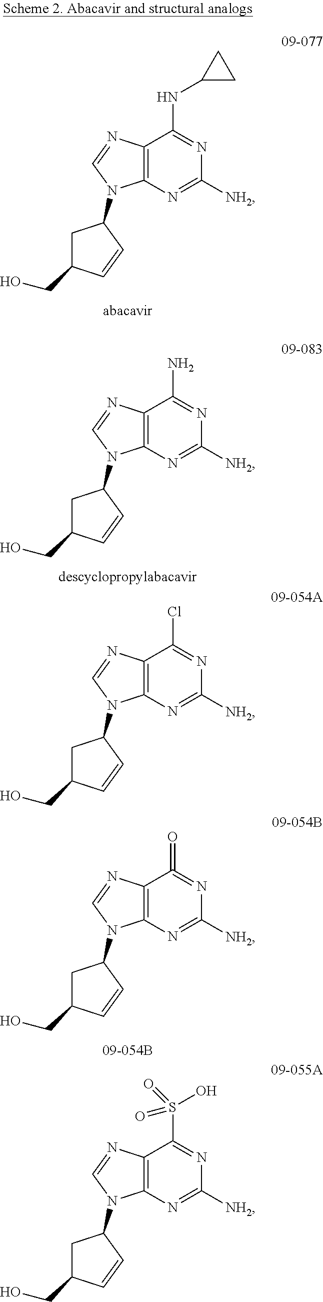

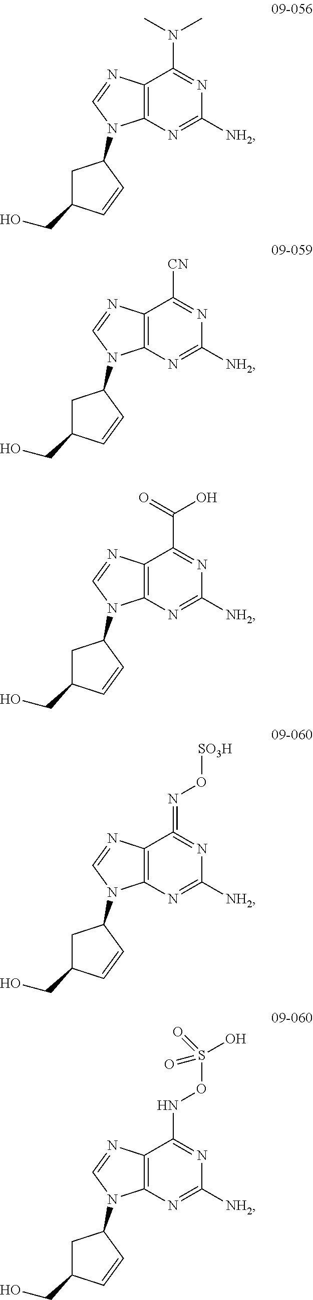

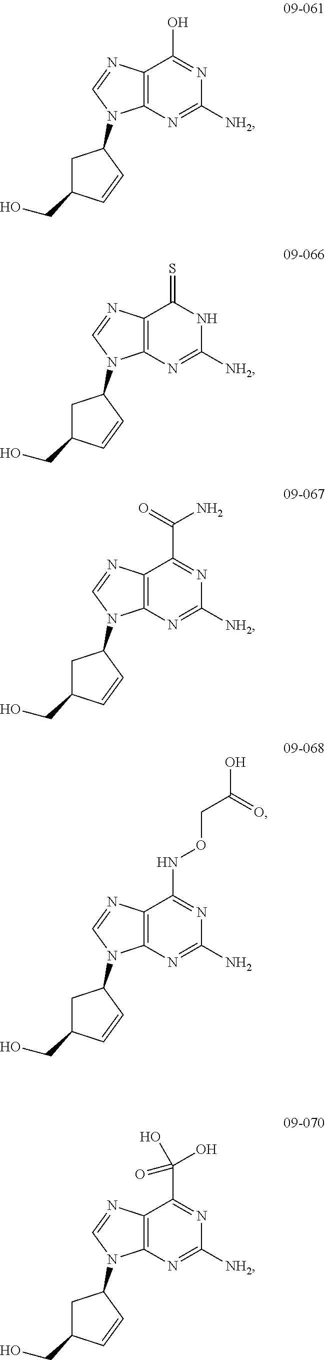

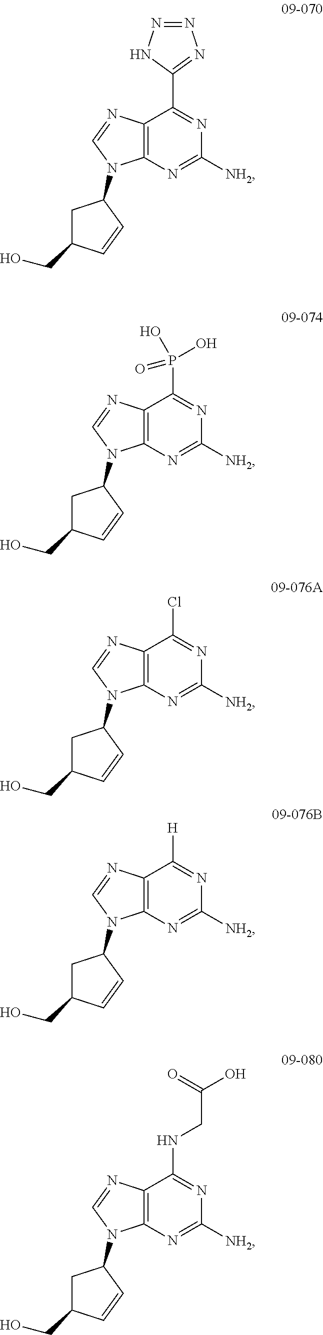 [SMILES: Nc1nc(-c2nnn[nH]2)c2ncn([C@H]3C=C[C@@H](CO)C3)c2n1 ; Nc1nc(P(=O)(O)O)c2ncn([C@H]3C=C[C@@H](CO)C3)c2n1 ; Nc1nc(Cl)c2ncn([C@H]3C=C[C@@H](CO)C3)c2n1 ; Nc1ncc2ncn([C@H]3C=C[C@@H](CO)C3)c2n1 ; Nc1nc(NCC(=O)O)c2ncn([C@H]3C=C[C@@H](CO)C3)c2n1]

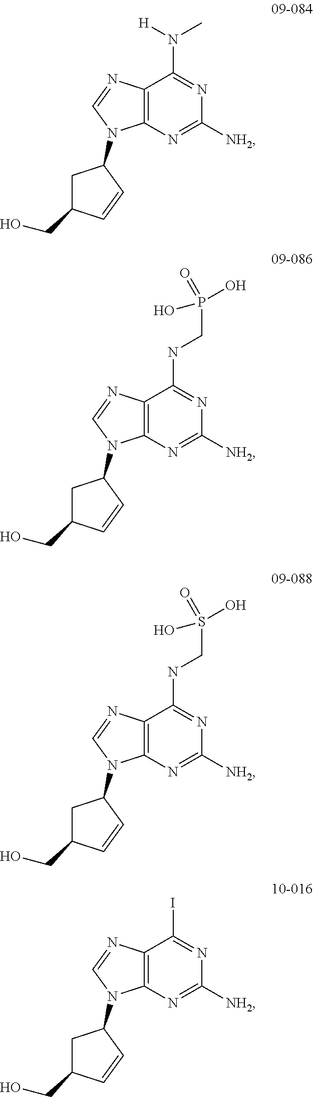

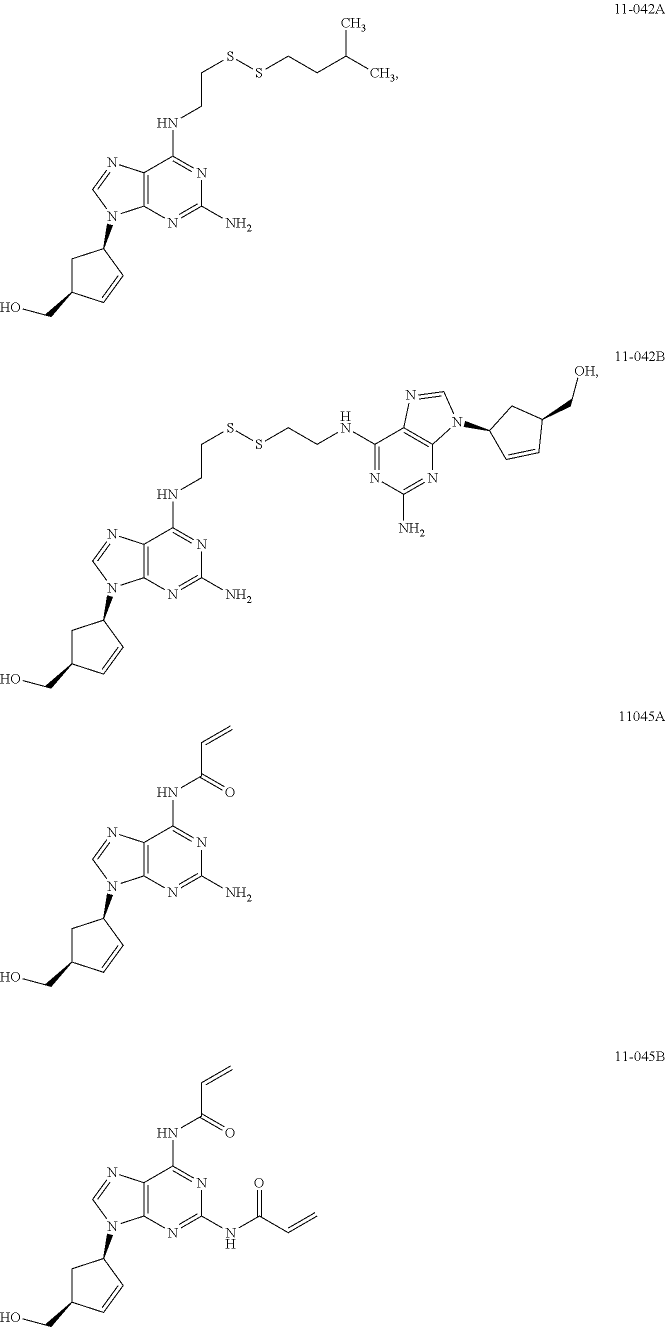 [SMILES: CC(C)CCSSCCNc1nc(N)nc2c1ncn2[C@H]1C=C[C@@H](CO)C1 ; Nc1nc(NCCSSCCNc2nc(N)nc3c2ncn3[C@H]2C=C[C@@H](CO)C2)c2ncn([C@@H]3C=C[C@H](CO)C3)c2n1 ; C=CC(=O)Nc1nc(N)nc2c1ncn2[C@H]1C=C[C@@H](CO)C1 ; C=CC(=O)Nc1nc(NC(=O)C=C)c2ncn([C@H]3C=C[C@@H](CO)C3)c2n1]

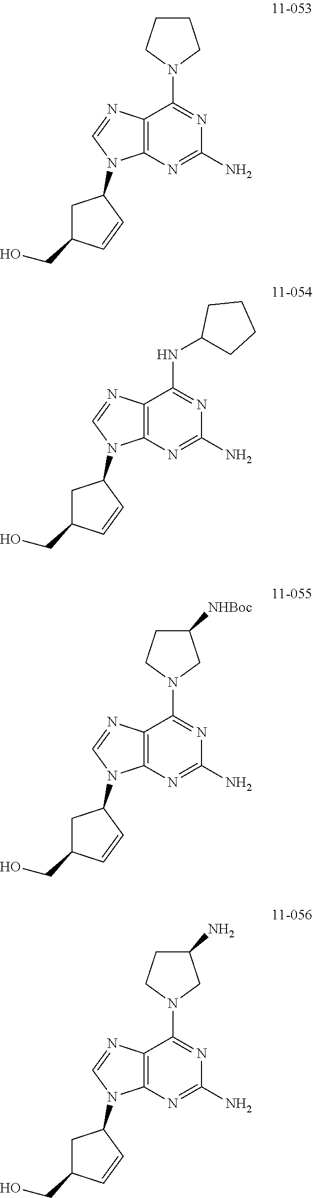 [SMILES: Nc1nc(N2CCCC2)c2ncn([C@H]3C=C[C@@H](CO)C3)c2n1 ; Nc1nc(NC2CCCC2)c2ncn([C@H]3C=C[C@@H](CO)C3)c2n1 ; CC(C)(C)OC(=O)N[C@@H]1CCN(c2nc(N)nc3c2ncn3[C@H]2C=C[C@@H](CO)C2)C1 ; Nc1nc(N2CC[C@@H](N)C2)c2ncn([C@H]3C=C[C@@H](CO)C3)c2n1]

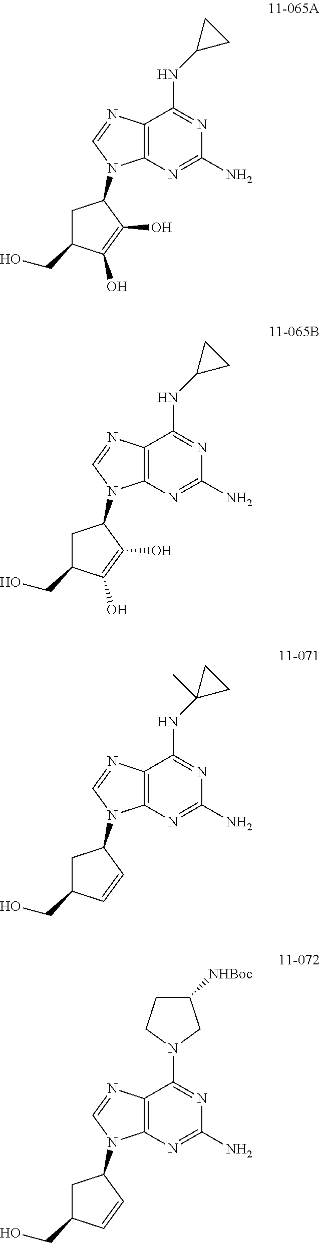

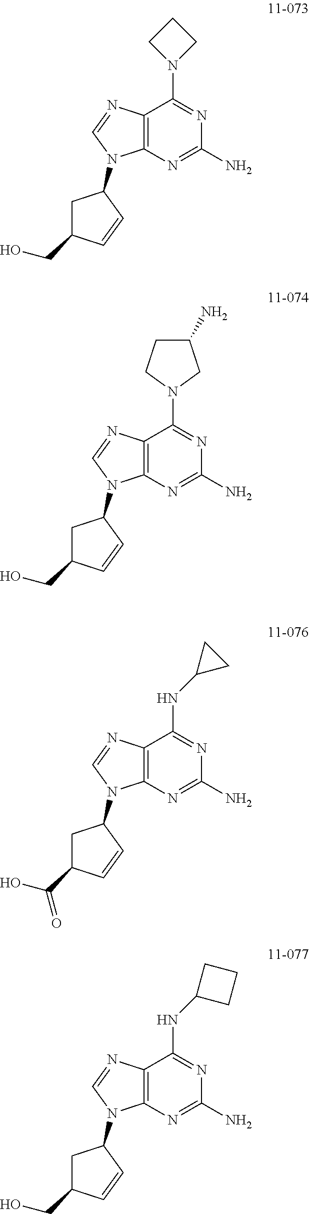 [SMILES: Nc1nc(N2CCC2)c2ncn([C@H]3C=C[C@@H](CO)C3)c2n1 ; Nc1nc(N2CC[C@H](N)C2)c2ncn([C@H]3C=C[C@@H](CO)C3)c2n1 ; Nc1nc(NC2CC2)c2ncn([C@H]3C=C[C@@H](C(=O)O)C3)c2n1 ; Nc1nc(NC2CCC2)c2ncn([C@H]3C=C[C@@H](CO)C3)c2n1]

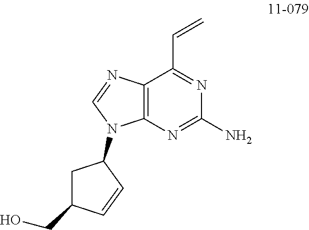 [SMILES: C=Cc1nc(N)nc2c1ncn2[C@H]1C=C[C@@H](CO)C1]

### Example 6: Exemplary Synthetic Methods for Pazopanib Analogs

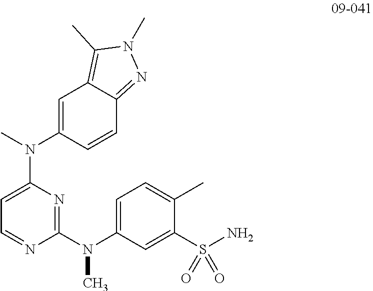

Methyl iodide (14 μL, 0.23 mmol) was added via syringe to a suspension of pazopanib (20 mg, 0.046 mmol) in DMF (0.20 mL). The mixture was warmed to 40° C. for 1 h. The reaction mixture was diluted with 50% acetonitrile-water to a volume of 3.0 mL, and the solution was filtered through a 0.45 μM PTFE syringe filter. The filtrate was purified by reverse-phase HPLC (Waters XBridge C18 column 5 μm particle size 30×250 mm, 20-40% acetonitrile−water+0.1% formic acid, 40 min, 20 mL/min) to afford the product as a white solid (11.1 mg, 54%). ¹H NMR (400 MHz, DMSO) δ 8.23 (s, 1H), 7.90 (d, J=2.2 Hz, 1H), 7.77 (d, J=8.7 Hz, 1H), 7.57 (d, J=7.5 Hz, 1H), 7.43 (d, J=1.7 Hz, 1H), 7.38 (dd, J=8.1, 2.3 Hz, 1H), 7.15 (d, J=11.6 Hz, 3H), 6.86 (dd, J=8.8, 1.8 Hz, 1H), 5.33 (d, J=7.5 Hz, 1H), 4.07 (s, 3H), 3.40 (s, 3H), 3.35 (s, 3H), 2.63 (s, 3H). HRMS (ESI): Calcd for (C₂₂H₂₆N₇O₂S+H)⁺: 452.1869, Found: 452.1868.

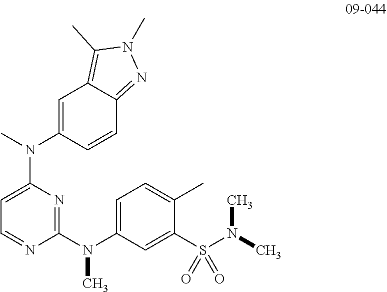

An oven-dried 1-dram vial was charged with pazopanib (50 mg, 0.11 mmol), DMF (0.20 mL) and magnetic stir bar. Pazopanib did not fully dissolved in DMF. Sodium hydride (19 mg, 0.46 mmol) was added as a solid. The mixture was stirred for 30 min, then methyl iodide (36 μL, 0.57 mmol) was added to the reaction mixture via syringe. After 1 h at 23° C., saturated ageuous ammonium chloride solution (1 mL) was added, and the mixture was extracted with ethyl acetate (3×1 mL). The combined organic layers were dried over sodium sulfate and concentrated under reduced pressure. The residue was diluted with 50% acetonitrile-water to a volume of 3.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.10% formic acid, 40 min, 20 mL/min) to afford the product as a white solid (33 mg, 60%). ¹H NMR (400 MHz, DMSO) δ 7.81 (d, J=2.4 Hz, 1H), 7.77 (d, J=5.9 Hz, 1H), 7.73 (d, J=8.8 Hz, 1H), 7.58 (dd, J=8.2, 2.4 Hz, 1H), 7.44-7.37 (m, 2H), 6.85 (dd, J=8.8, 1.8 Hz, 1H), 5.73 (d, J=6.0 Hz, 1H), 4.05 (s, 3H), 3.49 (s, 3H), 3.34 (s, 3H), 2.73 (s, 6H), 2.62 (s, 3H), 2.54 (s, 3H). HRMS (ESI): Calcd for (C₂₄H₂₉N₇O₂S+H)⁺: 480.2182, Found: 480.2171.

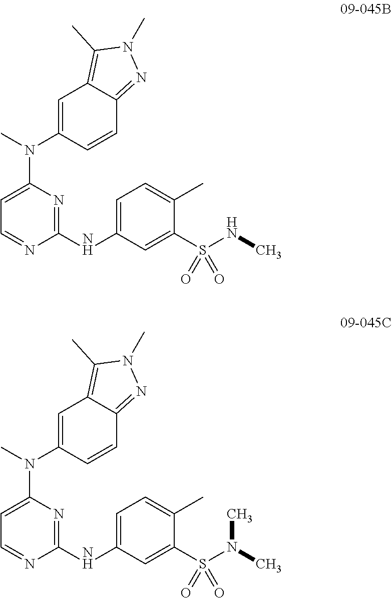

An oven-dried 1-dram vial was charged with pazopanib (50 mg, 0.11 mmol), DMF (0.20 mL) and a magnetic stir bar. The starting material did not fully dissolve. Sodium hydride (5.0 mg, 0.13 mmol) was added as a solid. The mixture was stirred for 30 min, then methyl iodide (14 μL, 0.23 mmol) was added to the reaction mixture via syringe. In 1 h, the reaction was quenched by addition of 0.1 mL of water. The reaction mixture was partitioned between saturated sodium ammonium chloride solution (1 mL) and ethyl acetate (1 mL). The layers were separated, and the aqueous layer was extracted with ethyl acetate (2×1 mL). The combined organic layers were dried over sodium sulfate. The dried solution was filtered, and the filtrate was concentrated. The residue was diluted with 50% acetonitrile-water to a volume of 4.7 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.10% formic acid, 40 min, 20 mL/min) to afford 09-045B (17.9 mg, 35%) and 09-045C (15.4 mg, 29%). 09-045B: ¹H NMR (400 MHz, DMSO) δ 9.42 (s, 1H), 8.56 (s, 1H), 7.84 (d, J=5.9 Hz, 1H), 7.81-7.71 (m, 2H), 7.46 (d, J=1.7 Hz, 1H), 7.31 (d, J=5.0 Hz, 1H), 7.20 (d, J=8.3 Hz, 1H), 6.89 (dd, J=8.7, 1.8 Hz, 1H), 5.76 (d, J=6.0 Hz, 1H), 4.07 (s, 3H), 3.51 (s, 3H), 2.64 (s, 3H), 2.48 (s, 3H), 2.43 (d, J=4.9 Hz, 3H). HRMS (ESI): Calcd for (C₂₂H₂₆N₇O₂S+H)⁺: 452.1869, Found: 452.1868. 09-045C: ¹H NMR (400 MHz, DMSO) δ 9.46 (s, 1H), 8.45 (s, 1H), 7.88-7.82 (m, 2H), 7.78 (d, J=8.8 Hz, 1H), 7.46 (d, J=1.7 Hz, 1H), 7.24 (d, J=8.4 Hz, 1H), 6.89 (dd, J=8.8, 1.8 Hz, 1H), 5.78 (d, J=6.1 Hz, 1H), 4.07 (s, 3H), 3.51 (s, 3H), 2.75 (s, 6H), 2.64 (s, 3H), 2.47 (s, 3H). HRMS (ESI): Calcd for (C₂₃H₂₈N₇O₂S+H)⁺: 466.2024, Found: 466.2049.

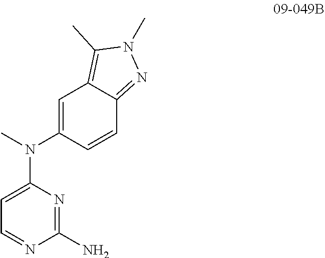 [SMILES: Cc1c2cc(N(C)c3ccnc(N)n3)ccc2nn1C]

A suspension of N-(2-chloropyrimidin-4-yl)-N,2,3-trimethyl-indazol-5-amine (50. mg, 0.1738 mmol) in 30% aqueous ammonium hydroxide (0.5 mL) was heated at 140° C. for 16 h in a microwave reactor. The reaction mixture was concentrated under reduced pressure. The residue was dissolved in 50% acetonitrile-water+1% TFA (4.0 mL). The resulting solution was fitlered through a 0.45-μM PTFE syringe filter, and the filtrate was purified by reverse-phase HPLC (Waters XBridge C18 column 5 μm particle size 30×250 mm, 5-95% acetonitrile-water+0.1% formic acid, 40 min, 20 mL/min) to afford the product as a white solid (31 mg, 66%). ¹H NMR (400 MHz, DMSO) δ 7.95 (d, J=6.1 Hz, 1H), 7.81 (dd, J=8.8, 0.8 Hz, 1H), 7.51 (dd, J=1.8, 0.8 Hz, 1H), 6.89 (dd, J=8.8, 1.8 Hz, 1H), 6.25 (d, J=6.1 Hz, 1H), 4.07 (s, 3H), 3.43 (s, 3H), 2.63 (s, 3H). HRMS (ESI): Calcd for (C₁₄H₁₆N₆+H)⁺: 269.1515, Found: 267.1524.

General Procedure C

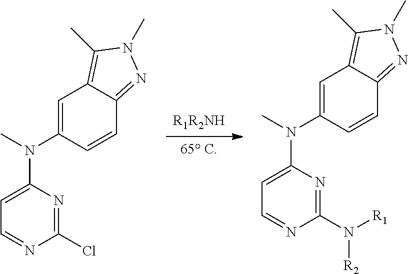

Amine (0.34 mmol, 2 equiv.) and 1 N HCl (0.1 mL) was added to a suspension of N-(2-chloropyrimidin-4-yl)-N,2,3-trimethyl-indazol-6-amine (50 mg, 0.17 mmol). The mixture was heated at 65° C. for 16 h. The reaction mixture was partitioned between saturated sodium bicarbonate solution (1 mL) and ethyl acetate (1 mL). The layers were separated, and the aqueous layer was extracted with ethyl acetate (2×1 mL). The combined organic layers were dried over sodium sulfate. The dried solution was filtered, and the filtrate was concentrated. The residue was purified by flash column chromatography (0-20% methanol-dichloromethane) to afford the product as a white solid.

All compounds synthesized using General Procedure C were characterized and confirmed using, for example, ¹H NMR and/or Mass Spectrometry.

### Example 7: Exemplary Synthetic Methods for Abacavir Analogs

Abacavir, 09-054A, 09-083 and 11-076 were purchased from commercial vendors.

General Method A

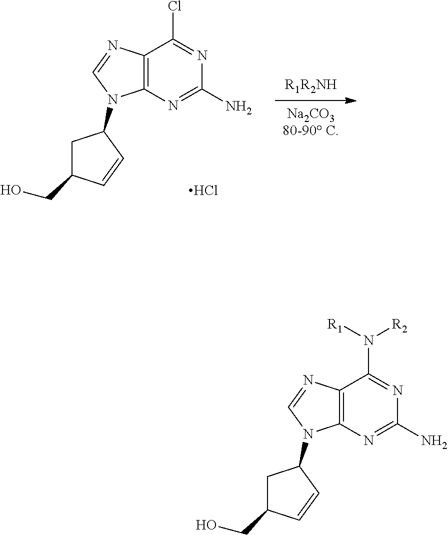

A one-dram vial was charged with [(1S,4R)-4-(2-amino-6-chloro-purin-9-yl)cyclopent-2-en-1-yl]methanol hydrochloride (50 mg, 0.17 mmol), sodium carbonate (53 mg, 0.50 mmol), ethanol (0.5000 mL), and a magnetic stir bar. Amine (0.25 mmol) was added, and the reaction mixture was heated at 80° C. until LC-MS analysis showed full consumption of the starting material. Typically, the reaction was complete in 30 min for sterically unhindered amines. For sterically hindered amines, the reaction time was extended up to 48 h, and in certain cases, the reaction temperature was increased to 90° C. The reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (0-10% methanol-dichloromethane, 4-g RediSep(R) Rf column, Teledyne ISCO, Lincoln, Nebr.) to afford the product as a white powder.

General Method B

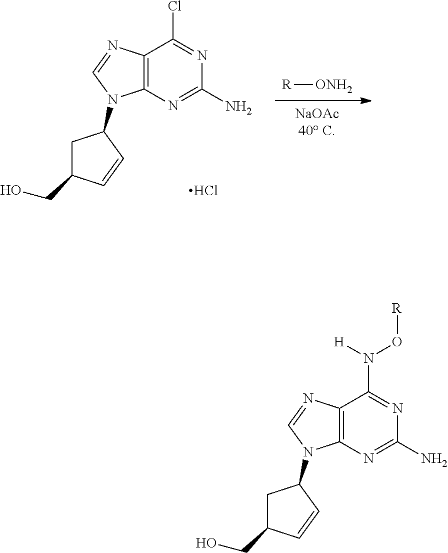

A 20-mL vial was charged with [(1S,4R)-4-(2-amino-6-chloro-purin-9-yl)cyclopent-2-en-1-yl]methanol hydrochloride (50 mg, 0.17 mmol), hydroxylamine (0.66 mmol), sodium acetate (54 mg, 0.66 mmol), and a magnetic stir bar. Trifluoroethanol (1.0 mL) was added by syringe, and the resulting mixture was stirred at 40° C. for 20 h or until LCMS analysis showed full consumption of the starting material. The reaction mixture was evaporated to dryness, the residue was diluted with 50% acetonitrile-water to a volume of 3.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% trifluoroacetic acid, 40 min, 20 mL/min) to afford the product as a white solid.

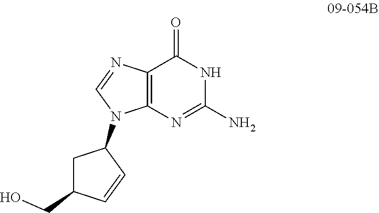 [SMILES: Nc1nc2c(ncn2[C@H]2C=C[C@@H](CO)C2)c(=O)[nH]1]

To a solution of [(1S,4R)-4-(2-amino-6-chloro-purin-9-yl)cyclopent-2-en-1-yl]methanol hydrochloride (60 mg, 0.20 mmol) in MeCN (1.0 mL) was added a 1.0 M aqueous solution of sodium hydroxide (0.60 mL, 0.60 mmol). The mixture was heated at 90° C. for 3 h in a microwave reactor. The reaction mixture was acidified with trifluoroacetic acid to pH=2, diluted with 50% acetonitrile-water to a volume of 3.0 mL, and filtered through a PTFE syringe filter. The filtrate was purified by reverse-phase HPLC (Waters XBridge C18 column 5 μm particle size 30×250 mm, 5-95% acetonitrile-water+0.10% trifluoroacetic acid, 40 min, 20 mL/min) to afford the product as a white solid (27 mg, 55%). HRMS (ESI): Calcd for (C₁₁H₄N₅O₂+H)⁺: 248.1147, Found: 248.0910.

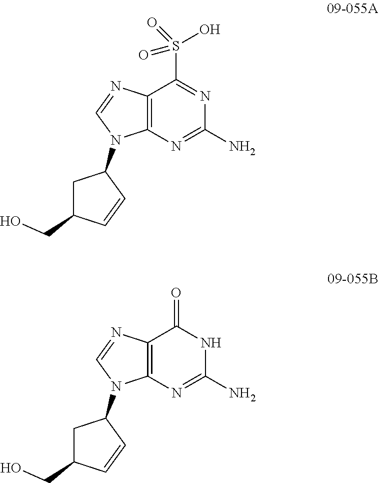 [SMILES: Nc1nc(S(=O)(=O)O)c2ncn([C@H]3C=C[C@@H](CO)C3)c2n1 ; Nc1nc2c(ncn2[C@H]2C=C[C@@H](CO)C2)c(=O)[nH]1]

To a solution of [(1S,4R)-4-(2-amino-6-chloro-purin-9-yl)cyclopent-2-en-1-yl]methanol hydrochloride (53 mg, 0.17 mmol) in MeCN (0.85 mL) was added a 1.0 M aqueous solution of sodium sulfite (0.52 mL, 0.52 mmol). The mixture was heated at 90° C. for 4 h in a microwave reactor. The reaction mixture was acidified with trifluoroacetic acid to pH=2, diluted with 50% acetonitrile-water to a volume of 3.0 mL, and filtered through a PTFE syringe filter. The filtrate was purified by reverse-phase HPLC (Waters XBridge C18 column 5 μm particle size 30×250 mm, 5-95% acetonitrile-water+0.10% trifluoroacetic acid, 40 min, 20 mL/min) to afford the 09-055A (5.3 mg, 9%) and 09-055B (4.6 mg, 11%). 09-055B was found to be identical to 09-054B. 09-055A: HRMS (ESI): Calcd for (C₁₁H₁₄N₅O₄S+H)⁺: 312.0766, Found: 312.0538. 09-055B: HRMS (ESI): Calcd for (C₁₁H₁₄N₅O₂+H)⁺: 248.1147, Found: 248.0910.

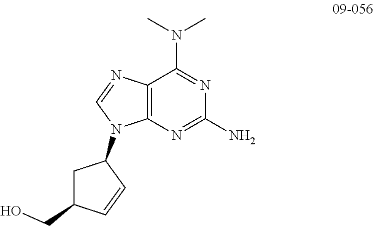 [SMILES: CN(C)c1nc(N)nc2c1ncn2[C@H]1C=C[C@@H](CO)C1]

Prepared using general method A. White solid (23 mg, 51%). ¹H NMR (400 MHz, DMSO) δ 7.62 (s, 1H), 6.12 (dt, J=5.6, 2.1 Hz, 1H), 5.86 (dt, J=5.7, 2.2 Hz, 1H), 5.81 (s, 2H), 5.41 (ddt, J=9.0, 5.7, 2.1 Hz, 1H), 4.74 (t, J=5.5 Hz, 1H), 3.44 (t, J=5.5 Hz, 3H), 3.36 (s, 6H), 2.87 (td, J=6.0, 3.0 Hz, 1H), 2.60 (dt, J=13.8, 8.7 Hz, 1H), 1.55 (dt, J=13.7, 5.7 Hz, 1H). HRMS (ESI): Calcd for (C₁₃H₁₈N₆O+H)⁺: 275.1620, Found: 275.1601.

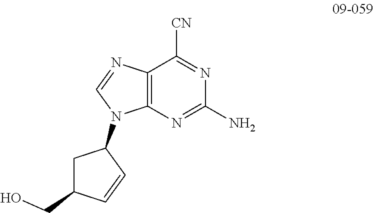 [SMILES: N#Cc1nc(N)nc2c1ncn2[C@H]1C=C[C@@H](CO)C1]

Tetrabutylammonium cyanide (89 mg, 0.33 mmol) was added as a solution in acetonitrile (0.5 mL) to a stirred suspension of [(1S,4R)-4-(2-amino-6-chloro-purin-9-yl)cyclopent-2-en-1-yl]methanol hydrochloride (50 mg, 0.17 mmol) and DABCO (37 mg, 0.33 mmol) in MeCN (0.85 mL). The mixture was stirred at 23° C. for 2 h. The reaction mixture was partitioned between saturated aqueous sodium bicarbonate solution (5 mL) and ethyl acetate (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (50-100% ethyl acetate-hexanes, 4-g RediSep(R) Rf column, Teledyne ISCO, Lincoln, Nebr.) to afford the product as a white powder (27 mg, 64%). ¹H NMR (400 MHz, DMSO) δ 8.27 (s, 1H), 7.09 (s, 2H), 6.18 (dt, J=5.6, 2.1 Hz, 1H), 5.93 (dt, J=5.7, 2.2 Hz, 1H), 5.49 (ddt, J=7.2, 5.3, 2.0 Hz, 1H), 4.73 (t, J=5.3 Hz, 1H), 3.54-3.41 (m, 2H), 2.90 (dqt, J=9.0, 3.5, 1.9 Hz, 1H), 2.64 (dt, J=13.8, 8.8 Hz, 1H), 1.67 (dt, J=13.9, 5.4 Hz, 1H). HRMS (ESI): Calcd for (C₁₂H₁₂N₆O+H)⁺: 257.1151, Found: 257.1127.

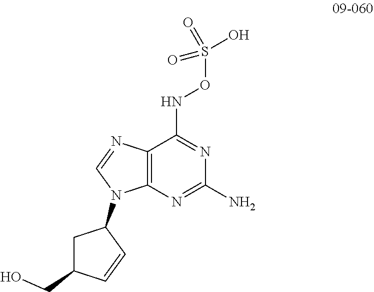 [SMILES: Nc1nc(NOS(=O)(=O)O)c2ncn([C@H]3C=C[C@@H](CO)C3)c2n1]

Prepared using general method B. White solid (7.9 mg, 14%). ¹H NMR (400 MHz, DMSO) δ 11.10 (s, 1H), 8.34 (s, 1H), 7.97 (s, OH), 7.44 (s, 2H), 6.62 (s, 1H), 6.17 (ddt, J=13.7, 5.6, 2.1 Hz, 1H), 5.90 (ddt, J=12.2, 5.6, 2.1 Hz, 1H), 5.51-5.30 (m, 1H), 3.50-3.38 (m, 2H), 2.94-2.85 (m, 1H), 2.70-2.57 (m, 1H), 1.79-1.54 (m, 1H). HRMS (ESI): Caled for (C₁₁H₁₄N₆O₅S+H)⁺: 343.0825, Found: 343.0782.

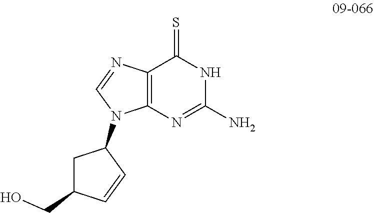 [SMILES: Nc1nc2c(ncn2[C@H]2C=C[C@@H](CO)C2)c(=S)[nH]1]

A 1-dram vial was charged with [(1S,4R)-4-(2-amino-6-chloro-purin-9-yl)cyclopent-2-en-1-yl]methanol (50 mg, 0.19 mmol), DMF (0.2000 mL) and Triethylamine (0.080 mL, 0.56 mmol). Methyl 3-sulfanylpropanoate (0.020 mL, 0.19 mmol) was added via pipette. The mixture was warmed to 80° C. for 12 h. The reaction mixture was acidified with trifluoroacetic acid to pH=2, diluted with 50% acetonitrile-water to a volume of 3.0 mL, and filtered through a PTFE syringe filter. The filtrate was purified by reverse-phase HPLC (Waters XBridge C18 column 5 μm particle size 30×250 mm, 5-95% acetonitrile-water+0.1% trifluoroacetic acid, 40 min, 20 mL/min) to afford the product (11 mg, 22%). HRMS (ESI): Calcd for (C₁₁H₁₄N₅OS+H)⁺: 264.0919, Found: 264.0887.

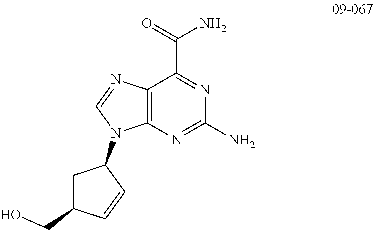 [SMILES: NC(=O)c1nc(N)nc2c1ncn2[C@H]1C=C[C@@H](CO)C1]

A 6.0 M aqueous sodium hydroxide solution (0.50 mL) was added to a suspension of 09-059 (10 mg, 0.039 mmol) in ethanol (0.5 mL). The mixture was heated to 90° C. for 1 h. The reaction mixture was acidified with TFA to pH 3 and then filtered through a 0.45 μm PTFE syring filter. The filtrate was purified by reverse-phase HPLC (Waters XBridge C18 column 5 μm particle size 30×250 mm, 5-95% acetonitrile-water+0.10% trifluoroacetic acid, 40 min, 20 mL/min) to afford the product as a white solid (2.9 mg, 27%). The major byproduct of this reaction was identical to 09-054B, likely formed by the nucleophilic displacement of the cyanide by water. ¹H NMR (400 MHz, DMSO) δ 11.06 (s, 1H), 8.49 (d, J=7.0 Hz, 1H), 7.15 (s, 2H), 6.01 (dt, J=5.7, 2.1 Hz, 1H), 5.85-5.78 (m, 1H), 5.15 (d, J=7.3 Hz, 1H), 2.77 (d, J=6.5 Hz, 1H), 2.50-2.41 (m, 1H), 1.46 (dt, J=13.1, 6.5 Hz, 1H). HRMS (ESI): Caled for (C₁₂H₁₄N₆O₂+H)⁺: 275.1256, Found: 275.1198.

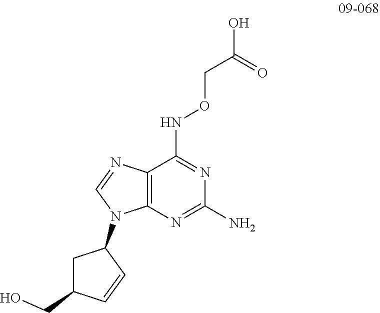 [SMILES: Nc1nc(NOCC(=O)O)c2ncn([C@H]3C=C[C@@H](CO)C3)c2n1]

Prepared using general method B. White solid (9.1 mg, 19%). ¹H NMR (400 MHz, DMSO) δ 11.06 (s, 1H), 8.49 (d, J=7.0 Hz, 1H), 7.15 (s, 2H), 6.01 (dt, J=5.7, 2.1 Hz, 1H), 5.85-5.78 (m, 1H), 5.15 (d, J=7.3 Hz, 1H), 2.77 (d, J=6.5 Hz, 1H), 2.50-2.41 (m, 1H), 1.46 (dt, J=13.1, 6.5 Hz, 1H). HRMS (ESI): Caled for (C₁₃H₁₆N₆O₄+H)⁺: 321.1311, Found: 321.1268.

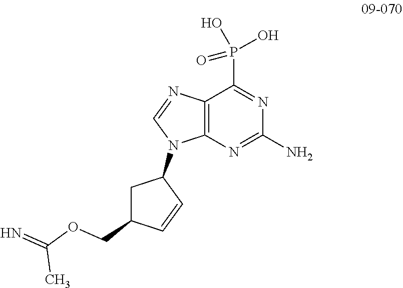 [SMILES: CC(=N)OC[C@@H]1C=C[C@H](n2cnc3c(P(=O)(O)O)nc(N)nc32)C1]

A suspension of [(1S,4R)-4-(2-amino-6-chloro-purin-9-yl)cyclopent-2-en-1-yl]methanol (50 mg, 0.19 mmol) in triisopropyl phosphite (0.5 mL) was heated in a microwave reactor at 180° C. for 30 min. The reaction mixture was concentrated under reduced pressure. The residue was purified by column chromatography (0-10% methanol-dichloromethane, 4-g RediSep(R) Rf column, Teledyne ISCO, Lincoln, Nebr.) to afford a phosphonate intermediate [1S,4R)-4-(2-amino-6-diisopropoxyphosphoryl-purin-9-yl)cyclopent-2-en-1-yl]methanol. The intermediate was dissolved in MeCN (0.25 mL) and trimethylsilyl bromide (0.030 mL, 0.25 mmol) was added dropwise at 23° C. The reaction mixture was stirred at 23° C. for 16 h. The reaction mixture was diluted with water to 2.5 mL, and the solution was filtered through a 0.45 μM PTFE syringe filter. The filtrate was purified by reverse-phase HPLC (Waters XBridge C₁₈ column 5 μm particle size 30×250 mm, 5-95% acetonitrile−water+0.1% trifluoroacetic acid, 40 min, 20 mL/min) to afford the product as a white solid (3.9 mg, 11%). ¹H NMR (400 MHz, DMSO) δ 8.24 (s, 1H), 7.23 (s, 2H), 6.17 (dt, J=5.6, 2.1 Hz, 1H), 6.01 (dt, J=5.6, 2.2 Hz, 1H), 5.52 (d, J=8.6 Hz, 1H), 4.10 (dd, J=6.1, 2.5 Hz, 2H), 3.12 (s, 1H), 2.73 (dt, J=14.1, 8.9 Hz, 1H), 2.01 (s, 3H), 1.70 (dt, J=13.8, 5.9 Hz, 1H). HRMS (ESI): Calcd for (C₁₃H₁₇N₆O₄P+H)⁺: 353.1127, Found: 353.1189.

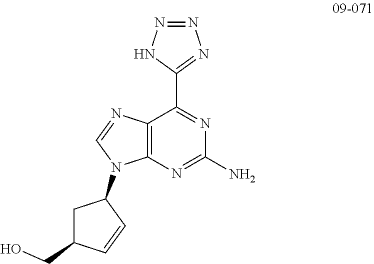 [SMILES: Nc1nc(-c2nnn[nH]2)c2ncn([C@H]3C=C[C@@H](CO)C3)c2n1]

Sodium azide (3.0 mg, 0.047 mmol) was added to a stirred mixture of 09-059 (6.0 mg, 0.023 mmol), ammonium chloride (2.5 mg, 0.047 mmol) and DMF (0.500 mL). The mixture was heated to 90° C. for 18 h. The reaction mixture was diluted with 50% acetonitrile-water to a volume of 3.0 mL, and the solution was filtered through a 0.45 μM PTFE syringe filter. The filtrate was purified by reverse-phase HPLC (Waters XBridge C18 column 5 μm particle size 30×250 mm, 5-95% acetonitrile−water+0.1% trifluoroacetic acid, 40 min, 20 mL/min) to afford the product as a white solid (3.4 mg, 49%). ¹H NMR (400 MHz, DMSO) δ 8.25 (s, 1H), 6.89 (s, 2H), 6.20 (dt, J=5.6, 2.1 Hz, 1H), 5.97 (dt, J=5.6, 2.1 Hz, 1H), 5.63-5.51 (m, 1H), 3.48 (dt, J=7.7, 3.9 Hz, 2H), 2.92 (s, 1H), 2.74-2.62 (m, 1H), 1.71 (dt, J=13.9, 5.4 Hz, 1H). HRMS (ESI): Calcd for (C₁₂H₁₃N₉O+H)⁺: 300.1321, Found: 300.1280.

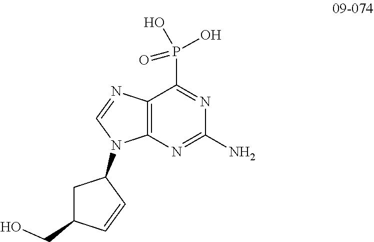 [SMILES: Nc1nc(P(=O)(O)O)c2ncn([C@H]3C=C[C@@H](CO)C3)c2n1]

A suspension of [(1S,4R)-4-(2-amino-6-chloro-purin-9-yl)cyclopent-2-en-1-yl]methanol (50 mg, 0.19 mmol) in triisopropyl phosphite (0.5 mL) was heated in a microwave reactor at 180° C. for 30 min. The reaction mixture was concentrated under reduced pressure. The residue was purified by column chromatography (0-10% methanol-dichloromethane, 4-g RediSep(R) Rf column, Teledyne ISCO, Lincoln, Nebr.) to afford a phosphonate intermediate [1S,4R)-4-(2-amino-6-diisopropoxyphosphoryl-purin-9-yl)cyclopent-2-en-1-yl]methanol. The intermediate was dissolved in dichloromethane (0.25 mL) and trimethylsilyl bromide (0.030 mL, 0.25 mmol) was added dropwise at 23° C. The reaction mixture was stirred at 23° C. for 16 h. The reaction mixture was evaporated under reduced pressure and the residue was diluted with water to 3.0. The solution was filtered through a 0.45 μM PTFE syringe filter. The filtrate was purified by reverse-phase HPLC (Waters XBridge C18 column 5 μm particle size 30×250 mm, 5-95% acetonitrile−water+0.10% trifluoroacetic acid, 40 min, 20 mL/min) to afford the product as a white solid (3.8 mg, 6.5%). ¹H NMR (400 MHz, DMSO) δ 8.36 (s, 1H), 7.41 (s, 2H), 6.23-6.13 (m, 1H), 5.97-5.90 (m, 1H), 5.59-5.46 (m, 1H), 4.49 (t, J=6.1 Hz, 2H), 3.47 (h, J=5.2 Hz, 2H), 2.91 (s, 1H), 2.70-2.60 (m, 1H), 1.70 (dt, J=13.9, 5.3 Hz, 1H). HRMS (ESI): Calcd for (C₁₁H₁₄N₅O₄P+H)⁺: 312.0862, Found: 312.0827.

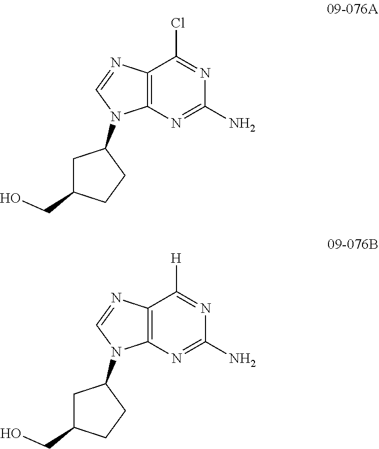 [SMILES: Nc1nc(Cl)c2ncn([C@H]3CC[C@@H](CO)C3)c2n1 ; Nc1ncc2ncn([C@H]3CC[C@@H](CO)C3)c2n1]

Palladium on carbon (10 wt %, 20 mg, 0.019 mmol) was added to a solution of [(1S,4R)-4-(2-amino-6-chloro-purin-9-yl)cyclopent-2-en-1-yl]methanol hydrochloride (50 mg, 0.19 mmol) in methanol (1 mL) at 23° C. The vial was flushed with argon, and then fitted with a rubber septum. The system was quickly evacuated and flushed with hydrogen (balloon). The cycle was repeated three times, then the mixture was stirred under hydrogen atmosphere. After 2 h, the vial was evacuated and flushed with argon. The cycle was repeated three times before removal of the rubber septum. The reaction mixture was filtered through a tightly packed pad of Celite in a glass pipette. The bed was rinsed with 5 mL methanol. The combined filtrate was concentrated in vacuo, and the residue was purified by column chromatography (0-20% methanol-dichloromethane) to afford 09-076A (31 mg, 62%) and 09-076B (10 mg, 23%). 09-076A: ¹H NMR (400 MHz, DMSO) δ 8.26 (s, 1H), 6.87 (s, 2H), 4.77-4.56 (m, 2H), 3.50-3.38 (m, 2H), 2.33-2.06 (m, 3H), 2.05-1.89 (m, 1H), 1.84-1.59 (m, 3H). HRMS (ESI): Caled for (C₁₁H₁₄ClN₅O+H)⁺: 268.0965, Found: 268.0924. 09-076B: ¹H NMR (400 MHz, DMSO) δ 8.56 (s, 1H), 8.18 (s, 1H), 6.46 (s, 3H), 4.70 (p, J=8.3 Hz, 1H), 4.62 (t, J=5.2 Hz, 1H), 3.49-3.39 (m, 2H), 2.29-2.05 (m, 3H), 2.04-1.90 (m, 1H), 1.87-1.56 (m, 4H). HRMS (ESI): Caled for (C₁₁H₁₅N₅O+H)⁺: 234.1355, Found: 234.1316.

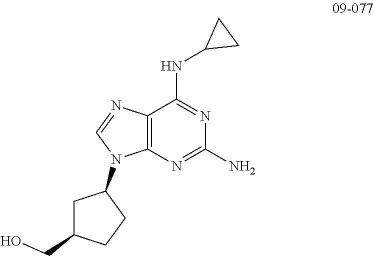 [SMILES: Nc1nc(NC2CC2)c2ncn([C@H]3CC[C@@H](CO)C3)c2n1]

Palladium on carbon (10 wt %, 14 mg, 0.013 mmol) was added to a solution of abacavir hemisulfate (9.0 mg, 0.013 mmol) in 1:1 water:methanol (1.0 mL) at 23° C. The vial was flushed with argon, and then fitted with a rubber septum. The system was quickly evacuated and flushed with hydrogen (balloon). The cycle was repeated three times, then the mixture was stirred under hydrogen atmosphere. After 1 h, the vial was evacuated and flushed with argon. The cycle was repeated three times before removal of the rubber septum. The reaction mixture was filtered through a tightly packed pad of Celite in a glass pipette. The bed was rinsed with 5 mL methanol. The combined filtrate was concentrated in vacuo to afford the product (6.0 mg, 67%). ¹H NMR (400 MHz, DMSO) δ 7.87 (s, 1H), 6.54 (s, 1H), 4.68-4.55 (m, 2H), 3.42 (s, 2H), 3.00 (s, 1H), 2.29-2.01 (m, 2H), 2.01-1.85 (m, 1H), 1.76 (dt, J=13.1, 7.9 Hz, 1H), 1.64 (dt, J=12.0, 9.3 Hz, 2H), 0.76-0.54 (m, 4H). HRMS (ESI): Calcd for (C₁₄H₂₀N₆O+H)⁺: 289.1777, Found: 289.1721.

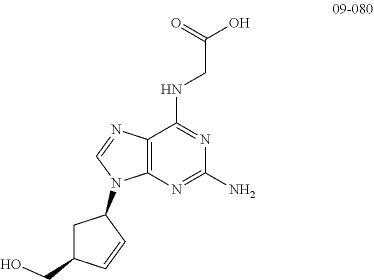 [SMILES: Nc1nc(NCC(=O)O)c2ncn([C@H]3C=C[C@@H](CO)C3)c2n1]

Prepared using general method A. White solid (41.2 mg, 72%). HRMS (ESI): Caled for (C₁₃H₁₇N₆O₃+H)⁺: 305.1362, Found: 305.1350.

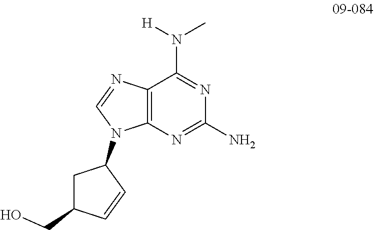 [SMILES: CNc1nc(N)nc2c1ncn2[C@H]1C=C[C@@H](CO)C1]

Prepared using general method A with 20 mg starting material. White solid (17.5 mg, 89%). ¹H NMR (400 MHz, DMSO) δ 7.59 (s, 1H), 7.16 (s, 1H), 6.11 (dt, J=5.6, 2.1 Hz, 1H), 5.87 (dt, J=5.7, 2.2 Hz, 1H), 5.82 (s, 2H), 5.43-5.34 (m, 1H), 4.75 (t, J=5.3 Hz, 1H), 3.45 (t, J=5.5 Hz, 2H), 3.33 (s, 4H), 2.94-2.80 (m, 5H), 2.60 (dt, J=13.7, 8.7 Hz, 1H). HRMS (ESI): Caled for (C₁₂H₁₇N₆O+H)⁺: 261.1464, Found: 261.1461.

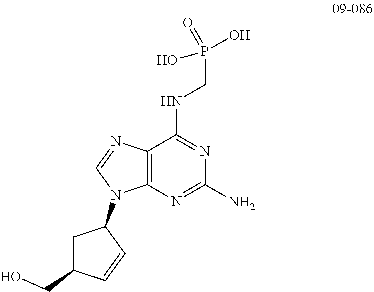 [SMILES: Nc1nc(NCP(=O)(O)O)c2ncn([C@H]3C=C[C@@H](CO)C3)c2n1]

Prepared using general method A. White solid (9.6 mg, 15%). ¹H NMR (400 MHz, DMSO) δ 7.92 (s, 1H), 6.67 (s, 2H), 6.20-6.13 (m, 1H), 5.90 (dt, J=5.6, 2.2 Hz, 1H), 5.44-5.38 (m, 1H), 3.70 (s, 4H), 3.46 (dd, J=5.7, 1.6 Hz, 2H), 2.95-2.84 (m, 1H), 2.70-2.58 (m, 1H), 1.62 (dt, J=13.7, 5.5 Hz, 1H). HRMS (ESI): Caled for (C₁₂H₁₈N₆O₄P+H)⁺: 341.1127, Found: 341.1093.

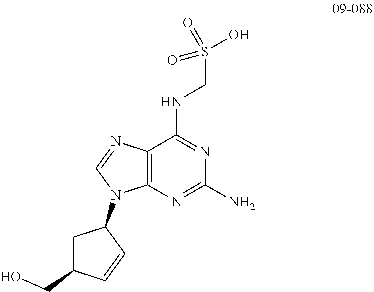 [SMILES: Nc1nc(NCS(=O)(=O)O)c2ncn([C@H]3C=C[C@@H](CO)C3)c2n1]

Prepared using general method A. White solid (6.1 mg, 10%). HRMS (ESI): Caled for (C₁₂H₁₇N₆O₄S+H)⁺: 341.1032, Found: 341.0980.

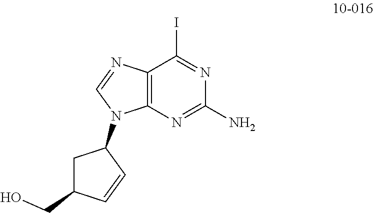 [SMILES: Nc1nc(I)c2ncn([C@H]3C=C[C@@H](CO)C3)c2n1]

Aqueous hydriodic acid (57%) (0.94 mL, 8.3 mmol) was added directly to [(1S,4R)-4-(2-amino-6-chloro-purin-9-yl)cyclopent-2-en-1-yl]methanol hydrochloride (50 mg, 0.17 mmol) solid at 23° C. and the resulting mixture was stirred at 23° C. while the reaction progress was monitored by LC-MS. After 1 h, the reaction mixture was neutralized with saturated aqueous bicarbonate solution (5 mL) followed by solid sodium bicarbonate until gas evolution subsided. The reaction mixture was partitioned between saturated aqueous sodium bicarbonate solution (5 mL) and ethyl acetate (5 mL). Lots of black precipitate formed. The mixture was filtered through a sintered plastic funnel to remove the precipitates (which were rinsed with ethyl acetate but did not dissolve). The layers were separated, and the aqueous layer was extracted with ethyl acetate (2×5 mL). The combined organic layers were dried over sodium sulfate. The dried solution was filtered, and the filtrate was concentrated to afford a yellow solid (60 mg, 100%). ¹H NMR (400 MHz, DMSO) δ 8.00 (s, 1H), 6.83 (s, 2H), 6.15 (dt, J=5.6, 2.1 Hz, 1H), 5.91 (dt, J=5.6, 2.2 Hz, 1H), 5.42 (ddt, J=7.3, 5.4, 2.0 Hz, 1H), 4.73 (t, J=5.3 Hz, 1H), 3.45 (t, J=5.1 Hz, 2H), 2.88 (qd, J=7.7, 6.3, 3.8 Hz, 1H), 2.62 (dt, J=13.8, 8.7 Hz, 1H), 1.63 (dt, J=13.8, 5.5 Hz, 1H). HRMS (ESI): Caled for (C₁₁H₁₂IN₅O+H)⁺: 358.0165, Found: 358.0175.

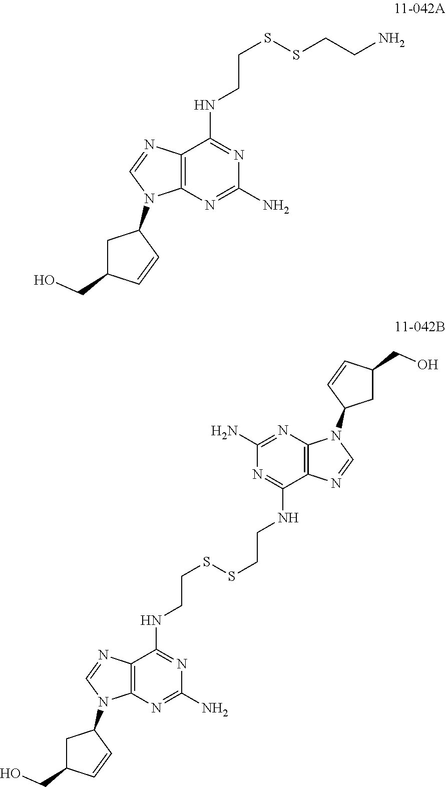 [SMILES: NCCSSCCNc1nc(N)nc2c1ncn2[C@H]1C=C[C@@H](CO)C1 ; Nc1nc(NCCSSCCNc2nc(N)nc3c2ncn3[C@H]2C=C[C@@H](CO)C2)c2ncn([C@H]3C=C[C@@H](CO)C3)c2n1]

A one-dram vial was charged with cystamine dihydrochloride (212 mg, 0.941 mmol), [(1S,4R)-4-(2-amino-6-chloro-purin-9-yl)cyclopent-2-en-1-yl]methanol (50 mg, 0.19 mmol) and a magnetic stir bar. Isopropanol (0.38 mL) and N,N-diisopropylethylamine (0.49 mL, 2.8 mmol) were added sequentially via syringe. The mixture was warmed to 60° C. After 3 h, the reaction mixture was concentrated to dryness and the residue was diluted with 50% acetonitrile-water to a volume of 4.3 mL, and the solution was filtered through a 0.45 μM PTFE syringe filter. The filtrate was purified by reverse-phase HPLC (Waters XBridge C18 column 5 μm particle size 30×250 mm, 5-95% acetonitrile-water+0.1% formic acid, 40 min, 20 mL/min) to afford 11-042A (23.7 mg, 21%) and 11-042B (10.6 mg, 15%). 11-042A: ¹H NMR (400 MHz, MeOD) δ 7.77 (s, 1H), 6.20 (dt, J=5.6, 2.1 Hz, 1H), 5.92 (dt, J=5.6, 2.2 Hz, 1H), 5.54 (ddq, J=9.7, 5.8, 2.1 Hz, 1H), 3.90 (t, J=6.7 Hz, 2H), 3.67 (dd, J=10.9, 5.3 Hz, 1H), 3.61 (dd, J=10.9, 5.1 Hz, 1H), 3.30 (t, J=6.8 Hz, 2H), 3.07 (t, J=6.6 Hz, 2H), 2.99 (t, J=6.7 Hz, 2H), 2.80 (dt, J=13.9, 8.8 Hz, 1H), 1.72 (dt, J=13.9, 5.8 Hz, 1H). HRMS (ESI): Calcd for (C₁₅H₂₃N₇OS₂+H)⁺: 382.1484, Found: 382.1470. 11-042B: ¹H NMR (400 MHz, MeOD) δ 7.76 (s, 2H), 6.19 (dtd, J=7.1, 4.6, 2.5 Hz, 2H), 5.91 (dt, J=5.7, 2.2 Hz, 2H), 5.52 (ddq, J=9.7, 5.8, 2.1 Hz, 2H), 3.87 (s, 4H), 3.76-3.54 (m, 4H), 3.03 (t, J=6.7 Hz, 6H), 2.88-2.72 (m, 2H), 1.82-1.66 (m, 2H). HRMS (ESI): Calcd for (C₂₆H₃₄N₁₂O₂S₂+H)⁺: 611.2447, Found: 611.2488.

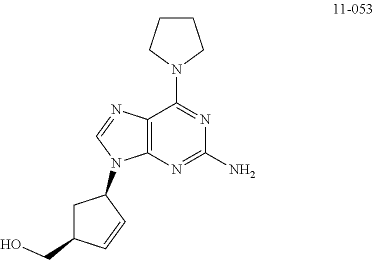 [SMILES: Nc1nc(N2CCCC2)c2ncn([C@H]3C=C[C@@H](CO)C3)c2n1]

Prepared using general method A. White solid (44 mg, 89%). ¹H NMR (400 MHz, DMSO) δ 7.60 (s, 1H), 6.12 (dt, J=5.6, 2.1 Hz, 1H), 5.87 (dt, J=5.6, 2.1 Hz, 1H), 5.77 (s, 2H), 5.41 (ddt, J=9.0, 5.7, 2.0 Hz, 1H), 4.75 (s, 1H), 3.93 (br s, 2H), 3.61 (br s, 2H), 3.48-3.36 (m, 2H), 2.87 (ddt, J=8.3, 4.2, 2.2 Hz, 1H), 2.60 (dt, J=13.6, 8.7 Hz, 1H), 1.56 (dt, J=13.7, 5.7 Hz, 1H). HRMS (ESI): Caled for (C₁₅H₂₀N₆O+H)⁺: 301.1777, Found: 301.1786.

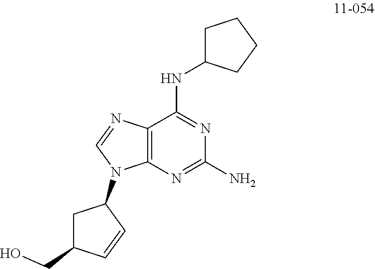 [SMILES: Nc1nc(NC2CCCC2)c2ncn([C@H]3C=C[C@@H](CO)C3)c2n1]

Prepared using general method A. White solid (31 mg, 60%). ¹H NMR (400 MHz, DMSO) δ 7.60 (s, 1H), 7.01 (s, 1H), 6.11 (dt, J=5.6, 2.1 Hz, 1H), 5.87 (dt, J=5.6, 2.2 Hz, 1H), 5.79-5.74 (m, 2H), 5.39 (ddt, J=9.0, 5.8, 2.0 Hz, 1H), 4.75 (s, 1H), 4.52 (s, 1H), 3.48-3.42 (m, 2H), 2.87 (ddt, J=6.2, 4.4, 2.5 Hz, 1H), 2.60 (dt, J=13.6, 8.7 Hz, 1H), 1.93-1.87 (m, 2H), 1.74-1.64 (m, 2H), 1.64-1.46 (m, 5H). HRMS (ESI): Caled for (C₁₆H₂₂N₆O+H)⁺: 315.1933, Found: 315.1933.

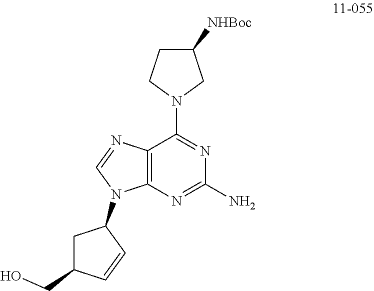 [SMILES: CC(C)(C)OC(=O)N[C@@H]1CCN(c2nc(N)nc3c2ncn3[C@H]2C=C[C@@H](CO)C2)C1]

Prepared using general method A. White solid (55 mg, 80%). HRMS (ESI): Caled for (C₂₀H₂₉N₇O₃+H)⁺: 416.2410, Found: 416.2423.

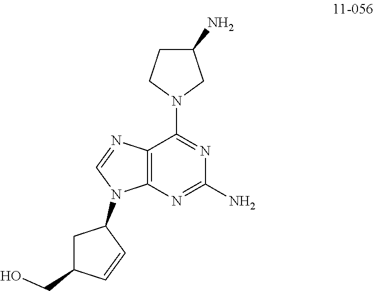 [SMILES: Nc1nc(N2CC[C@@H](N)C2)c2ncn([C@H]3C=C[C@@H](CO)C3)c2n1]

11-055 (55 mg, 0.13 mmol) was dissolved in trifluoroacetic Acid (0.5000 mL):DCM (0.5000 mL) at 23° C. and the resulting solution was allowed to stand at 23° C. for 1 h. The solution was concentrated to dryness to afford the product as a white solid (57 mg, 100%). ¹H NMR (400 MHz, MeOD) δ 7.95 (s, 1H), 6.30-6.21 (m, 1H), 5.99-5.86 (m, 1H), 5.65-5.57 (m, 1H), 4.17-4.11 (m, 2H), 3.82 (dd, J=10.8, 4.1 Hz, 1H), 3.73-3.57 (m, 1H), 3.12-3.08 (m, 1H), 3.00-2.74 (m, 1H), 2.60-2.50 (m, 2H), 2.32-2.17 (m, 2H), 1.91-1.71 (m, 2H). HRMS (ESI): Calcd for (C₁₅H₂₁N₇O+H)⁺: 316.1886, Found: 316.1899.

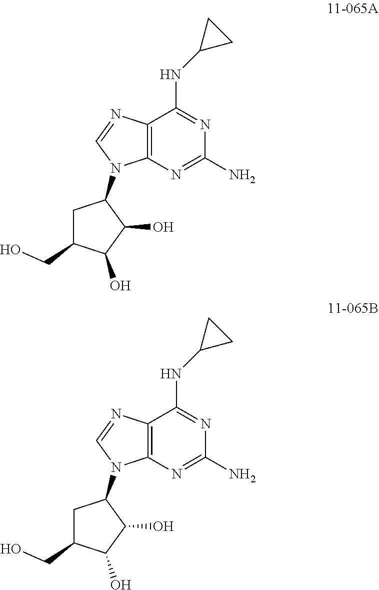 [SMILES: Nc1nc(NC2CC2)c2ncn([C@@H]3C[C@H](CO)[C@H](O)[C@@H]3O)c2n1 ; Nc1nc(NC2CC2)c2ncn([C@@H]3C[C@H](CO)[C@@H](O)[C@H]3O)c2n1]

N-methylmorpholine-N-oxide (32 mg, 0.27 mmol) and Potassium osmate (9.1 mg, 0.027 mmol) were added sequentially to a stirred solution of tert-butyl [(1S,4R)-4-(2-amino-6-chloro-purin-9-yl)cyclopent-2-en-1-yl]methyl carbonate (50 mg, 0.14 mmol) in 4:1 acetone:water (0.20 mL). The reaction mixture was stirred at 23° C. and the reaction progress was monitored by LC-MS. After 24 h, the reaction mixture was partitioned between saturated aqueous sodium bicarbonate solution (5 mL) and dichloromethane (5 mL). The layers were separated, and the aqueous layer was extracted with dichloromethane (2×5 mL). The combined organic layers were dried over sodium sulfate. The dried solution was filtered, and the filtrate was concentrated. The residue was purified by column chromatography (20-100% ethyl acetate-hexanes, 4-g RediSep(R) Rf column, Teledyne ISCO, Lincoln, Nebr.) to afford the product as a yellow powder (a mixture of diastereomers, 47 mg, 86%).

To this residue was added isopropanol (0.50 mL), cyclopropylamine (0.08 mL, 1.2 mmol) and a magnetic stir bar. The mixture was heated at 90° C. for 30 min. The reaction mixture was concentrated to dryness. The crude product was dissolved in 1:1 trifluoroacetic acid:dichloromethane (1 mL) and the resulting solution was allowed to stand at 23° C. for 30 min. The reaction mixture was concentrated under reduced pressure. The residue was diluted with water to a volume of 4.3 mL, and the solution was filtered through a 0.45 μM PTFE syringe filter. The filtrate was purified by reverse-phase HPLC (Waters XBridge C18 column 5 μm particle size 30×250 mm, 1-30% acetonitrile−water+0.1% formic acid, 40 min, 20 mL/min) to afford 11-065B (5.5 mg, 15%) and 11-065A (11.9 mg, 32%). 11-065A: ¹H NMR (400 MHz, MeOD) δ 7.83 (s, 1H), 4.68 (dt, J=10.1, 8.6 Hz, 1H), 4.43 (dd, J=8.8, 5.4 Hz, 1H), 4.03 (dd, J=5.5, 3.1 Hz, 1H), 3.71 (dd, J=5.7, 2.2 Hz, 2H), 2.96-2.89 (m, 1H), 2.45 (dt, J=13.1, 8.7 Hz, 1H), 2.28-2.19 (m, 1H), 1.96-1.84 (m, 1H), 0.91-0.80 (m, 2H), 0.67-0.59 (m, 2H). HRMS (ESI): Calcd for (C₁₄H₂₀N₆O₃+H)⁺: 321.1675, Found: 321.1671. 11-065B: ¹H NMR (400 MHz, MeOD) δ 8.07 (s, 1H), 4.96 (q, J=8.7 Hz, 1H), 4.31-4.21 (m, 2H), 3.87 (dd, J=10.8, 6.8 Hz, 1H), 3.74 (dd, J=10.8, 5.9 Hz, 1H), 2.94 (s, 1H), 2.38 (ddd, J=12.7, 8.8, 7.4 Hz, 1H), 2.29-2.13 (m, 1H), 2.12-1.99 (m, 1H), 1.05-0.93 (m, 2H), 0.87-0.74 (m, 2H). HRMS (ESI): Calcd for (C₁₄H₂₀N₆O₃+H)⁺: 321.1675, Found: 321.1671.

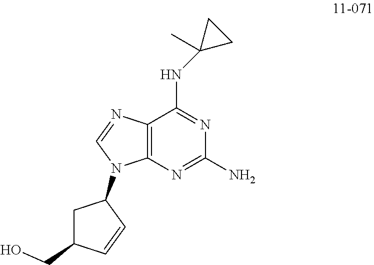 [SMILES: CC1(Nc2nc(N)nc3c2ncn3[C@H]2C=C[C@@H](CO)C2)CC1]

Prepared using general method A. White solid (51 mg, 100%). ¹H NMR (400 MHz, CDCl₃) δ 7.49 (s, 1H), 6.13 (dt, J=5.6, 2.2 Hz, 1H), 6.03 (s, 1H), 5.77 (dt, J=5.6, 2.3 Hz, 1H), 5.48-5.38 (m, 1H), 4.87 (s, 2H), 3.95 (dd, J=10.9, 3.7 Hz, 1H), 3.20-3.10 (m, 0H), 2.81 (dt, J=14.7, 9.7 Hz, 1H), 2.25 (dt, J=14.6, 5.6 Hz, 1H), 1.52 (s, 3H), 0.91-0.80 (m, 2H), 0.78-0.70 (m, 2H). HRMS (ESI): Caled for (C₁₅H₂₀N₆O+H)⁺: 301.1777, Found: 301.1786.

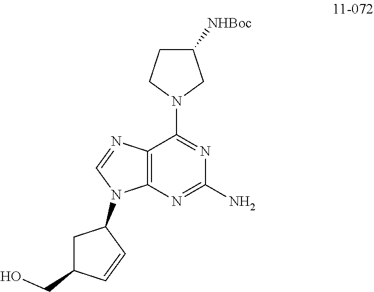 [SMILES: CC(C)(C)OC(=O)N[C@H]1CCN(c2nc(N)nc3c2ncn3[C@H]2C=C[C@@H](CO)C2)C1]

Prepared using general method A. White solid (72 mg, 100%). ¹H NMR (400 MHz, CDCl₃) δ 7.51 (s, 1H), 6.13 (dt, J=5.6, 2.2 Hz, 1H), 5.77 (dt, J=5.7, 2.3 Hz, 1H), 5.44 (ddd, J=9.9, 4.9, 2.3 Hz, 1H), 4.71 (s, 2H), 4.36 (s, 1H), 3.95 (dd, J=10.8, 3.6 Hz, 1H), 3.83 (dd, J=10.8, 2.9 Hz, 1H), 3.15 (s, 1H), 2.80 (dt, J=14.7, 9.8 Hz, 1H), 2.34-2.19 (m, 2H), 1.98 (s, 1H), 1.47 (s, 9H). HRMS (ESI): Caled for (C₂₀H₂₉N₇O₃+H)⁺: 416.2410, Found: 416.2423.

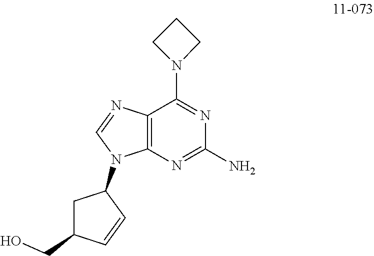 [SMILES: Nc1nc(N2CCC2)c2ncn([C@H]3C=C[C@@H](CO)C3)c2n1]

Prepared using general method A. White solid (25 mg, 53%). ¹H NMR (400 MHz, DMSO) δ 7.60 (s, 1H), 6.12 (dt, J=5.6, 2.1 Hz, 1H), 5.92-5.78 (m, 3H), 5.43-5.34 (m, 1H), 4.74 (t, J=5.3 Hz, 1H), 4.25 (s, 5H), 3.44 (t, J=5.6 Hz, 3H), 2.92-2.82 (m, 2H), 2.59 (dt, J=13.7, 8.7 Hz, 1H), 2.43-2.33 (m, 2H), 1.55 (dt, J=13.7, 5.7 Hz, 1H). HRMS (ESI): Caled for (C₁₄H₁₈N₆O+H)⁺: 287.1620, Found: 287.1635.

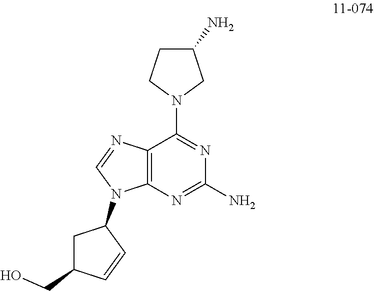 [SMILES: Nc1nc(N2CC[C@H](N)C2)c2ncn([C@H]3C=C[C@@H](CO)C3)c2n1]

11-072 (62 mg, 0.15 mmol) was dissolved in 1:1 dichloromethane:trifluoroacetic acid (1.0 mL), and the resulting solution was allowed to stand at 23° C. In 30 min, LC-MS analysis showed full conversion to the deprotected material. The reaction mixture was concentrated under reduced pressure to afford the product as white solid, which was further triturated with ether to remove residual trifluoroacetic acid. White solid (64 mg, 100%). ¹H NMR (400 MHz, MeOD) δ 7.93 (d, J=2.2 Hz, 1H), 6.25 (dd, J=4.9, 2.9 Hz, 1H), 5.90 (dt, J=5.6, 2.2 Hz, 1H), 5.61 (s, 1H), 4.13 (s, 2H), 3.88-3.76 (m, 1H), 3.67 (dd, J=10.8, 4.3 Hz, 1H), 3.16-3.03 (m, 2H), 2.86 (dt, J=14.3, 9.1 Hz, 1H), 2.54 (dd, J=14.4, 7.0 Hz, 1H), 2.33-2.21 (m, 1H), 1.82 (dt, J=14.2, 5.6 Hz, 1H). HRMS (ESI): Calcd for (C₁₅H₂₁N₇O+H)⁺: 316.1886, Found: 316.1898.

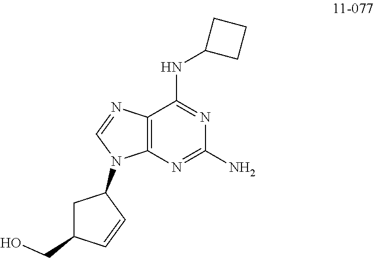 [SMILES: Nc1nc(NC2CCC2)c2ncn([C@H]3C=C[C@@H](CO)C3)c2n1]

Prepared using general method A. White solid (47 mg, 95%). ¹H NMR (400 MHz, CDCl₃) δ 7.51 (s, 1H), 6.13 (dt, J=5.6, 2.2 Hz, 1H), 5.87 (s, 1H), 5.77 (dt, J=5.7, 2.3 Hz, 1H), 5.48-5.38 (m, 1H), 4.81 (s, 2H), 3.95 (dd, J=10.9, 3.7 Hz, 1H), 3.83 (dd, J=10.8, 2.9 Hz, 1H), 3.16 (d, J=7.7 Hz, 1H), 2.81 (dt, J=14.6, 9.7 Hz, 1H), 2.45 (d, J=8.4 Hz, 2H), 2.24 (dt, J=14.7, 5.6 Hz, 1H), 2.05-1.91 (m, 2H), 1.91-1.67 (m, 2H). HRMS (ESI): Caled for (C₁₅H₂₀N₆O+H)⁺: 301.1777, Found: 301.1786.

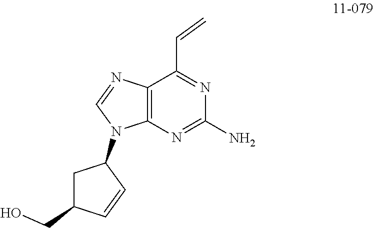 [SMILES: C=Cc1nc(N)nc2c1ncn2[C@H]1C=C[C@@H](CO)C1]

An oven-dried one-dram vial was charged with tert-butyl [(1S,4R)-4-[2-[bis(tert-butoxycarbonyl)amino]-6-chloro-purin-9-yl]cyclopent-2-en-1-yl]methyl carbonate (30 mg, 0.053 mmol), DCE (0.27 mL) and a magnetic stir bar. Vinyltributyltin (0.030 mL, 0.1060 mmol) was added via pipette. Argon was bubbled through the reaction solution for 5 min, then tetrakis(triphenylphosphine)palladium(0) (6.1 mg, 0.0053 mmol) was added in one portion. The reaction mixture was heat to 80° C. and the reaction progress was monitored by LC-MS. In 4 h, the reaction mixture was cooled to 23° C. and directly loaded onto a silica gel cartridge (˜2 g). Purification by column chromatography (20-50% ethyl acetate-hexanes, 4-g RediSep Rf Column, Teledyne ISCO, Lincoln, Nebr.) afforded the product as a colorless oil. This intermediate was dissolved in 1:1 dichloromethane:trifluoroacetic acid (1.0 mL). The resulting solution was allowed to stand at 23° C. for 30 min, then was concentrated under reduced pressure to afford the product as a yellow solid (20 mg, 99%). HRMS (ESI): Calcd for (C₁₃H₁₅N₅O+H)⁺: 258.1355, Found: 258.1378.

All compounds synthesized using General Procedures A and B were characterized and confirmed using, for example, ¹H NMR and/or Mass Spectrometry.

### SEQUENCE LISTING

TABLE 5
List of Peptide Sequences (peptide antigens)

| Peptide Name | Sequence |
|---|---|
| G12V 8-16 | VVGAVGVGK (SEQ ID NO: 1) |
| G12V 7-16 | VVVGAVGVGK (SEQ ID NO: 2) |
| G12D 8-16 | VVGADGVGK (SEQ ID NO: 3) |
| G12D 7-16 | VVVGADGVGK (SEQ ID NO: 4) |
| G12V 8-16 W16 | VVGAVGVGW (SEQ ID NO: 5) |
| WT 8-16 | VVGAGGVGK (SEQ ID NO: 6) |
| WT7-16 | VVVGAGGVGK (SEQ ID NO: 7) |
| V7G16 | VVVGAVGVGG (SEQ ID NO: 8) |
| V7A16 | VVVGAVGVGA (SEQ ID NO: 9) |
| V7V16 | VVVGAVGVGV (SEQ ID NO: 10) |
| V7I16 | VVVGAVGVGI (SEQ ID NO: 11) |
| V7K16 | VVVGAVGVGK (SEQ ID NO: 12) |
| (K-Ras^{G12V} 7-16) | |
| V7W16 | VVVGAVGVGW (SEQ ID NO: 13) |
| (Postive Control) | |
| Pep V | HSITYLLPV (SEQ ID NO: 14) |
| (Ostrov et. Al) | |
| LF9 | LSSPVTKSF (SEQ ID NO: 15) |
| P53 R175H 168-176 | HMTEVVRHC (SEQ ID NO: 16) |
| P53 WT 168-176 | HMTEVVRRC (SEQ ID NO: 17) |
| P53 WT 168-176W | HMTEVVRRW (SEQ ID NO: 18) |
| P53 R175H 168-176W | HMTEVVRHW (SEQ ID NO: 19) |
| G12C 7-16 | VVVGACGVGK (SEQ ID NO: 20) |
| QAFWIDLFETIG | QAFWIDLFETIG (SEQ ID NO: 21) |
| Peptide | |
| | ARAAAAAAA (SEQ ID NO: 22) |

### REFERENCES

1. Fritsch, E. F., et al. (2018) United States Patent Publication No. US 2018/0153975 A1.
2. Fritsch, E. F., et al. (2016) WIPO International Publication No. WO 2016/187508 A2.
3. Ostrov, D. A., et al. Drug hypersensitivity caused by alteration of the MHC-presented self-peptide repertoire. PNAS. 2012, 109 (25), 9959-9964.
4. Yasuda, H., et al. EGFR exon 20 insertion mutations in non-small-cell lung cancer: preclinical data and clinical implications. Lancet Oncol. 2012, 13, e23-31.

What is claimed is:

1. A method of identifying a candidate compound that stabilizes binding of an MHC protein to a peptide antigen, the method comprising:
a. contacting an MHC protein with a peptide antigen and a candidate compound thereby forming an MHC-peptide-compound complex;
b. detecting an increased stability of said MHC-peptide-compound complex relative to the stability of an MHC-peptide complex, wherein said MHC-peptide complex comprises said MHC protein and said peptide antigen in the absence of said candidate compound, thereby identifying said candidate compound as stabilizing binding of said MHC protein to said peptide antigen.

2. The method of claim 1, wherein said MHC protein binds to said peptide antigen with a Kd of greater than 1 micromolar.

3. The method of claim 1, wherein the MHC protein in step a is unfolded.

4. The method of claim 1, wherein the MHC protein is an MHC class I protein or an MHC class II protein.

5. The method of claim 1, wherein the MHC protein is the MHC class I heavy chain protein.

6. The method of claim 1, wherein the MHC protein is HLA-B*57:01.

7. A method of treating cancer in a subject in need thereof, the method comprising:
(a) detecting an MHC allele of an MHC protein of said subject;
(b) detecting a driver oncogene mutation in said subject;
(c) administering an effective amount of a MHC-peptide antigen stabilizing compound.

8. The method of claim 7, wherein said MHC-peptide antigen stabilizing compound was identified by a method comprising:
(i) contacting said MHC protein in vitro with a peptide cancer antigen and said MHC-peptide antigen stabilizing compound thereby forming an MHC-peptide-compound complex;
(ii) detecting an increased stability of said MHC-peptide-compound complex relative to the stability of an MHC-peptide complex, wherein said MHC-peptide complex comprises said MHC protein and said peptide cancer antigen in the absence of said MHC-peptide antigen stabilizing compound.

9. The method of claim 7, wherein said MHC-peptide antigen stabilizing compound has a molecular weight of less than 750 g/mol.

10. A method of identifying a modified peptide-MHC protein allele binding pair, the method comprising:
a. contacting a plurality of different modified peptides with a plurality of different MHC protein alleles;
b. detecting or computationally predicting binding of a first modified peptide to a first MHC protein allele, thereby identifying a modified peptide-MHC protein allele binding pair.

11. The method of claim 10, wherein the first modified peptide is modified with a tryptophan.

12. The method of claim 10, wherein the plurality of different modified peptides are modified with a tryptophan on the last residue.

13. The method of claim 10, wherein the plurality of different modified peptides are derived from a driver oncogene protein.

14. The method of claim 10, wherein the plurality of different modified peptides are derived from a K-Ras protein.

15. The method of claim 10, wherein the plurality of different modified peptides are derived from a mutant K-Ras protein.

16. The method of claim 15, wherein the mutant K-Ras protein is KRAS p.G12V.

17. The method of claim 1, wherein the MHC-peptide antigen stabilizing compound has the formula: 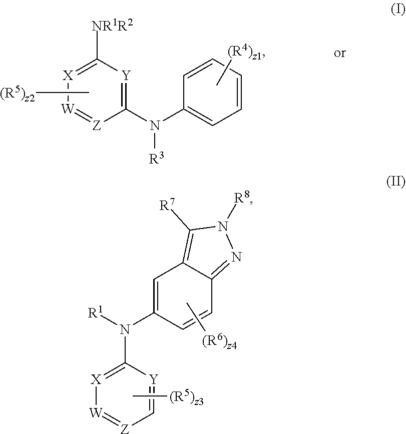
thereof;
wherein,
W, X, Y and Z are each independently C or N;
R¹ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂,
NO₂, —SH, —SOₙ₁R^{1A}, —SOᵥ₁NR^{1A}R^{1B}, —POₘ₁R^{1A}, —POᵣ₁NR^{1A}R^{1B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R² is hydrogen,
halogen, —CCl₃, —CBr₃, —CF₃, —CI₃, —CH₂Cl, —CH₂Br, —CH₂F, —CH₂I,
—CHCl₂, —CHBr₂, —CHF₂, —CHI₂, —CN, —OH, —NH₂, —COOH, —CONH₂, —NO₂, —S H,
—SOₙ₂R^{2A}, —SOᵥ₂NR^{2A}R^{2B}, —POₘ₂R^{2A}, —POᵣ₂NR^{2A}R^{2B}, —NHNH₂, —ONH₂, —NHC(O)NHNH₂, —NHC(O)NH₂, —NHSO₂H, —NHC(O)H, —NHC(O)OH, —NHOH,
OCCl₃, —OCF₃, —OCBr₃, —OCI₃,—OCHCl₂, —OCHBr₂, —OCHI₂, —OCHF₂, —N₃, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR, —NH₂, —COOH, —CONH₂,
—NO₂, —SH, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁴ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{4A}, —NR^{4A}R^{4B}, —COOR^{4A},
—CONR^{4A}R^{4B}, —NO₂, —SR^{4A}, —SOₙ₄R^{4A}, —SOᵥ₄NR^{4A}R^{4B}, —PO(OH)₂, —POₘ₄R^{4A},
POᵣ₄NR^{4A}R^{4B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁵ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{5A}, —NR^{5A}R^{5B}, —COOH,
CONH₂, —NO₂, —SH, —SOₙ₅R^{5A}, —SOᵥ₅NR^{5A}R^{5B}, —PO(OH)₂, —POₘ₅R^{5A},
POᵣ₅NR^{5A}R^{5B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁶ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{6A}, —NH₂, —COOH, —CONH₂,
NO₂, —SH, —SOₙ₆R^{6A}, —SOᵥ₆NR^{6A}R^{6B}, —PO(OH)₂, —POₘ₆R^{6A}, —POᵣ₆NR^{6A}R^{6B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁷ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OR^{7A}, —NH₂, —COOH, —CONH₂,
NO₂, —SH, —SOₙ₇R^{7A}, —SOᵥ₇NR^{7A}R^{7B}, —PO(OH)₂, —POₘ₇R^{7A}, —POᵣ₇NR^{7A}R^{7B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
R⁸ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂,
NO₂, —SH, —SOₙ₈R^{5A}, —SOᵥ₈NR^{8A}R^{8B}, —PO(OH)₂, —POₘ₈R^{8A}, —POᵣ₈NR^{8A}R^{8B}, substituted or unsubstituted alkyl, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
each R^{1A}, R^{1B}, R^{2A}, R^{2B}, R^{4A}, R^{4B}, R^{5A}, R^{5B}, R^{6A}, R^{6B}, R^{7A}, R^{7B}, R^{8A}, and R^{8B} is independently hydrogen, —CX₃, —CHX₂, —CH₂X, —CN, —OH,
—NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl; R^{5A} and R^{5B} substituents bonded to the same nitrogen atom may optionally be joined to form a substituted or unsubstituted heterocycloalkyl or substituted or unsubstituted heteroaryl; R^{6A} and R^{6B} substituents bonded to the same nitrogen atom may optionally be joined to form a substituted or unsubstituted heterocycloalkyl or substituted or unsubstituted heteroaryl; R^{7A} and R^{7B} substituents bonded to the same nitrogen atom may optionally be joined to form a substituted or unsubstituted heterocycloalkyl or substituted or unsubstituted heteroaryl; R^{8A} and R^{8B} substituents bonded to the same nitrogen atom may optionally be joined to form a substituted or unsubstituted heterocycloalkyl or substituted or unsubstituted heteroaryl;
X is independently —Cl, —Br, —I or —F;
each n1, n2, n4, n5, n6, n7, and n8 is independently an integer from 0 to 4;
each v1, v2, v4, v5, v6, v7, and v8 is independently 1 or 2;
each m1, m2, m4, m5, m6, m7, and m8 is independently an integer from 0 to 3;
each r1, r2, r4, r5, r6, r7, and r8 is independently 1 or 2;
each z1 and z3 is independently an integer from 0 to 5;
z2 is an integer from 0 to 4; and
z4 is an integer from 0 to 3.

18. The method of claim 17, wherein R¹ is hydrogen or unsubstituted alkyl;
R³ is hydrogen or unsubstituted alkyl;
R² is hydrogen, substituted or unsubstituted alkyl, substituted or unsubstituted aryl, substituted or unsubstituted heteroaryl, or substituted or unsubstituted cycloalkyl;
R⁵ is hydrogen, oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NR^{5A}R^{5B}, —COOH, —CONH₂,
NO₂, —SH, substituted or unsubstituted heteroalkyl, or substituted or unsubstituted alkyl;
R⁴ is hydrogen, substituted or unsubstituted alkyl or —SO₂NR^{4A}R^{4B};
R⁶ is hydrogen, halogen, or substituted or unsubstituted alkyl;
R⁷ is hydrogen or substituted or unsubstituted alkyl;
R⁸ is hydrogen or substituted or unsubstituted alkyl; and
each R^{4A}, R^{4B}, R^{5A} and R^{5B} is independently hydrogen or substituted or unsubstituted alkyl.

19. The method of claim 17, wherein R¹ is hydrogen or methyl;
R³ is hydrogen or methyl;
R² is methyl, unsubstituted cycloalkyl, unsubstituted aryl or substituted heteroaryl;
R⁵ is hydrogen, oxo, methyl, halogen, unsubstituted heteroalkyl or —NR^{5A}R^{5B};
R⁴ is hydrogen, methyl or —SO₂NR^{4A}R^{4B};
R⁶ is hydrogen or methyl;
R⁷ is hydrogen or methyl;
R⁸ is hydrogen or methyl; and
each R^{4A}, R^{4B}, R^{5A} and R^{5B} is independently hydrogen or methyl.

20. The method of claim 17, wherein R¹ is hydrogen or methyl;
R³ is hydrogen or methyl;
R² is methyl, cyclopropyl, phenyl, or substituted 2H-indazole;
R⁵ is hydrogen, oxo, halogen, ethoxy or —NR^{5A}R^{5B};
R⁴ is hydrogen, methyl or —SO₂NR^{4A}R^{4B};
R⁶ is hydrogen or methyl;
R⁷ is methyl;
R⁸ is methyl; and
each R^{4A}, R^{4B}, R^{5A} and R^{5B} is independently hydrogen or methyl.

21. The method of claim 17, wherein Y and Z are N; and
W and X are C.

22. The method of claim 17, wherein X and Y are N; and
W and Z are C.

23. The method of claim 17, wherein the MHC-peptide antigen stabilizing compound has the formula: 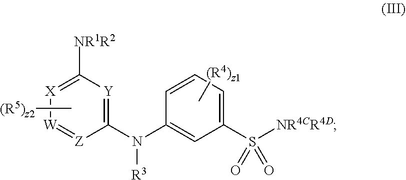
wherein
each R^{4C} and R^{4D} is independently hydrogen, —CX₃, —CHX₂, —CH₂X, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H,
NHC═(O)H, —NHC(O)OH, —NHOH, —OCX₃, —OCHX₂, —OCH₂X, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{4C} and R^{4D} substituents bonded to the same nitrogen atom may optionally be joined to form a substituted or unsubstituted heterocycloalkyl or substituted or unsubstituted heteroaryl;
R³ is hydrogen or methyl; and
z1 is an integer from 0 to 4.

24. The method of claim 23, wherein the MHC-peptide antigen stabilizing compound has the formula:

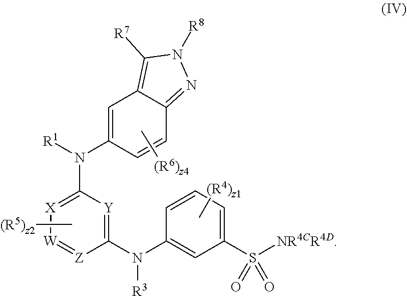

25. The method of claim 17, wherein the MHC-peptide antigen stabilizing compound is selected from a group consisting of: 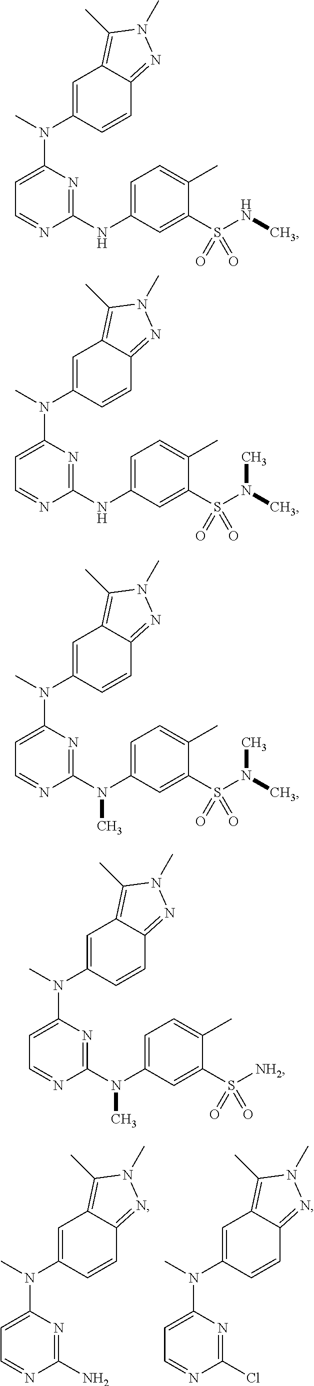 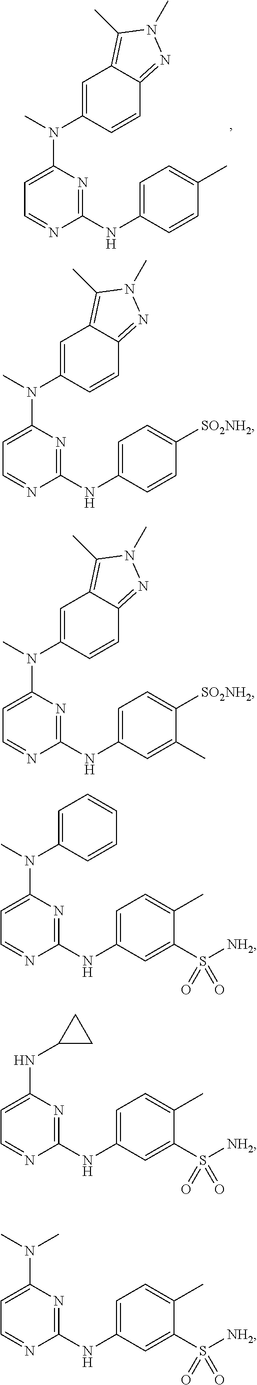 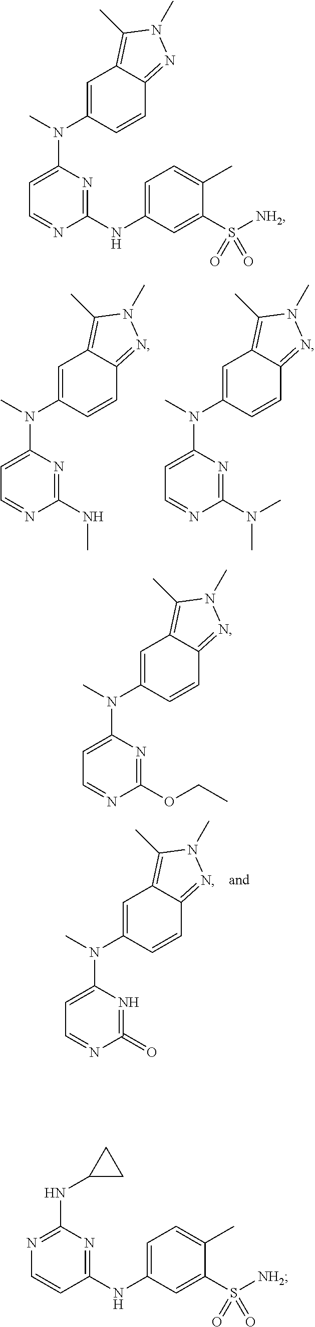
or a salt thereof.

26. The method of claim 1, wherein the MHC-peptide antigen stabilizing compound has the formula: 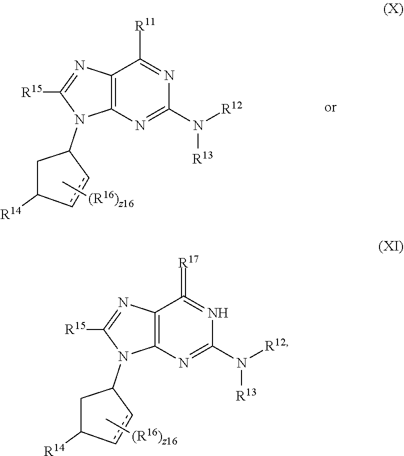
or salts thereof;
wherein,
R¹¹ is hydrogen, halogen, —CX¹¹₃, —CHX¹¹₂, —CH₂X¹¹, —OCX¹¹₃,
—OCH₂X¹¹, —OCHX¹¹₂, —CN, —SOₙ₁₁R^{11D}, —SOᵥ₁₁NR^{11A}R^{11B}, —NHC(O)NR^{11A}R^{11B}, —N(O)ₘ₁₁, —NR^{11A}R^{11B}, —C(O)R^{11C}, —C(O)—OR^{11C}, —C(O)NR^{11A}R^{11B}, —OR^{11D}, —NR^{11A}CH₂C(O)R^{11C}, —NR^{11A}C H₂SO₂R^{11D}, —NR^{11A}SO₂R^{11D}, —NR^{11A}C(O)R^{11C}, —NR^{11A}C(O)OR^{11C}, —NR^{11A}OR^{11C}, —NR^{11A}OS O₂R^{11D}, —NR^{11A}OCH₂C(O)R^{11C}, —NR^{11A}CH₂P(O)R^{11C}R^{11D}, —PO_{q11}R^{11A}, —POᵣ₁₁R^{11C}R^{11D}, —POᵣ₁₁NR^{11A}R^{11B}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹² is hydrogen,
halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹³ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —S H, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁴ is —CH₂OR^{14A}, —C(O)OR¹⁴B, or —CH₂OC(═NH)R^{14C};
R¹⁵ is hydrogen, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —S H, —OCCl₃, —OCBr₃, —OCF₃, —OCI₃, —OCH₂Cl, —OCH₂Br, —OCH₂F, —OCH₂I, —OCHCl₂, —OCHB r₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁶ is independently hydrogen,
halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —S H, —OCCl₃, —OCBr₃, —OCF₃, —OCI₃, —OCH₂Cl, —OCH₂Br, —OCH₂F, —OCH₂I, —OCHCl₂, —OCHB r₂, —OCHF₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R¹⁷ is ═O, ═S, or ═NR^{17A};
each R^{11A}, R^{11B}, R^{11C} and R^{11D} is independently
hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —OSO₃H, —NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl;
R^{14A} and R^{14B} are independently hydrogen or unsubstituted C₁-C₅ alkyl;
R^{14C} is unsubstituted C₁-C₅ alkyl;
R^{17A} is independently hydrogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —OSO₃H,
—NH₂, —COOH, —CONH₂, —NO₂, —SH, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹¹ is —Cl, —Br, —I or —F;
n11 is independently an integer from 0 to 4;
v11 is independently 1 or 2;
m11 is independently an integer from 0 to 3;
each q11 and r11 is independently 1 or 2; and
z16 is independently an integer from 0 to 8.

27. The method of claim 26, wherein R¹² is hydrogen.

28. The method of claim 26, wherein R¹³ is hydrogen.

29. The method of claim 26, wherein R¹⁵ is hydrogen.

30. The method of claim 26, wherein R¹⁴ is —CH₂OR^{14A}.

31. The method of claim 30, wherein R^{14A} is hydrogen.

32. The method of claim 26, wherein the MHC-peptide antigen stabilizing compound has the formula:

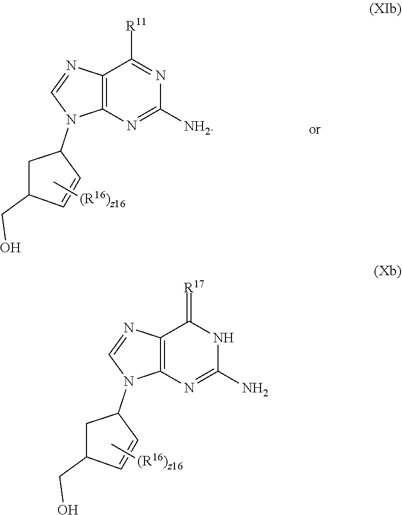

33. The method of claim 26, wherein the MHC-peptide antigen stabilizing compound has the formula:

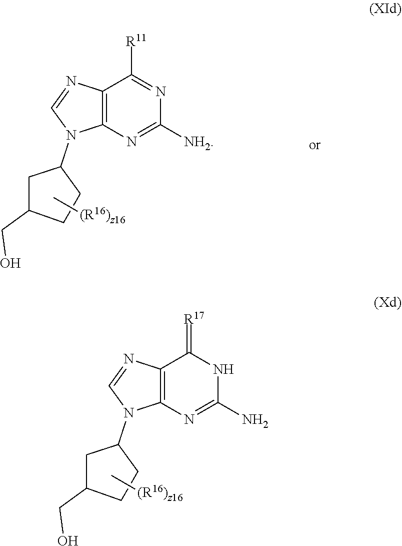

34. The method of claim 32, wherein R¹⁶ is independently hydrogen or —OH.

35. The method of claim 32, wherein z16 is 1 or 2.

36. The method of claim 32, wherein z16 is 0.

37. The method of claim 26, wherein R¹¹ is hydrogen,
halogen, —CCl₃, —CBr₃, —CF₃, —CI₃, —CH₂Cl, —CH₂Br,
CH₂F, —CH₂I, —CHCl₂, —CHBr₂, —CHF₂, —CHI₂, —CN, —OH,
—ONH₂, —NR^{11A}R^{11B}, —COOH, —COO(C₁-C₄ alkyl), —CONH₂, —NO₂, —SH, —SO₂OH, —SO₂NH₂, —PO(OH)₂, —OCCl₃, —OCF₃, —OCBr₃, —OCI₃,—OCHCl₂, —OCHBr₂, —OCHI₂, —OCHF₂, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

38. The method of claim 26 to 36, wherein R¹¹ is —NR^{11A}R^{11B}.

39. The method of claim 26 to 36, wherein R¹¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

40. The method of claim 38, wherein R^{11A} and R^{1B} are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{11A} and R^{11B} substituents bonded to the same nitrogen atom may optionally be joined to form a substituted or unsubstituted heterocycloalkyl or substituted or unsubstituted heteroaryl.

41. The method of claim 38, wherein R^{11A} and R^{11B} are independently hydrogen, substituted or unsubstituted C₁-C₄ alkyl, or substituted or unsubstituted C₃-C₆ cycloalkyl.

42. The method of claim 38, wherein R^{11A} and R^{11B} substituents bonded to the same nitrogen atom are joined to form a substituted or unsubstituted 4 to 6 membered heterocycloalkyl or substituted or unsubstituted 5 to 6 membered heteroaryl.

43. The method of claim 38, wherein R^{11A} and R^{11B} are independently hydrogen, —COCHCH₂, —CH₂COOH, —CH₂SO₂OH, —OSO₂OH, —CH₂P(O)(OH)₂, or —OCH₂COOH.

44. The method of claim 26, wherein the MHC-peptide antigen stabilizing compound is selected from a group consisting of:

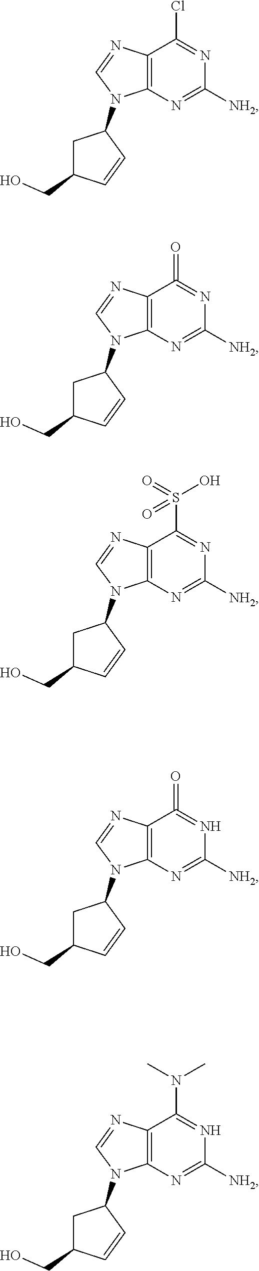

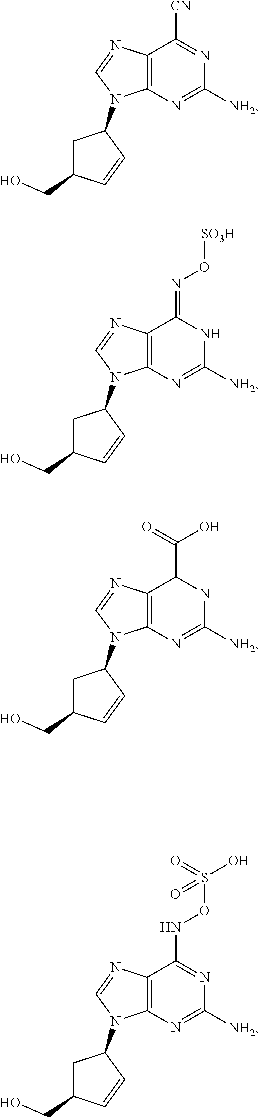 [SMILES: N#Cc1nc(N)nc2c1ncn2[C@H]1C=C[C@@H](CO)C1 ; Nc1nc2c(ncn2[C@H]2C=C[C@@H](CO)C2)/c(=N/OS(=O)(=O)O)[nH]1 ; NC1=Nc2c(ncn2[C@H]2C=C[C@@H](CO)C2)C(C(=O)O)N1 ; Nc1nc(NOS(=O)(=O)O)c2ncn([C@H]3C=C[C@@H](CO)C3)c2n1]

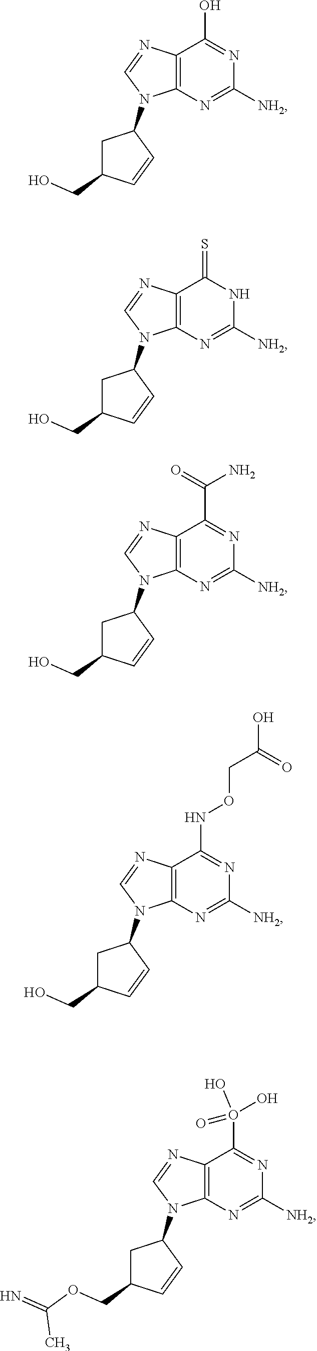

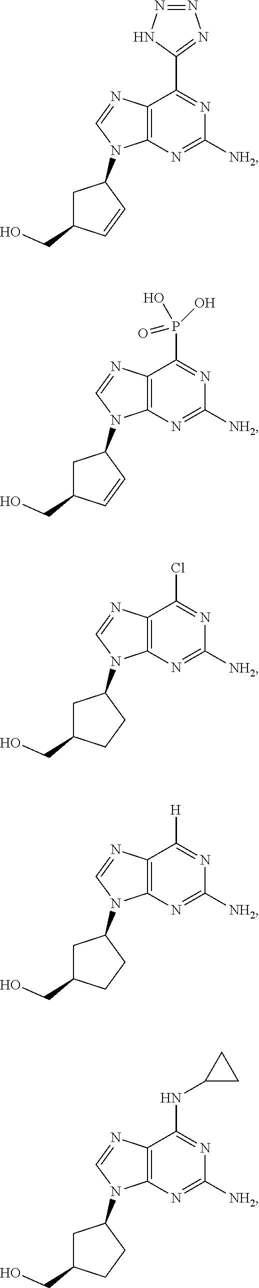 [SMILES: Nc1nc(-c2nnn[nH]2)c2ncn([C@H]3C=C[C@@H](CO)C3)c2n1 ; Nc1nc(P(=O)(O)O)c2ncn([C@H]3C=C[C@@H](CO)C3)c2n1 ; Nc1nc(Cl)c2ncn([C@H]3CC[C@@H](CO)C3)c2n1 ; Nc1ncc2ncn([C@H]3CC[C@@H](CO)C3)c2n1 ; Nc1nc(NC2CC2)c2ncn([C@H]3CC[C@@H](CO)C3)c2n1]

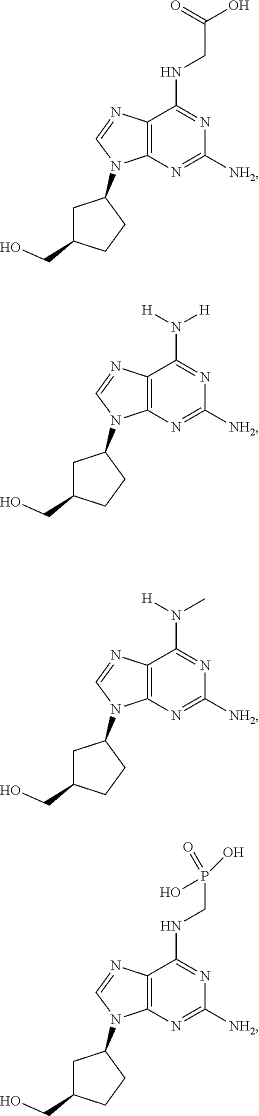 [SMILES: Nc1nc(NCC(=O)O)c2ncn([C@H]3CC[C@@H](CO)C3)c2n1 ; Nc1nc(N)c2ncn([C@H]3CC[C@@H](CO)C3)c2n1 ; CNc1nc(N)nc2c1ncn2[C@H]1CC[C@@H](CO)C1 ; Nc1nc(NCP(=O)(O)O)c2ncn([C@H]3CC[C@@H](CO)C3)c2n1]

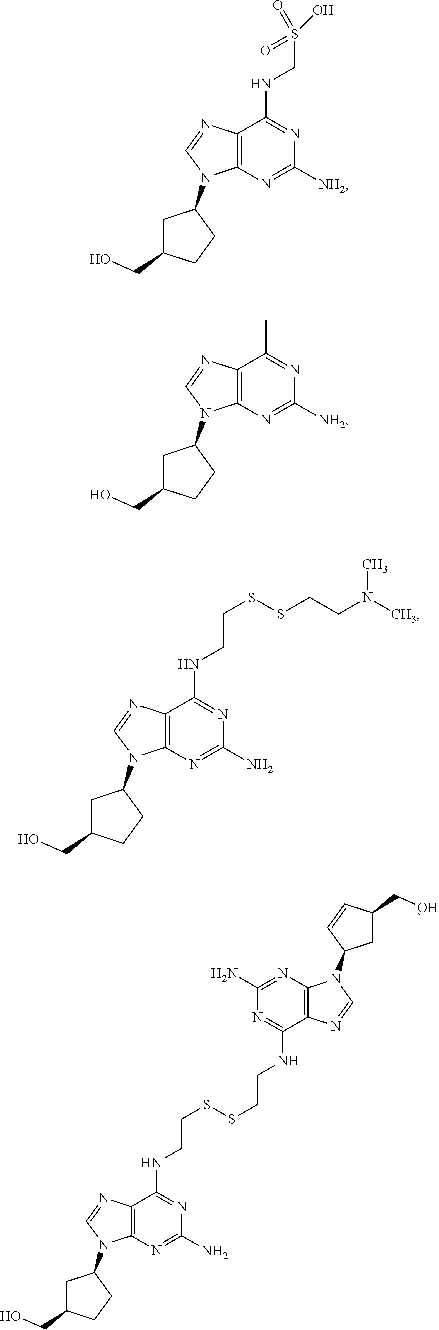 [SMILES: Nc1nc(NCS(=O)(=O)O)c2ncn([C@H]3CC[C@@H](CO)C3)c2n1 ; Cc1nc(N)nc2c1ncn2[C@H]1CC[C@@H](CO)C1 ; CN(C)CCSSCCNc1nc(N)nc2c1ncn2[C@H]1CC[C@@H](CO)C1 ; Nc1nc(NCCSSCCNc2nc(N)nc3c2ncn3[C@H]2C=C[C@@H](CO)C2)c2ncn([C@H]3CC[C@@H](CO)C3)c2n1]

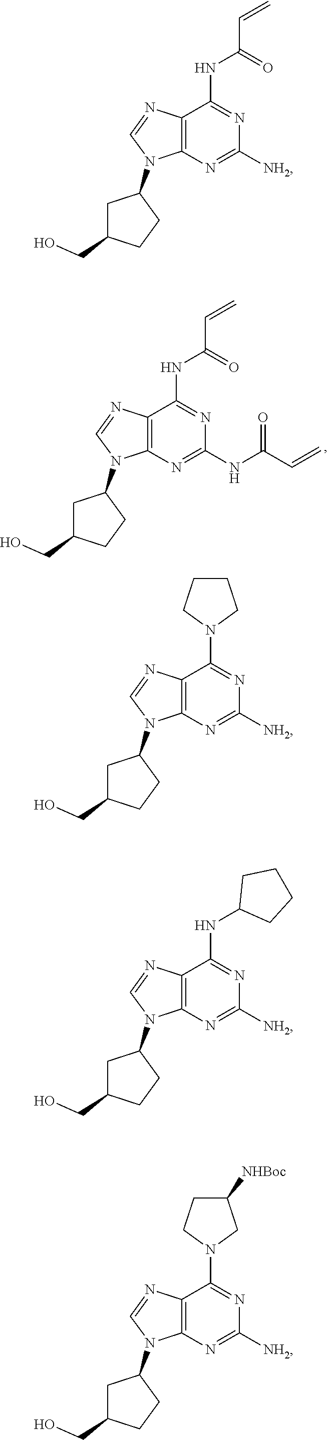 [SMILES: C=CC(=O)Nc1nc(N)nc2c1ncn2[C@H]1CC[C@@H](CO)C1 ; C=CC(=O)Nc1nc(NC(=O)C=C)c2ncn([C@H]3CC[C@@H](CO)C3)c2n1 ; Nc1nc(N2CCCC2)c2ncn([C@H]3CC[C@@H](CO)C3)c2n1 ; Nc1nc(NC2CCCC2)c2ncn([C@H]3CC[C@@H](CO)C3)c2n1 ; CC(C)(C)OC(=O)N[C@@H]1CCN(c2nc(N)nc3c2ncn3[C@H]2CC[C@@H](CO)C2)C1]

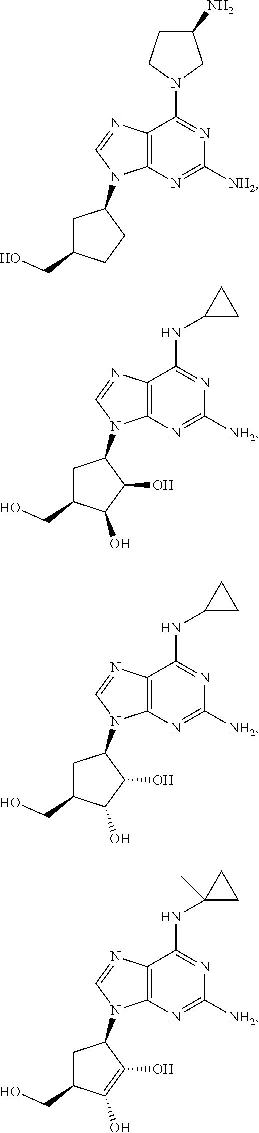

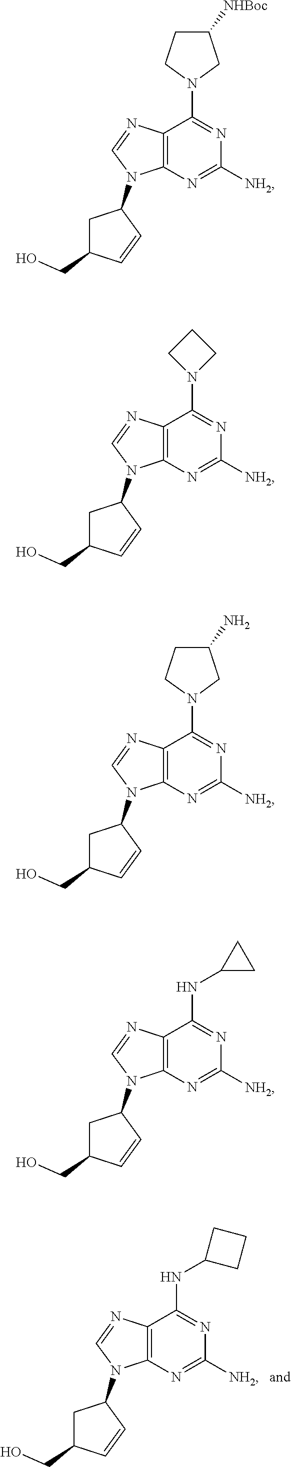 [SMILES: CC(C)(C)OC(=O)N[C@H]1CCN(c2nc(N)nc3c2ncn3[C@H]2C=C[C@@H](CO)C2)C1 ; Nc1nc(N2CCC2)c2ncn([C@H]3C=C[C@@H](CO)C3)c2n1 ; Nc1nc(N2CC[C@H](N)C2)c2ncn([C@H]3C=C[C@@H](CO)C3)c2n1 ; Nc1nc(NC2CC2)c2ncn([C@H]3C=C[C@@H](CO)C3)c2n1 ; Nc1nc(NC2CCC2)c2ncn([C@H]3C=C[C@@H](CO)C3)c2n1]

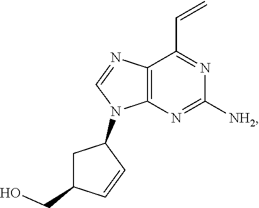 [SMILES: C=Cc1nc(N)nc2c1ncn2[C@H]1C=C[C@@H](CO)C1]

45. A method of vaccinating a subject for cancer, the method comprising administering:
a. a peptide cancer antigen; and
b. a compound that stabilizes binding of an MHC protein to said peptide cancer antigen.

46. A method of vaccinating a subject for cancer, the method comprising administering a peptide-compound conjugate, wherein the peptide-compound conjugate comprises a peptide cancer antigen that is linked to a compound via a chemical bond.

47. A method of vaccinating a subject for cancer, the method comprising administering:
a. a peptide cancer antigen; and
b. a compound that stabilizes binding of an MHC protein to said peptide cancer antigen; wherein said MHC-peptide cancer antigen stabilizing compound is identified by the method of claim 1.

48. The method of claim 45, wherein a vaccine is administered in a single formulation.

49. A composition comprising an MHC protein, a peptide antigen, and a compound; wherein, the MHC protein, the peptide antigen, and the compound are bound to form an MHC-peptide-compound complex and wherein the compound stabilizes the binding of the MHC protein to the peptide antigen relative to the absence of the compound.

50. The composition of claim 49, wherein the MHC protein is covalently bound to the peptide antigen.

51. The composition of claim 49, wherein the MHC protein is covalently bound to the peptide antigen through a disulfide bond.

52. The composition of claim 51, wherein a cysteine amino acid within the MHC protein forms a part of said disulfide bond.

53. The composition of claim 49, wherein the compound is covalently bound to the peptide antigen.

54. The composition of claim 53, wherein the compound is covalently bound to the peptide antigen through a reaction between an electrophilic moiety on the compound and a nucleophilic moiety on the peptide antigen.

55. The composition of claim 54, wherein the nucleophilic moiety is a cysteine sulfhydryl group.

56. The composition of claim 54, wherein the nucleophilic group is a lysine amine group.

57. A composition comprising an MHC protein which is covalently bound to a peptide antigen.

58. The composition of claim 57, wherein the MHC protein is covalently bound to the peptide antigen through a disulfide bond.

59. The composition of claim 58, wherein a cysteine amino acid within the MHC protein forms a part of said disulfide bond.

60. A composition comprising a peptide antigen, which is covalently bound to a compound.

61. The composition of claim 60, wherein the compound is covalently bound to the peptide antigen through a reaction between an electrophilic moiety on the compound and a nucleophilic moiety on the peptide antigen.

62. The composition of claim 61, wherein the nucleophilic moiety is a cysteine sulfhydryl group.

63. The composition of claim 61, wherein the nucleophilic group is a lysine amine group.

64. A compound having the formula: 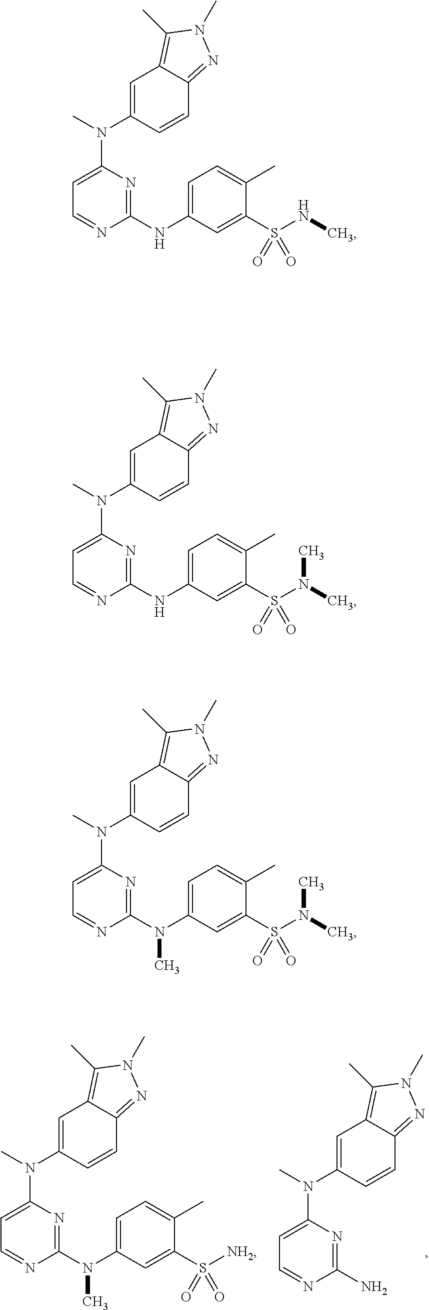 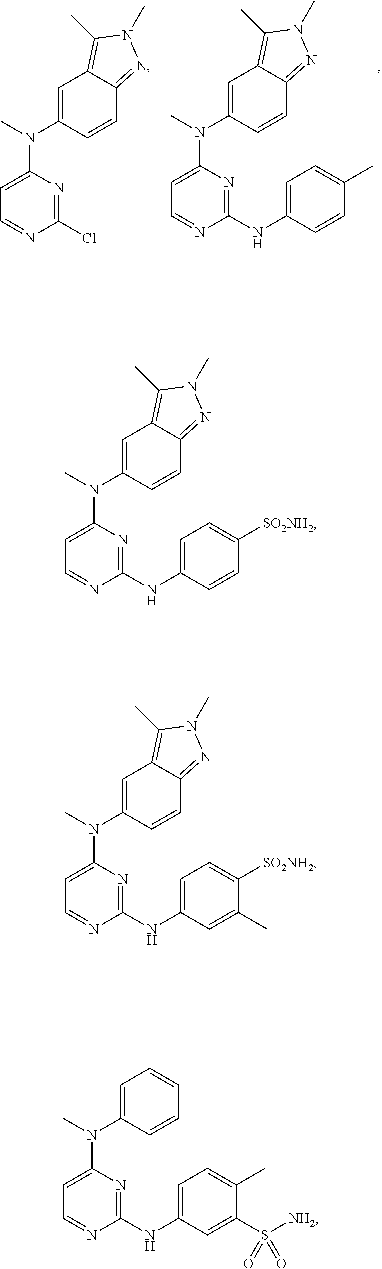 [SMILES: Cc1c2cc(N(C)c3ccnc(Cl)n3)ccc2nn1C ; Cc1ccc(Nc2nccc(N(C)c3ccc4nn(C)c(C)c4c3)n2)cc1 ; Cc1c2cc(N(C)c3ccnc(Nc4ccc(S(N)(=O)=O)cc4)n3)ccc2nn1C ; Cc1cc(Nc2nccc(N(C)c3ccc4nn(C)c(C)c4c3)n2)ccc1S(N)(=O)=O ; Cc1ccc(Nc2nccc(N(C)c3ccccc3)n2)cc1S(N)(=O)=O] 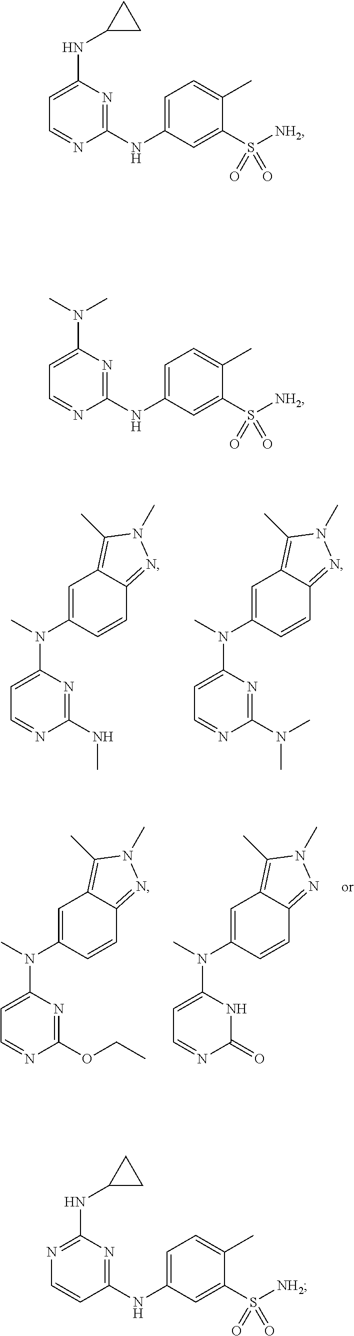
or a salt thereof.

65. A compound having the formula: 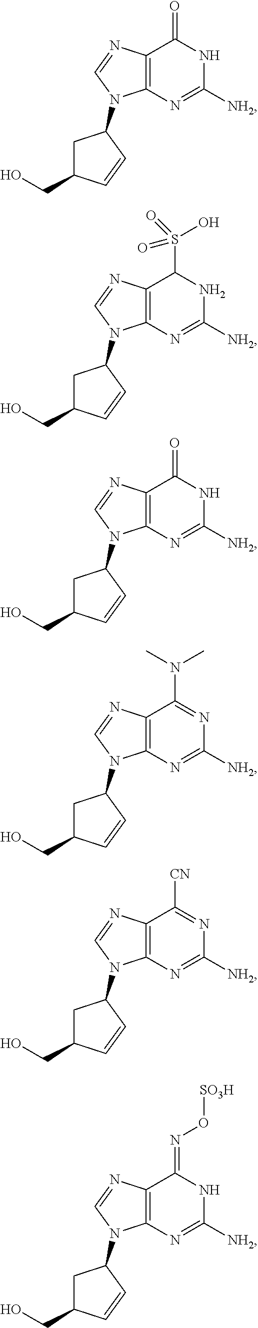 [SMILES: Nc1nc2c(ncn2[C@H]2C=C[C@@H](CO)C2)c(=O)[nH]1 ; NC1=Nc2c(ncn2[C@H]2C=C[C@@H](CO)C2)C(S(=O)(=O)O)N1 ; Nc1nc2c(ncn2[C@H]2C=C[C@@H](CO)C2)c(=O)[nH]1 ; CN(C)c1nc(N)nc2c1ncn2[C@H]1C=C[C@@H](CO)C1 ; N#Cc1nc(N)nc2c1ncn2[C@H]1C=C[C@@H](CO)C1 ; Nc1nc2c(ncn2[C@H]2C=C[C@@H](CO)C2)/c(=N/OS(=O)(=O)O)[nH]1] 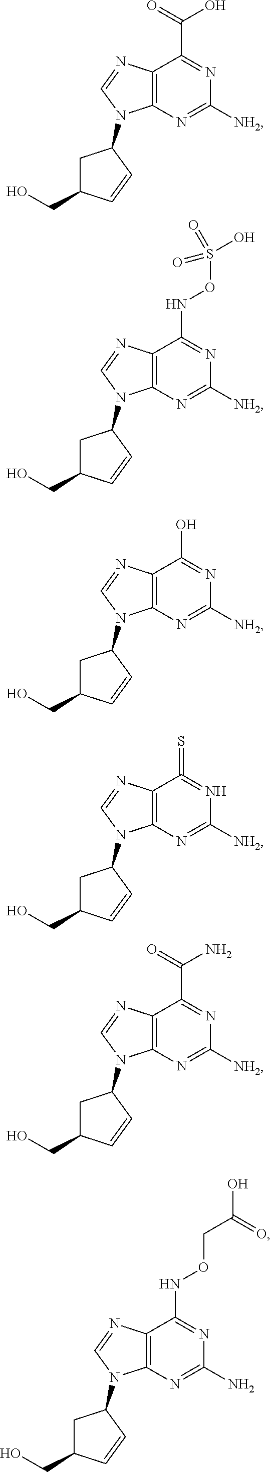 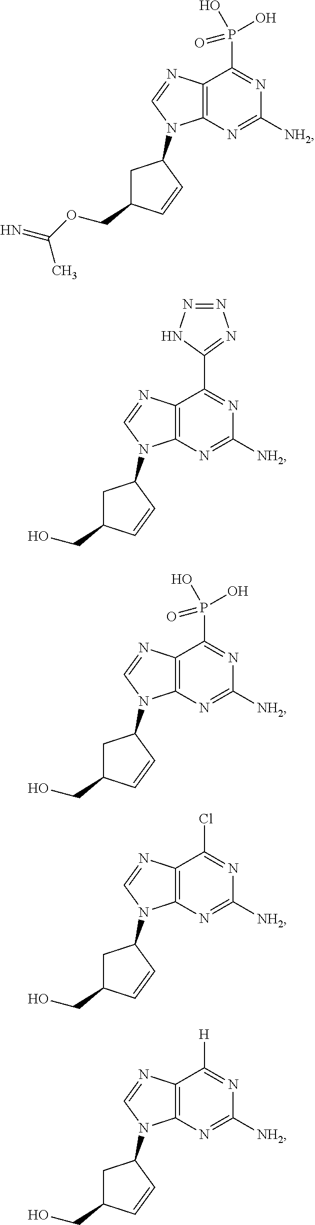 [SMILES: CC(=N)OC[C@@H]1C=C[C@H](n2cnc3c(P(=O)(O)O)nc(N)nc32)C1 ; Nc1nc(-c2nnn[nH]2)c2ncn([C@H]3C=C[C@@H](CO)C3)c2n1 ; Nc1nc(P(=O)(O)O)c2ncn([C@H]3C=C[C@@H](CO)C3)c2n1 ; Nc1nc(Cl)c2ncn([C@H]3C=C[C@@H](CO)C3)c2n1 ; Nc1ncc2ncn([C@H]3C=C[C@@H](CO)C3)c2n1] 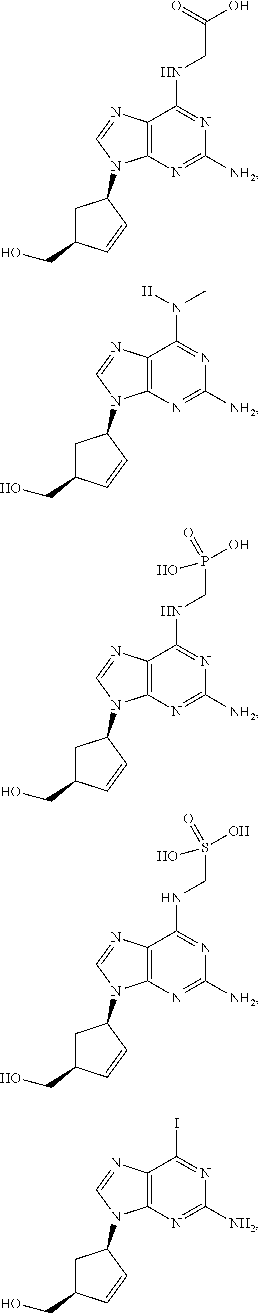 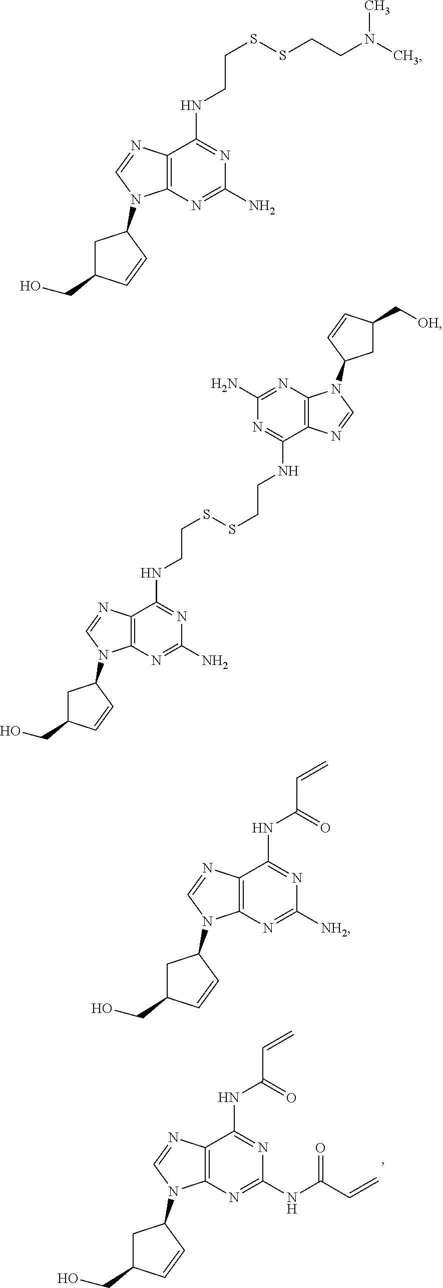 [SMILES: CN(C)CCSSCCNc1nc(N)nc2c1ncn2[C@H]1C=C[C@@H](CO)C1 ; Nc1nc(NCCSSCCNc2nc(N)nc3c2ncn3[C@H]2C=C[C@@H](CO)C2)c2ncn([C@H]3C=C[C@@H](CO)C3)c2n1 ; C=CC(=O)Nc1nc(N)nc2c1ncn2[C@H]1C=C[C@@H](CO)C1 ; C=CC(=O)Nc1nc(NC(=O)C=C)c2ncn([C@H]3C=C[C@@H](CO)C3)c2n1] 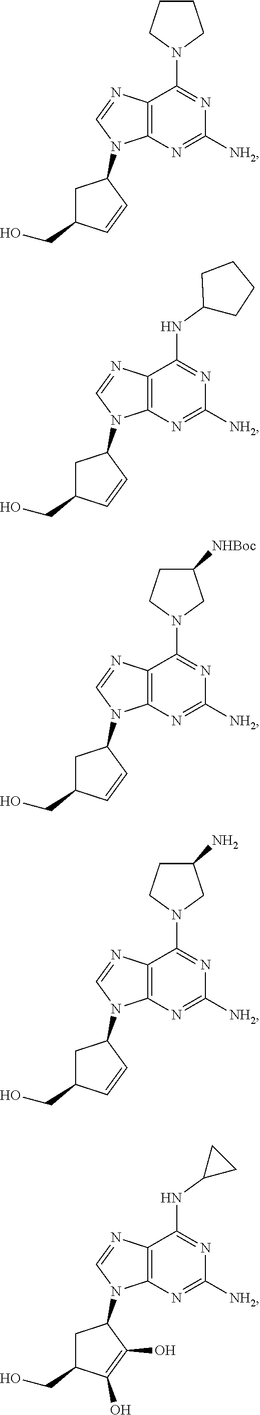 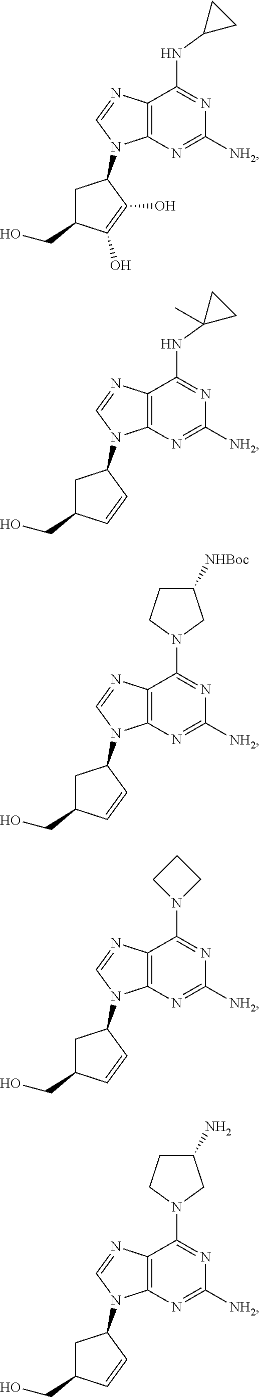 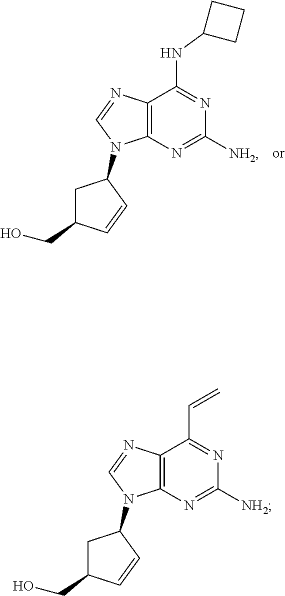 [SMILES: Nc1nc(NC2CCC2)c2ncn([C@H]3C=C[C@@H](CO)C3)c2n1 ; C=Cc1nc(N)nc2c1ncn2[C@H]1C=C[C@@H](CO)C1]
or a salt thereof.
